SEQUENCE LISTING (555 sequences)

SEQ ID NO: 1 - protein, 482 aa, synthetic construct
MGDGWLPPDCGPHNRSGGGGATAAPTGSRQVSAELLSQQWEAGMSLLMALVVLLIVAGNVLVIAAIGRTQRLQTLTNLFITSLACADLVMGLLVVPFGATLVVRGTWLWGSFLCECWTSLDVLCVTASIETLCVIAIDRYLAITSPFRYQSLMTRARAKVIICTVWAISALVSFLPIMMHWWRDEDPQALKCYQDPGCCDFVTNRAYAIASSIISFYIPLLIMIFVYLRVYREAKEQIRKIDRCEGRFYGSQEQPQPPPLPQHQPILGNGRASKRKTSRVMAMREHKALKTLGIIMGVFTLCWLPFFLVNIVNVFNRDLVPDWLFVFFNWLGYANSAFNPIIYCRSPDFRKAFKRECFPRKADRRLHAGGQPAPLPGGFISTLGSPEHSPGGTWSDCNGGTRGGSESSLEERHSKTSRSESKMEREKNILATTRFYCTFLGNGDKAVFCTVLRIVKLFEDATCTCPHTHKLKMKWRFKQHQA

SEQ ID NO: 2 - protein, 125 aa, synthetic construct
  VARIANT 73..85: note = X can be any naturally occurring amino acid.
QVKLEESGGGSVQTGGSLRLTCAASGRTSRSYGMGWFRQAPGKEREEFSGISTYADSVGMKGRFTISRYCAAXXXXXXXXXXXXXWGTASDFERYHLEWSEWDEGWWYEDFEEGSSALWEHHHHH

SEQ ID NO: 3 - protein, 7 aa, synthetic construct
MWRSGER

SEQ ID NO: 4 - protein, 9 aa, synthetic construct
MWGWGWGWR

SEQ ID NO: 5 - protein, 13 aa, synthetic construct
ERWSGRWGWRWGW

SEQ ID NO: 6 - protein, 124 aa, synthetic construct
  VARIANT 72..84: note = X can be any naturally occurring amino acid
QVKLEESGGGSVQTGGSLRLTCAASGRTSRSYGMGWFRQAPGKEREEFSGISTYADSVGKGRFTISRYCAAXXXXXXXXXXXXXWGTASDFERYHLEWSEWDEGWWYEDFEEGSSALWEHHHHH

SEQ ID NO: 7 - protein, 20 aa, synthetic construct
LAGBSERTIDPKQNFYMHWC

SEQ ID NO: 8 - protein, 124 aa, synthetic construct
  VARIANT 73..84: note = X can be any naturally occurring amino acid
QVKLEESGGGSVQTGGSLRLTCAASGRTSRSYGMGWFRQAPGKEREEFSGISTYADSVGKGRFTISRYCAAYXXXXXXXXXXXXWGTASDFERYHLEWSEWDEGWWYEDFEEGSSALWEHHHHH

SEQ ID NO: 9 - protein, 124 aa, synthetic construct
  VARIANT 74..84: note = X can be any naturally occurring amino acid.
QVKLEESGGGSVQTGGSLRLTCAASGRTSRSYGMGWFRQAPGKEREEFSGISTYADSVGKGRFTISRYCAAYYXXXXXXXXXXXWGTASDFERYHLEWSEWDEGWWYEDFEEGSSALWEHHHHH

SEQ ID NO: 10 - protein, 12 aa, synthetic construct
ARTGADIWGPGY

SEQ ID NO: 11 - protein, 12 aa, synthetic construct
ARYYFDGYHMDY

SEQ ID NO: 12 - protein, 12 aa, synthetic construct
ATDPDFNYYMDY

SEQ ID NO: 13 - protein, 12 aa, synthetic construct
ARADHYGFSLDY

SEQ ID NO: 14 - protein, 11 aa, synthetic construct
ARHRGSYYFDN

SEQ ID NO: 15 - protein, 13 aa, synthetic construct
ARHSDFYTWWFDY

SEQ ID NO: 16 - protein, 12 aa, synthetic construct
ARNRLRAYDFDY

SEQ ID NO: 17 - protein, 13 aa, synthetic construct
SRWDGDGFYAMDY

SEQ ID NO: 18 - protein, 12 aa, synthetic construct
ARTGVYMYYFDI

SEQ ID NO: 19 - protein, 11 aa, synthetic construct
ARHYGRFDFDY

SEQ ID NO: 20 - protein, 13 aa, synthetic construct
SRWGGDGFYAMDY

SEQ ID NO: 21 - protein, 13 aa, synthetic construct
ARSVFLIGYYFDY

SEQ ID NO: 22 - protein, 12 aa, synthetic construct
ARGWYPNHSFDL

SEQ ID NO: 23 - protein, 12 aa, synthetic construct
AKGTSSWYNFGV

SEQ ID NO: 24 - protein, 12 aa, synthetic construct
AGHWTYRFADTL

SEQ ID NO: 25 - protein, 12 aa, synthetic construct
AKISRYYYGMDY

SEQ ID NO: 26 - protein, 11 aa, synthetic construct
ARDGRYGYQDY

SEQ ID NO: 27 - protein, 11 aa, synthetic construct
AKHYDGWILAY

SEQ ID NO: 28 - protein, 12 aa, synthetic construct
AKPYYFNYDLGV

SEQ ID NO: 29 - protein, 11 aa, synthetic construct
AKNFLIGTRYW

SEQ ID NO: 30 - protein, 13 aa, synthetic construct
AKGWGVEPREFAY

SEQ ID NO: 31 - protein, 13 aa, synthetic construct
AKPYYYFSDGLEY

SEQ ID NO: 32 - protein, 12 aa, synthetic construct
AKYDYYIYDFDI

SEQ ID NO: 33 - protein, 11 aa, synthetic construct
AIGGYYYGGDL

SEQ ID NO: 34 - protein, 12 aa, synthetic construct
ARFDRYGYYLGT

SEQ ID NO: 35 - protein, 11 aa, synthetic construct
AMDFKFTRLDL

SEQ ID NO: 36 - protein, 12 aa, synthetic construct
AKRDFGRDWFAY

SEQ ID NO: 37 - protein, 12 aa, synthetic construct
ARFGISGYYWDV

SEQ ID NO: 38 - protein, 12 aa, synthetic construct
AKRNAYRGSLDY

SEQ ID NO: 39 - protein, 7 aa, synthetic construct
ABCDEFG

SEQ ID NO: 40 - protein, 7 aa, synthetic construct
BDGEACF

SEQ ID NO: 41 - protein, 12 aa, synthetic construct
ARWGNYYYYMDY

SEQ ID NO: 42 - protein, 12 aa, synthetic construct
ARYYYGFYYFDY

SEQ ID NO: 43 - protein, 14 aa, synthetic construct
ARYAGVERPGSFAY

SEQ ID NO: 44 - protein, 13 aa, synthetic construct
TRYFFNGWYYFDV

SEQ ID NO: 45 - protein, 13 aa, synthetic construct
AFADSGAYGIWSF

SEQ ID NO: 46 - protein, 13 aa, synthetic construct
ANDIYIQGYDLNR

SEQ ID NO: 47 - protein, 15 aa, synthetic construct
ARGYSGDWPYETFYV

SEQ ID NO: 48 - protein, 12 aa, synthetic construct
ARYDYGYYIYVS

SEQ ID NO: 49 - protein, 16 aa, synthetic construct
AKYPYYYGTSHWYFDV

SEQ ID NO: 50 - protein, 15 aa, synthetic construct
ASGNFSYPDDYGMDW

SEQ ID NO: 51 - protein, 13 aa, synthetic construct
ARDRGTTMVPFDY

SEQ ID NO: 52 - protein, 14 aa, synthetic construct
ARDWGQTWDSYFDY

SEQ ID NO: 53 - protein, 13 aa, synthetic construct
ARWGNYYYYYMDY

SEQ ID NO: 54 - protein, 8 aa, synthetic construct
GFNIKDTY

SEQ ID NO: 55 - protein, 8 aa, synthetic construct
IYPTNGYT

SEQ ID NO: 56 - protein, 8 aa, synthetic construct
GFNVSSSY

SEQ ID NO: 57 - protein, 8 aa, synthetic construct
IYPENGST

SEQ ID NO: 58 - protein, 13 aa, synthetic construct
SRYSYYGGYAFDY

SEQ ID NO: 59 - protein, 8 aa, synthetic construct
GFNISDYY

SEQ ID NO: 60 - protein, 8 aa, synthetic construct
IYPSSGYT

SEQ ID NO: 61 - protein, 13 aa, synthetic construct
ARDGGVGSYSMDV

SEQ ID NO: 62 - protein, 8 aa, synthetic construct
GFNIKDYY

SEQ ID NO: 63 - protein, 8 aa, synthetic construct
IYPSRGYT

SEQ ID NO: 64 - protein, 13 aa, synthetic construct
ARHGSEGRYTMDV

SEQ ID NO: 65 - protein, 8 aa, synthetic construct
GFNIKDTW

SEQ ID NO: 66 - protein, 8 aa, synthetic construct
IDPANGYT

SEQ ID NO: 67 - protein, 13 aa, synthetic construct
ARVGGRGYYYYDY

SEQ ID NO: 68 - protein, 8 aa, synthetic construct
GFNFKDTY

SEQ ID NO: 69 - protein, 8 aa, synthetic construct
IYPANGYT

SEQ ID NO: 70 - protein, 13 aa, synthetic construct
ASDGGSYSYAFDY

SEQ ID NO: 71 - protein, 8 aa, synthetic construct
IDPADGYT

SEQ ID NO: 72 - protein, 13 aa, synthetic construct
AREGGYGYYAMDY

SEQ ID NO: 73 - protein, 8 aa, synthetic construct
IYPSNGYT

SEQ ID NO: 74 - protein, 13 aa, synthetic construct
ARYGGSYAYYFYY

SEQ ID NO: 75 - protein, 13 aa, synthetic construct
ARYYGSSSYYFAY

SEQ ID NO: 76 - protein, 220 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEP

SEQ ID NO: 77 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 607 aa, synthetic construct
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPIN

SEQ ID NO: 79 - protein, 226 aa, synthetic construct
QVQLVESGGGLVKPGGSLRLSCAASGFTFSDYYMSWIRQAPGKGLEWVSYITYSGSTIYYADSVKGRFTISRDNAKSSLYLQMNSLRAEDTAVYYCARDRGTTMVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 80 - protein, 214 aa, synthetic construct
DIQMTQSPSSLSASVGDRVTITCQASQDITNYLNWYQQKPGKAPKLLIYAASNLETGVPSRFSGSGSGTDFTFTISGLQPEDIATYYCQQYDNLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 1285 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 82 - protein, 218 aa, synthetic construct
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSGTAALGCLVKDYFPEPVTVSWNSGALTSGVHTFPAVLQSSGLYSLSSVVTVPSSSLGTQTYICNVNHKPSNTKVDKKVEPK

SEQ ID NO: 83 - protein, 213 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 84 - protein, 94 aa, synthetic construct [sequence omitted]

SEQ ID NO: 85 - protein, 12 aa, synthetic construct
ARWSYGYYNFDY

SEQ ID NO: 86 - protein, 12 aa, synthetic construct
AVYSPDFYYLGY

SEQ ID NO: 87 - protein, 12 aa, synthetic construct
ARWGYYYYYMDY

SEQ ID NO: 88 - protein, 12 aa, synthetic construct
ARWGDYYYYMDY

SEQ ID NO: 89 - protein, 13 aa, synthetic construct
TRWLEGLSYFMDY

SEQ ID NO: 90 - protein, 13 aa, synthetic construct
ARWPFYGPYNYDY

SEQ ID NO: 91 - protein, 12 aa, synthetic construct
TRYGYGYYYFDY

SEQ ID NO: 92 - protein, 12 aa, synthetic construct
VRYGYGYYYFDY

SEQ ID NO: 93 - protein, 12 aa, synthetic construct
TRYYYGLYYFDY

SEQ ID NO: 94 - protein, 12 aa, synthetic construct
TRYGSYYYYFDY

SEQ ID NO: 95 - protein, 13 aa, synthetic construct
ARYEAYGLYPFGY

SEQ ID NO: 96 - protein, 13 aa, synthetic construct
ARWLGEGYYNFDY

SEQ ID NO: 97 - protein, 12 aa, synthetic construct
ARWLAYWYAMDY

SEQ ID NO: 98 - protein, 12 aa, synthetic construct
TRYGGGYYYFDY

SEQ ID NO: 99 - protein, 12 aa, synthetic construct
ARWGGYYYYMDY

SEQ ID NO: 100 - protein, 12 aa, synthetic construct
VRYGGGYYYFDV

SEQ ID NO: 101 - protein, 12 aa, synthetic construct
ARWGGNYYYMDY

SEQ ID NO: 102 - protein, 12 aa, synthetic construct
ARWGSGYYYMDY

SEQ ID NO: 103 - protein, 12 aa, synthetic construct
ATYSFGAYYYVF

SEQ ID NO: 104 - protein, 13 aa, synthetic construct
ARWPGYGSYYMDV

SEQ ID NO: 105 - protein, 12 aa, synthetic construct
VRWRSDFYYFDY

SEQ ID NO: 106 - protein, 12 aa, synthetic construct
TRYGYGYYYMDY

SEQ ID NO: 107 - protein, 12 aa, synthetic construct
ALWGFGWYYFDY

SEQ ID NO: 108 - protein, 13 aa, synthetic construct
VRYYGDGGYYFDY

SEQ ID NO: 109 - protein, 12 aa, synthetic construct
TRFRSFFYNFDY

SEQ ID NO: 110 - protein, 12 aa, synthetic construct
VRYGYGYYYMDY

SEQ ID NO: 111 - protein, 12 aa, synthetic construct
ARWGDGYYYMDY

SEQ ID NO: 112 - protein, 12 aa, synthetic construct
ARWYGAYYHMDY

SEQ ID NO: 113 - protein, 12 aa, synthetic construct
ARYRHWYYDYDY

SEQ ID NO: 114 - protein, 12 aa, synthetic construct
ARYGGFYYYFDY

SEQ ID NO: 115 - protein, 12 aa, synthetic construct
VRWGGYYYYFDY

SEQ ID NO: 116 - protein, 12 aa, synthetic construct
ARYGSYFYYMDY

SEQ ID NO: 117 - protein, 13 aa, synthetic construct
VRYGGWGFWYFDY

SEQ ID NO: 118 - protein, 13 aa, synthetic construct
TRYYYYGSYHFDY

SEQ ID NO: 119 - protein, 12 aa, synthetic construct
ARYYYYSYAMDY

SEQ ID NO: 120 - protein, 13 aa, synthetic construct
ARWGYSGWYVMDY

SEQ ID NO: 121 - protein, 13 aa, synthetic construct
ARWYYGYYYYFDV

SEQ ID NO: 122 - protein, 13 aa, synthetic construct
ARYFYDYYYYFDF

SEQ ID NO: 123 - protein, 13 aa, synthetic construct
ARYGDGYYYYFAY

SEQ ID NO: 124 - protein, 13 aa, synthetic construct
ARWYGYGGYYFDY

SEQ ID NO: 125 - protein, 12 aa, synthetic construct
ARWGFSMYSFDY

SEQ ID NO: 126 - protein, 13 aa, synthetic construct
TRNYYDYYYYFDY

SEQ ID NO: 127 - protein, 12 aa, synthetic construct
ATYGRDYYHFAS

SEQ ID NO: 128 - protein, 12 aa, synthetic construct
ARWAGDYYVMDY

SEQ ID NO: 129 - protein, 13 aa, synthetic construct
ARYYGYGGYYFDY

SEQ ID NO: 130 - protein, 13 aa, synthetic construct
ARWGGPGYYAFVW

SEQ ID NO: 131 - protein, 13 aa, synthetic construct
ARYYGYGGYWYDT

SEQ ID NO: 132 - protein, 13 aa, synthetic construct
ARWYYYGGYYFDY

SEQ ID NO: 133 - protein, 13 aa, synthetic construct
ARNYYDLYYYFDY

SEQ ID NO: 134 - protein, 13 aa, synthetic construct
ARYYYDYYYYFDY

SEQ ID NO: 135 - protein, 12 aa, synthetic construct
ARWPGGYYHMDY

SEQ ID NO: 136 - protein, 12 aa, synthetic construct
ARWFGGYYHMDY

SEQ ID NO: 137 - protein, 13 aa, synthetic construct
TSIFSGGYYGLDS

SEQ ID NO: 138 - protein, 12 aa, synthetic construct
ARWYGGYYHHDY

SEQ ID NO: 139 - protein, 12 aa, synthetic construct
ARYGYGYYVMDY

SEQ ID NO: 140 - protein, 13 aa, synthetic construct
TRYGSFHYYYFDV

SEQ ID NO: 141 - protein, 13 aa, synthetic construct
ARWGGYGYYYMDV

SEQ ID NO: 142 - protein, 12 aa, synthetic construct
ARYGGGYYYYDY

SEQ ID NO: 143 - protein, 13 aa, synthetic construct
TRYASGYFYAFDY

SEQ ID NO: 144 - protein, 11 aa, synthetic construct
APYGPGYWYGV

SEQ ID NO: 145 - protein, 13 aa, synthetic construct
ARWGEFGPYYFDY

SEQ ID NO: 146 - protein, 13 aa, synthetic construct
ARWTERGFYSFDI

SEQ ID NO: 147 - protein, 12 aa, synthetic construct
ARWGGDFYAMDY

SEQ ID NO: 148 - protein, 14 aa, synthetic construct
ARYGYAPGFYYMDV

SEQ ID NO: 149 - protein, 11 aa, synthetic construct
TRWGGYYYFDY

SEQ ID NO: 150 - protein, 13 aa, synthetic construct
ARYYGYGGYYMDY

SEQ ID NO: 151 - protein, 12 aa, synthetic construct
ARYGYYLYAMDY

SEQ ID NO: 152 - protein, 13 aa, synthetic construct
AKFAYYGPYHFDP

SEQ ID NO: 153 - protein, 12 aa, synthetic construct
ARYGYYSYAMDY

SEQ ID NO: 154 - protein, 12 aa, synthetic construct
VRYGYDYYWFDY

SEQ ID NO: 155 - protein, 12 aa, synthetic construct
ARYGLGYYYMDY

SEQ ID NO: 156 - protein, 12 aa, synthetic construct
ARFGNSGYQFDY

SEQ ID NO: 157 - protein, 12 aa, synthetic construct
AIYSDGAYFYGR

SEQ ID NO: 158 - protein, 12 aa, synthetic construct
VRYGYGYYAMDY

SEQ ID NO: 159 - protein, 13 aa, synthetic construct
ARWLGSGYYWFDY

SEQ ID NO: 160 - protein, 12 aa, synthetic construct
ARYTFGAYYYSD

SEQ ID NO: 161 - protein, 13 aa, synthetic construct
ARWYYYGRYYFDY

SEQ ID NO: 162 - protein, 12 aa, synthetic construct
ATWYGGNYHMYP

SEQ ID NO: 163 - protein, 12 aa, synthetic construct
ARYFGGSYAYVY

SEQ ID NO: 164 - protein, 12 aa, synthetic construct
ARWYGYYYYFDV

SEQ ID NO: 165 - protein, 12 aa, synthetic construct
TRFLIGSYYYDY

SEQ ID NO: 166 - protein, 13 aa, synthetic construct
SRYYAGGWYYFDY

SEQ ID NO: 167 - protein, 13 aa, synthetic construct
ARWYGGGYYYMDY

SEQ ID NO: 168 - protein, 12 aa, synthetic construct
ARWSSGYYYMDY

SEQ ID NO: 169 - protein, 13 aa, synthetic construct
TRYGGYGYYYMDY

SEQ ID NO: 170 - protein, 13 aa, synthetic construct
TRYRGPYYYYFDP

SEQ ID NO: 171 - protein, 13 aa, synthetic construct
VRWGVGAYYNFDY

SEQ ID NO: 172 - protein, 12 aa, synthetic construct
ARYGYGYYYMDY

SEQ ID NO: 173 - protein, 13 aa, synthetic construct
ARWYYYGGYYFDV

SEQ ID NO: 174 - protein, 12 aa, synthetic construct
STYARDYYHFGS

SEQ ID NO: 175 - protein, 12 aa, synthetic construct
ALYGNYSYGFAR

SEQ ID NO: 176 - protein, 12 aa, synthetic construct
ARWFYDYYYMDY

SEQ ID NO: 177 - protein, 12 aa, synthetic construct
ARWGGYYYYFDY

SEQ ID NO: 178 - protein, 12 aa, synthetic construct
ARFGSYYYYFDY

SEQ ID NO: 179 - protein, 12 aa, synthetic construct
ARWGGYYYYFDV

SEQ ID NO: 180 - protein, 13 aa, synthetic construct
ARWRGDGGYYFDY

SEQ ID NO: 181 - protein, 13 aa, synthetic construct
VRYGFDGSYYFDP

SEQ ID NO: 182 - protein, 13 aa, synthetic construct
ARYVGLGGYPLGY

SEQ ID NO: 183 - protein, 12 aa, synthetic construct
ARYGGGYYYMDY

SEQ ID NO: 184 - protein, 13 aa, synthetic construct
ARWGYYGNYYFDY

SEQ ID NO: 185 - protein, 12 aa, synthetic construct
ARYGSYNYYFGD

SEQ ID NO: 186 - protein, 12 aa, synthetic construct
ARWQGGYYYWDF

SEQ ID NO: 187 - protein, 12 aa, synthetic construct
ARWYYDYYYMDY

SEQ ID NO: 188 - protein, 13 aa, synthetic construct
ARYGSSYYYYFDY

SEQ ID NO: 189 - protein, 11 aa, synthetic construct
TRYGLTFDMDY

SEQ ID NO: 190 - protein, 13 aa, synthetic construct
ARHFGVASYSFDY

SEQ ID NO: 191 - protein, 13 aa, synthetic construct
ARYGGYGGYYFDY

SEQ ID NO: 192 - protein, 13 aa, synthetic construct
AKYGATGWYYFDY

SEQ ID NO: 193 - protein, 13 aa, synthetic construct
ARYGYYGPYYFDY

SEQ ID NO: 194 - protein, 11 aa, synthetic construct
ARYGGGYNMDY

SEQ ID NO: 195 - protein, 13 aa, synthetic construct
TRYGYGGPYWFDY

SEQ ID NO: 196 - protein, 13 aa, synthetic construct
ARYRGVWYYDFDN

SEQ ID NO: 197 - protein, 12 aa, synthetic construct
ARYGWGYYYFDY

SEQ ID NO: 198 - protein, 13 aa, synthetic construct
ARYLQLGPYYFDY

SEQ ID NO: 199 - protein, 13 aa, synthetic construct
AKYLGQEAYAYFS

SEQ ID NO: 200 - protein, 13 aa, synthetic construct
ARWWGGGAYYFDY

SEQ ID NO: 201 - protein, 14 aa, synthetic construct
ARYYYGYGGYYFDY

SEQ ID NO: 202 - protein, 13 aa, synthetic construct
ARWDSGGPYYFDS

SEQ ID NO: 203 - protein, 12 aa, synthetic construct
VRYGGTYYYFDY

SEQ ID NO: 204 - protein, 12 aa, synthetic construct
AKYGDDNYGFSH

SEQ ID NO: 205 - protein, 12 aa, synthetic construct
ATYGYGYYYFDY

SEQ ID NO: 206 - protein, 12 aa, synthetic construct
ARYGLYYYYFDY

SEQ ID NO: 207 - protein, 13 aa, synthetic construct
ARYGSGYYYYFDY

SEQ ID NO: 208 - protein, 13 aa, synthetic construct
ARYGSRYYYYFDY

SEQ ID NO: 209 - protein, 12 aa, synthetic construct
ARYGLGYYYFDY

SEQ ID NO: 210 - protein, 13 aa, synthetic construct
ARWLAGYNYYFDV

SEQ ID NO: 211 - protein, 13 aa, synthetic construct
ARYTYFGSFDFDY

SEQ ID NO: 212 - protein, 13 aa, synthetic construct
ARWLRALGYYMDY

SEQ ID NO: 213 - protein, 12 aa, synthetic construct
ARYGGYYYYMDY

SEQ ID NO: 214 - protein, 13 aa, synthetic construct
ARYWGLGGYYFDH

SEQ ID NO: 215 - protein, 13 aa, synthetic construct
VRYGFYGYYYFDY

SEQ ID NO: 216 - protein, 13 aa, synthetic construct
AKYINAYSYAFFD

SEQ ID NO: 217 - protein, 12 aa, synthetic construct
ATYSPGAYYYGP

SEQ ID NO: 218 - protein, 13 aa, synthetic construct
ARYSGYGLYPYAY

SEQ ID NO: 219 - protein, 13 aa, synthetic construct
AKYAYYGDFWGSV

SEQ ID NO: 220 - protein, 11 aa, synthetic construct
ARWYVSYDYDV

SEQ ID NO: 221 - protein, 12 aa, synthetic construct
ARYPGYYYYFDY

SEQ ID NO: 222 - protein, 12 aa, synthetic construct
TRYGAPYFYFDY

SEQ ID NO: 223 - protein, 13 aa, synthetic construct
TRYEYQDYYHFDY

SEQ ID NO: 224 - protein, 12 aa, synthetic construct
ARYGAYFYAMDA

SEQ ID NO: 225 - protein, 12 aa, synthetic construct
AKYGSNYYYLGS

SEQ ID NO: 226 - protein, 12 aa, synthetic construct
TRNSFGFYAMDY

SEQ ID NO: 227 - protein, 12 aa, synthetic construct
ARWYGGYYMYDY

SEQ ID NO: 228 - protein, 13 aa, synthetic construct
ARWGYGGYYYFDY

SEQ ID NO: 229 - protein, 12 aa, synthetic construct
AKYSFGGYYYGD

SEQ ID NO: 230 - protein, 13 aa, synthetic construct
ASYGLDGYYYFDV

SEQ ID NO: 231 - protein, 13 aa, synthetic construct
ARWGYDYYYAMDY

SEQ ID NO: 232 - protein, 13 aa, synthetic construct
ARYYSYGQYAFDP

SEQ ID NO: 233 - protein, 13 aa, synthetic construct
ARYGGGGRYYFDY

SEQ ID NO: 234 - protein, 13 aa, synthetic construct
ARYWYGGYYYFDY

SEQ ID NO: 235 - protein, 13 aa, synthetic construct
ARYAHYGPYYFDF

SEQ ID NO: 236 - protein, 12 aa, synthetic construct
AAYTNTFYYLVY

SEQ ID NO: 237 - protein, 13 aa, synthetic construct
VRYGSDVYYYFDI

SEQ ID NO: 238 - protein, 13 aa, synthetic construct
ARYYGYGYYYFHY

SEQ ID NO: 239 - protein, 12 aa, synthetic construct
ARYGVVRYDFAY

SEQ ID NO: 240 - protein, 13 aa, synthetic construct
ARWGSIGYYAFDH

SEQ ID NO: 241 - protein, 12 aa, synthetic construct
ALYNYYYYAFGF

SEQ ID NO: 242 - protein, 13 aa, synthetic construct
ARWGYGYSYYFDV

SEQ ID NO: 243 - protein, 12 aa, synthetic construct
ARYWMGVYEFDY

SEQ ID NO: 244 - protein, 11 aa, synthetic construct
ARWEDYYYMDY

SEQ ID NO: 245 - protein, 12 aa, synthetic construct
VRYGGDYYYFDY

SEQ ID NO: 246 - protein, 12 aa, synthetic construct
ARYGYGYFYMDY

SEQ ID NO: 247 - protein, 12 aa, synthetic construct
ARYGYDGYYFAY

SEQ ID NO: 248 - protein, 12 aa, synthetic construct
ARYPSVVYAFGI

SEQ ID NO: 249 - protein, 12 aa, synthetic construct
ARYGGGFYAMDY

SEQ ID NO: 250 - protein, 12 aa, synthetic construct
ATWHYGGYFMSG

SEQ ID NO: 251 - protein, 12 aa, synthetic construct
ARWGYYYYAMDY

SEQ ID NO: 252 - protein, 13 aa, synthetic construct
ATYSTYYNYAFGI

SEQ ID NO: 253 - protein, 13 aa, synthetic construct
TRYLHDYYYYFDY

SEQ ID NO: 254 - protein, 12 aa, synthetic construct
AVSADIMYGVSV

SEQ ID NO: 255 - protein, 13 aa, synthetic construct
VRYFYDGNYYFDL

SEQ ID NO: 256 - protein, 13 aa, synthetic construct
ARYRGYVLYDFAY

SEQ ID NO: 257 - protein, 13 aa, synthetic construct
ARENDGSPGSFEY

SEQ ID NO: 258 - protein, 12 aa, synthetic construct
ARYGYYGYYMDY

SEQ ID NO: 259 - protein, 13 aa, synthetic construct
AVTDLDGLHSYRY

SEQ ID NO: 260 - protein, 12 aa, synthetic construct
ARYGGGYWYFDY

SEQ ID NO: 261 - protein, 12 aa, synthetic construct
ARYPPGYYIMDF

SEQ ID NO: 262 - protein, 12 aa, synthetic construct
ARFGGYYYYFDY

SEQ ID NO: 263 - protein, 14 aa, synthetic construct
TRYYDGAGPYYMDY

SEQ ID NO: 264 - protein, 12 aa, synthetic construct
ARYGTIYFHFDY

SEQ ID NO: 265 - protein, 12 aa, synthetic construct
VTYGTTYYHFGS

SEQ ID NO: 266 - protein, 13 aa, synthetic construct
ARYDPYGFYHLDY

SEQ ID NO: 267 - protein, 13 aa, synthetic construct
VRYGYGDYYYFDY

SEQ ID NO: 268 - protein, 13 aa, synthetic construct
AKYGGVGYYAMDY

SEQ ID NO: 269 - protein, 12 aa, synthetic construct
ARWGYGYYYFDY

SEQ ID NO: 270 - protein, 13 aa, synthetic construct
ARYSAYGLYDFAY

SEQ ID NO: 271 - protein, 12 aa, synthetic construct
ARYGTGNYAMDY

SEQ ID NO: 272 - protein, 12 aa, synthetic construct
ARWGYDYYYFAY

SEQ ID NO: 273 - protein, 13 aa, synthetic construct
ARWGYDYYYYFDV

SEQ ID NO: 274 - protein, 12 aa, synthetic construct
ARYGRGYYYFDY

SEQ ID NO: 275 - protein, 11 aa, synthetic construct
VRYGNSYYYDY

SEQ ID NO: 276 - protein, 11 aa, synthetic construct
VRFYYDYYFDY

SEQ ID NO: 277 - protein, 12 aa, synthetic construct
ARYDIGYYDMDK

SEQ ID NO: 278 - protein, 13 aa, synthetic construct
AATYGGLGDMSSI

SEQ ID NO: 279 - protein, 13 aa, synthetic construct
ARYGVYPFYYFDY

SEQ ID NO: 280 - protein, 12 aa, synthetic construct
ARNYVYWYGMTV

SEQ ID NO: 281 - protein, 12 aa, synthetic construct
AKYGTSNYGFSH

SEQ ID NO: 282 - protein, 13 aa, synthetic construct
ARWERYGYYVLDV

SEQ ID NO: 283 - protein, 13 aa, synthetic construct
ARYGAYSSYYFGY

SEQ ID NO: 284 - protein, 12 aa, synthetic construct
ARYFGGSYAYGR

SEQ ID NO: 285 - protein, 12 aa, synthetic construct
ARWLYYGYYFDY

SEQ ID NO: 286 - protein, 12 aa, synthetic construct
ARYLSYSYAWNY

SEQ ID NO: 287 - protein, 13 aa, synthetic construct
TRYYGDGYYDFDY

SEQ ID NO: 288 - protein, 13 aa, synthetic construct
ARYFYDHYYYFDV

SEQ ID NO: 289 - protein, 12 aa, synthetic construct
ARYYDDTIYSFY

SEQ ID NO: 290 - protein, 13 aa, synthetic construct
ARYYYDYGYYFDV

SEQ ID NO: 291 - protein, 12 aa, synthetic construct
ARGYYYYFYYNY

SEQ ID NO: 292 - protein, 13 aa, synthetic construct
ARWGSYGYYYFDY

SEQ ID NO: 293 - protein, 13 aa, synthetic construct
ARHYDYGRYYMDV

SEQ ID NO: 294 - protein, 13 aa, synthetic construct
ARWGGFGYYYFDY

SEQ ID NO: 295 - protein, 13 aa, synthetic construct
ARYGGGYYYYFDV

SEQ ID NO: 296 - protein, 12 aa, synthetic construct
TRTTLGFYAMDY

SEQ ID NO: 297 - protein, 12 aa, synthetic construct
ARYGGAGYYLDY

SEQ ID NO: 298 - protein, 13 aa, synthetic construct
ARWGSEAFYWFDY

SEQ ID NO: 299 - protein, 13 aa, synthetic construct
APWWSGWYYFFDT

SEQ ID NO: 300 - protein, 12 aa, synthetic construct
TRYYDGYYAMDY

SEQ ID NO: 301 - protein, 13 aa, synthetic construct
ARYPNYGFYMFDY

SEQ ID NO: 302 - protein, 12 aa, synthetic construct
ARGYYYYQWYYY

SEQ ID NO: 303 - protein, 12 aa, synthetic construct
ARWRDYYYYFDY

SEQ ID NO: 304 - protein, 13 aa, synthetic construct
ARWVSGARYYMDV

SEQ ID NO: 305 - protein, 12 aa, synthetic construct
AAYGPQGYAFAV

SEQ ID NO: 306 - protein, 12 aa, synthetic construct
ARYYGGLYYFDY

SEQ ID NO: 307 - protein, 13 aa, synthetic construct
ARFGGAGGYYFDY

SEQ ID NO: 308 - protein, 12 aa, synthetic construct
AGYWYGSYSFDY

SEQ ID NO: 309 - protein, 13 aa, synthetic construct
TRYRGAHGYYFDY

SEQ ID NO: 310 - protein, 13 aa, synthetic construct
ARYGDADFYAFDP

SEQ ID NO: 311 - protein, 13 aa, synthetic construct
ARYIFGGPYYFDY

SEQ ID NO: 312 - protein, 12 aa, synthetic construct
ASYYDYYYAFGT

SEQ ID NO: 313 - protein, 12 aa, synthetic construct
ARFPVSSYAYGF

SEQ ID NO: 314 - protein, 13 aa, synthetic construct
VRYPEVYGYYFDY

SEQ ID NO: 315 - protein, 13 aa, synthetic construct
ANVGSSLGIAALD

SEQ ID NO: 316 - protein, 13 aa, synthetic construct
SYPDFGYYVYFAY

SEQ ID NO: 317 - protein, 12 aa, synthetic construct
AKYAFGGWSYSP

SEQ ID NO: 318 - protein, 12 aa, synthetic construct
ARASWYWYGGDK

SEQ ID NO: 319 - protein, 13 aa, synthetic construct
AKYTRGFSYGFGY

SEQ ID NO: 320 - protein, 13 aa, synthetic construct
ARWGYGYYYSFDY

SEQ ID NO: 321 - protein, 13 aa, synthetic construct
ARYGRNGSYSYDY

SEQ ID NO: 322 - protein, 13 aa, synthetic construct
AKFERGYYSFDFG

SEQ ID NO: 323 - protein, 13 aa, synthetic construct
ARYGYGYYYYFDY

SEQ ID NO: 324 - protein, 12 aa, synthetic construct
ARYADYQFHFDN

SEQ ID NO: 325 - protein, 12 aa, synthetic construct
AASADFMYGVIR

SEQ ID NO: 326 - protein, 13 aa, synthetic construct
ARYPGGGIYDFDP

SEQ ID NO: 327 - protein, 13 aa, synthetic construct
ARYGGYGYYWFDY

SEQ ID NO: 328 - protein, 13 aa, synthetic construct
ARWLRGLGYYWDY

SEQ ID NO: 329 - protein, 12 aa, synthetic construct
ARYPEDFYYFDV

SEQ ID NO: 330 - protein, 13 aa, synthetic construct
ARYGDSYYYYFDY

SEQ ID NO: 331 - protein, 13 aa, synthetic construct
ARYGPYYYYAMDY

SEQ ID NO: 332 - protein, 11 aa, synthetic construct
AINLRGYWMDN

SEQ ID NO: 333 - protein, 13 aa, synthetic construct
TRYYGGYGYYFDY

SEQ ID NO: 334 - protein, 13 aa, synthetic construct
ARYGGYGNYWFDT

SEQ ID NO: 335 - protein, 13 aa, synthetic construct
ARGGYGYYTWFDY

SEQ ID NO: 336 - protein, 13 aa, synthetic construct
ARYGTGTYYYFLH

SEQ ID NO: 337 - protein, 13 aa, synthetic construct
AGSSASGWYHYTT

SEQ ID NO: 338 - protein, 13 aa, synthetic construct
ARWEGYGSYEFAY

SEQ ID NO: 339 - protein, 12 aa, synthetic construct
ARYLSYSYAWDN

SEQ ID NO: 340 - protein, 13 aa, synthetic construct
ARYYYDFGYYFDY

SEQ ID NO: 341 - protein, 13 aa, synthetic construct
ARYGGLGGYTLAY

SEQ ID NO: 342 - protein, 13 aa, synthetic construct
AQNWDRGSYFYTY

SEQ ID NO: 343 - protein, 12 aa, synthetic construct
AANAAIFAAWGA

SEQ ID NO: 344 - protein, 12 aa, synthetic construct
TRTRSGAYFFDY

SEQ ID NO: 345 - protein, 13 aa, synthetic construct
ARYGARSGYAYEY

SEQ ID NO: 346 - protein, 11 aa, synthetic construct
ARDGHYFTYDY

SEQ ID NO: 347 - protein, 13 aa, synthetic construct
ARNYYYGAYYFDY

SEQ ID NO: 348 - protein, 13 aa, synthetic construct
ASVDLGGFYDFRY

SEQ ID NO: 349 - protein, 12 aa, synthetic construct
ARYYGYYYAFDY

SEQ ID NO: 350 - protein, 13 aa, synthetic construct
ARWQGRSGYYMDY

SEQ ID NO: 351 - protein, 12 aa, synthetic construct
ARMSYYFYYMDY

SEQ ID NO: 352 - protein, 11 aa, synthetic construct
AAWYSTYYFDY

SEQ ID NO: 353 - protein, 15 aa, synthetic construct
AYTGAPHGGYHYYAY

SEQ ID NO: 354 - protein, 15 aa, synthetic construct
ARSGGHGVNGHWRSF

SEQ ID NO: 355 - protein, 13 aa, synthetic construct
ARYGYDYYYYFDV

SEQ ID NO: 356 - protein, 11 aa, synthetic construct
AKYVLVGSYGV

SEQ ID NO: 357 - protein, 12 aa, synthetic construct
SIYGTDYYWFAD

SEQ ID NO: 358 - protein, 13 aa, synthetic construct
ARFGPAYFYGFYY

SEQ ID NO: 359 - protein, 13 aa, synthetic construct
ARYMRPAFYPFLY

SEQ ID NO: 360 - protein, 14 aa, synthetic construct
ARGGYYGYWYYFDY

SEQ ID NO: 361 - protein, 12 aa, synthetic construct
ARYGSGYYGLDY

SEQ ID NO: 362 - protein, 12 aa, synthetic construct
SRWYGDYFAMDY

SEQ ID NO: 363 - protein, 12 aa, synthetic construct
ATNAFGLWSYGF

SEQ ID NO: 364 - protein, 12 aa, synthetic construct
ARYGVYGYAMDY

SEQ ID NO: 365 - protein, 13 aa, synthetic construct
ASYGSGSYFSFPL

SEQ ID NO: 366 - protein, 13 aa, synthetic construct
ARYGYGAYYYFDY

SEQ ID NO: 367 - protein, 12 aa, synthetic construct
ARYLSYSYAWDY

SEQ ID NO: 368 - protein, 12 aa, synthetic construct
ARHGSYYYDFDF

SEQ ID NO: 369 - protein, 12 aa, synthetic construct
TRYFYDFPYMDY

SEQ ID NO: 370 - protein, 13 aa, synthetic construct
ARYGGLSYYSFDL

SEQ ID NO: 371 - protein, 13 aa, synthetic construct
AKFHWGEDWLSDN

SEQ ID NO: 372 - protein, 12 aa, synthetic construct
ARYGYYGYWFDY

SEQ ID NO: 373 - protein, 12 aa, synthetic construct
ARYLSYSFAWDY

SEQ ID NO: 374 - protein, 12 aa, synthetic construct
VKHGIDYYWYGD

SEQ ID NO: 375 - protein, 13 aa, synthetic construct
AGSVAEGWYHYRD

SEQ ID NO: 376 - protein, 13 aa, synthetic construct
ARSGYPGYGSFAY

SEQ ID NO: 377 - protein, 12 aa, synthetic construct
ARYHNTGYFWGA

SEQ ID NO: 378 - protein, 12 aa, synthetic construct
ARYYDYGYTFDY

SEQ ID NO: 379 - protein, 12 aa, synthetic construct
ASYMGIVGYYPG

SEQ ID NO: 380 - protein, 13 aa, synthetic construct
ARTVVGWYGRFDY

SEQ ID NO: 381 - protein, 13 aa, synthetic construct
ARNSGTGIWAFAY

SEQ ID NO: 382 - protein, 12 aa, synthetic construct
ARYGTYGYAMDY

SEQ ID NO: 383 - protein, 13 aa, synthetic construct
ARWPPYYWWAFDY

SEQ ID NO: 384 - protein, 13 aa, synthetic construct
ARAGGTSISLGHG

SEQ ID NO: 385 - protein, 13 aa, synthetic construct
ARYGDAFIMSFGV

SEQ ID NO: 386 - protein, 13 aa, synthetic construct
APWGTYRYGYFDH

SEQ ID NO: 387 - protein, 12 aa, synthetic construct
ARYSYGAFYYSI

SEQ ID NO: 388 - protein, 13 aa, synthetic construct
AVYGPYGGFSYDS

SEQ ID NO: 389 - protein, 12 aa, synthetic construct
ARYYVGYYAMDY

SEQ ID NO: 390 - protein, 13 aa, synthetic construct
AIWSMYDVGYYPF

SEQ ID NO: 391 - protein, 13 aa, synthetic construct
ARYGLYGSYYFRY

SEQ ID NO: 392 - protein, 12 aa, synthetic construct
ARYYGYYYYMDY

SEQ ID NO: 393 - protein, 11 aa, synthetic construct
ARYSYYRYYFE

SEQ ID NO: 394 - protein, 13 aa, synthetic construct
ARYLTDADFRLHY

SEQ ID NO: 395 - protein, 12 aa, synthetic construct
ARYPYGYYAMDY

SEQ ID NO: 396 - protein, 13 aa, synthetic construct
TRWRWGYGYYFDF

SEQ ID NO: 397 - protein, 12 aa, synthetic construct
ARYWYGSYVFDY

SEQ ID NO: 398 - protein, 11 aa, synthetic construct
AADGYRGTYSF

SEQ ID NO: 399 - protein, 13 aa, synthetic construct
SRYGRGVYYYFDY

SEQ ID NO: 400 - protein, 12 aa, synthetic construct
AQSGSYFYPFSY

SEQ ID NO: 401 - protein, 13 aa, synthetic construct
ARPSYDVGGDMLF

SEQ ID NO: 402 - protein, 13 aa, synthetic construct
ARYVTANTYSYDP

SEQ ID NO: 403 - protein, 12 aa, synthetic construct
ARYYYSGYYMSV

SEQ ID NO: 404 - protein, 12 aa, synthetic construct
ALYKRYDYAFGY

SEQ ID NO: 405 - protein, 13 aa, synthetic construct
ARWGTYTYGYFDY

SEQ ID NO: 406 - protein, 13 aa, synthetic construct
ARWARGAGYYWDY

SEQ ID NO: 407 - protein, 13 aa, synthetic construct
ARYPSGYYYYFDY

SEQ ID NO: 408 - protein, 12 aa, synthetic construct
ARASWNWYGGDY

SEQ ID NO: 409 - protein, 11 aa, synthetic construct
AFPWAGHYMER

SEQ ID NO: 410 - protein, 12 aa, synthetic construct
ARGYYHYYHYQY

SEQ ID NO: 411 - protein, 12 aa, synthetic construct
VRYYRGYYYFDY

SEQ ID NO: 412 - protein, 13 aa, synthetic construct
ARYGDGTYYFFAH

SEQ ID NO: 413 - protein, 13 aa, synthetic construct
ARYDYYGYYYFDY

SEQ ID NO: 414 - protein, 12 aa, synthetic construct
ARYQAYDNSYGV

SEQ ID NO: 415 - protein, 13 aa, synthetic construct
ARSYAYLYGSWDY

SEQ ID NO: 416 - protein, 12 aa, synthetic construct
ARYHFSGYFMDA

SEQ ID NO: 417 - protein, 13 aa, synthetic construct
ARYYSYGGYYFDY

SEQ ID NO: 418 - protein, 12 aa, synthetic construct
ARYGRDYYYFDV

SEQ ID NO: 419 - protein, 11 aa, synthetic construct
AKYVASGYSLA

SEQ ID NO: 420 - protein, 13 aa, synthetic construct
ARYYSDYSYTFSY

SEQ ID NO: 421 - protein, 12 aa, synthetic construct
ARYHYSGYFMDS

SEQ ID NO: 422 - protein, 12 aa, synthetic construct
AAYWYGVYVFRY

SEQ ID NO: 423 - protein, 12 aa, synthetic construct
ARSEWFWGSFAY

SEQ ID NO: 424 - protein, 14 aa, synthetic construct
ARSHYGYYDYYTDF

SEQ ID NO: 425 - protein, 12 aa, synthetic construct
TLNGPLYWMWTL

SEQ ID NO: 426 - protein, 13 aa, synthetic construct
ARWWYGGRNYFDI

SEQ ID NO: 427 - protein, 12 aa, synthetic construct
ARYPDYYYAMDY

SEQ ID NO: 428 - protein, 15 aa, synthetic construct
ATWPHINTRIYAFDP

SEQ ID NO: 429 - protein, 12 aa, synthetic construct
ATGPYTVYSYPY

SEQ ID NO: 430 - protein, 14 aa, synthetic construct
ARYRGGYGGYVFDP

SEQ ID NO: 431 - protein, 12 aa, synthetic construct
ARNYYGSYYFDY

SEQ ID NO: 432 - protein, 13 aa, synthetic construct
ARFSGYGAYDYAY

SEQ ID NO: 433 - protein, 13 aa, synthetic construct
ARDGYPQYGGYGY

SEQ ID NO: 434 - protein, 13 aa, synthetic construct
AGYEGYGLYPFAY

SEQ ID NO: 435 - protein, 12 aa, synthetic construct
ARGYFGGYYFAY

SEQ ID NO: 436 - protein, 13 aa, synthetic construct
ARWWTPANYPFLY

SEQ ID NO: 437 - protein, 12 aa, synthetic construct
AKYGRSDYGFEH

SEQ ID NO: 438 - protein, 12 aa, synthetic construct
ARFLYGSYYFDY

SEQ ID NO: 439 - protein, 13 aa, synthetic construct
ASYYAPGSYGFDP

SEQ ID NO: 440 - protein, 13 aa, synthetic construct
ARYGLNHYYVYPY

SEQ ID NO: 441 - protein, 13 aa, synthetic construct
ARSYFRFTGDFFY

SEQ ID NO: 442 - protein, 13 aa, synthetic construct
SRYYDYNYYFMDY

SEQ ID NO: 443 - protein, 15 aa, synthetic construct
VRGYGYNFYYHGFSY

SEQ ID NO: 444 - protein, 12 aa, synthetic construct
ARHHYSGYFHDV

SEQ ID NO: 445 - protein, 13 aa, synthetic construct
ARTTSSIPSSFDY

SEQ ID NO: 446 - protein, 11 aa, synthetic construct
ARFDLVGAFDY

SEQ ID NO: 447 - protein, 13 aa, synthetic construct
ARDGYPFYSGMDY

SEQ ID NO: 448 - protein, 13 aa, synthetic construct
ATSGSVDRYDYVY

SEQ ID NO: 449 - protein, 13 aa, synthetic construct
ARYNPFDGFYFDV

SEQ ID NO: 450 - protein, 11 aa, synthetic construct
ATGYSYLRFYD

SEQ ID NO: 451 - protein, 13 aa, synthetic construct
ARGYYQDSQSWGY

SEQ ID NO: 452 - protein, 13 aa, synthetic construct
ASSGRLDLYDFRL

SEQ ID NO: 453 - protein, 13 aa, synthetic construct
ATGYGYLLYSDHD

SEQ ID NO: 454 - protein, 12 aa, synthetic construct
ARGYWWLYGFAY

SEQ ID NO: 455 - protein, 12 aa, synthetic construct
ARWSWPGYEFEF

SEQ ID NO: 456 - protein, 12 aa, synthetic construct
ARSTDYWHASQY

SEQ ID NO: 457 - protein, 13 aa, synthetic construct
ATGSGGGGFSYGY

SEQ ID NO: 458 - protein, 14 aa, synthetic construct
ARLAFDSVSFYSDY

SEQ ID NO: 459 - protein, 13 aa, synthetic construct
AQYGRGGYWYFDY

SEQ ID NO: 460 - protein, 13 aa, synthetic construct
ARSYAFLYGRMDY

SEQ ID NO: 461 - protein, 11 aa, synthetic construct
ASYAGYSPFDY

SEQ ID NO: 462 - protein, 12 aa, synthetic construct
ARSYYGAYYFDY

SEQ ID NO: 463 - protein, 13 aa, synthetic construct
ARDSPYYGSLYLY

SEQ ID NO: 464 - protein, 12 aa, synthetic construct
AKYGISDYGFDH

SEQ ID NO: 465 - protein, 12 aa, synthetic construct
ARSGEGYYAFDY

SEQ ID NO: 466 - protein, 12 aa, synthetic construct
ARYLEYTYAWDY

SEQ ID NO: 467 - protein, 12 aa, synthetic construct
ASGDLTAGSSQT

SEQ ID NO: 468 - protein, 13 aa, synthetic construct
ARWYGLGSYGLTY

SEQ ID NO: 469 - protein, 13 aa, synthetic construct
ARWEQASVGSFGY

SEQ ID NO: 470 - protein, 13 aa, synthetic construct
ARWRYGGAYQFAL

SEQ ID NO: 471 - protein, 13 aa, synthetic construct
ARDGGMYRYGLYG

SEQ ID NO: 472 - protein, 12 aa, synthetic construct
TRNGPLYWHWDL

SEQ ID NO: 473 - protein, 12 aa, synthetic construct
ARTTISSSSYTG

SEQ ID NO: 474 - protein, 13 aa, synthetic construct
ARGHYSLGYSFDF

SEQ ID NO: 475 - protein, 12 aa, synthetic construct
ALLYGYYPSGVG

SEQ ID NO: 476 - protein, 11 aa, synthetic construct
ATGYSYLRYYD

SEQ ID NO: 477 - protein, 13 aa, synthetic construct
ARDVYVGSPVFDY

SEQ ID NO: 478 - protein, 13 aa, synthetic construct
ARSNNWYSYSFQF

SEQ ID NO: 479 - protein, 13 aa, synthetic construct
AYMGSHTIYSFEH

SEQ ID NO: 480 - protein, 12 aa, synthetic construct
ASDRYGSYSLVS

SEQ ID NO: 481 - protein, 13 aa, synthetic construct
ATSGTASWQHYGV

SEQ ID NO: 482 - protein, 12 aa, synthetic construct
ARYYGWYYYFDY

SEQ ID NO: 483 - protein, 11 aa, synthetic construct
AVEGGTRALEP

SEQ ID NO: 484 - protein, 13 aa, synthetic construct
ARLGASHGYLYDD

SEQ ID NO: 485 - protein, 11 aa, synthetic construct
ARLYYSSFNGM

SEQ ID NO: 486 - protein, 13 aa, synthetic construct
ARDYDFGLSVGAV

SEQ ID NO: 487 - protein, 13 aa, synthetic construct
ARYHYRTIGSFGY

SEQ ID NO: 488 - protein, 13 aa, synthetic construct
AWASGYGGYGMDY

SEQ ID NO: 489 - protein, 12 aa, synthetic construct
ARYHYSGFFMDA

SEQ ID NO: 490 - protein, 12 aa, synthetic construct
ARYLSDSYAWDY

SEQ ID NO: 491 - protein, 13 aa, synthetic construct
ATGLSPSGSNGPY

SEQ ID NO: 492 - protein, 13 aa, synthetic construct
VRSGYDYYSWFAY

SEQ ID NO: 493 - protein, 13 aa, synthetic construct
ARGYAYYVAGREY

SEQ ID NO: 494 - protein, 11 aa, synthetic construct
TRRDFGYYMAY

SEQ ID NO: 495 - protein, 13 aa, synthetic construct
ARNYYGAAYYFDY

SEQ ID NO: 496 - protein, 12 aa, synthetic construct
ARGYGVGASFVY

SEQ ID NO: 497 - protein, 15 aa, synthetic construct
ARTGWGYDNISGYEY

SEQ ID NO: 498 - protein, 13 aa, synthetic construct
ARSGYYYSYSFDY

SEQ ID NO: 499 - protein, 13 aa, synthetic construct
ARYRGSSSYYFDY

SEQ ID NO: 500 - protein, 13 aa, synthetic construct
ARYYGGSMYYYGY

SEQ ID NO: 501 - protein, 13 aa, synthetic construct
ARSSGYYSYSYDY

SEQ ID NO: 502 - protein, 13 aa, synthetic construct
AREGGVGYYSFAY

SEQ ID NO: 503 - protein, 13 aa, synthetic construct
ASSGGGYSYYYTY

SEQ ID NO: 504 - protein, 8 aa, synthetic construct
IDPENGYT

SEQ ID NO: 505 - protein, 13 aa, synthetic construct
ARVGGYGYYYYDY

SEQ ID NO: 506 - protein, 13 aa, synthetic construct
ASSGGSSSYYYNY

SEQ ID NO: 507 - protein, 8 aa, synthetic construct
IYSSDGYT

SEQ ID NO: 508 - protein, 13 aa, synthetic construct
ARSGGSSYYYYDY

SEQ ID NO: 509 - protein, 8 aa, synthetic construct
IYPEDGST

SEQ ID NO: 510 - protein, 13 aa, synthetic construct
ARSGGSSTYAYDY

SEQ ID NO: 511 - protein, 13 aa, synthetic construct
ASSGGRYYYYYDY

SEQ ID NO: 512 - protein, 13 aa, synthetic construct
ARSRGSSSYMMDY

SEQ ID NO: 513 - protein, 13 aa, synthetic construct
ASSGGSSSYSYYY

SEQ ID NO: 514 - protein, 13 aa, synthetic construct
ARSYGSGSYSFDY

SEQ ID NO: 515 - protein, 13 aa, synthetic construct
ARYSYYGGGYFDY

SEQ ID NO: 516 - protein, 13 aa, synthetic construct
ARDRGVSGYYLYY

SEQ ID NO: 517 - protein, 13 aa, synthetic construct
ARYGGSYFYYYSY

SEQ ID NO: 518 - protein, 13 aa, synthetic construct
ARYGGSSNYYYDY

SEQ ID NO: 519 - protein, 13 aa, synthetic construct
ARSGGSSFYGMDY

SEQ ID NO: 520 - protein, 13 aa, synthetic construct
ARTGGSSYYAYDY

SEQ ID NO: 521 - protein, 13 aa, synthetic construct
SRYGYSGYGAFDY

SEQ ID NO: 522 - protein, 13 aa, synthetic construct
ARSGGSYSYSFDV

SEQ ID NO: 523 - protein, 13 aa, synthetic construct
ARSGGSGYYSYNY

SEQ ID NO: 524 - protein, 13 aa, synthetic construct
ARSGGSGYYYYDY

SEQ ID NO: 525 - protein, 13 aa, synthetic construct
ARDGGGGVYAFTY

SEQ ID NO: 526 - protein, 13 aa, synthetic construct
ARSSGSSYYGFDY

SEQ ID NO: 527 - protein, 13 aa, synthetic construct
ARYGGDYYYYYDY

SEQ ID NO: 528 - protein, 13 aa, synthetic construct
ARYGGSSYYYFDY

SEQ ID NO: 529 - protein, 13 aa, synthetic construct
ARYGGSSYYYYDY

SEQ ID NO: 530 - protein, 13 aa, synthetic construct
TRYGSFGYYALDS

SEQ ID NO: 531 - protein, 13 aa, synthetic construct
ARYGGGYYYYYDY

SEQ ID NO: 532 - protein, 13 aa, synthetic construct
ARYRGRGFYSFDY

SEQ ID NO: 533 - protein, 13 aa, synthetic construct
ARYGGRGYYSFDY

SEQ ID NO: 534 - protein, 13 aa, synthetic construct
ARDGGGGFYAYDY

SEQ ID NO: 535 - protein, 13 aa, synthetic construct
ARDGGSSSYSYSY

SEQ ID NO: 536 - protein, 13 aa, synthetic construct
ARYSGSSYYYYDY

SEQ ID NO: 537 - protein, 13 aa, synthetic construct
SRYSYSGGGSFDY

SEQ ID NO: 538 - protein, 13 aa, synthetic construct
ARDGGRSYYSFYY

SEQ ID NO: 539 - protein, 13 aa, synthetic construct
ARVGGYGYYSYDY

SEQ ID NO: 540 - protein, 13 aa, synthetic construct
ARSSGYYYYGFVY

SEQ ID NO: 541 - protein, 13 aa, synthetic construct
ARDGGRGRYAMDY

SEQ ID NO: 542 - protein, 13 aa, synthetic construct
ARYSSSGGSAFDY

SEQ ID NO: 543 - protein, 13 aa, synthetic construct
ARYSYYGGGAFDY

SEQ ID NO: 544 - protein, 13 aa, synthetic construct
ARSGGYYHYGFVY

SEQ ID NO: 545 - protein, 13 aa, synthetic construct
ARYGGRSSYYFDY

SEQ ID NO: 546 - protein, 13 aa, synthetic construct
ARSGGGSYYYYDY

SEQ ID NO: 547 - protein, 13 aa, synthetic construct
ARSTGSGFYSYDY

SEQ ID NO: 548 - protein, 13 aa, synthetic construct
ARSGGYYTYGFDY

SEQ ID NO: 549 - protein, 13 aa, synthetic construct
TRYSYYGYHAMDY

SEQ ID NO: 550 - protein, 13 aa, synthetic construct
ARYSGDSYYYYDY

SEQ ID NO: 551 - protein, 13 aa, synthetic construct
ARSGGRYTYGFDY

SEQ ID NO: 552 - protein, 13 aa, synthetic construct
ARVGGRGYYAYDY

SEQ ID NO: 553 - protein, 14 aa, synthetic construct
DVNTAVSASFHYTT

SEQ ID NO: 554 - protein, 15 aa, synthetic construct
NVWDWVPASSGWYIA

SEQ ID NO: 555 - protein, 18 aa, synthetic construct
DIPRSISGYVWGSYHYTT

(19) United States
(12) Patent Application Publication
(10) Pub. No.: US 2025/0218531 A1
(43) Pub. Date: Jul. 3, 2025

(54) UNLOCKING DE NOVO ANTIBODY DESIGN WITH GENERATIVE ARTIFICIAL INTELLIGENCE

(71) Applicant: ABSCI CORPORATION, Vancouver, WA (US)

(72) Inventors: Joshua Meier, Vancouver, WA (US); Gregory Hannum, Vancouver, WA (US); Sean McClain, Vancouver, WA (US)

(21) Appl. No.: 18/837,170

(22) PCT Filed: Feb. 9, 2023
(86) PCT No.: PCT/US23/62331

§ 371 Date: Aug. 8, 2024

Related U.S. Application Data
(60) Provisional application No. 63/478,933, filed on Jan. 7, 2023.
(60) Provisional application No. 63/308,495, filed on Feb. 9, 2022.

(51) Int. Cl. G16B 15/30; G06N 3/09; G16B 15/20; G16B 40/20

(52) U.S. Cl. CPC G16B 15/30; G06N 3/09; G16B 15/20; G16B 40/20

(57) ABSTRACT

A computing system for generating structural information of a biomolecule includes a processor; and one or more non-transitory computer-readable media having stored thereon instructions that, when executed by the one or more processors, cause the computing system to: receive training inputs; process the training inputs with a machine-learned biomolecule prediction model; evaluate a loss function; and modify parameters of the machine-learned model. A computing system for generating structural information of a target biomolecule includes a processor and one or more non-transitory computer-readable media having stored thereon a machine-learned biomolecule prediction model and instructions that, when executed by the one or more processors, cause the computing system to receive a target input; and predict the structural information of the target biomolecule. A computing system for predicting an affinity of a target biomolecule includes a processor and a computer-readable media having stored thereon: a machine-learned affinity prediction artificial neural network, including: (i) one or more biomolecule prediction layers trained to predict biomolecule structural information from target inputs; (ii) one or more docking layers trained to generate docked complexes from two or more input three-dimensional biomolecules; and (iii) one or more affinity prediction layers trained to predict affinity from input docked complexes; wherein the one or more biomolecule prediction layers, the one or more docking layers, and the one or more affinity prediction layers are connected; and instructions that, when executed by the one or more processors, cause the computing system to: receive a target input; and process the target input using the affinity prediction artificial neural network to generate a docked complex corresponding to the target input and a corresponding structural affinity value.

### INCORPORATION BY REFERENCE OF MATERIAL SUBMITTED ELECTRONICALLY

The Sequence Listing, which is a part of the present disclosure, is submitted concurrently with the specification in computer readable format. The name of the file containing the Sequence Listing is “57581_Seglisting.xml”, which was created on Feb. 9, 2023 and is 489,494 bytes in size. The subject matter of the Sequence Listing is incorporated herein in its entirety by reference.

### CROSS-REFERENCE TO RELATED APPLICATIONS

The present application claims priority to U.S. Provisional Patent Application No. 63/478,933, entitled UNLOCKING DE NOVO ANTIBODY DESIGN WITH GENERATIVE ARTIFICIAL INTELLIGENCE, filed Jan. 7, 2023, and U.S. Provisional Patent Application No. 63/308,495, entitled STRUCTURE-BASED DESIGN OF BINDING PARTNER-TARGETED BIOMOLECULES, filed Feb. 9, 2022. The contents of each are incorporated herein by reference in their entireties.

### BACKGROUND

Antibodies are diverse proteins used by the immune system to naturally bind and neutralize foreign objects, such as viruses, fungi, and bacteria. Accordingly, antibodies have the potential to serve as drug candidates for many infectious diseases and cancers. Conventional techniques for in silico automatic design and/or improvement of antibodies are lacking, for example, because such deep learning-based protein design techniques design proteins unconditionally, use only open source data, and do not enable targeted design to a specified binding partner. Such techniques have also not demonstrated the ability to condition design on other criteria/aims. Conventional laboratory-based techniques are time-consuming and expensive. For example, traditional de novo antibody discovery requires time and resource intensive screening of large immune or synthetic libraries. These methods also offer little control over the output sequences, which can result in lead candidates with sub-optimal binding and poor developability attributes. Conventional generative antibody design techniques have not demonstrated de novo antibody design with experimental validation. Thus, improved techniques for protein design are needed.

### SUMMARY

In one aspect, a computing system for training a machine learning model to generate structural information of a target biomolecule includes one or more processors; and one or more non-transitory computer-readable media having stored thereon instructions that, when executed by the one or more processors, cause the computing system to: (1) receive one or more training inputs, including one or more of (i) input biomolecule structural information, (ii) input biomolecule binding partner structural information or (iii) input biomolecule-input binding partner binding complex structural information; (2) process the one or more training inputs with a machine-learned biomolecule prediction model to generate predicted biomolecule structural information; (3) evaluate a loss function that compares the predicted biomolecule structural information to a ground truth value; and (4) modify one or more values of one or more parameters of the machine-learned model based at least in part on the loss function.

In another aspect, a computing system for generating structural information of a target biomolecule includes one or more processors; and one or more non-transitory computer-readable media having stored thereon: a machine-learned biomolecule prediction model trained to predict structural information of biomolecules based on an input; and instructions that, when executed by the one or more processors, cause the computing system to: (1) receive a target input including one or more of a target binding partner primary sequence, three-dimensional coordinates of a target binding partner, a target binding partner epitope primary sequence, or three-dimensional coordinates of a target binding partner epitope primary sequence, or a fragment or portion of any of the foregoing; and (2) predict the structural information of the target biomolecule by processing the target input with the machine-learned biomolecule prediction model.

In yet another aspect, a computing system for predicting an affinity of a target biomolecule includes one or more processors; and one or more non-transitory computer-readable media having stored thereon: a machine-learned affinity prediction artificial neural network, including: (i) one or more biomolecule prediction layers trained to predict biomolecule structural information from target inputs; (ii) one or more docking layers trained to generate docked complexes from two or more input three-dimensional biomolecules; and (iii) one or more affinity prediction layers trained to predict affinity from input docked complexes; wherein the one or more biomolecule prediction layers, the one or more docking layers, and the one or more affinity prediction layers are connected; and instructions that, when executed by the one or more processors, cause the computing system to: (1) receive a target input comprising one or more of a target binding partner sequence, a target binding partner, or a target epitope; and (2) process the target input using the affinity prediction artificial neural network to generate a docked complex corresponding to the target input and a corresponding structural affinity value.

### BRIEF DESCRIPTION OF THE DRAWINGS

FIG. 1A depicts zero-shot generative AI for de novo antibody design, wherein deep learning models trained on antibody-antigen interactions combined with ultra-high throughput wet lab experimentation enable the design of binders to never-before-seen antigens without the need for further affinity maturation or lead optimization.

FIG. 1B depicts an exemplary computing environment for performing the present techniques, according to some aspects.

FIG. 2A depicts an exemplary computer-implemented method for training a machine learning model to generate structural information of a biomolecule, according to some aspects.

FIG. 2B depicts a data flow block diagram for training and operating one or more machine learning models, according to some aspects.

FIG. 2C depicts a computer-implemented method for generating structural information of a target biomolecule, according to some aspects.

FIG. 3A depicts a block flow diagram of an exemplary computer-implemented method for performing de novo biomolecule discovery in silico, according to some aspects.

FIG. 3B depicts an exemplary block diagram, according to some aspects.

FIG. 3C depicts a block diagram of exemplary CDR3 in silico discovery inputs and outputs, according to some aspects, wherein block 310b includes SEQ ID NO: 1, and wherein block 310c includes SEQ ID NO: 2.

FIG. 3D depicts an exemplary block diagram of machine learning-based drug candidate design, according to some aspects.

FIG. 3E depicts an exemplary block flow diagram of antigen encoding, according to some aspects.

FIG. 3F depicts an exemplary block flow diagram of antibody generation training, according to some aspects, wherein block 322a includes SEQ ID NO: 3 and SEQ ID NO: 4; and wherein block 322c includes SEQ ID NO: 5.

FIG. 3G depicts an exemplary block flow diagram of antibody generation training, according to some aspects, including SEQ ID NO: 6, SEQ ID NO: 7 and SEQ ID NO: 8.

FIG. 3H depicts an exemplary block flow diagram of antibody generation training, according to some aspects, including SEQ ID NO: 8 and SEQ ID NO: 9.

FIG. 3I depicts an exemplary block flow diagram of antibody generation training, according to some aspects, including SEQ ID NO: 3, SEQ ID NO: 4 and SEQ ID NO: 5.

FIG. 3J depicts an exemplary block flow diagram of antibody generation training, according to some aspects, including SEQ ID NO: 3 and SEQ ID NO: 4.

FIG. 3K depicts an exemplary block flow diagram of antibody generation training, according to some aspects, including SEQ ID NO: 3, SEQ ID NO: 4 and SEQ ID NO: 5.

FIG. 3L depicts an exemplary block diagram depicting supervised machine learning model output, according to some aspects.

FIG. 4 depicts an example graphical user interface including visualized ground truth structural information and predicted structural information, according to some aspects.

FIG. 5A depicts an example block flow diagram of fully in silico biomolecule design, according to some aspects.

FIG. 5B depicts an example block diagram of a fully in silico de novo biomolecule design artificial intelligence model, according to some aspects.

FIG. 5C depicts an example block flow diagram of in silico biomolecule design pipelines, according to some aspects.

FIG. 6A depicts exemplary in silico biomolecule engineering including binder prediction based on optimization of CDRH3 while keeping remaining CDRs invariant, according to some aspects including SEQ ID NOs: 553-555.

FIG. 6B depicts exemplary ranibizumab and trastuzumab bH1 visualizations, according to some aspects.

FIG. 6C depicts exemplary sensorgrams corresponding to surface plasmon response results generated for predicted in silico binding of trastuzumab-bH1 CDR3 variants binding to VEGF, according to some aspects.

FIG. 6D depicts exemplary tabular data corresponding to surface plasmon response results generated for the predicted in silico binding of trastuzumab-bH1 CDR3 variants binding to VEGF of FIG. 6C, according to some aspects, including SEQ ID NOs: 10-22.

FIG. 6E depicts exemplary sensorgrams corresponding to surface plasmon response results generated for predicted in silico binding of trastuzumab CDR3 variants binding to Her2, according to some aspects.

FIG. 6F depicts exemplary tabular data corresponding to surface plasmon response results generated for the predicted in silico binding of trastuzumab CDR3 variants binding to Her2 of FIG. 6E, according to some aspects, including SEQ ID NOs: 23-38.

FIG. 7A depicts an exemplary block diagram of initial laboratory validation of modeling predictions, according to some aspects.

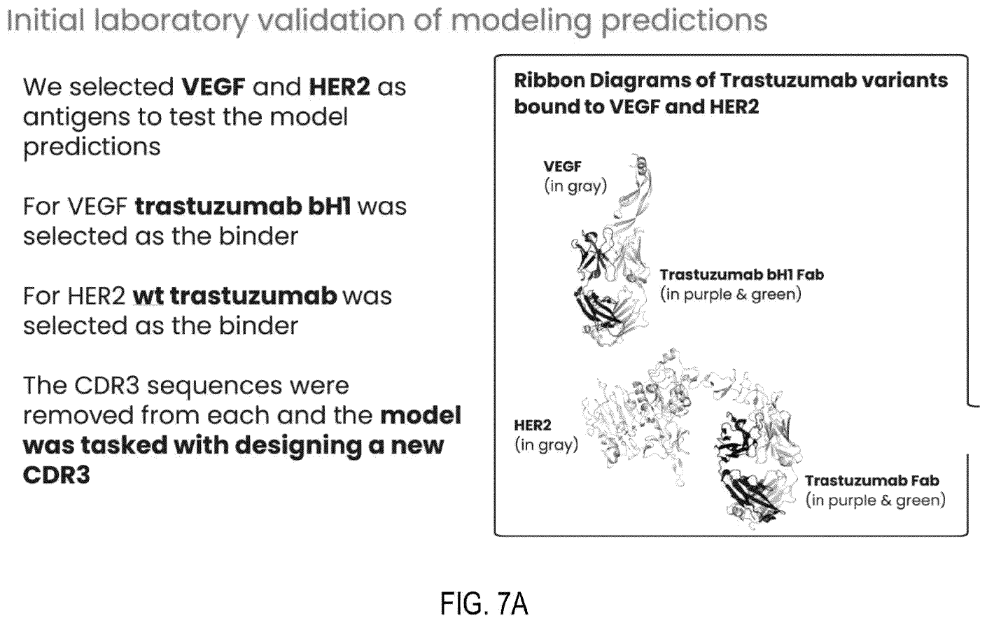

FIG. 7B depicts an exemplary block diagram of in silico generations of de novo designed trastuzumab CDR3, having comparable or improved binding affinity to VEGF, along with no polyspecificity, according to some aspects.

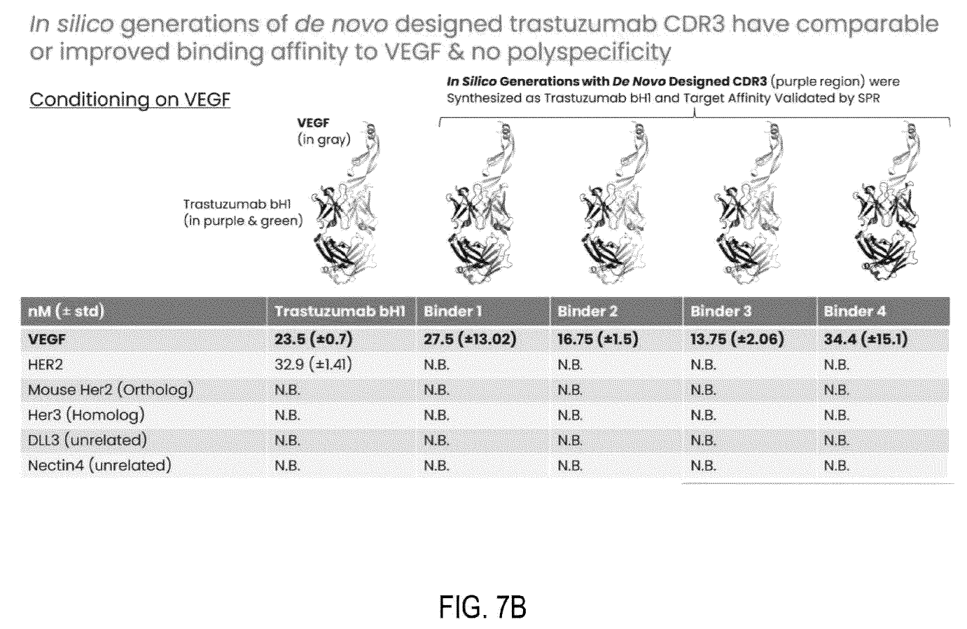

FIG. 7C depicts an exemplary block diagram of in silico generations of de novo designed trastuzumab CDR3 binding to HER2 with high specificity, according to some aspects.

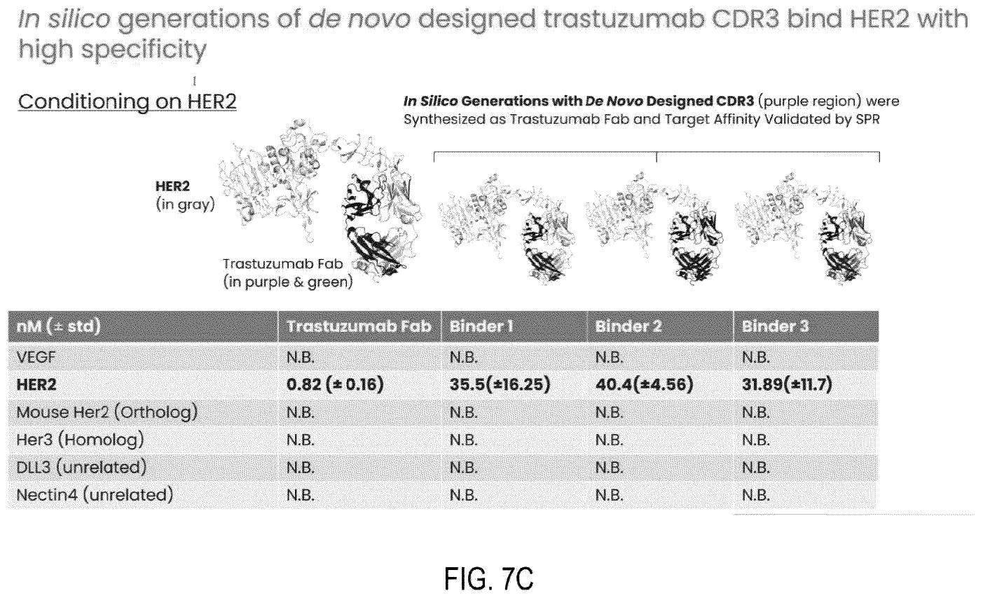

FIG. 7D depicts an exemplary block diagram of negative controls with scrambled CDR3 sequences that do not bind antigens, according to some aspects, including SEQ ID NO: 39 and SEQ ID NO: 40.

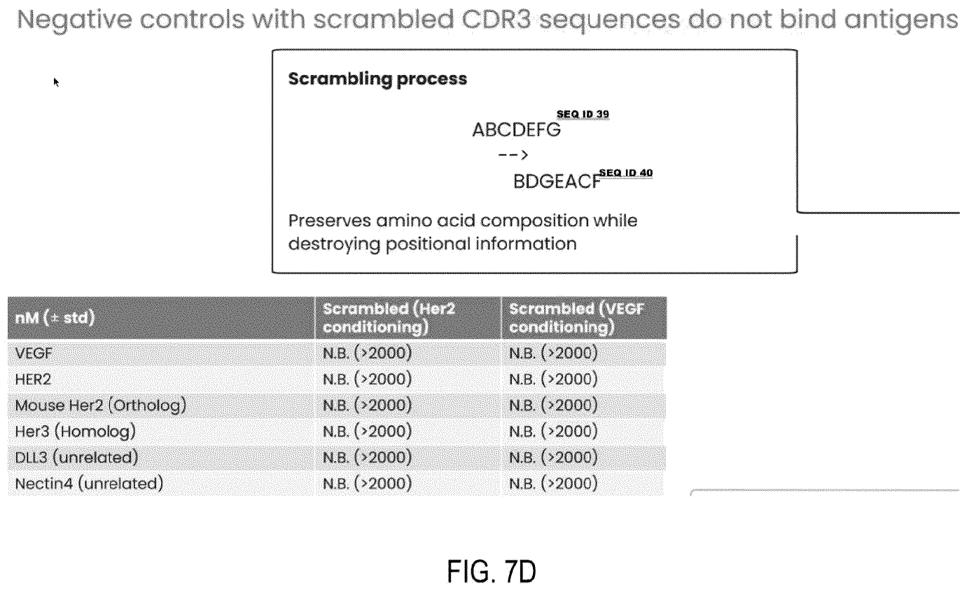

FIG. 7E depicts an exemplary block diagram of generated CDR3 sequences that exhibit high diversity, according to some aspects, including SEQ ID NO: 20.

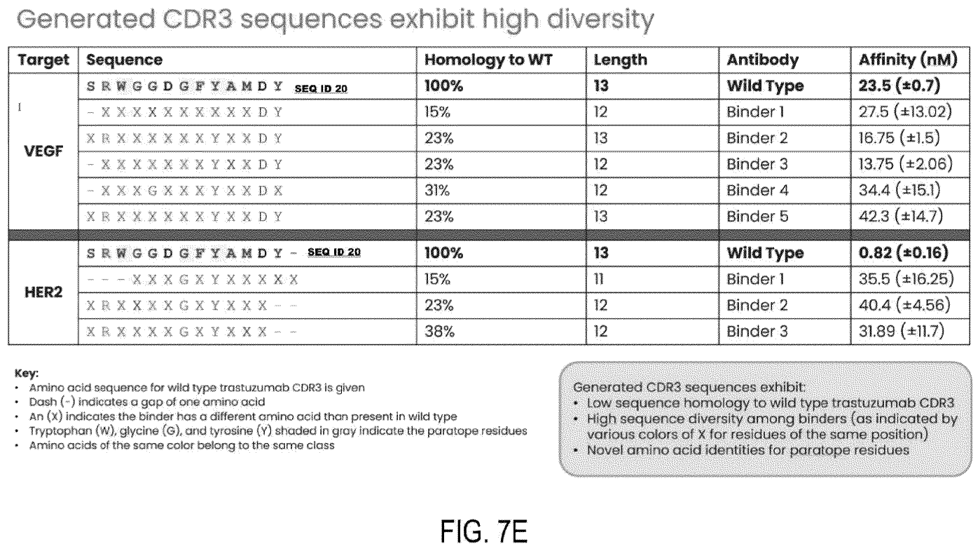

FIG. 7F depicts an exemplary tree diagram showing that in silico generations of de novo designed variants are highly diverse, according to some aspects.

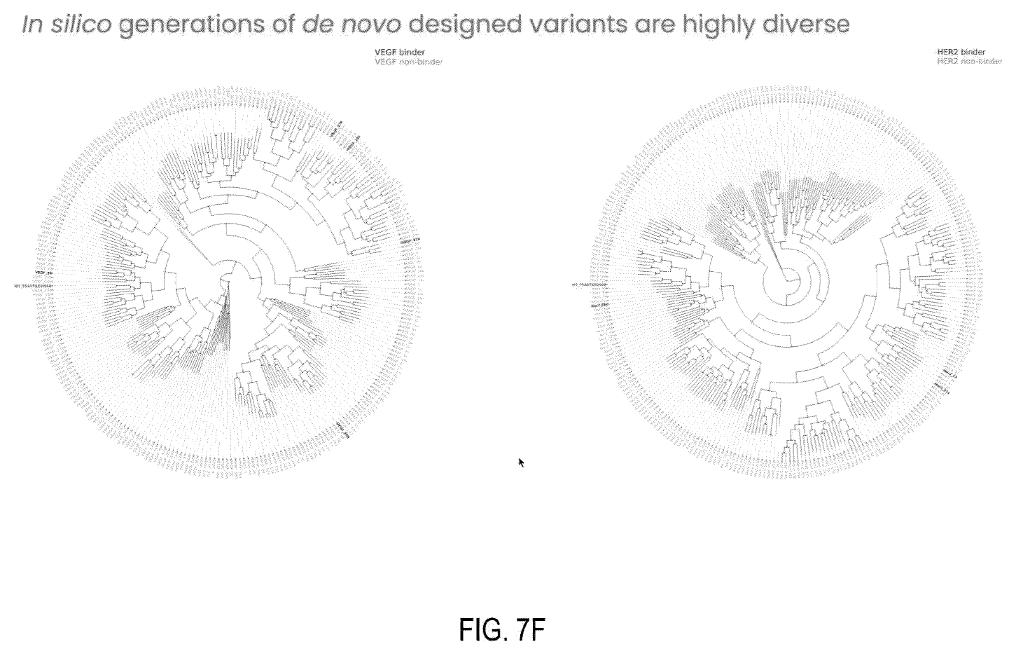

FIG. 8A depicts a logo plot of HCDR3s of 421 binding trastuzumab variants, wherein the greater diversity observed in the centers of the CDRs (hypervariable domain) corresponds to the D germline gene, and whereas the tails correspond to the J and V germline genes, which tend to be less diverse.

FIG. 8B depicts binding affinities of AI-generated zero-shot binders, including 71 designs with comparable affinity (<10 nM) to trastuzumab and three with tighter binding, according to some aspects.

FIG. 8C depicts designed variant binding affinities vs. edit distance to trastuzumab, wherein edit distances range from 2 mutations (84.6% sequence identity) to 12 mutations (7.7% sequence identity), illustrating the novelty of the designs, and wherein while binding affinity tends to drop as the edit distance grows, the present techniques still identify binders with comparable (within 0.5 log) or higher binding affinities in every group.

FIG. 8D depicts pairwise edit distances between binders (e.g., maximum of 15 mutations, median of 8, mean of 7.7, standard deviation of 2.1).

FIG. 8E depicts a pairwise edit distance distribution (on a log scale) of pairwise HCDR3 edit distances for AI-designed binders to HER2, according to some aspects.

FIG. 8F depicts Lengths Distribution of HCDR3 lengths for AI-designed binders to HER2

FIG. 9A depicts binder minimum edit distance to training data HCDR3s, wherein across the designs, binders up to eight mutations away from any HCDR3 in the training data are observed.

FIG. 9B depicts binder minimum edit distance to any HCDR3 in SAbDab for AI-assigned binders to HER2, according to some aspects.

FIG. 9C depicts binder minimum edit distance of OAS HCDR3s, according to some aspects.

FIG. 9D depicts Naturalness measures of the present designed binders versus their diversity to trastuzumab, according to some aspects.

FIG. 9E depicts naturalness of designed binders vs. controls, according to some aspects.

FIG. 9F depicts distribution of minimum sum of edit distances to HCDR3s in OAS for AI designed binders to HER2 as well as trastuzumab HCDR1 and HCDR2 to OAS HCDR1 and HCDR2 sequences, respectively.

FIG. 9G depicts Naturalness Score vs. Minimum OAS ED Distribution of Naturalness scores for de novo designed HER2 binders vs. minimum edit distance to the HCDR3s in OAS.

FIG. 9H depicts Naturalness Scores vs. minimum edit distance to any HCDR123 in OAS.FIG. 9I depicts distribution of minimum edit distance to HCDR3s in OAS for multi-step multi-HCDR AI-designed binders to HER2.

FIG. 9I depicts a minimum edit distance to any HCDR3 in OAS, according to some aspects.

FIG. 9J depicts distribution of minimum edit distance to HCDR1s, HCDR2s, and HCDR3s in OAS for multi-step multi-HCDR AI-designed binders to HER2.

FIG. 10A depicts conformational flexibility of de novo designed HCDR3 Her2 binders, including SEQ ID NOs: 41-48.

FIG. 10B depicts space-filling representation of HCDR3 loops interacting with epitope residues within 5 Å of HCDR3 atoms. Trastuzumab HCDR3 loop is colored red and de novo Her2 binder HCDR3 loops are colored blue; two distinct epitope pockets that differentially interact with residues of each HCDR3; and the interacting surfaces of epitope changes based on HCDR3 sequence and conformation.

FIG. 10C depicts a comparison of trastuzumab-Her2 structure with de novo designed HCDR3-Her2 complexes, showing superimposition of the trastuzumab-Her2 structure with de novo designed HCDR3-Her2 complexes showing conformational differences in HCDR3 backbone; main chain backbone is depicted as ribbons and spatial conserved side chains are shown as sticks.

FIG. 10D depicts Stick representations of HCDR3 loops interacting with epitope residues within 5 Å of HCDR3 atoms. Trastuzumab HCDR3 loop is colored red and de novo Her2 binders HCDR3 loops are colored blue. Epitope residues are labeled according to crystal structure 1N8Z.* denotes novel epitope residues in de novo HCDR3 complexes not observed in trastuzumab-Her2 complex.

FIG. 11A depicts alignment of designed VEGF Binder to the ranibizumab sequence, including SEQ ID NO: 49 and SEQ ID NO: 50.

FIG. 11B depicts alignment of SARS-CoV-2 Omicron binder to casirivimab. The designed HCDR3 is diverse and novel as it 6 mutations away from casirivimab's HCDR3, at least 2 mutations away from any HCDR3 in OAS, and at least 4 mutations away from any HCDR3 in CoV-AbDab (a database of antibodies capable of binding coronaviruses), including SEQ ID NO: 51 and SEQ ID NO: 52.

FIG. 12A depicts designed VEGF binder structure, according to some aspects.

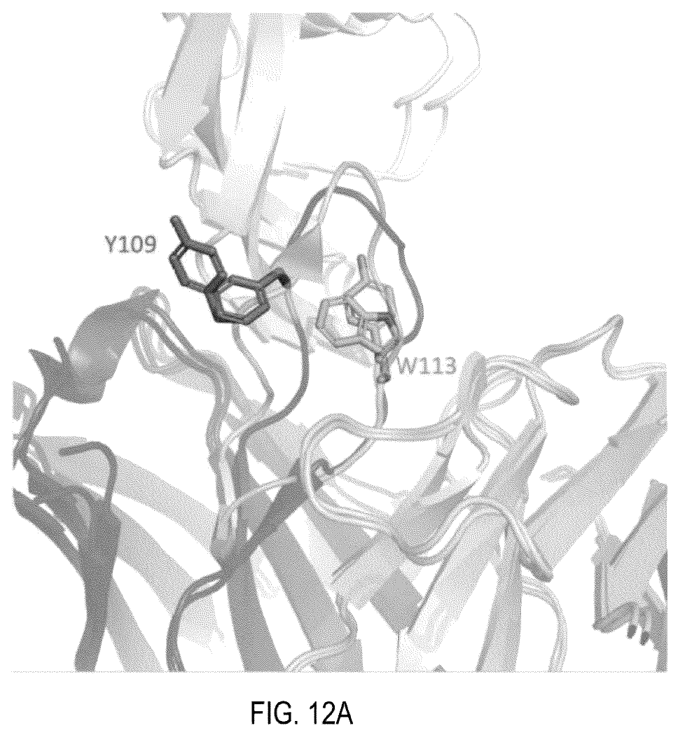

FIG. 12B depicts wildtype binder structure, according to some aspects.

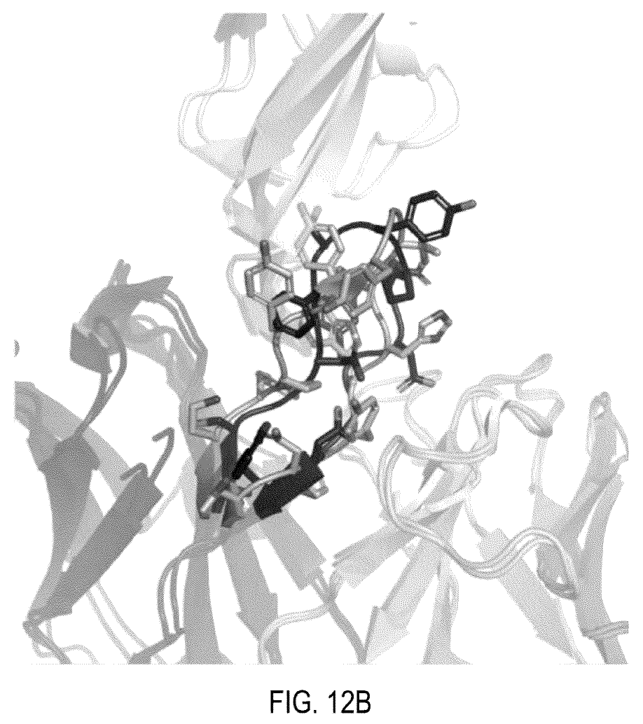

FIG. 13A depicts sensorgrams, according to some aspects, including SEQ ID NOs: 41-48.

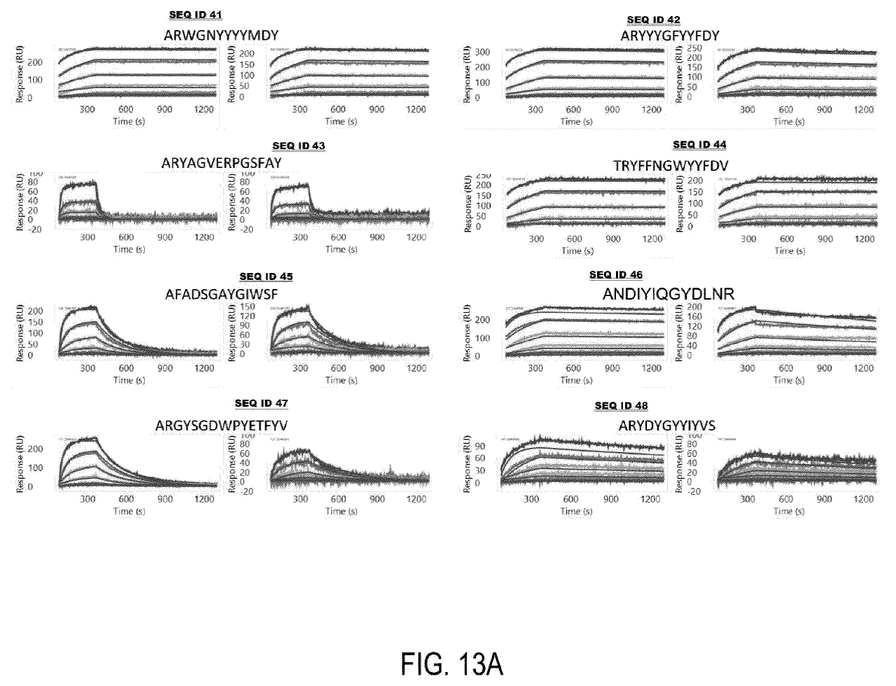

FIG. 13B depicts sensorgrams, according to some aspects.

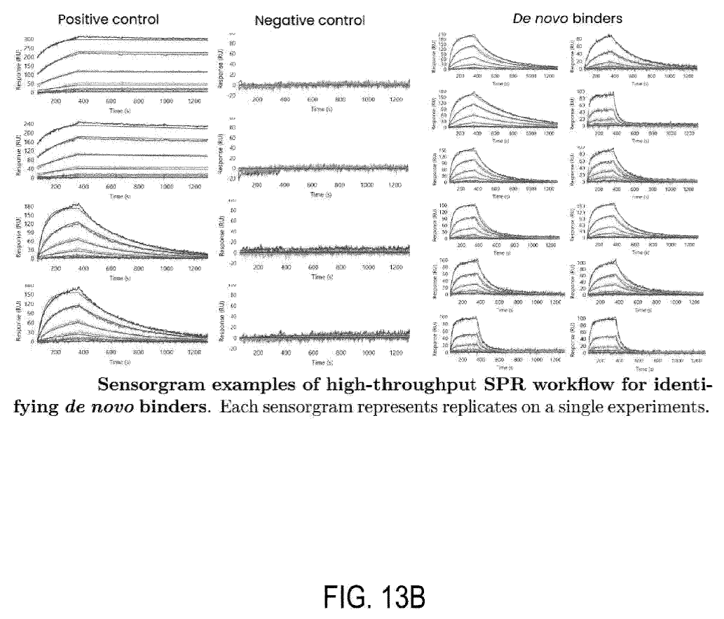

FIG. 13C depicts sensorgrams, according to some aspects.

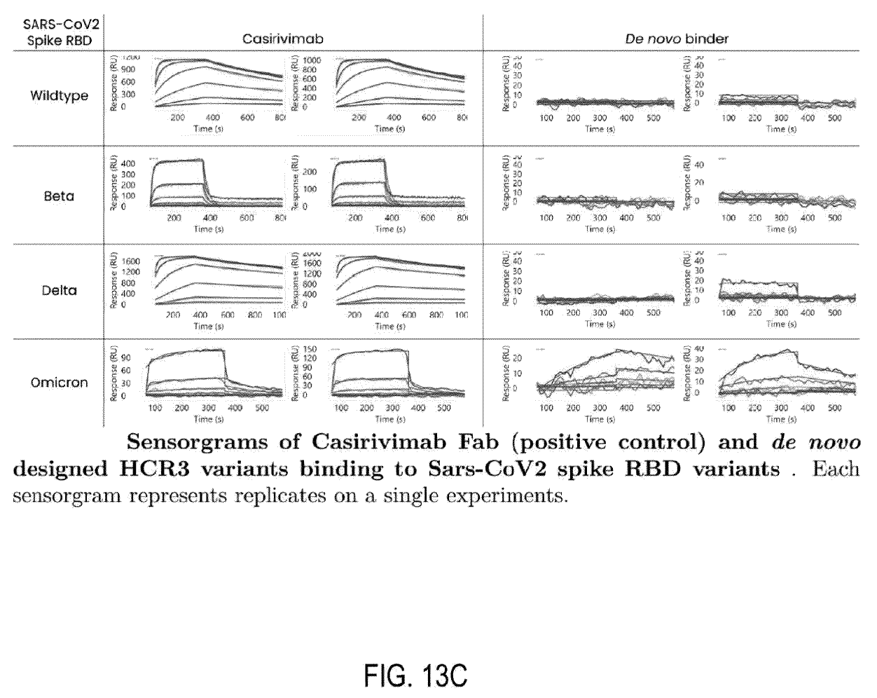

FIG. 13D depicts sensorgrams, according to some aspects.

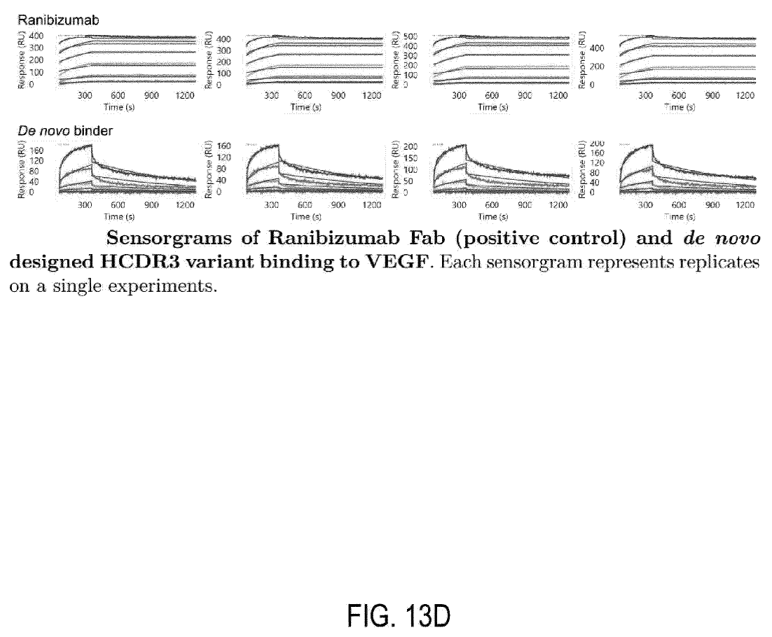

### DETAILED DESCRIPTION

The present disclosure addresses the need for artificial intelligence (AI) and machine learning (ML) models trained to predict biomolecule sequences using known biomolecule/binding partner complexes. In particular, generative AI has the potential to greatly increase the speed, quality and controllability of biomolecule (e.g., antibody) design. The present techniques include using de novo generative deep learning models to de novo design antibodies against three distinct targets in a zero-shot fashion, where all designs are the result of a single round of model generations with no follow-up optimization. In particular, the present techniques may screen a large number (e.g., 400,000 or more) of antibody variants designed for binding to human epidermal growth factor receptor 2 (HER2) using high-throughput wet lab capabilities.

From these screens, the present techniques are able to further characterize 421 binders biophysicallly using surface plasmon resonance (SPR), finding that three bind tighter than the therapeutic antibody trastuzumab. The binders are highly diverse and have low sequence identity to known antibodies. Additionally, these binders score highly on our previously introduced Naturalness metric (see Bachas S, Rakocevic G, Spencer D, Sastry A V, Haile R, Sutton J M, et al. Antibody optimization enabled by artificial intelligence predictions of binding affinity and naturalness. bioRxiv. 2022; doi:10.1101/2022.08.16.504181.), indicating that they are likely to possess desirable developability profiles and low immunogenicity. These results unlock a path to accelerated drug creation for novel therapeutic targets using generative AI combined with high throughput experimentation.

A particularly difficult aspect of antibody drug creation is the initial step of lead candidate identification due to the labor intensive and uncontrolled nature of traditional screening methods. Generative AI-based de novo design has the potential to disrupt these shortcomings of the current drug discovery process. The zero-shot nature of AI design approach obviates the need for cumbersome library screening to identify binding molecules, generating large time and cost savings. Furthermore, the controllable nature of model-based design allows for the creation of proteins optimized for developability and immunogenicity characteristics, mitigating downstream developability risks.

The present techniques provide steps towards fully de novo antibody design by demonstrating the ability to generate, in a zero-shot fashion, novel antibody variants that confer binding and natural sequence characteristics comparable and, in some cases, superior to, the parent antibody. The AI-generated sequences are distinct from any observed in the model training set and the vast majority are distinct from the known sequences in the Observed Antibody Space (OAS) database, yet maintain high Naturalness scores, showing the model can design antibody sequences along a biologically feasible manifold. Furthermore, the designed sequences are highly dissimilar from one another, indicating the ability to design a diverse solution set of binding molecules. Additionally, we demonstrate progress in designing multiple CDRs de novo by creating and validating binders with up to 3 novel heavy chain CDRs using a modified multi-step approach. The present techniques are generalizable, as demonstrated by deployment of the present generative design methods to distinct antigens. Additionally, developing epitope-specificity across multiple antigens for antibody designs may allow for precise interaction with biologically relevant target regions associated with disease mechanisms of action. In addition to advancements on the generative modeling front, the speed and scale of wet lab validation for AI-generated designs will progressively increase as the time and cost of DNA synthesis continues to decline.

Antibodies in particular are a growing class of therapeutic molecules due to their attractive drug-like properties, including high target selectivity and minimal immunogenic effects. Antibody drug development commonly begins with initial lead molecule discovery. Existing approaches for lead discovery typically consist of randomly searching through a massive combinatorial sequence space by screening large libraries of antibody variants against a target antigen. Techniques such as phage, yeast display, immunization coupled with hybridoma screening or B-cell sequencing are typically employed for initial discovery, followed by further molecule development. These methods are time and resource intensive, lack control over the properties of the designed antibodies, and often produce sub-optimal leads. Applying generative artificial intelligence (AI) to design de novo antibodies in a zero-shot and controllable fashion, rather than screening and developing lead molecule may drastically reduce the time and resources necessary for therapeutic antibody development.

The application of AI methods to antibody design, and more generally protein therapeutic design, is compelling given the availability of large protein sequence and structure databases that can fuel model training. Indeed, recent work has shown that models trained on these data could be used for the de novo design of certain classes of proteins. These works screen dozens to thousands of protein designs, representing two to four orders of magnitude fewer proteins than are validated in our study. Moreover, no method has yet achieved de novo design of antibodies with wet-lab validation, despite the immense therapeutic relevance of antibody-based therapeutics which accounted for 30% of FDA approved biologics in 2022. De novo antibody design is of particular interest as the key determinants of antibody function emerge from the complementary determining regions (CDRs) of the sequence. These hyper-variable regions interact directly with the antigen and among these, heavy chain CDR3 (HCDR3) is often most critical to binding but also most variable, making it particularly challenging to model. Several works with experimental validation have attempted to optimize antibodies using supervised learning, though none have attempted zero-shot or de novo design.

Many groups have recognized the potential of zero-shot generative AI to impact antibody design. Several promising methods have recently emerged, leveraging ideas from language modeling to geometric learning, for the design of antibodies. However, no such method has been able to demonstrate de novo antibody design in a zero-shot fashion with validation in the lab. The present techniques integrate generative modeling ideas with high-throughput experimentation capabilities in the wet lab. Recent advancements in DNA synthesis and sequencing, E coli. based antibody expression, and fluorescence-activated cell sorting have made it possible to experimentally assess hundreds of thousands of individual designs rapidly and in parallel.

The present techniques demonstrate zero-shot antibody design with extensive wet lab experimentation. As a first step towards fully de novo antibody design, the present techniques show that HCDR3 can be designed with generative AI methods using a model system of trastuzumab and its target antigen, human epidermal growth factor receptor 2 (HER2), as a model system. All antibodies binding HER2 or homologs of HER2 may be removed from the training set, in some aspects. The present techniques may include de novo design of many (e.g., approximately 440,000 or more) unique HCDR3 variants of trastuzumab and screened for binding to HER2 using a proprietary Activity-specific Cell-Enrichment (“ACE”) assay. As used herein, the term “quantitative affinity Activity-specific Cell-Enrichment or gaACE assay” refers to a high throughput assay for obtaining affinity and sequence data of biomolecule variants (U.S. Provisional Application No. 63/371,474, filed Aug. 15, 2022, and PCT/US23/60167, filed on Jan. 5, 2023, each incorporated by reference in its entirety).

For example, quantitative affinity ACE (“qaACE”) and the ACE analyses described further herein, and known as “de novo ACE” or “dnACE,” are methods for sampling the binding of antibody variants at high throughput using flow cytometry and next generation sequencing. The main goal of this method is to generate high throughput binding information and/or training data for an AI model to perform sequence-based binding predictions. This method can be applied to any antibody format, mabs, fabs, scFv, scFAB, VHH, nanobody etc. and could conceivably be applied to other binding drug formats as well.

In one embodiment, the first step in the gaACE process is to generate a mutationally diverse antibody library, that evenly sample the sequence space around the starting point antibody molecule. This library contains variants that span a range in mutational distance from the original sequence.

In some embodiments including the Examples herein, the method provides a flow cytometry read out of an antibody, expressed in SoluPro E. coli, binding to a fluorescently labeled antigen probe. In the gaACE assay, setting expression of the antibody molecule is normalized such that a change in fluorescent signal in a cell will be due to different affinities of the expressed antibody variants in the cells binding to the fluorescent antigen probe. This normalization is accomplished via a generic target molecule probe that will bind to all variants and whose signal will be in an orthogonal fluorescent channel to the antigen probe. In this setting we show that the fluorescent signal of a variant is proportional to the measured KD of an antibody variant within a range. Given this proportionality, using FACS, cells containing antibody variants can be sorted that span a range (e.g., a distribution) of affinities.

After sorting across a range of affinity values with gating across the library population distribution, the cell material is sequenced and quantified for the prevalence of observed variants across the affinity gates (bins, tubes). Using the quantifications, an enrichment score is calculated for each variant. The enrichment scores generated via gaACE are an ideal data type for AI modeling purposes because of the accuracy and throughput.

In one exemplary workflow, the present disclosure provides a gaACE assay that comprises some or all of the following general steps:
1) Generation of an antibody or other drug molecule library for screening through gaACE expressed in a host cell such as SoluPro E. coli.
2) Identification of an antigen or binding partner probe that is fluorescently labeled for use in, for example, FACS via initial cytometry development process.
3) Use of generic probe to the target molecule variants that will allow for detection of expression level within a cell. This expression signal is used to gate a uniformly expression population to disambiguate affinity and expression signal related to epitope binding signal.
4) Sorting of cells across the affinity distribution.
5) Sequencing of cells sorted across the affinity distribution.
6) During the sequencing DNA barcodes or UMIs may be added via PCR amplification of the region of interest. These UMIs will enable absolute quantification of variants retrieved from the gates.
7) Generation of affinity correlated enrichment scores for each observed variant.
8) AI model training using enrichment score and antibody variant sequence.

Additional ACE Assay Analyses are discussed further below.

From these designs, the present techniques may be used to functionally validate many (e.g., 421 or more) binders using SPR and estimate the presence of yet many more (e.g., approximately 4,000) binders in total. Not only are the designed binders sequence diverse from those found in the training dataset, but they are also highly diverse and dissimilar to anything previously observed in structural antibody databases or massive datasets of known antibodies. Furthermore, according to the previously described Naturalness metric (Bachas S et al.), the designed binders are likely to be developable and possess favorable immunogenicity characteristics. We show the extensibility of our approach by designing and validating binding molecules to two additional antigens; human vascular endothelial growth factor A (VEGF-A) and the SARS-CoV-2 spike protein (COVID-19 Omicron variant).

While the primary focus of this work is the in silico design of HCDR3, fully de novo antibody design will require the generation of multiple antibody CDR regions. We show initial progress toward this goal with a multi-step generative AI approach for designing antibodies with any antibody domain/sequence (e.g., all three heavy chain CDRs (HCDR1, HCDR2, HCDR3), any light chain, etc.) distinct from those of the parental antibody. Taken together, this work paves the way for rapid progress toward fully de novo antibody design using generative AI, which has the potential to revolutionize the availability of therapeutics for patients.

The present techniques represent an important advancement in in silico antibody design with the potential to revolutionize the availability of effective therapeutics for patients. Generative AI-designed antibodies will significantly reduce development timelines by generating molecules with desired qualities without the need for further optimization. Additionally, the controllability of AI-designed antibodies will enable creation of customized molecules for specific disease targets, leading to safer and more efficacious treatments than would be possible by traditional development approaches for patients. The core platform of generative AI design methods and ultra-high throughput wet lab screening capabilities will continue to drive progress on this front, unlocking new capabilities in the rapidly accelerating field of protein therapeutic design.

### Screening Hundreds of Thousands of Model Generated Sequences for Binding

The present techniques leverage our previously described ACE assay to screen massive antibody variant libraries containing hundreds of thousands of members expressed in Fragment antigen-binding (Fab) format. The present techniques validate the ACE assay for the de novo discovery workflow by sampling sequences for follow-up analysis by SPR, a gold standard in binding affinity measurement and detection. Empirical evidence finds that the ACE assay is able to correctly classify binders with nearly 60% precision and >95% recall (Table S3).

TABLE S3
Precision and recall for binary ACE (bACE) assay. The bACE assay
predicted 353 variants would bind in SPR and 209 were confirmed
binders (precision of 59.2%). An additional four binders were
not predicted to bind by bACE (recall of 98.1%)

| Predicted | Verified | Missed | | |
|---|---|---|---|---|
| Binders | Binders | Binders | Precision | Recall |
| 353 | 209 | 4 | 0.592 | 0.981 |

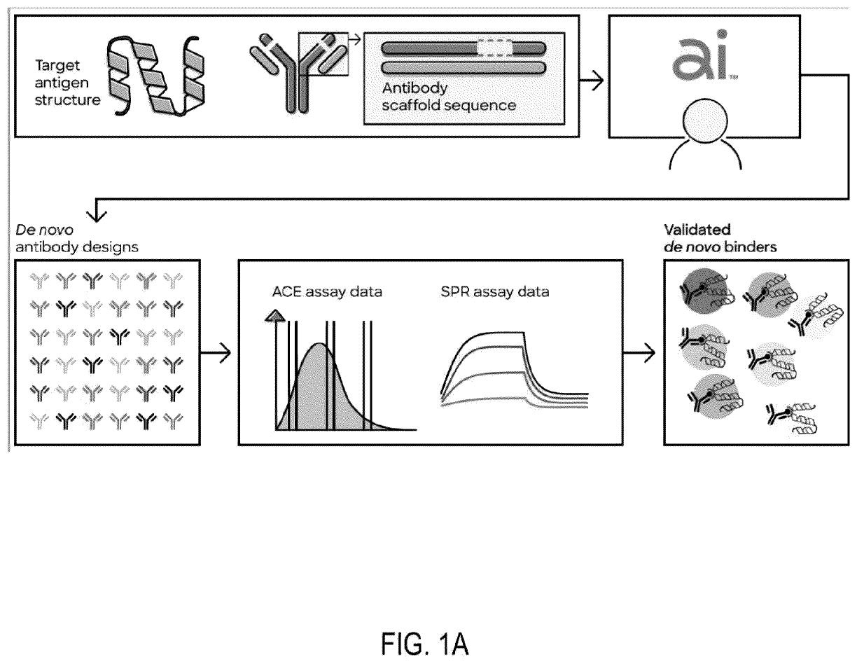

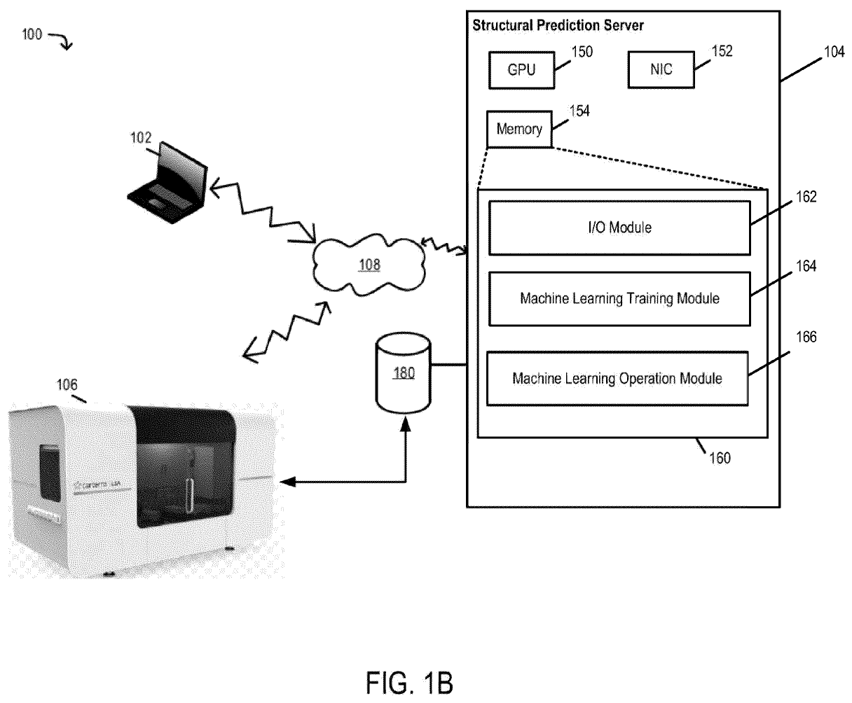

This enables a powerful workflow where a large population of predictions can be initially screened by the ACE assay, and the expected binding population can be subsequently screened via SPR to remove false positives and collect high quality binding affinity measurements (FIG. 1A).

### Exemplary Computer-Implemented Machine Learning Training and Operation

FIG. 1B depicts an exemplary computing environment 100 for training and/or operating one or more machine learning (ML) models, according to some aspects. The environment 100 includes a client computing device 102, a structural prediction server 104, an assay device 106 and an electronic network 108. Some aspects may include a plurality of client devices 102, a plurality of molecular modeling servers 104, and/or a plurality of assay devices 106. Generally, the one or more molecular modeling servers 104 operates to perform training and operation of full or partial in silico molecular modeling as described herein.

The client computing device 102 may be an individual server, a group (e.g., cluster) of multiple servers, or another suitable type of computing device or system (e.g., a collection of computing resources). For example, the client computing device 102 may be any suitable computing device (e.g., a server, a mobile computing device, a smart phone, a tablet, a laptop, a wearable device, etc.). In some aspects, one or more components of the client device 102 may be embodied by one or more virtual instances (e.g., a cloud-based virtualization service) and/or may be included in a respective remote data center (e.g., a cloud computing environment, a public cloud, a private cloud, hybrid cloud, etc.). The client computing device 102 includes a processor and a network interface controller (NIC). The processor may include any suitable number of processors and/or processor types, such as CPUs and one or more graphics processing units (GPUs). Generally, the processor is configured to execute software instructions stored in a memory. The memory may include one or more persistent memories (e.g., a hard drive/solid state memory) and stores one or more set of computer executable instructions/modules. For example, the executable instructions may receive and/or display results generated by the server 104.

The client computing device 102, may include a respective input device and a respective output device. The respective input devices may include any suitable device or devices for receiving input, such as one or more microphone, one or more camera, a hardware keyboard, a hardware mouse, a capacitive touch screen, etc. The respective output devices may include any suitable device for conveying output, such as a hardware speaker, a computer monitor, a touch screen, etc. In some cases, the input device and the output device may be integrated into a single device, such as a touch screen device that accepts user input and displays output. The NIC of the client computing device may include any suitable network interface controller(s), such as wired/wireless controllers (e.g., Ethernet controllers), and facilitate bidirectional/multiplexed networking over the network between the client computing device 102 and other components of the environment 100.

The structural prediction server 104 includes a processor 150, a network interface controller (NIC) 152 and a memory 154. The structural prediction server 104 may further include a data repository 180. The data repository 180 may be a structured query language (SQL) database (e.g., a MySQL database, an Oracle database, etc.) or another type of database (e.g., a not only SQL (NoSQL) database). In some aspects, the data repository 180 may comprise file system (e.g., an EXT filesystem, Apple file system (APFS), a networked filesystem (NFS), local filesystem, etc.), an object store (e.g., Amazon Web Services S3), a data lake, etc. The data repository 180 may include a plurality of data types, such as pretraining data sourced from public data sources (e.g., SAbDab data, OAS data), pre-training data, and fine-tuning data. Fine-tuning data may be proprietary affinity data that is sourced from a quantitative assay ACE, Carterra, or any other suitable source. The data repository 180 may include machine learning model training data represented in any suitable data format(s), such as protein data bank (PDB) format, JavaScript Object Notation (JSON) format, eXtensible Markup Language (XML) format, etc.

The server 104 may include a library of client bindings for accessing the data repository 180. In some aspects, the data repository 180 is located remote from the structural prediction server 104. For example, the data repository 180 may be implemented using a RESTdb.IO database, an Amazon Relational Database Service (RDS), etc. in some aspects. In some aspects, the structural prediction server 104 may include a client-server platform technology such as Python, PHP, ASP.NET, Java J2EE, Ruby on Rails, Node.js, a web service 77 or online API, responsive for receiving and responding to electronic requests. Further, the structural prediction server 104 may include sets of instructions for performing machine learning operations, as discussed below, that may be integrated with the client-server platform technology.

The assay device 106 may be a Surface Plasmon Resonance (SPR) machine, for example, such as a Carterra SPR machine. The device 106 may be physically connected to either the structural prediction server 104 or the data repository 180, as depicted. The device 106 may be located in a laboratory, and may be accessible from one or more computers within the laboratory (not depicted) and/or from the structural prediction server 104. The device 106 may generate data and upload that data to the data repository 180, directly and/or via the laboratory computer(s). The assay device 106 may include instructions for receiving one or more sequences (e.g., mutated sequences) and for synthesizing those sequences. The synthesis may sometimes be performed via another technique (e.g., via a different device or via a human). In some aspects, the device 106 may be configured not as a device, but as an alternative assay that can measure protein-protein interactions as listed in other sections of this application. For example, the device 106 may instead be configured as a suite of devices/workflows, including plates and liquid handling. In general, the device 106 may be substituted with suitable hardware and/or software optionally including human operators to generate affinity data.

The network 108 may be a single communication network, or may include multiple communication networks of one or more types (e.g., one or more wired and/or wireless local area networks (LANs), and/or one or more wired and/or wireless wide area networks (WANs) such as the Internet). The network 108 may enable bidirectional communication between the client computing device 102 and the structural prediction server 104, for example.

The processor 150 may include any suitable number of processors and/or processor types, such as one or more graphics processing units (GPUs), one or more central processing units (CPUs), etc. Generally, the processor 150 is configured to execute software instructions stored in the memory 154. The memory 154 may include one or more persistent memories (e.g., a hard drive/solid state memory) and stores one or more set of computer executable instructions/modules 160, including an input/output (I/O) module 162, a machine learning training module 164 and a machine learning operation module 166. In some aspects, more or fewer modules may be included, and in some aspects, one or more of the models may be combined or aggregated into a fewer number of modules.

Each of the modules 160 implements specific functionality related to the present techniques, as will be described further, below. The modules 160 may store machine readable instructions, including one or more application(s), one or more software component(s), and/or one or more APIs, which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein. In some aspects, a plurality of the modules 160 may act in concert implement a particular technique. For example, the machine learning operation module 166 may load information from one or more other models prior to, during and/or after initiating an inference operation. Thus, the modules 160 may exchange data via suitable techniques, e.g., via inter-process communication (IPC), a Representational State Transfer (REST) API, etc. within a single computing device, such as the structural prediction server 104. In some aspects one or more the modules 160 may be implemented in a plurality of computing devices (e.g., a plurality of servers 104). The modules 160 may exchange data among the plurality of computing devices via a network such as the network 108. The modules 160 of FIG. 1B will now be described in greater detail.

Generally, the I/O module 162 includes instructions that enable a user (e.g., an employee of the company) to access and operate the structural prediction server 104 (e.g., via the client computing device 102). For example, the employee may be a software developer who trains one or more ML models using the ML training module 164 in preparation for using the one or more trained ML models to generate outputs used in an antibody prediction project, a docking complex prediction project, and/or an affinity value prediction project. Once the one or more ML models are trained, the same user (or another) may access the structural prediction server 104 via the I/O module to cause the molecular modeling process to be initiated. The I/O module 162 may include instructions for generating one or more graphical user interfaces (GUIs) (not depicted) that collect and store parameters related to biomolecular modeling, such as a user selection of a particular reference protein, biomolecule, binding partner, etc. from a list stored in the data repository 180.

In general, a computer program or computer based product, application, or code (e.g., the model(s), such as machine learning models, or other computing instructions described herein) may be stored on a computer usable storage medium, or tangible, non-transitory computer-readable medium (e.g., standard random access memory (RAM), an optical disc, a universal serial bus (USB) drive, or the like) having such computer-readable program code or computer instructions embodied therein, wherein the computer-readable program code or computer instructions may be installed on or otherwise adapted to be executed by the processor(s) 150 (e.g., working in connection with the respective operating system in memory 154) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in any desired program language, and may be implemented as machine code, assembly code, byte code, interpretable source code or the like (e.g., via Golang, Python, C, C++, C#, Objective-C, Java, Scala, ActionScript, JavaScript, HTML, CSS, XML, etc.).

The present techniques may include a generative model to automatically design or improve antibodies against a specific target of interest (e.g., a target biomolecule). The model may be trained on antibody-antigen structures or a large collection of extant antibody sequences. The model may also be trained using proprietary high-throughput binding interaction data. The model may be validated, in some aspects, by designing antibodies that bind to a receptor protein implicated in cancer. The present techniques may enable zero-shot generation of antibodies, tailored to bind to any target. The present techniques enable redesigning the complementarity-determining regions (CDRs) of antibodies, in some aspects, which confer binding specificity to the target. The present generative model may automatically design or improve antibodies against a specific target of interest, and may be pre-trained on a collection of antibody-antigen complexes mined from public data sources (e.g., the Protein Data Bank).

In some aspects, a model trained according to the present techniques may be trained to receive an antigen structure, and to predict the structure, sequence, and/or affinity of the antibody based on the received antigen. In some aspects, the prediction may be performed by iteratively refining the model's prediction of the structure while autoregressively unraveling the sequence. In such cases, generalized autoregressive or bidirectional training may be used to capture long-range patterns in the sequence. The antibody and antigen modules may capture independent SE(3) equivariance, enabling data efficient protein-protein rigid body docking.

In some aspects, high GPU memory of 80 GB A100s may be used to achieve unravelling of long structural graphs. Scalable implementations of pairwise SE(3) equivariance can be helpful to improve training efficiency, in some aspects. After pre-training, the present techniques may fine-tune a model to predict the binding affinity of an antibody to an antigen of interest. An affinity prediction module may use structural and sequence features inferred by the model to predict the binding affinity of the antibody to the antigen. In particular, embeddings at each residue may be inferred and propagated to the affinity prediction module with attention. The affinity prediction module may combine high-throughput binding interaction data (e.g., data generated on Absci's Integrated Drug Creation™ platform). Thus, advantageously, the present techniques achieve a novel coupling of structural models of antibody binding with real-world binding affinity data.

The present techniques may validate the trained model by designing antibodies to neutralize Her2, an important target in breast cancer. Starting with trastuzumab, a well understood Her2 binder, the present techniques may include designing libraries containing trastuzumab variants with single, double and higher-order mutations. Binding affinity of variants in the library may be measured by high-throughput screening, and the library scored using generative models, to demonstrate a high correlation between model predictions and the high-throughput data. In addition to pre-training on structural data, or alternatively, the present techniques may pre-train a language model on a large database of extant antibody sequences. The language model and predictive model may be effectively ensembled to further improve predictions of the binding affinity data. After training, the model can generate diverse new antibodies against an unseen antigen by iteratively decoding a sequence autoregressively. In contrast to prior work on deep learning-based protein design in which proteins are designed unconditionally, our model enables targeted design to a specified antigen.

In some aspects, the ML training module 164 may include a set of computer-executable instructions implementing machine learning training, configuration, parameterization and/or storage functionality. The ML training module 164 may initialize, train and/or store one or more ML models, as discussed herein. The trained ML models and their weights/parameters may be stored in the data repository 180 and/or in the memory 154, which is accessible or otherwise communicatively coupled to the structural prediction server 104.

For example, the ML training module 164 may train one or more ML models (e.g., an artificial neural network (ANN)). One or more training data sets may be used for model training in the present techniques, as discussed herein. The input data may have a particular shape that may affect the ANN network architecture. The elements of the training data set may comprise tensors scaled to small values (e.g., in the range of (−1.0, 1.0)). In some aspects, a preprocessing layer may be included in training (and operation) which applies principal component analysis (PCA) or another technique to the input data. PCA or another dimensionality reduction technique may be applied during training to reduce dimensionality from a high number to a relatively smaller number. Reducing dimensionality may result in a substantial reduction in computational resources (e.g., memory and CPU cycles) required to train and/or analyze the input data.

In general, training an ANN may include establishing a network architecture, or topology, adding layers including activation functions for each layer (e.g., a “leaky” rectified linear unit (ReLU), softmax, hyperbolic tangent, etc.), loss function, and optimizer. In an aspect, the ANN may use different activation functions at each layer, or as between hidden layers and the output layer. A suitable optimizer may include Adam and Nadam optimizers. In an aspect, a different neural network type may be chosen (e.g., a graph convolutional neural network, a message passing neural network, a geometric vector perceptron network, a recurrent neural network, a deep learning neural network, etc.). Training data may be divided into training, validation, and testing data. For example, 20% of the training data set may be held back for later validation and/or testing. In that example, 80% of the training data set may be used for training. In that example, the training data set data may be shuffled before being so divided. Dividing the dataset may also be performed in a cross-validation setting, e.g., when the data set is small. Data input to the artificial neural network may be encoded in an N-dimensional tensor, array, matrix, and/or other suitable data structure. In some aspects, training may be performed by successive evaluation (e.g., looping) of the network, using labeled training samples. The process of training the ANN may cause weights, or parameters, of the ANN to be altered. The weights may be initialized to random values. The weights may be adjusted as the network is successively trained, by using one or more gradient descent algorithms, to reduce loss and to cause the values output by the network to converge to expected, or “learned”, values. In an aspect, a regression may be used which has no activation function. Therein, input data may be normalized by mean centering, and a mean squared error loss function may be used, in addition to mean absolute error, to determine the appropriate loss as well as to quantify the accuracy of the outputs.

In some aspects, the ML training module 164 may include computer-executable instructions for performing ML model pre-training, ML model fine-tuning and/or ML model self-supervised training. Model pre-training may be known as transfer learning, and may enable training of a base model that is universal, in the sense that it can be used as a common grammar for all antibody sequences, for example. In some examples, pre-training may be used to train multiple models of independent artificial neural networks, and/or multiple respective layers of a single artificial neural network (e.g., an artificial neural network used to predict affinity values from biological structural information). The term “pre-training” may be used to describe scenarios wherein a second training may occur (i.e., when the model may be “fine-tuned”). Transfer learning refers to the ability of the model to leverage the result (weights) of a first pre-training to better initialize the second training, which may otherwise require a random initialization. The second training, i.e., fine-tuning, may be performed using affinity data as discussed herein. The technique of combining pre-training and fine-tuning advantageously boosts performance, in that the result of the training on affinity data performs better after pre-training (e.g., using natural antibody structures from SAbDab) than when no pre-training is performed. Model fine-tuning may be performed with respect to given antibody-antigen pairs, in some aspects. In some aspects, ML model self-supervised learning may be performed to endow the model with an understanding of the antibody grammar during pre-training.

Generally, an ML model may be trained as described herein using a supervised, semi-supervised or unsupervised machine learning program or algorithm. The machine learning program or algorithm may employ a neural network, which may include one or more of a graph convolutional neural network, a message passing neural network, a geometric vector perceptron network, a recurrent neural network, a deep learning neural network, a convolutional neural network, a deep learning neural network, transformer, autoencoder and/or a combined learning module or program that learns in two or more features or feature datasets (e.g., structured data, unstructured data, etc.). The machine learning programs or algorithms may also include natural language processing, semantic analysis, automatic reasoning, regression analysis, support vector machine (SVM) analysis, decision tree analysis, random forest analysis, K-Nearest neighbor analysis, naive Bayes analysis, clustering, reinforcement learning, and/or other machine learning algorithms and/or techniques (e.g., generative algorithms, genetic algorithms, etc.).

In some aspects, an ML algorithm or techniques may be chosen for a particular input based on the problem set size of the input. In some aspects, the artificial intelligence and/or machine learning based algorithms may be based on, or otherwise incorporate aspects of one or more machine learning algorithms included as a library or package executed on the server(s) 104. For example, libraries may include the TensorFlow based library, the Pytorch library (e.g., PyTorch Lightning), the Keras libraries, the Jax library, the HuggingFace ecosystem (e.g., transformers, datasets and/or tokenizer libraries therein), and/or the scikit-learn Python library. However, these popular open source libraries are a nicety, and are not required. The present techniques may be implemented using other frameworks/languages.

Machine learning may involve identifying and recognizing patterns in existing data (e.g., structural information, docking capabilities, binding affinity, etc.) in order to facilitate making predictions, classifications, and/or identifications for subsequent data (such as using the trained models to generate an antibody based on an input antibody, predict the ability of the generated antibody to dock to the input antigen (or other antigen(s)) and/or predict the binding affinity of the generated antibody). Machine learning model(s), may be created and trained based upon example data (e.g., “training data”) inputs or data (which may be termed “features” and “labels”) in order to make valid and reliable predictions for new inputs. In supervised machine learning, a machine learning program operating on a server, computing device, or otherwise processor(s), may be provided with example inputs (e.g., “features”) and their associated, or observed, outputs (e.g., “labels”) in order for the machine learning program or algorithm to determine or discover rules, relationships, patterns, or otherwise machine learning “models” that map such inputs (e.g., “features”) to the outputs (e.g., labels), for example, by determining and/or assigning weights or other metrics to the model across its various feature categories. Such rules, relationships, or otherwise models may then be provided subsequent inputs in order for the model, executing on the server, computing device, or otherwise processor(s), to predict, based on the discovered rules, relationships, or model, an expected output.

For example, the ML training module 164 may analyze labeled data at an input layer of a model having a networked layer architecture (e.g., an artificial neural network, a convolutional neural network, a deep neural network, etc.) to generate ML models. The training data may be, for example, structural information of antibodies. In some aspects, outputs may be the sequence of a new protein. During training, the labeled data may be propagated through one or more connected deep layers of the ML model to establish weights of one or more nodes, or neurons, of the respective layers. Initially, the weights may be initialized to random values, and one or more suitable activation functions may be chosen for the training process, as will be appreciated by those of ordinary skill in the art. The ML training module 164 may include training a respective output layer of the one or more machine learning models. The output layer may be trained to output a prediction. For example, the ML models trained herein are able to predict structural information of an antibody by analyzing the labeled examples provided during training. In some aspects, the binding affinity may be expressed as a real number (e.g., in a regression analysis). In some aspects, the binding affinity may be expressed as a boolean value (e.g., in classification). In some aspects, multiple ANNs may be separately trained and/or operated. For example, an individual model may be fine-tuned (i.e., trained) based on a pre-trained model, using transfer learning, for a plurality of different antibody-antigen pairs.

In unsupervised or semi-supervised machine learning, the server, computing device, or otherwise processor(s), may be required to find its own structure in unlabeled example inputs, where, for example multiple training iterations are executed by the server, computing device, or otherwise processor(s) to train multiple generations of models until a satisfactory model is generated. In the present techniques, semi-supervised learning may be used, inter alia, for natural language processing purposes and to learn a grammar of antibody sequences using an objective, such as a masked language model objective. Supervised learning and/or unsupervised machine learning may also comprise retraining, relearning, or otherwise updating models with new, or different, information, which may include information received, ingested, generated, or otherwise used over time. In various aspects, training the ML models herein may include generating an ensemble model comprising multiple models or sub-models, comprising models trained by the same and/or different AI algorithms, as described herein, and that are configured to operate together.

Once the model machine learning training module 164 has initialized the one or more ML models, which may be ANNs or regression networks, for example, the model machine learning training module 164 trains the ML models by inputting labeled data into the models (e.g., antibody/antigen complexes; docked biomolecules; etc.). The trained ML model provides accurate predictions given inputs previously unseen by the model (i.e., not used during training).

The model machine learning training module 164 may divide training data into a respective training data set and testing data set. The model machine learning training module 164 may train the ANN using the labeled data. The model machine learning training module 164 may compute accuracy/error metrics (e.g., cross entropy) using the test data and test corresponding sets of labels. The model machine learning training module 164 may serialize the trained model and store the trained model in a database (e.g., the data repository 180). Of course, it will be appreciated by those of ordinary skill in the art that the model machine learning training module 164 may train and store more than one model. For example, the model machine learning training module 164 may train an individual model for performing antibody generation, another for performing antibody docking, and still another for performing affinity prediction. It should be appreciated that the structure of the network as described may differ, depending on the embodiment. For example, in some aspects, antibody generation, antibody docking and affinity prediction may each correspond to respective layers of a monolithic machine learning model. For example, in some aspects, the trained ML model(s) predict many pieces of data: the sequence of the antibody, the affinity of the antibody, the docking configuration. The present techniques enable supervising any one of these components and the remaining components can via training improve. This capability is an advantageous result of the end-to-end training of the present techniques.

The machine learning operation module 166 may include a set of computer-executable instructions implementing machine learning loading, configuration, initialization and/or operation functionality. The ML operation module 166 may include instructions for storing trained models (e.g., in the electronic data repository 180, as a pickled binary, etc.). Once trained, a trained ML model may be operated in inference mode, whereupon when provided with de novo input that the model has not previously been provided, the model may output one or more predictions, classifications, etc. as described herein. In some aspects, a loss minimization function may be used, for example, to teach a ML model to generate output that resembles known output (i.e., ground truth exemplars).

Once the model(s) are trained by the model machine learning training module 164, the model operation module 166 may load one or more trained models (e.g., from the data repository 180). The model operation module 166 generally applies new data that the trained model has not previously analyzed to the trained model. For example, the model operation module 166 may load a serialized model, deserialize the model, and load the model into the memory 154. The model operation module 166 may load new data (e.g., binding partner structural information) that was not used to train the trained model. For example, the new data may include data (e.g., antigen sequence data, etc.) as described herein, encoded as input tensors. The model operation module 166 may apply the one or more input tensor(s) to the trained ML model. The model operation module 166 may receive output (e.g., tensors, feature maps, etc.) from the trained ML model. The output of the ML model may include structural information of one or more biomolecules (e.g., antibodies) predicted to have good docking and high binding affinity with the input binding partner (e.g., an antigen). The output of the ML model may include docking complexes and binding affinity values. In this way, the present techniques advantageously provide a means, for example, of quantitatively designing a target antibody corresponding to an input target antigen, while also quantifying docking of the input antigen and designed target antibody, and quantifying the binding affinity of the two. These techniques are far more accurate and data rich than conventional industry practices. By using ML, the present techniques avoid the expense and time consuming process of laboratory experimentation due to in silico performance, rather than requiring continued use of the wet lab.

The model operation module 166 may be accessed by another element of the structural prediction server 104 (e.g., a web service). The ML operation module 166 may pass its output to another module for further processing/analysis. In some aspects, a user may interact with the ML model during training and/or operation using a command line tool, an Application Programming Interface (API), a software development kit (SDK), a Jupyter notebook, etc.

Regarding the modules 160, it will be appreciated by those of ordinary skill in the art that in some aspects, the software instructions comprising the module 160 may be organized differently, and more/fewer modules may be included. For example, one or more of the modules 160 may be omitted or combined. In some aspects, additional modules may be added (e.g., a localization module). In some aspects, software libraries implementing one or more modules (e.g., Python code) may be combined, such that, for example, the ML training module 164 and ML operation module 166 are a single set of executable instructions used for training and making predictions.

### Exemplary ACE Assay Analysis

In operation, the assay device 106 may be used to produce binding scores from reads. For example, in an embodiment, the following procedures may be used:

1. Paired-end reads may be merged using FLASH2 with a maximum allowed overlap set according to the amplicon size and sequencing reads length (e.g., 150 bases for all the libraries described herein).
2. Primers may be removed from both ends of the merged read using cutadapt tool, and reads were discarded where primers were not detected.
3. Reads may be aggregated across all FACS sorting gates and aligned to the reference sequence (parental version of the amplicon) in amino acid space. Alignment may be performed using the Needleman-Wunsch algorithm implemented in Biopython. In an example, the following parameters may be used: PairwiseAligner, mode=global, match_score=5, mismatch_score=−4, open_gap_score=−20, extend_gap_score=−1. Parameters were may be chosen by manual inspection across a number of processed libraries, in some aspects.
4. Reads may then be discarded if (1) the mean base quality is below a number (e.g., 20), or (2) a sequence (in DNA space) is seen in fewer than a number (e.g., 10) of reads across all gates.
5. The present techniques may further include flagging: (1) sequences that align to the reference with a low score (e.g., defined as less than 0.6 of the score obtained by aligning the reference to itself); (2) sequences containing stop codons outside of the region of interest and (3) sequences containing frame-shifting insertions or deletions. Flagged sequences may not be included in any mutation-related statistics, but may be used for count normalization for binding score calculations, in some aspects. FastQC and MultiQC may be used to generate sequencing quality control metrics.
6. For each gate, the prevalence of each sequence (read count relative to the total number of reads from all sequences in that gate) may be normalized to a number (e.g., 1 million) of counts.
7. The binding score (e.g., ACE score) may be assigned to each unique DNA sequence by taking a weighted average of the normalized counts across the sorting gates. In all experiments, weights may be assigned linearly using an integer scale: the gate capturing the lowest fluorescence signal was assigned a weight of 1, the next lowest gate was assigned a weight of 2, etc.
8. Any detected sequence which was not present in the originally designed and synthesized library may be dropped.
9. ACE scores may be averaged across independent FACS sorts, dropping sequences for which the standard deviation of replicate measurements is greater than 1.25. An amino acid variant may be retained only if the present techniques collect a number (e.g., at least three) independent QC-passing observations between synonymous DNA variants and replicate FACS sorts.
Exemplary Binary Assay (dnACE) Analysis

Enrichment scores may be calculated for individual variants screened by a binary version of the ACE assay. E.g., the following procedure may be used, in some embodiments:

1. Paired-end reads may be merged using Fastp with quality filtering and base correction in merged regions enabled.
2. Primers may be removed from both ends of the merged read using cutadapt tool, and reads discarded where primers were not detected.
3. Unique sequences may be tallied to provide raw counts of each variant observed in each sample. Sequences that do not match a designed sequence in the library may be discarded.
4. For each sample, proportional abundances may be calculated for each variant. Enrichment scores may be calculated by dividing the proportional abundance of each variant in a gate by its proportional abundance in the unsorted library sample.

### ACE Assay Expected Binding Rate Computation

ACE scores are discretized into a set of 6 bins and for each bin a set of variants belonging to that bin are screened via SPR. The percentage of a bin's variants that bind is used to estimate the expected binding rate for said bin. These values can be seen in Table S2.

TABLE S2
SPR binding rates corresponding to ACE scores of sequences in HDL2.
The binding rate in SPR increases as the sequences fall into higher
ACE bins. This validates the ACE assay as a useful method to select
binders (i.e. select sequences from the highest gates). The data can
also be used to infer the total number of binders in each bin.

| | Sequences screened | Number of | |
|---|---|---|---|
| ACE score range | in SPR | binders | Binding rate in SPR |
| [0, 1) | 6 | 134 | 0.045 |
| [1, 2) | 13 | 67 | 0.194 |
| [2, 3) | 24 | 46 | 0.522 |
| [3, 4) | 41 | 44 | 0.932 |
| [4, 5) | 117 | 128 | 0.914 |
| [5, 6) | 180 | 191 | 0.942 |

### Exemplary Surface Plasmon Resonance (SPR) Aspects

As discussed above, SPR may be used for various techniques, including functional validation of binders.

### Surface Preparation

For example, in some aspects, post induction samples may be transferred to plates (e.g., 96-well plates (e.g., Greiner Bio-One)), pelleted and lysed in 50 μL lysis buffer (e.g., 1× BugBuster protein extraction reagent containing 0.01 KU Benzonase Nuclease and 1× Protease inhibitor cocktail). Plates may be incubated for 15-20 min at 30° C. then centrifuged to remove insoluble debris. After lysis, samples may be adjusted with 200 μL SPR running buffer (e.g., 10 mM HEPES, 150 mM NaCl, 3 mM EDTA, 0.01% w/v Tween-20, 0.5 mg/mL BSA) to a final volume of 260 μL and filtered into 96-well plates. Lysed samples may then be transferred from 96-well plates to larger plates (e.g., 384-well plates) for high-throughput SPR, for example, using a Hamilton STAR automated liquid handler. Colonies may be prepared in two sets of independent replicates prior to lysis and each replicate measured in two separate experimental runs. In some instances, single replicates may be used, as indicated.

### SPR

High-throughput SPR experiments may be conducted on a microfluidic Carterra LSA SPR instrument using SPR running buffer (e.g., 10 mM HEPES, 150 mM NaCl, 3 mM EDTA, 0.01% w/v Tween-20, 0.5 mg/mL BSA) and SPR wash buffer (e.g., 10 mM HEPES,

150 mM NaCl, 3 mM EDTA, 0.01% w/v Tween-20). Carterra LSA SAD200M chips may be pre-functionalized with 20 μg/mL biotinylated antibody capture reagent for 600 s prior to conducting experiments. Lysed samples in 384-well blocks may be immobilized onto chip surfaces for 600 s followed by a 60 s washout step for baseline stabilization.

Antigen binding may be conducted using the non-regeneration kinetics method with a 300 s association phase followed by a 900 s dissociation phase. For analyte injections, six leading blanks may be introduced to create a consistent baseline prior to monitoring antigen binding kinetics. After the leading blanks, five concentrations of HER2 extracellular domain antigen (e.g., ACRO Biosystems, prepared in three-fold serial dilution from a starting concentration of 500 nM), may be injected into the instrument and the time series response recorded. In some experiments, measurements on individual DNA variants may be repeated four times. Each experiment run may consist of two complete measurement cycles (ligand immobilization, leading blank injections, analyte injections, chip regeneration) which may provide two duplicate measurement attempts per clone per run. In some experiments, technical replicates measured in separate runs may further double the number of measurement attempts per clone to four.

### Exemplary Sequencing Techniques

### Low-Diversity Library Sequencing

To identify the DNA sequence of individual antibody variants evaluated by SPR, duplicate plates may be provided for sequencing. A portion of the pelleted material may be transferred into 96 well PCR (e.g., Thermo-Fisher) plate via pinner (e.g., Fisher Scientific) which may contain reagents for performing an initial phase PCR of a two-phase PCR for addition of Illumina adapters and sequencing. Reaction volumes used may be 12.5 μl, for example. During the initial PCR phase, partial Illumina adapters may be added to the amplicon via 4 PCR cycles. The second phase PCR may add the remaining portion of the Illumina sequencing adapter and the Illumina i5 and i7 sample indices. The initial PCR reaction may be used, for example, 0.45 μM UMI primer concentration, 6.25 μl Q5 2× master mix (NEB). Reactions may be initially denatured at 98° C. for 3 min, followed by 4 cycles of 98° C. for 10 s; 59° C. for 30 s; 72° C. for 30 s; with a final extension of 72° C. for 2 min. Following the initial PCR, 0.5 μM of the secondary sample index primers may be added to each reaction tube.

Reactions may then be denatured at 98° C. for 3 min, followed by 29 cycles of 98° C. for 10 s; 62° C. for 30 s; 72° C. for 15 s; with a final extension of 72° C. for 2 min. Reactions may then be pooled into a 1.5 mL tube (Eppendorf). Pooled samples may be size selected with a 1× AMPure XP (Beckman Coulter) bead procedure. Resulting DNA samples may be quantified by Qubit fluorometer. Pool size may be verified via Tapestation 1000 HS and sequenced on an Illumina MiSeq Micro (2×150 nt) for HCDR3 libraries or an Illumina MiSeq Reagent Kit v3 (2×300 nt) for HCDR1-HCDR3 libraries with 20% PhiX, in some aspects.

After sequencing, amplicon reads may be merged using Fastp, trimmed by cutadapt [44] and each unique sequence enumerated. Next, custom R scripts may be applied to calculate sequence frequency ratios between the most abundant and second-most abundant sequence in each sample. Levenshtein distance may be calculated between the two sequences. These distance values may be used for downstream filtering to ensure a clonal population was measured by SPR. The dominant sequence within each sample may be compared to the designed sequences and discarded if it does not match any expected sequence. Dominant sequences may then be combined with their companion Carterra SPR measurements.

### Exemplary Computer-Implemented Methods

In some aspects, the present techniques may include training one or more machine learning models to generate target biomolecule structures and/or sequences.

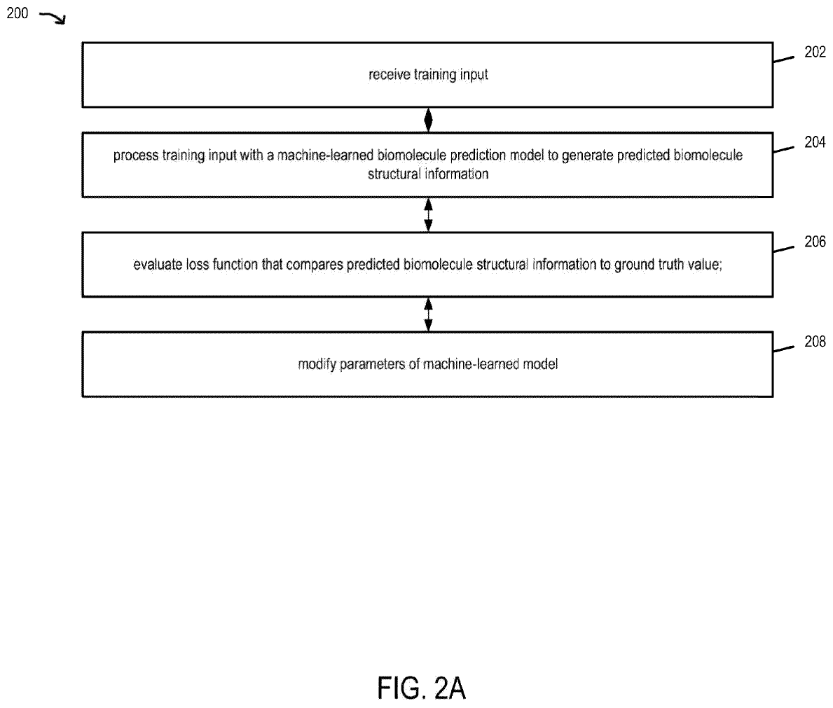

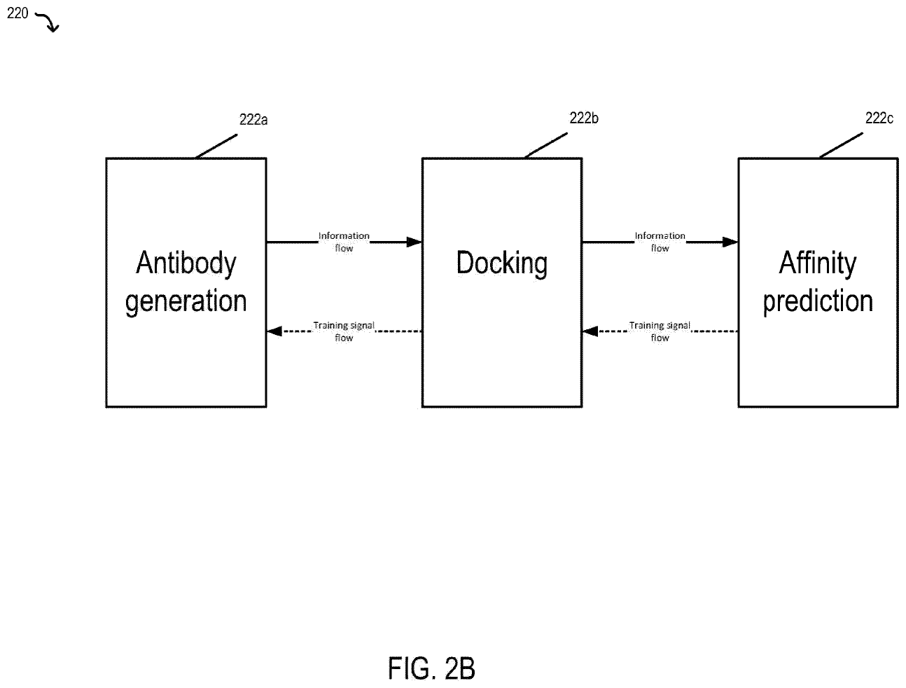

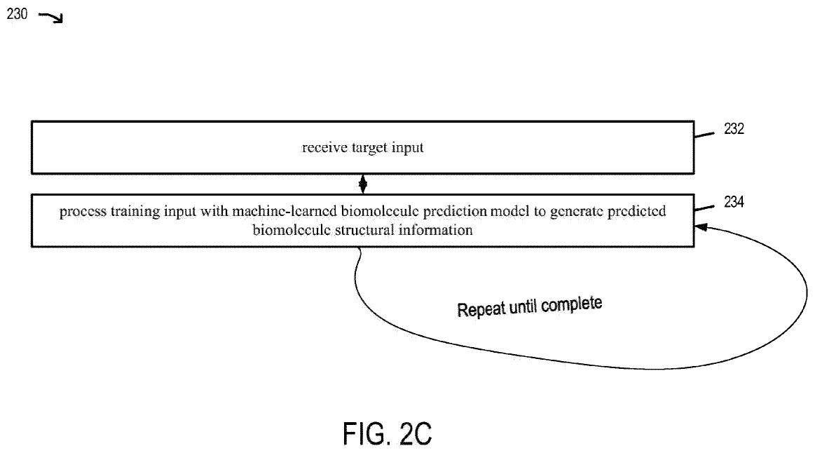

FIG. 2A depicts an exemplary computer-implemented method 200 for training a machine learning model to generate structural information of a biomolecule, according to some aspects. The method 200 may include receiving one or more training inputs, including one or more of (i) input biomolecule structural information, (ii) input biomolecule binding partner structural information or (iii) input biomolecule-input binding partner binding complex structural information (block 202).

Generally, the one or more training inputs may include data representing one or more of: (i) an amino acid; (ii) a peptide sequence; (iii) a polypeptide sequence; (iv) a primary sequence; (v) one or more secondary structures; (vi) one or more tertiary structures; (vii) one or more quaternary structures; or (viii) three-dimensional coordinates of a primary sequence, corresponding to an input biomolecule, an input biomolecule binding partner, or an input biomolecule-input binding partner binding complex.

The method 200 may include processing the one or more training inputs with a machine-learned biomolecule prediction model to generate predicted biomolecule structural information (block 204). The method 200 may include evaluating a loss function that compares the predicted biomolecule structural information to a ground truth value (block 206). The method 200 may include modifying one or more values of one or more parameters of the machine-learned model based at least in part on the loss function (block 208). The method 200 may be performed by the ML training module 164 of FIG. 1, in some aspects.

In some aspects, evaluating the loss function that compares the predicted biomolecule structural information to the ground truth value to train the machine-learned model at block 206 may include configuring a second model acting as a critic to train the machine-learned model to predict according to the ground truth data. Such techniques as adversarial architectures (e.g., generative adversarial networks) may be used in this context.

In some aspects, the biomolecule may be an antibody or protein. In some aspects, the biomolecule structural information comprises three-dimensional coordinates of a primary sequence. In some aspects, the biomolecule binding partner is an antigen, receptor, ligand or a cell membrane. In some aspects, the training inputs are represented, respectively, as at least one of: (i) a protein data bank (PDB) data format, (ii) a JSON data format or (iii) an XML data format. Other suitable formats may be used, in some aspects. In some aspects, the biomolecule binding partner is a protein, and the biomolecule binding partner structural information comprises three-dimensional structure. In some aspects, the method 200 may further include receiving the training inputs from a database comprising antibody structures and/or antigen structures. For example, the training inputs may be received/retrieved from a structural antibody database (SAbDab). It will be appreciated by those of ordinary skill in the art that data processing steps may be helpful for performing the present techniques. For example, to assess whether an ML model can generalize to particular binding partners (e.g., antigens) that are very different from what the model has been trained with, all biomolecules in the training set may be removed that are within 40% sequence identity to any protein in corresponding validation and testing sets.

FIG. 2B depicts a data flow block diagram 220 for training and operating one or more machine learning models, according to some aspects of the present techniques. In the diagram 220, antibody generation step occurs at block 222a. During training, as depicted, training information flows from the antibody generation block 222a to the docking block 222b, wherein a docking step occurs. Training information also flows from the docking block 222b to the affinity prediction block 222c, wherein affinity prediction occurs. During training, training signals also flows in reverse, as depicted. For example, FIG. 2B may depict the flow of data during training using the method 200 of FIG. 2A. In some aspects, training and/or operation of the one or more antibody generation machine learning models may be performed as discussed in “Iterative Refinement Graph Neural Network for Antibody Sequence-Structure Co-design” Jin et al., arXiv:2110.04624 [q-bio.BM]. However, as will be appreciated by those of ordinary skill in the art, this technique lacks the use of the input binding partner (e.g., antigen). In some aspects, docking may be performed as discussed in “Independent SE(3)-Equivariant Models for End-to-End Rigid Protein Docking,” Ganea et al., arXiv:2111.07786 [cs.AI].

FIG. 2C depicts a computer-implemented method 230 for generating structural information of a target biomolecule, according to some aspects. The method 230 may include receiving a target input including one or more of a target binding partner sequence, a target binding partner, or a target epitope (block 232). The method 230 may further include predicting the structural information of the target biomolecule by processing the target input with the biomolecule prediction model (block 234). In some aspects, the structural information is represented in the computer-readable media as at least one of: (i) a protein data bank (PDB) data format, (ii) a JSON data format or (iii) an XML data format. In some aspects, the machine-learned biomolecule prediction model of the method 200 and/or the method 230 is an artificial neural network. The artificial neural network may include at least one of a geometric neural network, a transformer network or a geometric vector perceptron network.

Generally, the structural information of the target biomolecule includes data representing one or more of: (i) an amino acid of the target biomolecule; (ii) a peptide sequence of the target biomolecule; (iii) a polypeptide sequence of the target biomolecule; (iv) a primary sequence of the target biomolecule; (v) one or more secondary structures of the target biomolecule; (vi) one or more tertiary structures of the target biomolecule; (vii) one or more quaternary structures of the target biomolecule; or (viii) three-dimensional coordinates of a primary sequence of the target biomolecule.

The method 230 may include storing (e.g., in the memory 154, the data repository 180, etc.) and/or outputting data representing a portion or all of the structural information of the biomolecule. For example, the data may represent the one or more of: (i) the amino acid of the target biomolecule; (ii) the peptide sequence of the target biomolecule; (iii) the polypeptide sequence of the target biomolecule; (iv) the primary sequence of the target biomolecule; (v) the one or more secondary structures of the target biomolecule; (vi) the one or more tertiary structures of the target biomolecule; (vii) the one or more quaternary structures of the target biomolecule; or (viii) the three-dimensional coordinates of a primary sequence of the target biomolecule. In some aspects, the polypeptide sequence of the target biomolecule corresponds to a complementarity determining region of an antibody. In some aspects, the structural information of the target biomolecule is selected from the group consisting of: an alpha helix, a beta pleated sheet, and a coil. In some aspects, the target biomolecule is an antibody and the target binding partner is an antigen. The method 230 may include repeating block 234 until the structural information of the target biomolecule is complete. In some aspects, the method 230 may be performed iteratively.

In some aspects, the method 230 may include receiving an input parameter specifying a desired length of amino acids of the target biomolecule; and generating a target biomolecule having the desired length. In some aspects, the machine-learned biomolecule prediction model may determine the length by predicting an end-of-sequence token. In some aspects, the method 230 may include updating the structural information of the target biomolecule by folding one or more proteins. In some aspects, the target binding partner is at least one of (i) a primary amino acid sequence, (ii) an antigen epitope and/or (iii) a known three-dimensional structure of an antigen. In some aspects, the structural information of the target biomolecule is predicted in at least one of (i) N-terminus to C-terminus order, (ii) C-terminus to N-terminus order, or (iii) via random sampling. Advantageously, the present techniques are not limited to predicting in any fixed order.

In some aspects, the method 230 may further include operating and/or training a machine-learned docking model trained to generate docked complexes from two or more input three-dimensional biomolecules. In some aspects, the method 230 may further include processing the target input and the predicted structural information of the target biomolecule using the docking model to generate a docked complex comprising the target input and the target biomolecule. In some aspects, the method 230 may include providing the docked complex as a docked complex output. In some aspects, the method 230 may further include operating and/or training a machine-learned binding affinity prediction model trained to predict binding affinity from input docked complexes. The method 230 may include processing the docked complex using the trained affinity prediction model to generate a binding affinity value. In some aspects, the machine-learned model is an artificial neural network. In some aspects, the artificial neural network is at least one of a graph convolutional neural network, a message passing neural network, or a geometric vector perceptron network. In some aspects, the method 230 may include summarizing node information contained within the artificial neural network using a pooling operation to generate the binding affinity value. In some aspects, the method 230 may include providing the docked complex as a docked complex output. In some aspects, the machine learned model may be trained to predict binding specificity in addition to, or alternate from, binding affinity.

In some aspects, the method 230 may include training the machine-learned biomolecule prediction model by providing sequence data as input to teacher force the biomolecule prediction model; training the docking model by providing antibody structure data as input to teacher force the docking model; and/or training the affinity prediction model by providing sequence data as input to teacher force the affinity prediction model. Specifically, the biomolecule prediction model may be designing one or more new antibodies. Early in training, the design capabilities may not be performant (e.g., the model may never get to a point where the training signal is meaningful). With teacher forcing, the problem is made easier. At each stage, the model is trained to look at the last ground truth position, causing the model to focus on the most important information at teach training stage.

The models may be trained using antibody/antigen structures (e.g., antibody/antigen complexes in a protein databank, such as SAbDab). The models may be configured to emit an initial guess as to a drug molecule or antibody. The guess may be generated by instructions that iteratively output one residue or structure at a time, based on an input binding partner (e.g., antigen). Initially, the trained model may predict a first structure of the target biomolecule without knowing the corresponding sequence, but while knowing the target antigen (e.g., provided as input to the trained model). The ML model may iteratively predict a first amino acid and update the target biomolecule's structure, then a second amino acid, and so on until the target biomolecule's structure is complete, or until another stopping condition occurs. Stopping conditions may include the trained model having made a prediction for each corresponding input, a convergence criteria, filling of a portion of a CDR, etc. The predictions of the trained ML model may work in any direction, including (i)N-terminus to C-terminus order, (ii) C-terminus to N-terminus order, or (iii) via random sampling. The antigen provided to the trained ML model as input may be a primary amino acid sequence, an antigen epitope and/or a known antigen three-dimensional structure, optionally including an antigen and epitope. The model is capable of designing an entire structure, or a portion thereof (e.g., a CDR3). However, empirical research may demonstrate that better results have shown that coarsening the residues or averaging the residues may be less performant, that is, not comparable to aspects of the present techniques that analyze the entire structure (e.g., an entire antibody).

In some aspects, the method 230 may include training a machine-learned affinity prediction artificial neural network, including: (i) one or more biomolecule prediction layers trained to predict biomolecule structural information from target inputs; (ii) one or more docking layers trained to generate docked complexes from two or more input three-dimensional biomolecules; and (iii) one or more affinity prediction layers trained to predict affinity from input docked complexes. The one or more biomolecule prediction layers, the one or more docking layers, and the one or more affinity prediction layers may be connected (fully or otherwise). The method 230 may include receiving a target input comprising one or more of a target binding partner sequence, a target binding partner, or a target epitope; and processing the target input using the affinity prediction artificial neural network to generate a docked complex corresponding to the target input and a corresponding structural affinity value. In some aspects, the method 230 may include receiving a sequence affinity prediction value from an affinity sequencing prediction model; and average the structural affinity value with the sequence affinity prediction value to generate an ensemble affinity prediction value.

In some aspects, the method 230 may include training the machine learned affinity prediction artificial neural network (or layers thereof) to make diverse generations. For example, given an antigen, the prediction ANN may make millions of antibodies. In order to rank/filter those and choose the ones that we think are most likely to work, the present techniques may include ranking the antibodies using other techniques, including those described in U.S. Provisional Patent Application No. 63/297,679; incorporated by reference in its entirety herein.

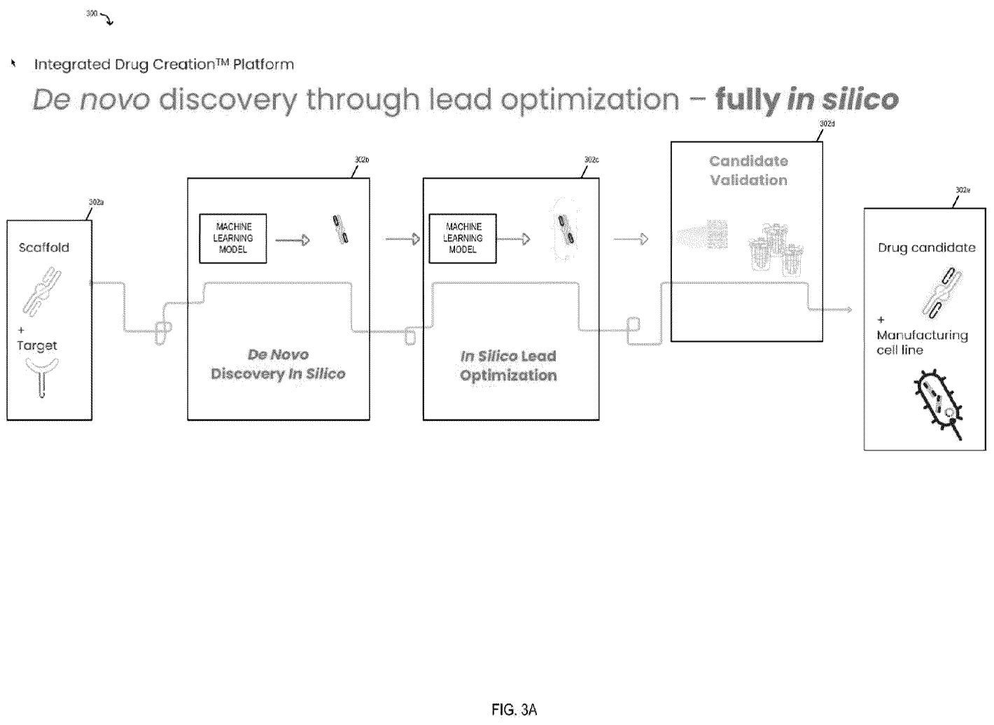

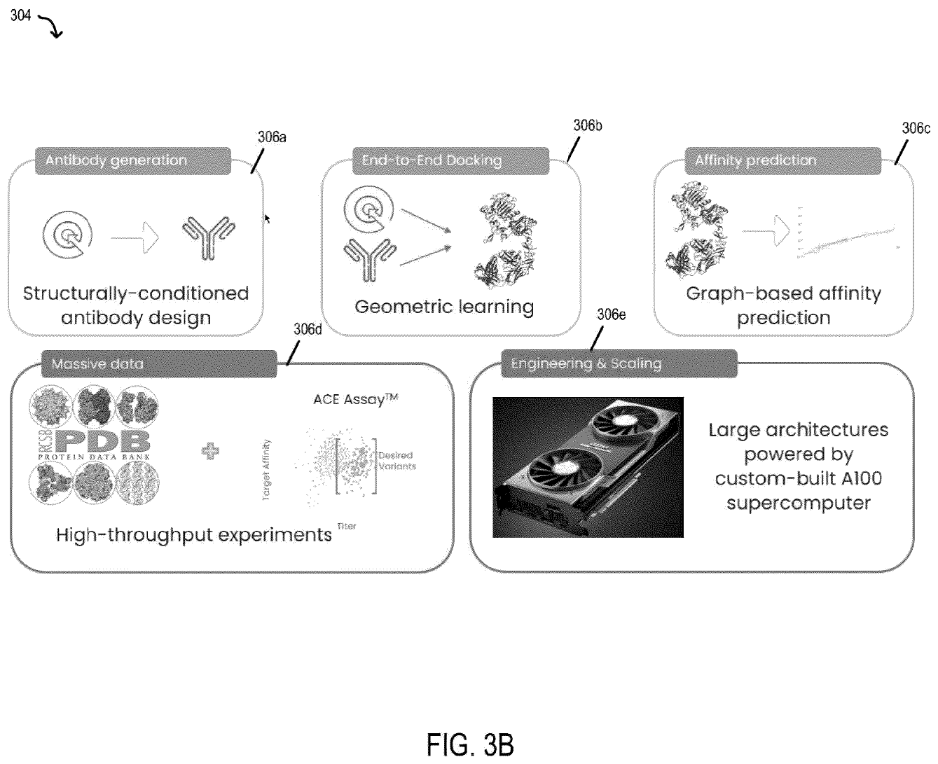

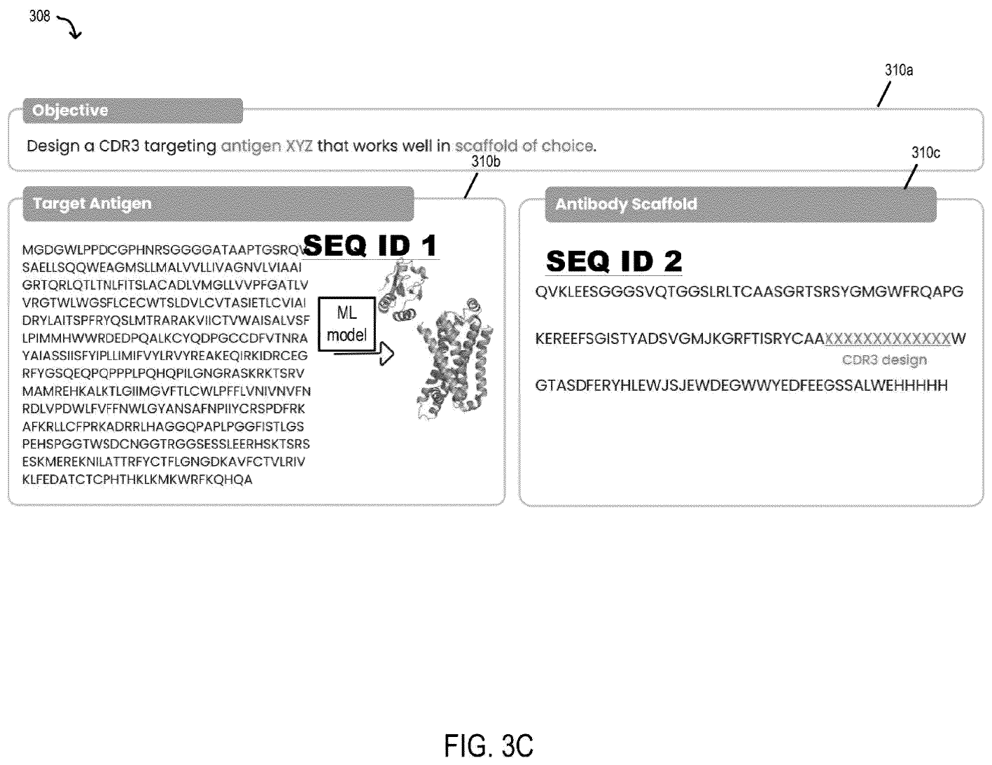

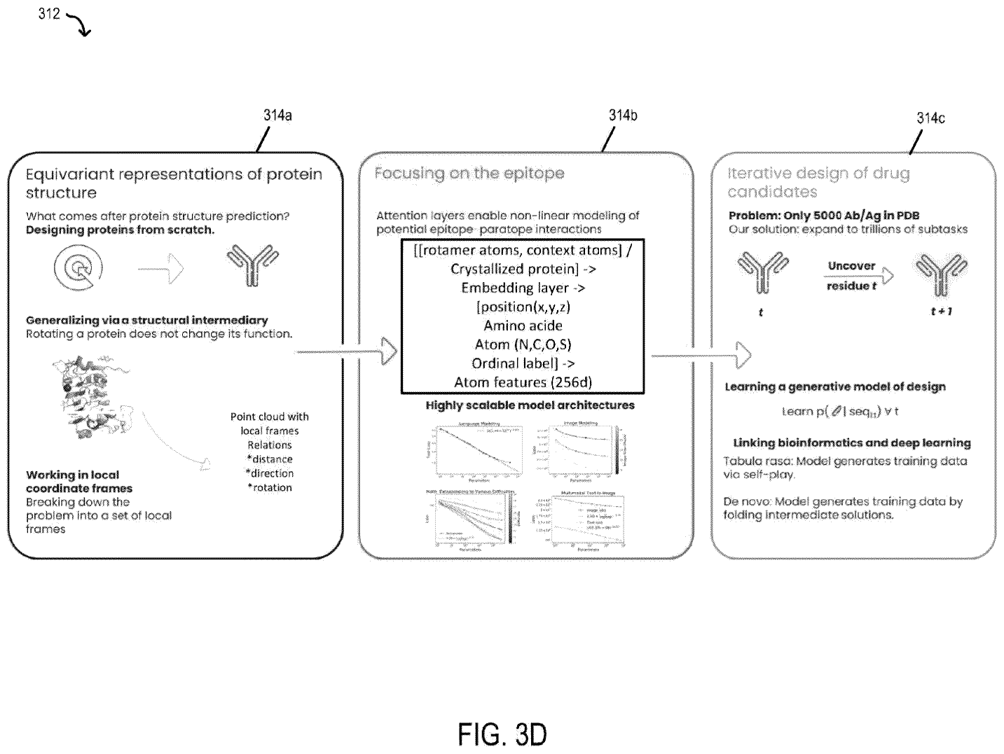

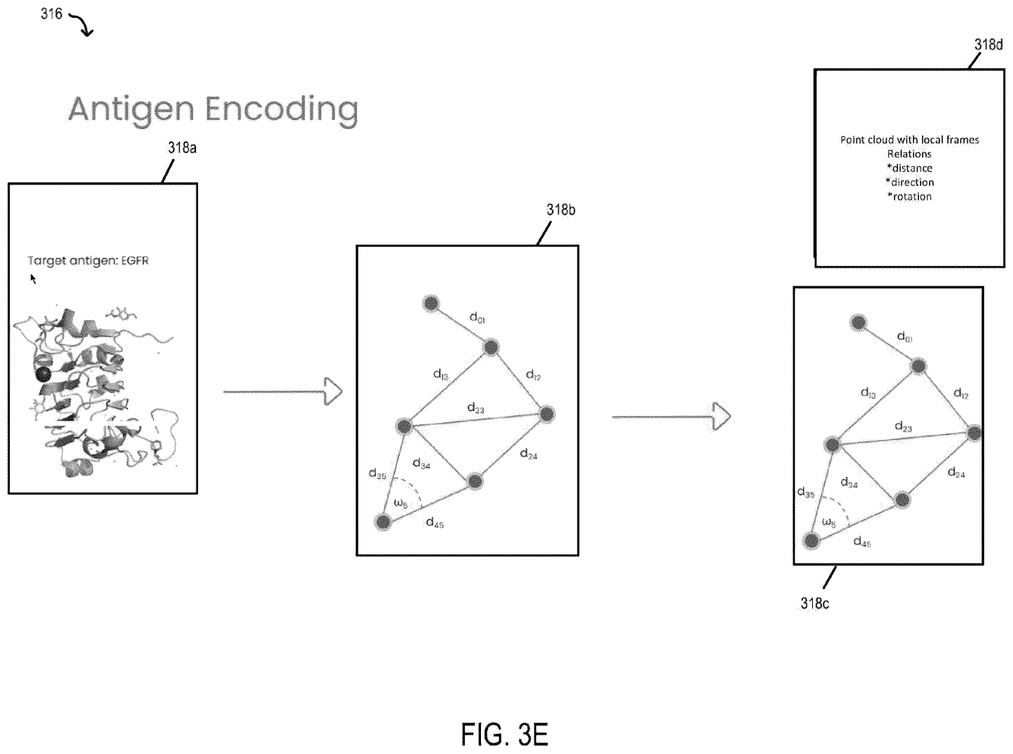

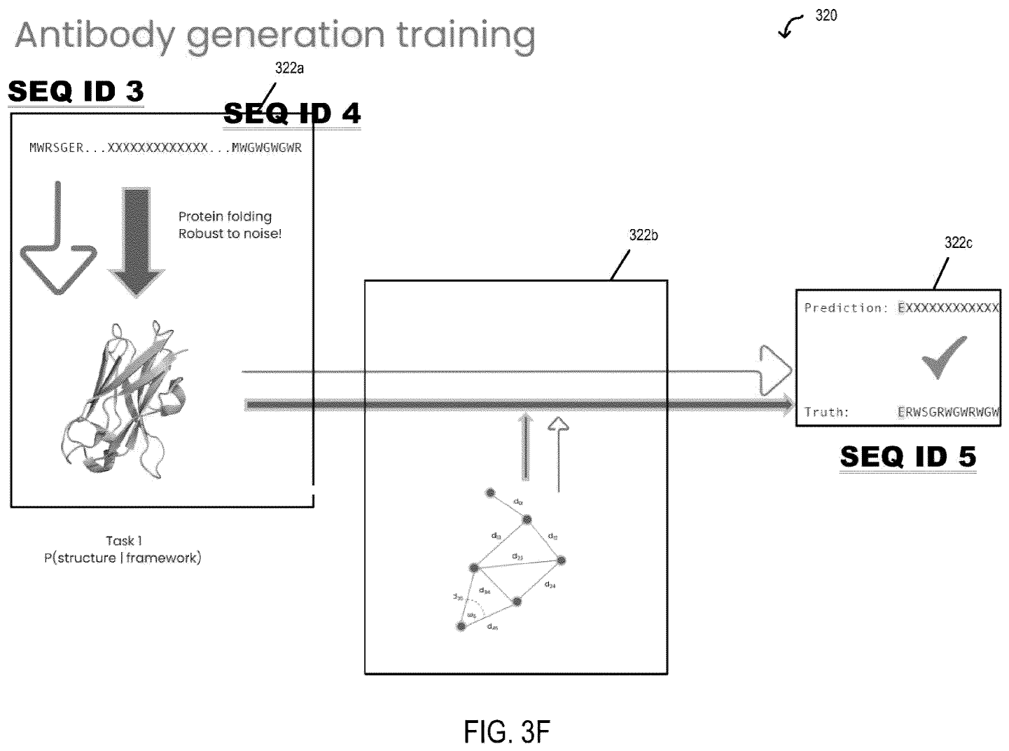

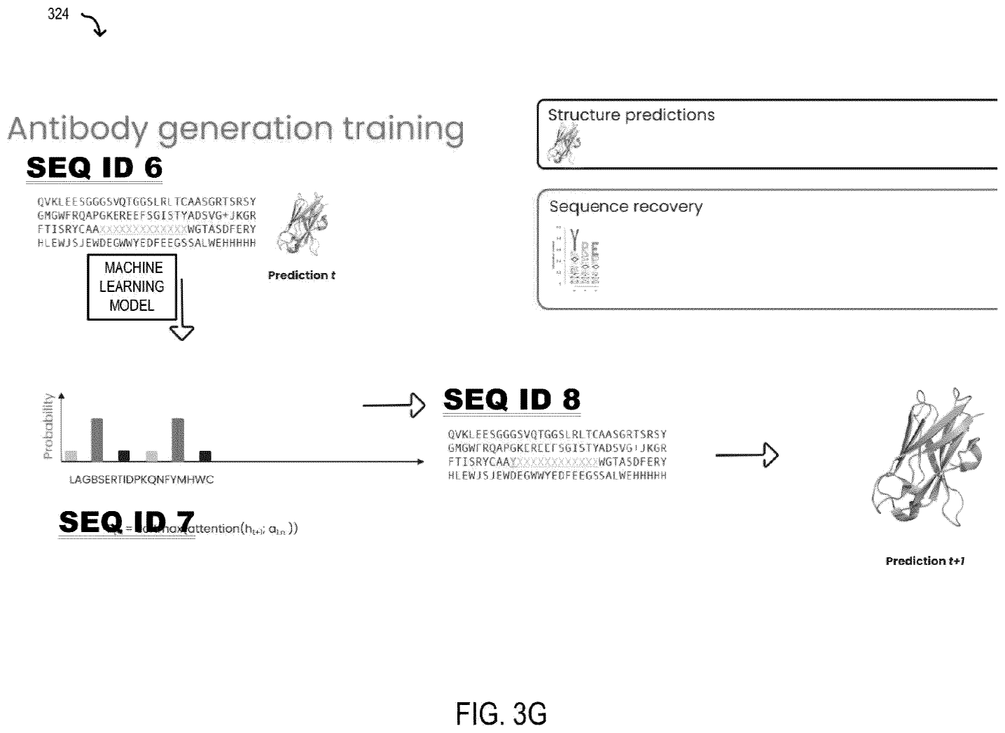

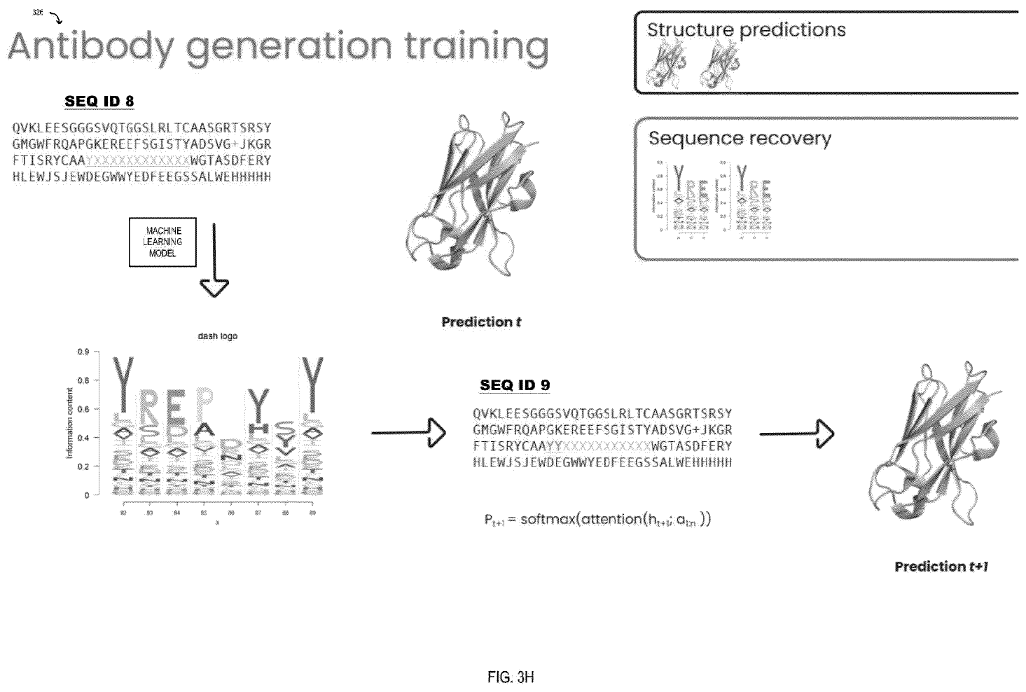

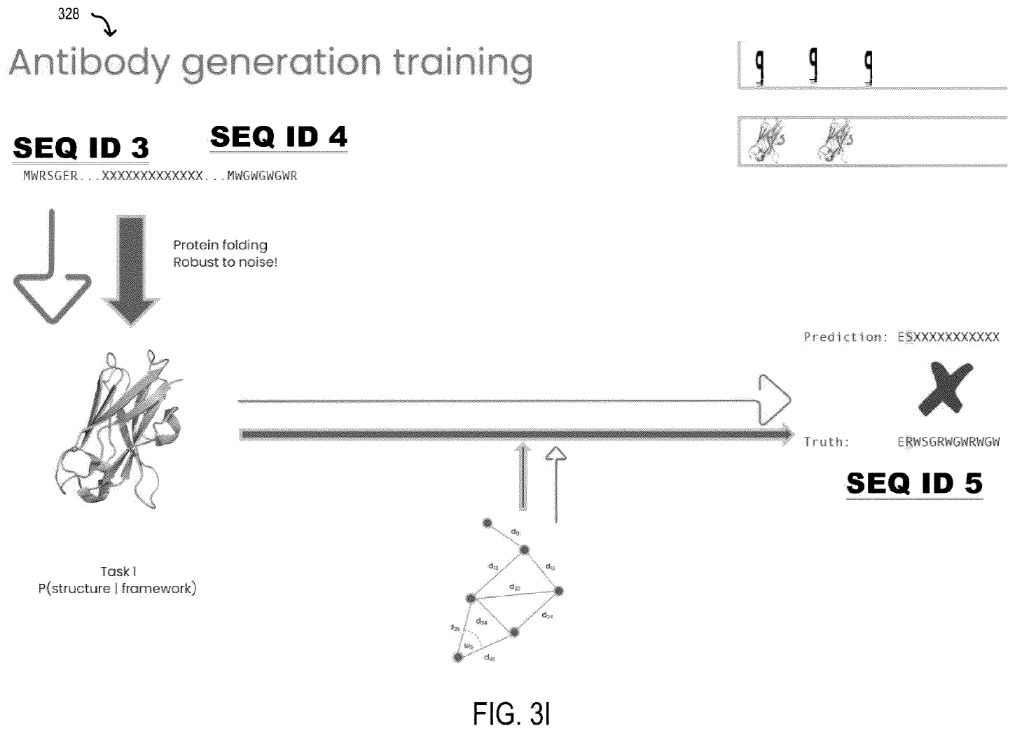

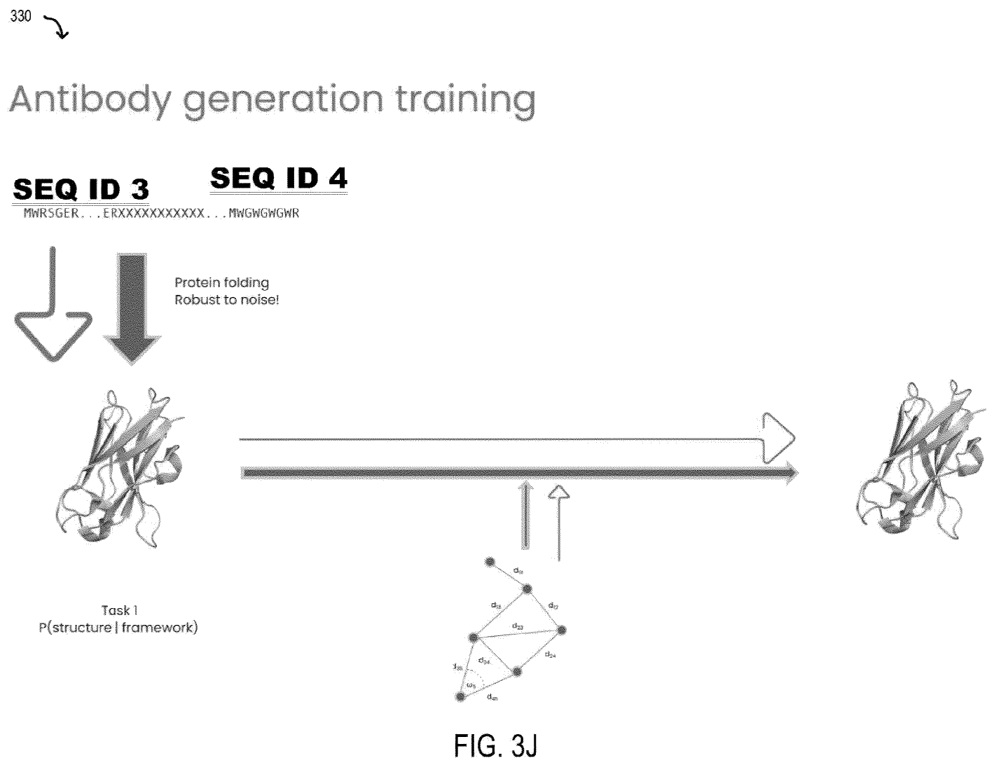

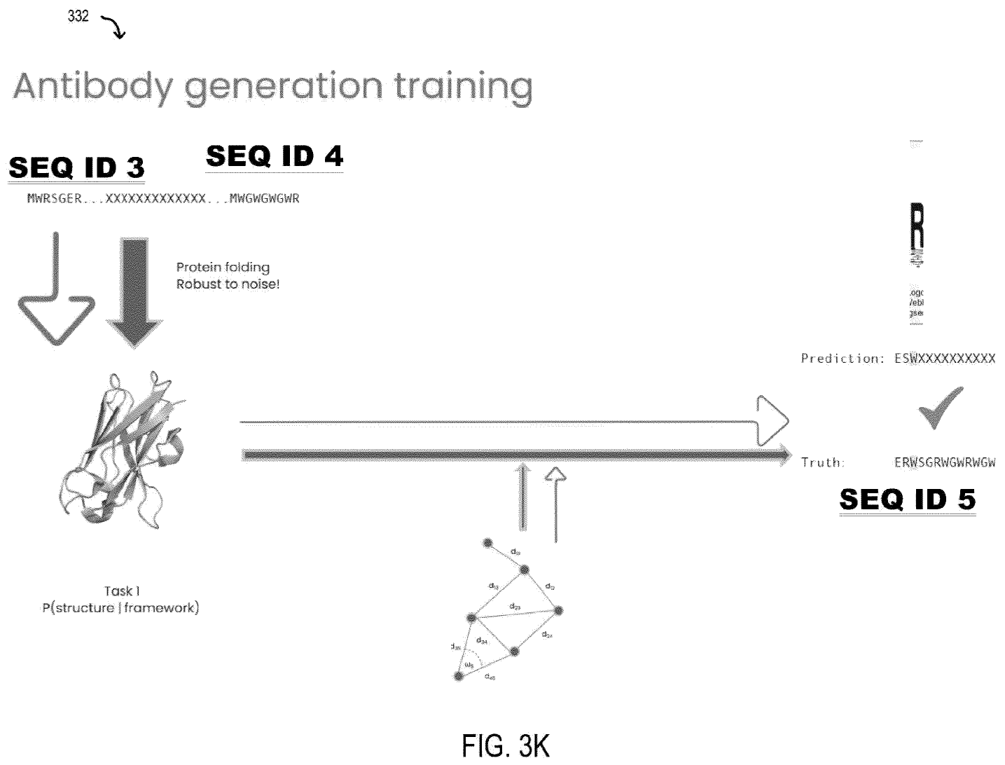

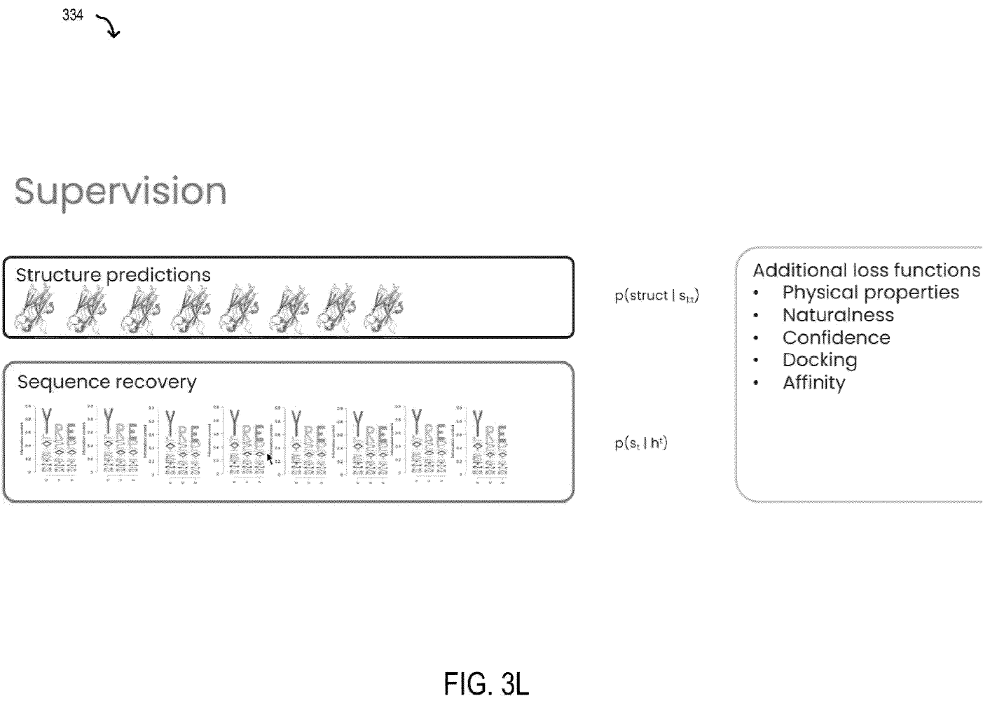

In some aspects, the method 230 may include pre-training the one or more biomolecule prediction layers using bound or unbound structures; pre-training the one or more biomolecule prediction layers and the one or more docking layers using bound structures; and/or pre-training the machine-learned affinity prediction artificial neural network using affinity training data. In some aspects, the method 230 may include receiving an external computationally docked complex corresponding to the target input; and compare the external computationally docked complex to the generated docked complex. Thus, the present techniques may advantageously be used for verification purposes. In some aspects, the method 230 may include the affinity training data being derived from an assay (e.g., an ACE assay). The affinity training data may include an affinity score that is proportional to activity. The method 230 may include controlling a gradient flow of the machine-learned affinity prediction artificial neural network by applying a stop gradient function in at least one of the one or more biomolecule prediction layers, the one or more docking layers, or the one or more affinity prediction layers. For example, in PyTorch, a stop gradient may be implemented using ‘.detacho’ which detaches the relevant variable from the computational graph, thereby creating a new graph. In aspects wherein the same network weights are applied in each iteration, stop gradients may be employed such that each iteration is a separate graph. Gradients may then be averaged. In this context, conceptually, the stop gradients may be thought of as a strategy to augment limited data. Generally, the method 230 may be used to perform de novo discovery of biomolecules (e.g., a drug candidate such as an antibody) in silico. FIG. 3A depicts a block flow diagram of an exemplary computer-implemented method 300 for performing de novo in silico biomolecule discovery, according to some aspects. The method 300 may include receiving a scaffold and input target (block 302a). The method 300 may include analyzing the scaffold and the input target (e.g., via the method 200) to generate one or more biomolecules (e.g., one or more antibodies) using a trained machine learning model (e.g., via the method 230). The method 230 may further include analyzing the output of the machine learning model at block 302b using a lead optimization machine learning model (block 302c). The lead optimization machine learning model may generate one or more candidates that the method 230 may validate (block 302d). The method 230 may further include generating one or more drug candidates and/or one or more respective manufacturing cell lines (block 302e). In FIG. 3A, the ML model(s) at block 302(b) that design biomolecules (e.g., one or more antibodies) to the target of choice (e.g., a target antigen) may include binders. These binders may be input to the machine learning model at block 302c to further optimize the potential candidates. FIG. 3B depicts an exemplary block diagram 304, according to some aspects. The blocks of the block diagram 304 may correspond to various elements of the computing environment 100 of FIG. 1, for example. The block diagram 304 includes an antibody generation block 306a. At block 306a, representing antibody generation, structurally-conditioned antibody design may occur. For example, block 306 may correspond to training/operation of one or more biomolecule generation machine learning model at block 222a of FIG. 2B. The block diagram 304 includes an end-to-end docking block 306b, that may include geometric learning (i.e., graph-based learning), in some aspects. For example, block 306b may correspond to training/operation of one or more docking (e.g., antibody-antigen docking) machine learning models at block 222b of FIG. 2B. The block diagram 304 may include an affinity (e.g., binding affinity) prediction block 306c, that may perform graph-based affinity prediction. In some aspects, the block 306c may correspond to the affinity prediction machine learning model training/operation at block 222c of FIG. 2B. In some aspects, the block diagram 304 may include a data sources block 306d, that may include both public data sources (e.g., public data bank) and proprietary assay data for conducting high-throughput experiments. The block 306e depicts computer architecture that may be used to carry out the present computer-implemented techniques. For example, the block 306e depicts a GPU that may correspond to the GPU 150 of FIG. 1. FIG. 3C depicts a block diagram 308 of exemplary CDR3 in silico discovery inputs and outputs. The block diagram 308 includes an objective (block 310a). In the depicted example, the objective is to design a CDR3 targeting an epidermal growth factor receptor (EGFR) (see, e.g., https://www.rcsb.org/structure/4krl) that works well in a VHH scaffold. However, it will be appreciated by those of ordinary skill in the art that other objectives, including other binding partners (e.g., other antigens, epitopes, etc.) and other scaffolds are envisioned. The block diagram 308 further includes a target input including a target input including one or more of a target binding partner sequence, a target binding partner, or a target epitope (block 310b). At block 310b, one or more trained ML models may analyze the target input to predict structural information. Starting with the EGFR antigen and a given scaffold (e.g. VHH) the model may be trained to generate a CDR3 of interest. At this point, the model may never have seen (i.e., been provided with training data representing) a similar CDR3 or a similar antigen. In some aspects, even those sequences sharing more than a percentage (e.g., 40% sequence homology) with the target binding partner may be removed from training data. In some aspects, antigen structures of the same class, architecture, topology, or homologous superfamily with the target binding partner may be removed from training data. The block 310c may include an antibody scaffold. Given an input antigen and an antibody scaffold sequence, the ML model may output a desired sequence (e.g., a sequence of the CDR3 of a VHH scaffold to target EGFR).

FIG. 3D depicts an exemplary block diagram 312 of machine learning-based drug candidate design, according to an embodiment. The diagram 312 includes block 314a, that depicts equivariant representations of a biomolecule structure. In particular the block 314a depicts structural predictions, rotational equivariance, and local coordinate frames of biomolecule structural information. Further, block 314b depicts that attention layers may enable non-linear modeling of potential epitope-paratope interactions, and various modeling architectures that may be used. Still further, block 34c depicts iterative design of drug candidates.

FIG. 3E depicts an exemplary block flow diagram 316 of antigen encoding, according to some aspects. The block diagram 316 includes an ML model being trained by receiving one or more training inputs, that may be one or more of (i) input biomolecule structural information, (ii) input biomolecule binding partner structural information or (iii) input biomolecule-input binding partner binding complex structural information (the EGFR antigen, to continue the above example) (block 318a). The block diagram 316 may inject the antigen onto a graph (block 318b). The ML model may be trained (e.g., by the ML training module 164 of FIG. 1) by passing the training inputs around the graph. Thus, every “node” or residue of the graph at block 318b, for example, may be summarized by its neighbors. The ML model may hold onto this information and, over the course of training, will learn how to extract meaningful information, such as point cloud relations (block 318d).

FIG. 3F depicts an exemplary block flow diagram 320 of antibody generation training, according to some aspects. In the diagram 320, the ML model starts with an initial guess of the antibody structure based on an input sequence (e.g., a SAbDab sequence) (block 322a). The ML model may predict target biomolecule structural information (e.g., the next sequence of the antibody structure) based on the antigen encoding information learned at block flow diagram 316 (block 322b). The model may output a prediction that can be compared to ground truth data to adjust the model's weights, depending on whether the prediction is correct or incorrect (block 322c). For example, the ML model may evaluate a loss function at block 322c, and modify one or more parameters of the ML model based on the loss function. As discussed herein, the antibody training in the block flow diagram 320 may continue until the structural information of the target biomolecule is complete.

FIG. 3G depicts an exemplary block flow diagram 324 of antibody generation training/operation, according to some aspects. The diagram 324 may include analyzing an input antibody scaffolding including an unknown region (e.g., a CDR3 region), as depicted by the string including the letter “X.” The block diagram 324 may include making a first prediction using a trained ML model, wherein the output is a probability distribution as to which sequence comes next in the unknown region. In some aspects, an attention function may be used along with a softmax activation function. The first prediction may be a next sequence of a CDR3. FIG. 3H depicts an exemplary block flow diagram of antibody generation training/operation, according to some aspects. FIG. 3H depicts a second prediction (i.e., a prediction t+1) wherein a second sequence of the CDR3 is filled in by the model. In some aspects, the ML model may make all predictions in a single time step, and not iteratively. For example, in some aspects, the guessed structure is a random structure. In other aspects, the same beginning structure is used each time the model is run. In some aspects, other machine learning architectures may work, including those used in other disciplines/fields of art (e.g., a visual artificial intelligence/machine learning field). In particular, those of ordinary skill in the art will appreciate that diffusion models and other generative modeling techniques that work well on continuous systems (e.g., image data) may be adapted for use in the present techniques.

FIG. 3I depicts an exemplary block flow diagram of antibody generation training, according to some aspects. In particular, FIG. 3I depicts an example of a prediction that does match the known ground truth. Thus, FIG. 3I is an example of negative reinforcement. FIG. 3J depicts an exemplary block flow diagram of antibody generation training, according to some aspects. Specifically, FIG. 3J depicts an example of the generated antibody being a three-dimensional structure/framework. For example, the three-dimensional structure depicted may be rendered using three-dimensional coordinates corresponding to a primary sequence of the target biomolecule. FIG. 3K depicts an exemplary block flow diagram of antibody generation training, according to some aspects. In particular, FIG. 3K depicts a third correct, relative to the ground truth values of FIG. 3F and FIG. 3I, for example. FIG. 3L depicts an exemplary block diagram depicting supervised machine learning model output, according to some aspects. The block diagram 334 includes both predictions of structural information of a target biomolecule, and one or more sequences thereof. The block diagram 334 also depicts that additional loss functions may be applied, relative to physical properties, naturalness, confidence, docking, affinity, etc. of the structural information of the target biomolecule.

### Exemplary Results

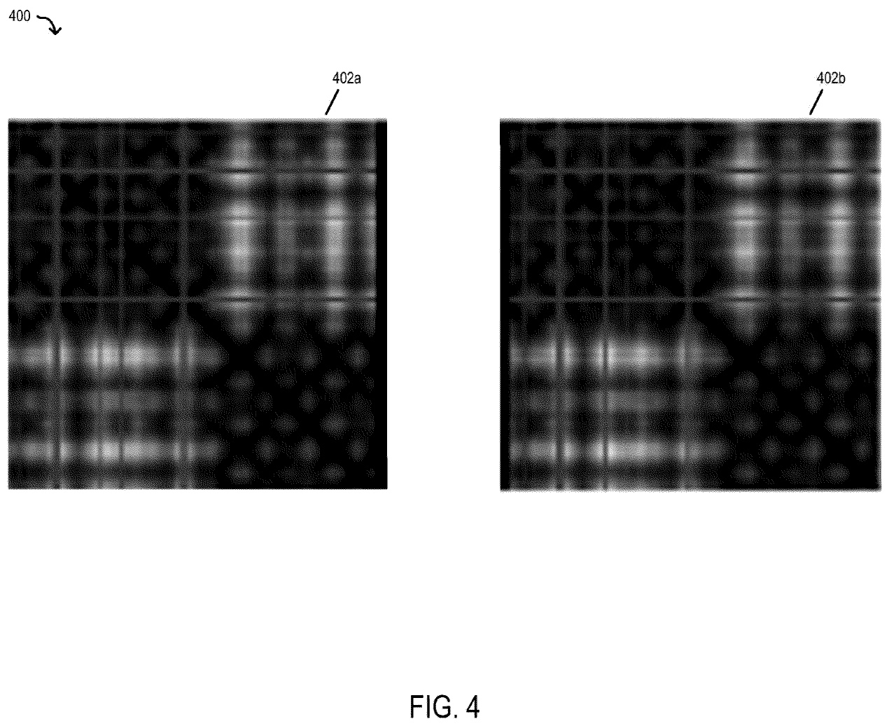

FIG. 4 depicts an example graphical user interface 400 including visualized predicted structural information 402a and visualized ground truth structural information 402b. The visualized predicted structural information 402a may correspond to the output of the affinity prediction block 222a. The visualized ground truth structural information 402b may correspond to data received from a database or other source (e.g., the protein data bank, wet lab crystallography, etc.). The visualized predicted structural information 402a may be generated by inputting target input including one or more of a target binding partner primary sequence, three-dimensional coordinates of a target binding partner, a target binding partner epitope primary sequence, or three-dimensional coordinates of a target binding partner epitope primary sequence, or a fragment or portion of any of the foregoing as described herein. For example, in some examples, the input to the ML model may be one or more protein sequences. The visualized predicted structural information 402a and visualized ground truth structural information 402b may be generated by computing a pairwise distance algorithm between individual points of the visualized predicted structural information 402a and visualized ground truth structural information 402b. Brighter values may indicate values that are farther apart, whereas values that are dark may indicate values that are closer together. Notably, the diagonal (coordinate i,i) represents the same residues.

Regarding the results of the present techniques, the fact that the visualized predicted structural information 402a and visualized ground truth structural information 402b appear visually similar indicates that the trained ML model/ANN is working as expected. In particular, the protein-folding component of the network is working, and designing the outputs along the way.

### Exemplary In Silico Biomolecule Design

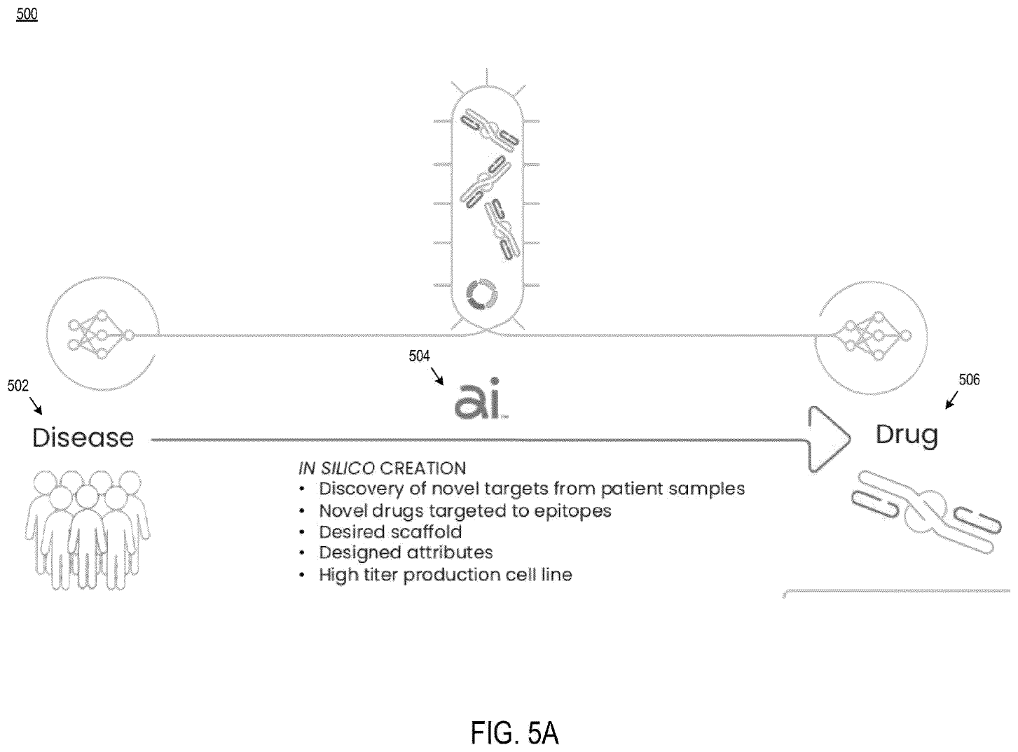

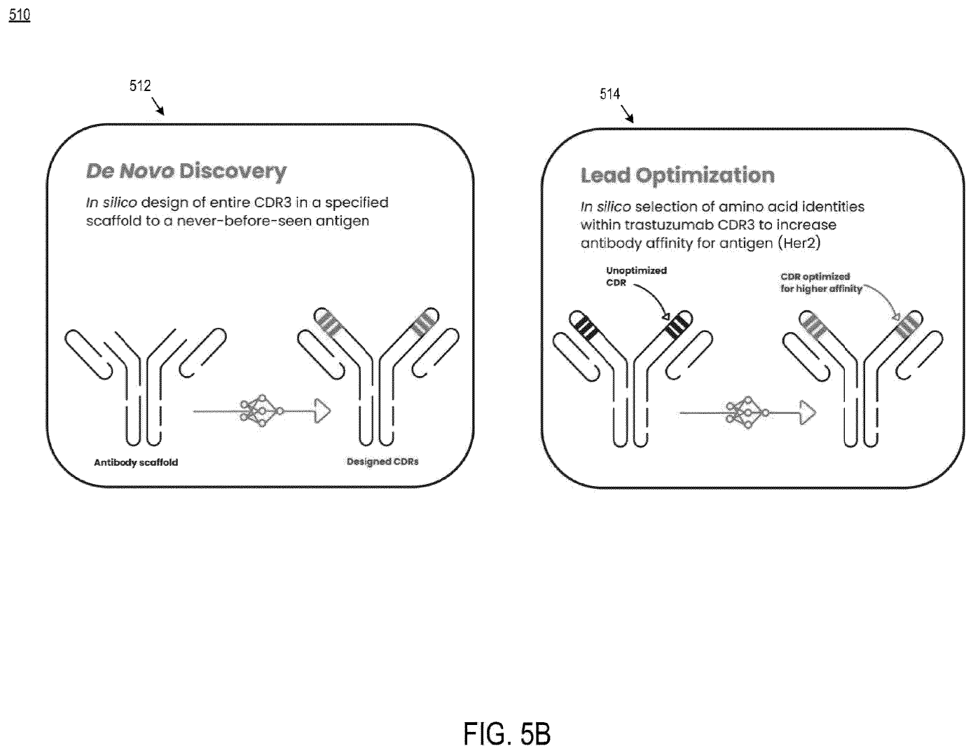

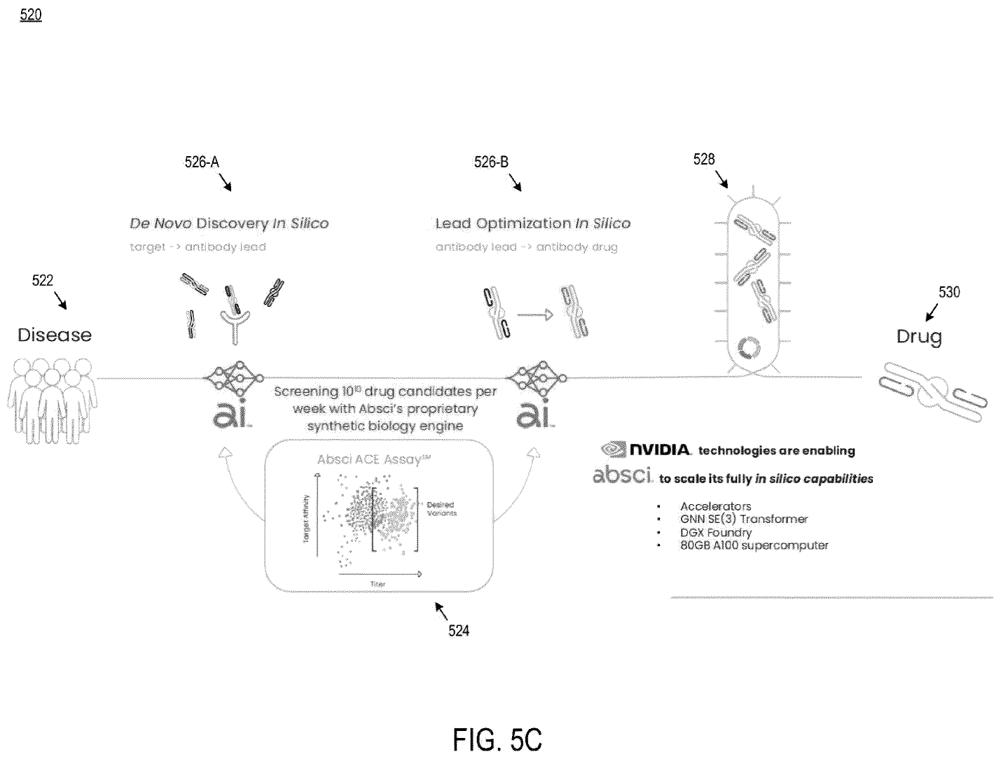

FIG. 5A depicts an example block flow diagram 500 of fully in silico biomolecule design, according to some aspects. Specifically, a group of patient samples may be collected, wherein each of the patients has one or more diagnosed diseases (block 502). One or more AI models may be applied to the patient samples to discover novel biomolecule targets (block 504, block 506). For example, the AI models may correspond to those trained by the ML training module 164 of FIG. 1.

FIG. 5B depicts an example block diagram 500 of a fully in silico de novo biomolecule design artificial intelligence model, according to some aspects. As discussed herein with respect to FIG. 3A, an antibody scaffold may be processed by an ML model to generate one or more designed CDRs 516 (e.g., corresponding to one or more respective drug candidates) (block 512). FIG. 5B further depicts a lead optimization model, including an in silico selection of amino acid identities within trastuzumab CDR3, to increase antibody affinity for antigen (Her2) (block 514).

FIG. 5C depicts an example block flow diagram 520 of in silico biomolecule design pipelines, according to some aspects. A group of patient samples may be collected, wherein the patients have one or more diagnosed diseases (block 522). De novo discovery may be performed in silico using one or more trained ML models as discussed herein, provided a target input, to predict structural information of a target biomolecule (block 526-A). In some aspects, the ML model may receive additional information (e.g., information from an ACE assay) (block 524). In some aspects, lead optimization may be performed using a lead optimization machine learning model as discussed herein (block 526-B). The ML model may output novel biomolecules targeted to specific epitopes that have desired scaffolds and/or desired attributes (block 528). These outputs may be used to craft high tier production cell lines and to create drug candidates (block 530). It will be appreciated by those of ordinary skill in the art that the present in silico design techniques may benefit from using supercomputing resources (e.g., accelerated neural network training environments such as GNN SE(3) transformers, hosted solutions for AI development (e.g., DGX foundry), and Graphics Processing Unit (GPU) supercomputers (e.g., A×100), etc.).

### Exemplary Machine Learning Modeling Results

As discussed herein, the present techniques include ML modeling to automatically design or improve biomolecules against a specific target of interest. For example, in some cases, the models may be pre-trained (e.g., using biomolecule complexes mined from the Protein Data Bank) to predict the structure, sequence and/or affinity of an antibody given an antigen structure. Recent testing has quantified that the present techniques are highly efficacious, even without the added benefit of pre-training the ML model(s) to predict the binding affinity of the target binding partner and predicted target biomolecule structural information.

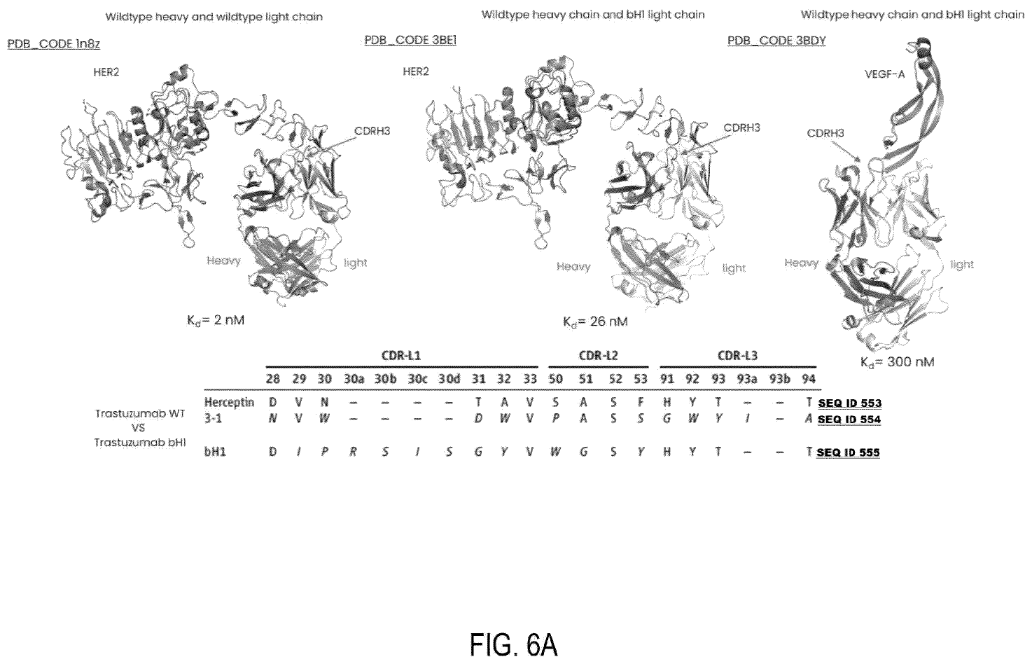

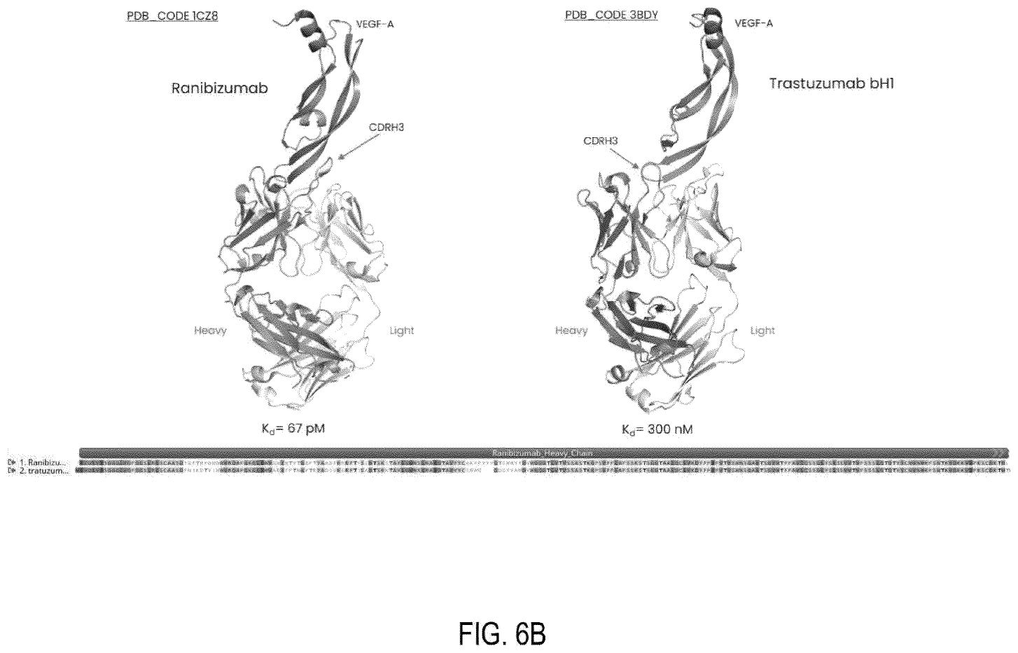

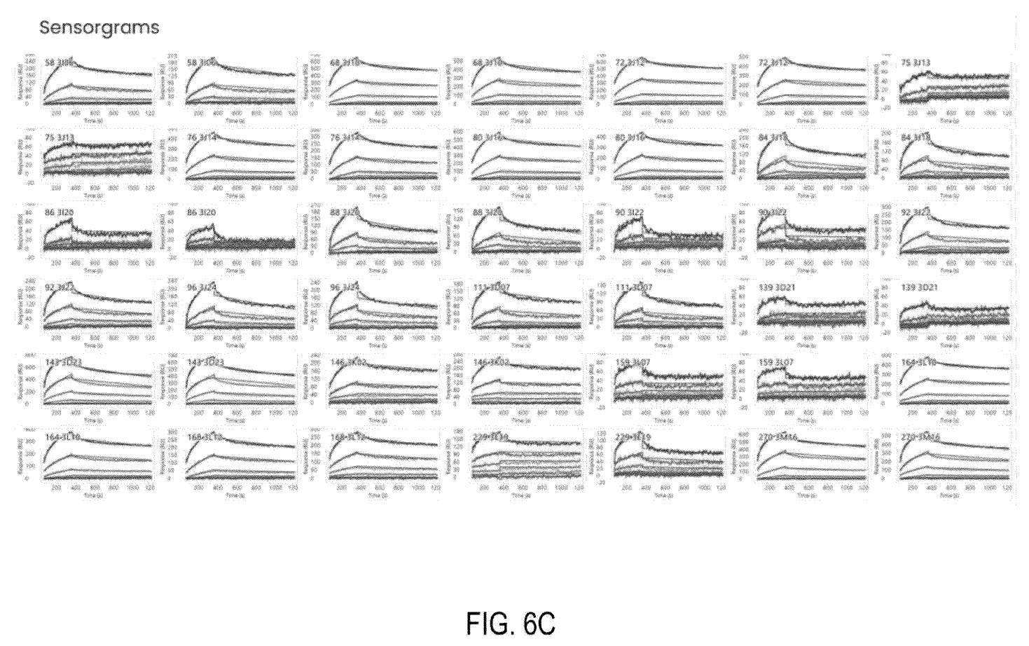

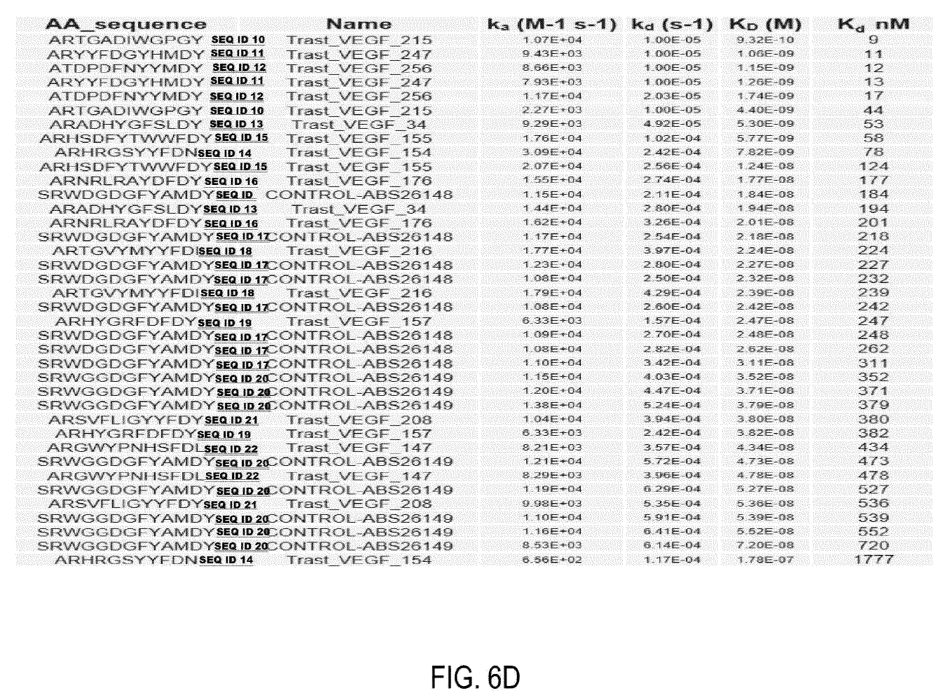

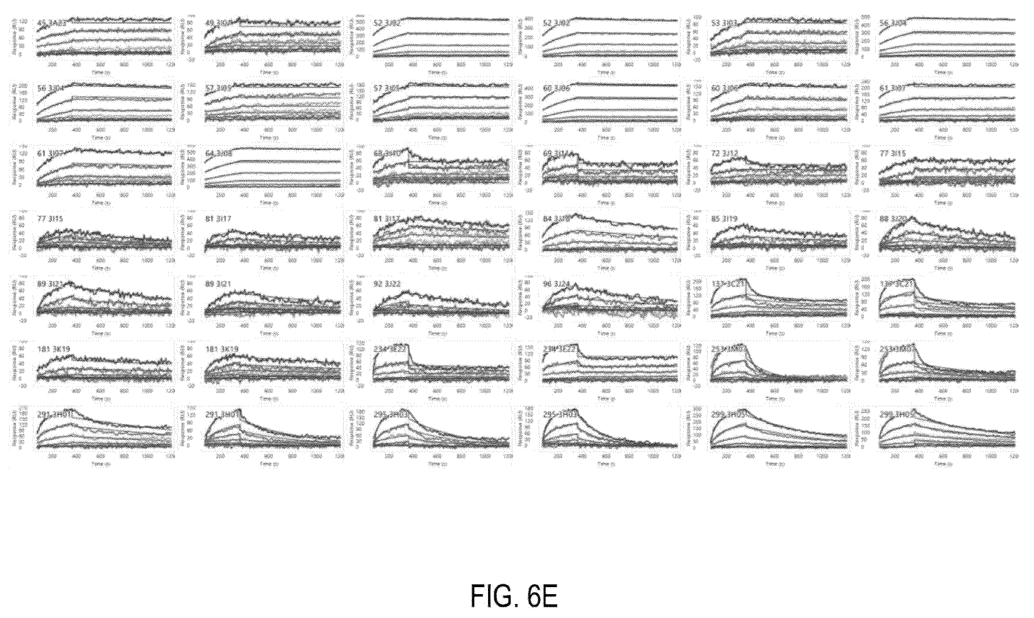

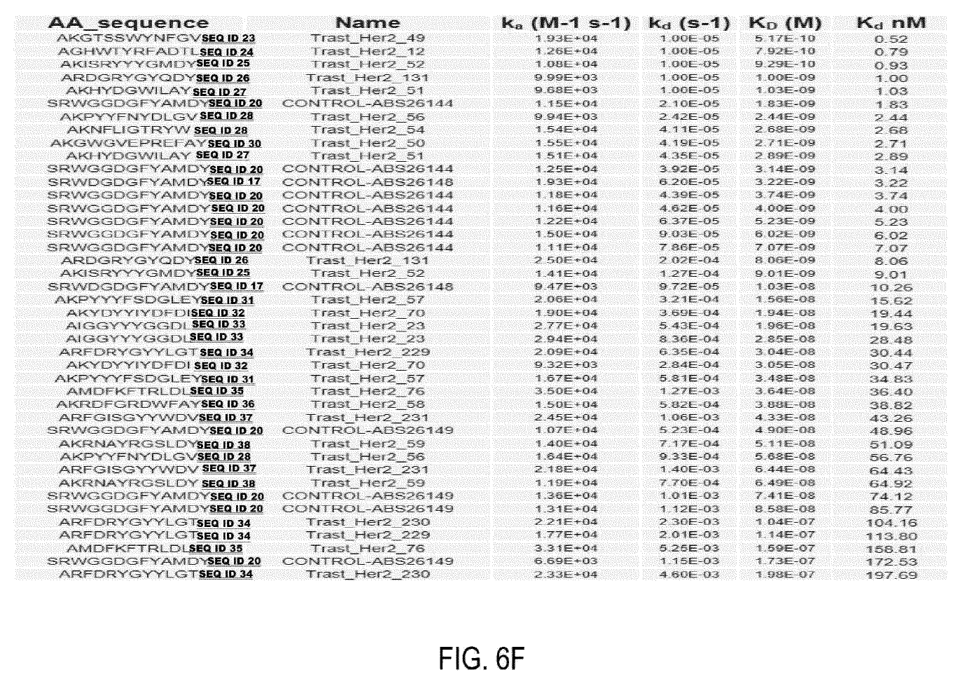

FIG. 6A depicts exemplary in silico biomolecule engineering including binder prediction based on optimization of CDRH3 while keeping remaining CDRs invariant, according to some aspects. Specifically, FIG. 6A depicts wildtype heavy biomolecules, namely trastuzumab wildtype light chain HER2, bH1 light chain HER2 and bH1 light chain VEGF-A. Each of these biomolecules has a distinct K_{d} value of 2 nM, 26 nM and 300 nM, respectively. FIG. 6B depicts exemplary ranibizumab and trastuzumab bH1 visualizations, according to some aspects.

FIG. 6C depicts exemplary sensorgrams corresponding to SPR results generated for predicted in silico binding of trastuzumab-bH1 CDR3 variants binding to VEGF, according to some aspects. FIG. 6D depicts exemplary tabular data corresponding to SPR results generated for the predicted in silico binding of trastuzumab-bH1 CDR3 variants binding to VEGF of FIG. 6C, according to some aspects. Specifically, FIG. 6C and FIG. 6D depict outputs corresponding to inputting CDRH1 and CDRH2 of trastuzumab-Bh1 and the structure of VEGF to a trained ML model, to design a CDR3s for trastuzumab-Bh1. The heavy chain CDRs may be the same for trastuzumab and trastuzumab-Bh1, but the light chain CDRs may differ. The results demonstrate that the predicted variants bind specifically to VEGF, as shown in FIG. 6D, wherein the name column depicts control biomolecules (e.g., ABS26144=trastuzumab and ABS26148=trast-bH1). Trastuzumab bH1 is a known variant of trastuzumab that was designed to bind to both HER2 and VEGF (see Bostrom J, Haber L, Koenig P, Kelley R F, Fuh G (2011) High Affinity Antigen Recognition of the Dual Specific Variants of Herceptin Is Entropy-Driven in Spite of Structural Plasticity. PLoS ONE 6(4): e17887. doi:10.1371/journal.pone.0017887)). Using the structure of bH1 bound to VEGF, CDR3 variants that would bind to VEGF were predicted. The name column also depicts predicted biomolecules, beginning with the prefix “Trast_.”

FIG. 6E depicts exemplary sensorgrams corresponding to SPR results generated for predicted in silico binding of trastuzumab CDR3 variants binding to Her2, according to some aspects. FIG. 6F depicts exemplary tabular data corresponding to SPR results generated for the predicted in silico binding of trastuzumab CDR3 variants binding to Her2 of FIG. 6E, according to some aspects. Specifically, FIG. 6E and FIG. 6F depict outputs corresponding to inputting CDRH1 and CDRH2 of trastuzumab and the structure of Her2 into a trained ML model to design CDR3s for trastuzumab. In particular, the CDR3 of the heavy chain (the other five CDRs are from trastuzumab) may be predicted, using the structure of trastuzumab bound to HER2 to predict CDR3 variants that would bind to HER2. The antibodies may be produced and affinity measured using SPR to validate the predicted binding. The results demonstrate that the predicted variants bind specifically to Her2.

In general, the data presented in FIGS. 6A-6D demonstrate that the present techniques for designing target biomolecules in silico are able to predict biomolecules that bind to the intended target, in some cases with significantly higher affinity than known controls, while delivering the advantages discussed herein, such as completely avoiding time-wasting and resource-intensive conventional wet lab approaches. Furthermore, the results identified thus far do not necessarily require the docking complex and affinity prediction techniques described herein for fine tuning. That is, the results in FIGS. 6A-6D are achievable via one or more biomolecule prediction layers trained to predict biomolecule structural information from target inputs. Adding fine tuning (e.g., one or more docking layers trained to generate docked complexes from two or more input three-dimensional biomolecules and one or more affinity prediction layers trained to predict affinity from input docked complexes) is expected to generate even higher quality results.

### Screening Hundreds of Thousands of Model-Generated Sequences for Binding

In some aspects, the present techniques leverage ACE assays (e.g., Bachas S et al.; Liu J. Activity-specific cell enrichment; Patent Publication No. WO2021/146626, 22.07.2021; etc.) to screen massive antibody variant libraries containing hundreds of thousands of members (e.g., expressed in Fab 64 format). After screening by ACE, the present techniques may validate the assay for the de novo discovery techniques described herein by sampling sequences for follow-up analysis by SPR, a gold standard in binding affinity measurement and detection. Empirical results have demonstrated that the ACE assay has high recall, correctly classifying >95% of the SPR binders. Moreover, the assay has high precision, with 60% of ACE binders validating in SPR. This may enable a powerful workflow where a large population of predictions can be initially screened by the ACE assay, and the expected binding population can be subsequently screened via SPR to remove false positives and collect high quality binding affinity measurements, as discussed herein.

The present techniques demonstrate the ability of generative AI to design de novo antibodies and antigens of interest, generate new HCDR3 sequences zero-shot for known antibodies. The present techniques focus on design of the HCDR3 region, a key determinant of antibody function, due to its high sequence diversity in immune repertoires and high density of paratope residues. For example, the present techniques may select trastuzumab, which binds to HER2, as a scaffold antibody to test HCDR3 designed sequences. HCDR3 designs are generated by a model conditioned on an antigen-only modified HER2 3-D structure (PDB:1N8Z (chain C)) and the sequence of the trastuzumab scaffold, excluding the HCDR3. The present techniques may remove, for models used herein, any antibody known to bind the target or any homolog (>40% sequence identity or part of the same homologous superfamily) to the target. In some settings, we instead remove all antibodies from the training set with >40% sequence identity to the wildtype antibody. In all cases, we observed binders. In total we generate and screen 440,354 antibody variants with the ACE assay to identify binding variants. The present techniques find approximately 4000 total estimated binders based on expected ACE assay binding rates and advance a subset for further characterization, in total confirming HER2 binding for 421 AI designs via SPR.

TABLE S5
Mean Naturalness scores across different groups
and p-values using trastuzumab scaffold.

| | | p-value (comparison |
|---|---|---|
| Group | Mean Naturalness | to de novo) |
| De Novo | 0.175 | N/A |
| Trastuzumab | 0.235 | 3.66 · 10⁻⁹⁵ |
| OAS | 0.194 | 3.63 · 10⁻¹⁶ |
| Frequency Baseline | 0.143 | 4.67 · 10⁻⁵³ |
| Phage Display Baseline | 0.136 | 1.11 · 10⁻⁸⁷ |
| Scrambled OAS | 0.101 | 8.05 · 10⁻³⁰⁰ |

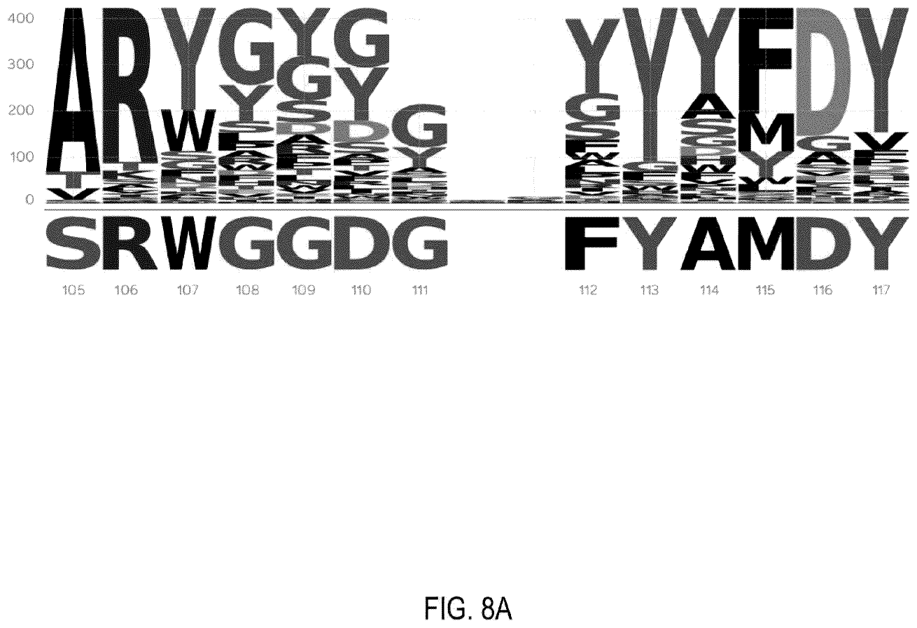

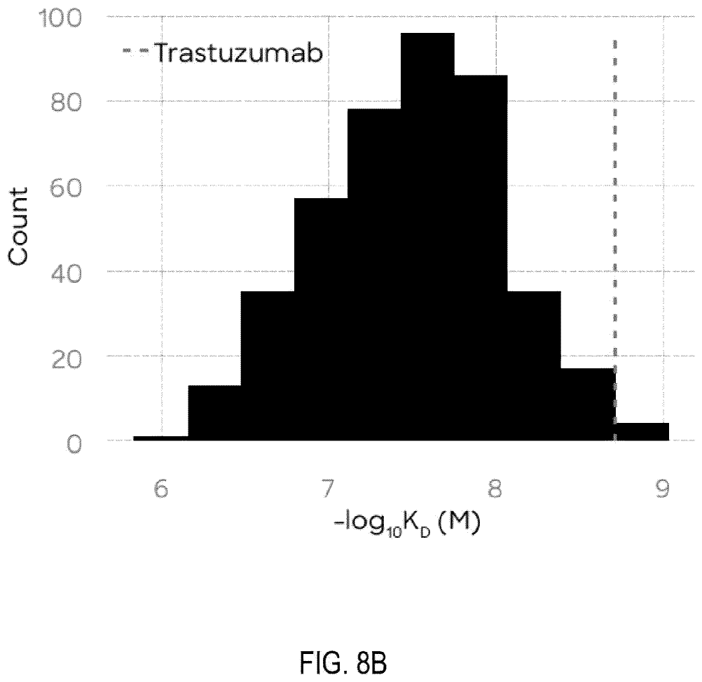

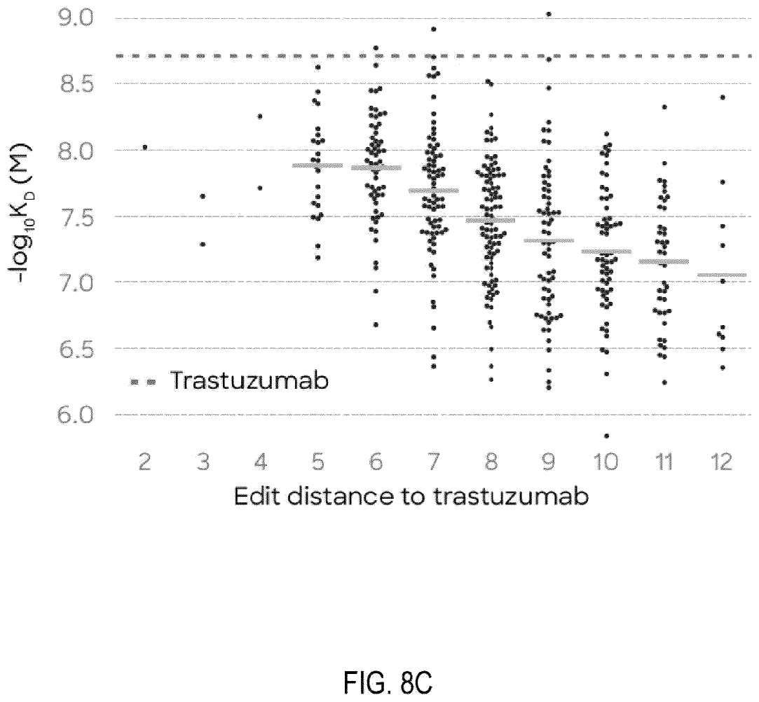

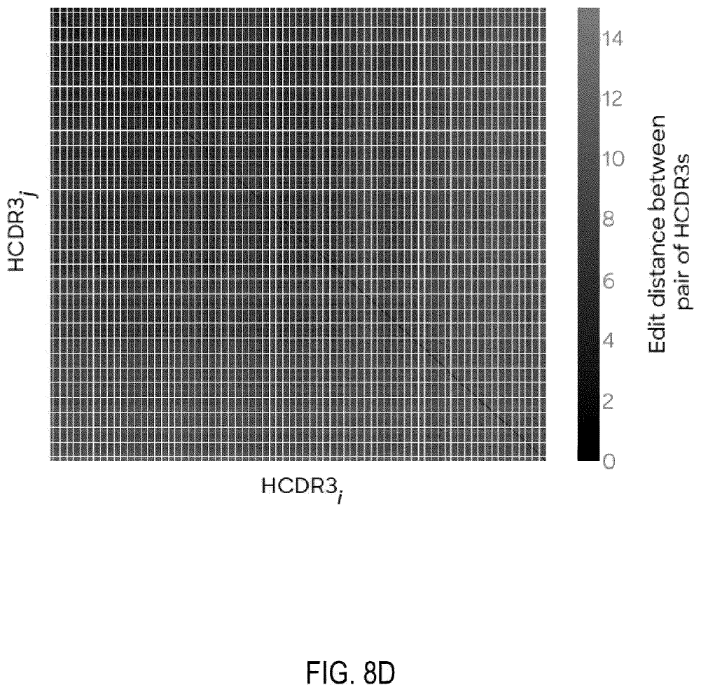

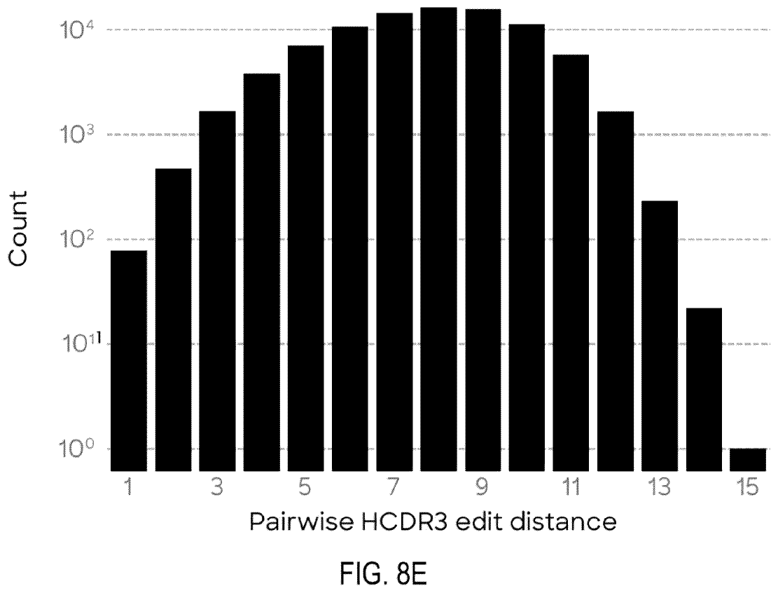

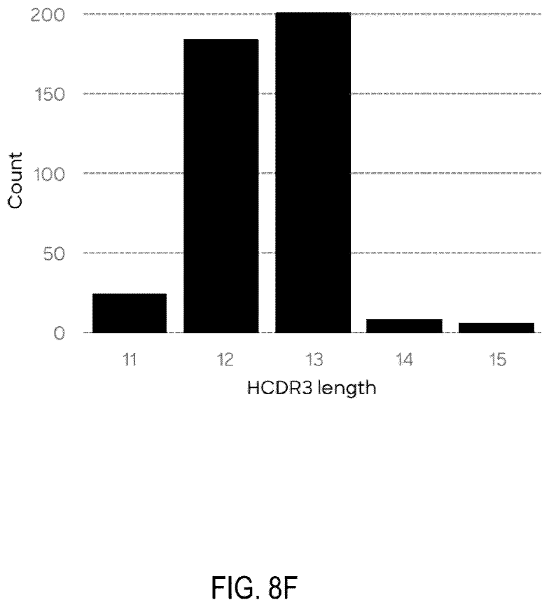

Confirmed binders show a range of affinity to HER2, with 71 designs exhibiting <10 nM affinity (FIG. 8A). Excitingly, three of the zero-shot designs display tighter binding than trastuzumab, with one binding in the sub-nanomolar affinity range. These high-affinity design examples are generated zero-shot from the model without any additional affinity maturation, therefore skipping a typically critical step in the development process of a therapeutic antibody. The ability to generate desirable antibodies that do not need additional optimization could significantly reduce development timelines.

FIG. 8A depicts binding affinities of AI-generated zero-shot binders, finding 71 designs with comparable affinity (<10 nM) to trastuzumab and 3 with tighter binding. FIG. 8B depicts designed variant binding affinities vs. edit distance to trastuzumab. Edit distances range from 2 mutations (84.6% sequence identity) to 12 mutations (7.7% sequence identity), illustrating the novelty of the designs. FIG. 8C depicts a logo plot of HCDR3s of 421 binding affinity trastuzumab variants. Greater diversity is observed the centers of the designed CDRs. Sequence logo below is trastuzumab HCDR3 sequence. FIG. 8D depicts pairwise edit distances between binders (minimum of 1, maximum of 15, median of 8, mean of 7.7, ±2.1 S.D.).

In addition to favorable affinity, AI model designs have high sequence diversity, both in terms of amino acid length and identity. Verified binders have HCDR3s ranging from 11 to 15 amino acids (FIG. 8F), compared to the trastuzumab length of 13. Designed sequences are also divergent from the trastuzumab antibody, with edit distances between 2 and 12 from the trastuzumab sequence (FIG. 8B). Average affinity decreases as edit distance increases from the trastuzumab sequence, but interestingly the present techniques find designs that still exhibit affinity less than 10 nM across all edit distances. The present techniques observed one design with an edit distance of 9 that exhibits higher affinity than the trastuzumab antibody. Additionally, the present techniques found higher diversity in the centers of the HCDRs, which corresponds to the more diverse D germline gene, compared to the less diverse flanking J and V germline genes (FIG. 8A). Designs are also sequence diverse from one another, with a mean edit distance of 7.7±2.1 SD (FIG. 8D, FIG. 8E). Inter-design diversity is noteworthy because it indicates model-generated binders are not converging to shared sequence motifs, as is often seen with traditional antibody screening methods like phage display.

### Designed Binders Display Sequence Novelty

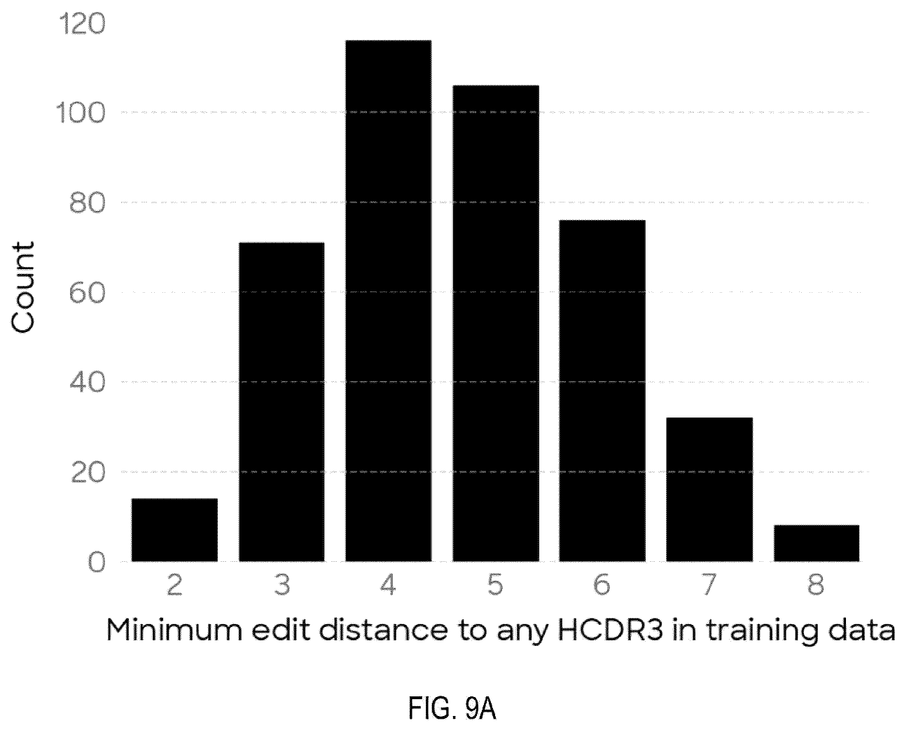

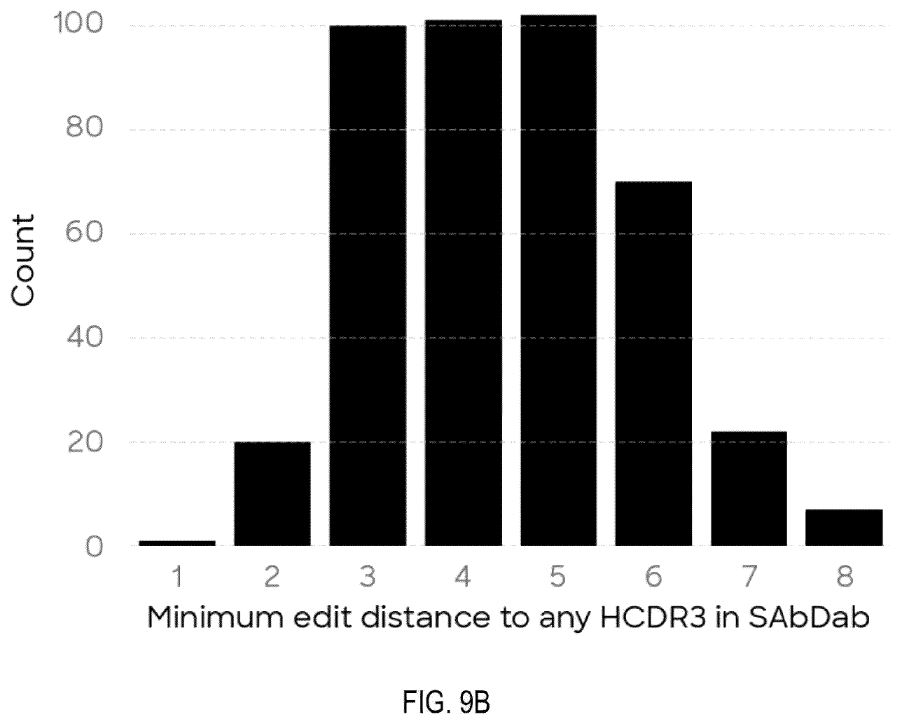

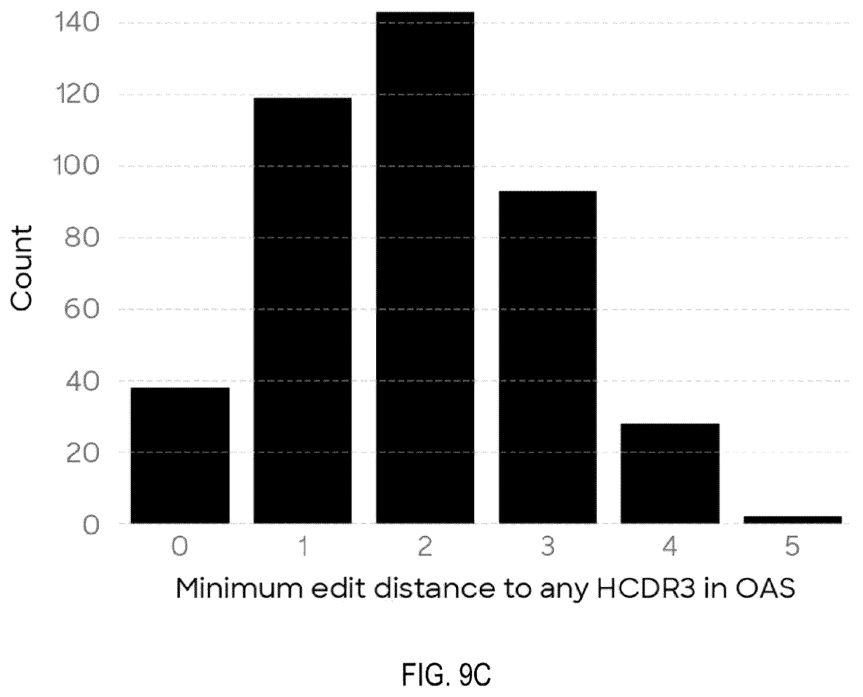

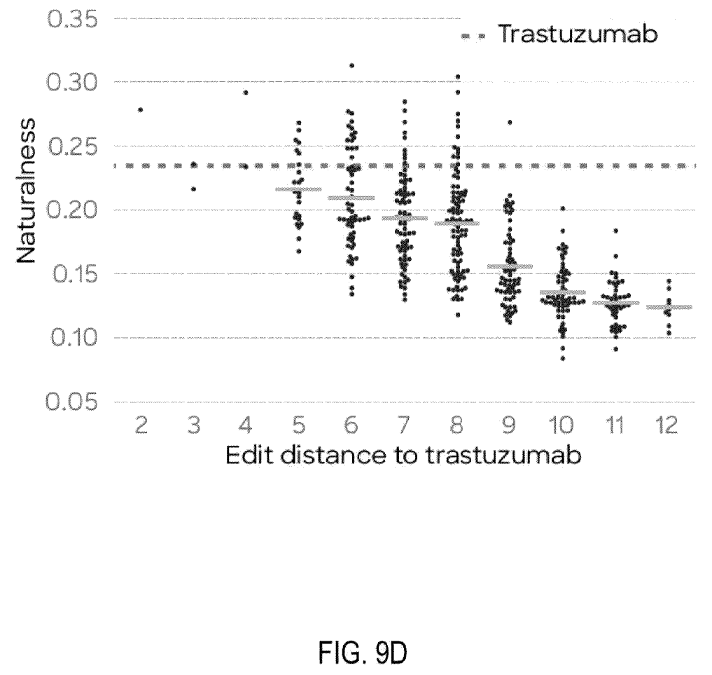

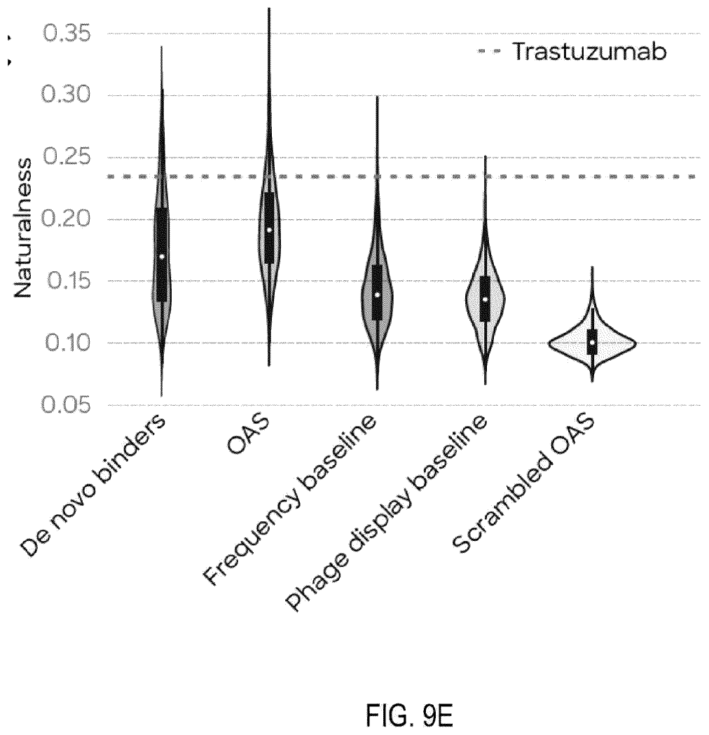

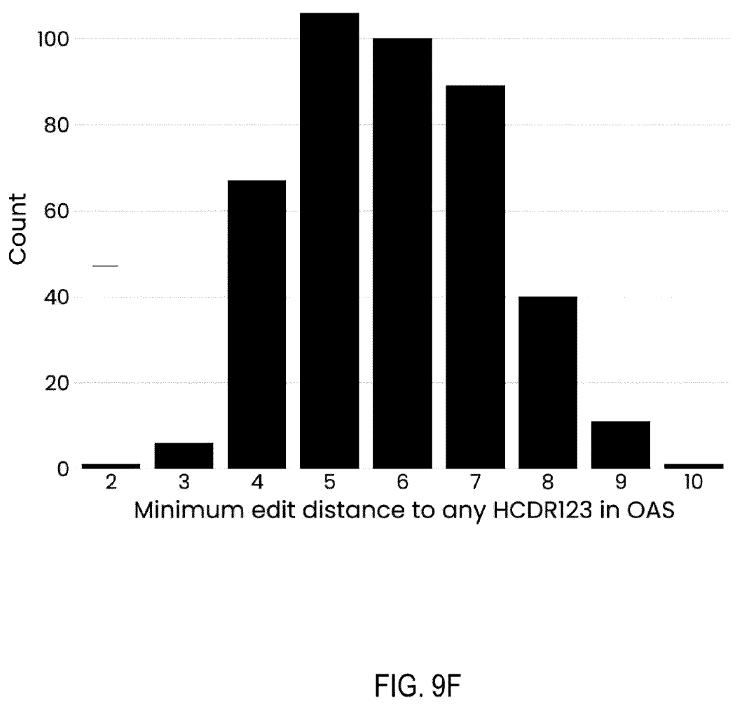

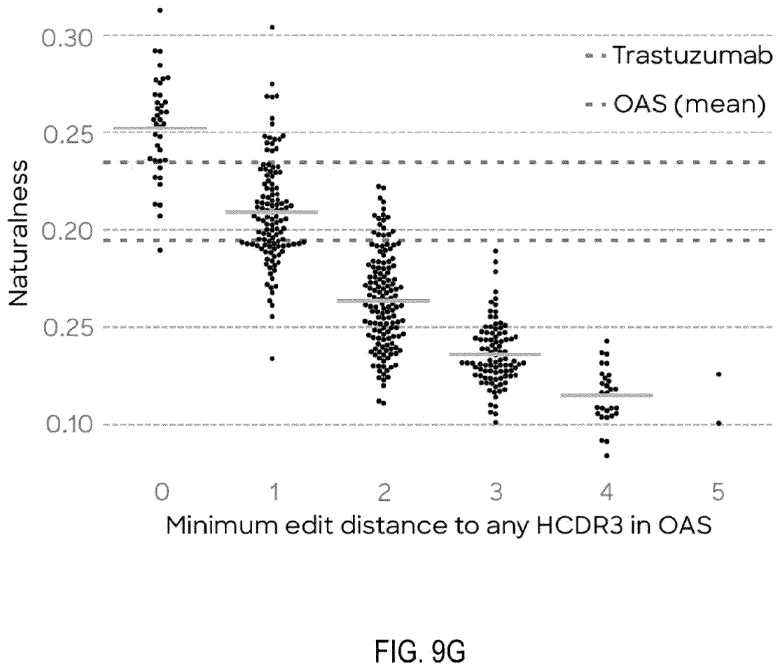

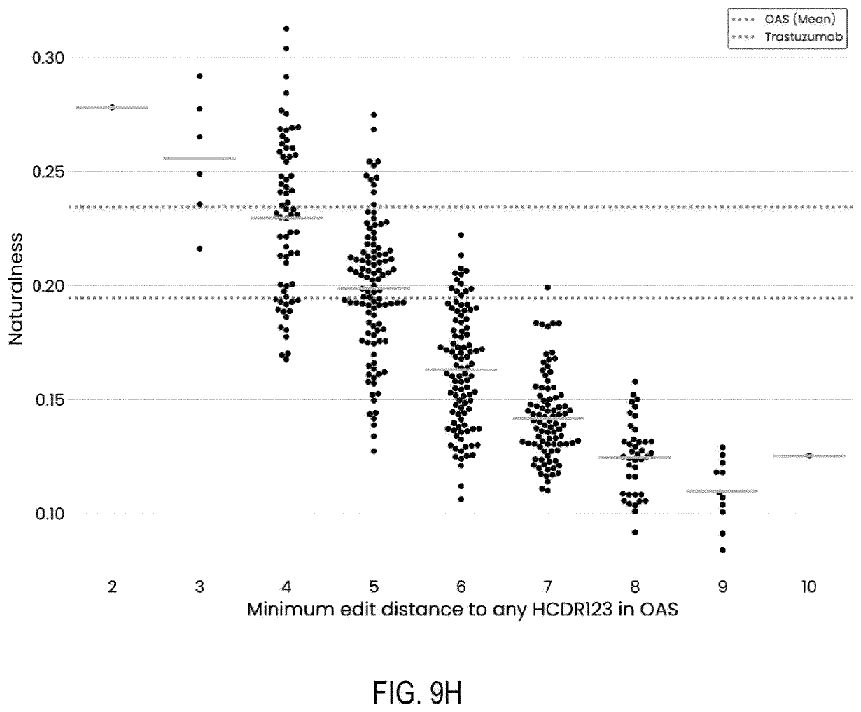

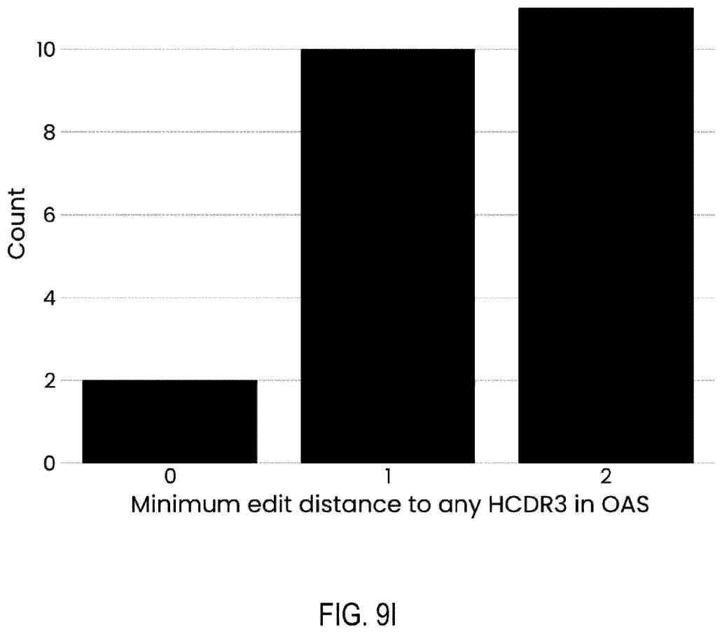

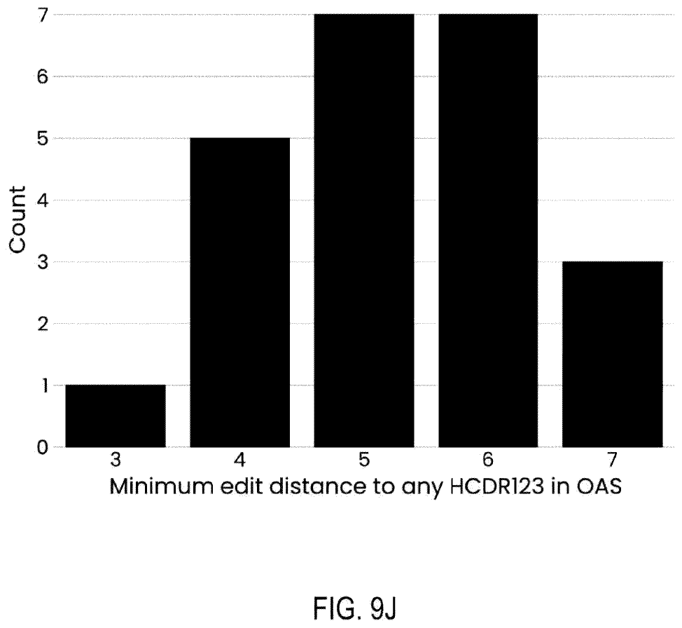

Despite the high sequence diversity of the 421 designed binders, to ensure model generations are novel sequences rather than simply reproduced training examples the present techniques compare model outputs to the training set. This phenomenon has been observed in machine learning models, and past methods have been critiqued for generating molecules that are similar to those previously known. The present techniques compute the minimum distance between the designed binders and all HCDR3s in the models training and validation sets, finding that designed binders are distinct from those observed during training (FIG. 9A). The present techniques next compute distances to all HCDR3s in the Structural Antibody Database (SAbDab), a database of antibody-antigen complexes, finding that binders are distant in sequence space from all antibodies in the database (FIG. 9B).

FIG. 9A depicts Minimum edit distance of binders to training data HCDR3s (minimum of 2, maximum of 8, median of 5, mean of 4.68±1.34 SD). FIG. 9C depicts Minimum edit distance of binders to OAS HCDR3s. 9.3% (38 out of 421) of the HCDR3 designs are contained in OAS. (minimum of 0, maximum of 5, median of 2, mean of 1.91±1.08 SD). FIG. 9D depicts Naturalness scores of designed binders vs. controls. de novo binders are those identified in our study. OAS refers to randomly selected HCDR3s from OAS. Frequency baseline samples amino acids at each position based on positional frequencies observed in OAS for HCDR3. The phage display baseline is a set of HCDR3s sampled from binding and non-binding antibodies from Liu et al. Scrambled OAS are randomly permuted versions of the OAS sequence set. AI designs have significantly higher Naturalness scores on average than control populations (p<10e-50) but on average have lower Naturalness scores than trastuzumab and sequences randomly sampled from OAS (p<10e-15). Red dashed line is the Naturalness score of trastuzumab. FIG. 9E depicts Naturalness scores of designed binders vs. edit distance to trastuzumab. Red dashed line is trastuzumab's Naturalness score.

The present techniques examined the sequence similarity of the model's output to sequences in the Observed Antibody Space (OAS), a database of immune repertoire sequencing studies. the present techniques found that some of the model's designs already existed in the OAS, while others were unique with minimum edit distances between 1-5 (FIG. 9B). This indicates the model is capable of generating sequences that achieve high Naturalness scores as well as sequences that are dissimilar to antibodies in observed immune repertoires.

Zero-Shot Designs have a High Naturalness Score

Therapeutic lead antibody candidates that are successful in the drug development process typically have high affinity and are developable with low immunogenicity. In previous work we described a language model that can assign a score to antibody sequences indicating the likelihood of finding a sequence in a typical immune repertoire. This metric is referred to as Naturalness. A high Naturalness score is associated with favorable developability and immunogenicity development outcomes. Using the Naturalness scoring model on our designs, we find our models can generate sequences with high Naturalness scores, with high affinity in a zero-shot manner, despite not training or sampling based on these qualities (FIG. 9E). Many designs exhibit Naturalness scores

FIG. 9D depicts Naturalness measures of the present designed binders versus their diversity to trastuzumab. Significantly, many designs depicted in FIG. 9D are more natural than the wildtype (depicted by the horizontal line) notwithstanding the fact that the wildtype corresponds to an FDA approved molecule. As with binding affinity, the naturalness of the binders falls as the edit distance to trastuzumab increases. However, empirical testing using the present techniques still identifies binders with high edit distance (5-9 mutations) that have higher naturalness than trastuzumab higher than trastuzumab. Table S5 shows the mean Naturalness scores across the different groups as well as p-values for the relevant statistical comparisons to the de novo binders. These results highlight the potential for zero-shot designs to bypass portions of the traditional lead optimization process, potentially saving time and resources in drug development.

3D Structural Representation of De Novo Designed HCDR3s and their Comparison to Trastuzumab-HER2 Complex

TABLE 1
Properties of diverse HCDR3 candidates
selected for 3D structural modeling.
HCDR3 candidates were selected based on
affinity, length, and edit distance to
Trastuzumab. RMSD values were calculated
over all main chain and side chain atoms
from alignment of HCDR3 residues. All
other atoms were excluded from calculations.
Grand average of hydropathy values for HCDR3
residues were calculated by averaging the
hydropathy values of each residue and
dividing by sequence length.⁵⁸

| SEQ | | | Binding | | |
|---|---|---|---|---|---|
| ID | | log₁₀ | Interface | RMSD | |
| NO: | HCDR3 | (K_{D}) | (Å²) | (Å) | GRAVY |
| 20 | SRWGGDGFYAMDY (0) | −8.71 | 771 | 0.0 | −10.5 |
| 53 | ARWGNYYYYYMDY (6) | −8.77 | 739 | 2.435 | −15.6 |
| 42 | ARYYYGFYYFDY (7) | −8.92 | 819 | 2.832 | −8.8 |
| 43 | ARYAGVERPGSFAY (11) | −6.24 | 764 | 1.107 | −5.9 |
| 44 | TRYFFNGWYYFDV (9) | −9.03 | 843 | 1.974 | −4.8 |
| 45 | AFADSGAYGIWSF (12) | −7.0 | 824 | 5.738 | 7.4 |
| 46 | ANDIYIQGYDLNR (12) | −8.4 | 833 | 5.506 | −10.4 |
| 47 | ARGYSGDWPYETFYV (10) | −7.01 | 863 | 6.767 | −11.4 |
| 48 | ARYDYGYYIYVS (10) | −8.02 | 718 | 3.032 | −5.2 |

The present techniques next predict the binding mechanisms of the present de novo designed HCDR3 variants to better understand the structural basis of the highly sequence diverse variants. To this end, the present techniques built structural models of eight diverse HCDR3 candidates bound to HER2 in Fab format. These eight variants are selected based on their edit distance to the trastuzumab HCDR3, diversity in length (ranging from 12-15 amino acids), and affinity range, spanning three orders of magnitude (Table 1). The present techniques use trastuzumab Fab-HER2 complex (PDB:1N8Z) as a starting template for structural modeling. The present techniques run local constrained backbone geometry and side chain rotamer optimization followed by relaxation of Fab-HER2 complexes to correct global conformational ambiguities, steric clashes, and sub-optimal loop geometry. As a control, the experimental trastuzumab-Fab complex is optimized using the same protocol and used as a reference for comparing final HCDR3 structural models. The present techniques use the lowest free energy poses of the de novo HCDR3 models for structural analyses and comparisons.

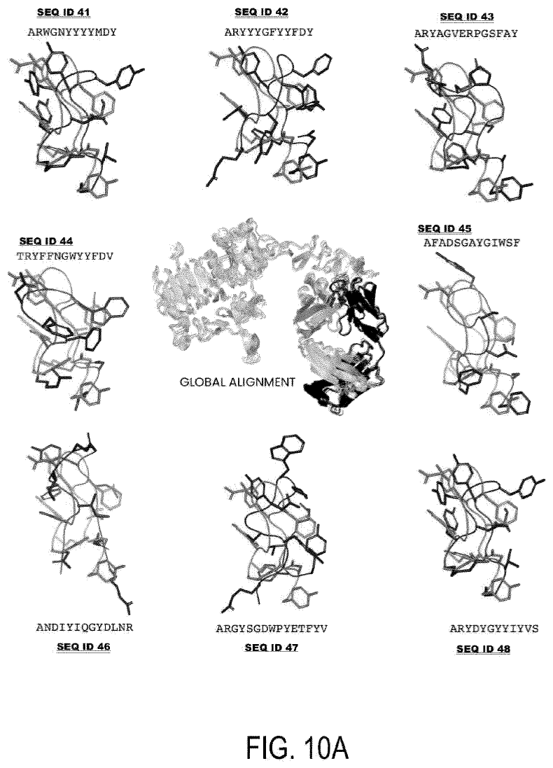

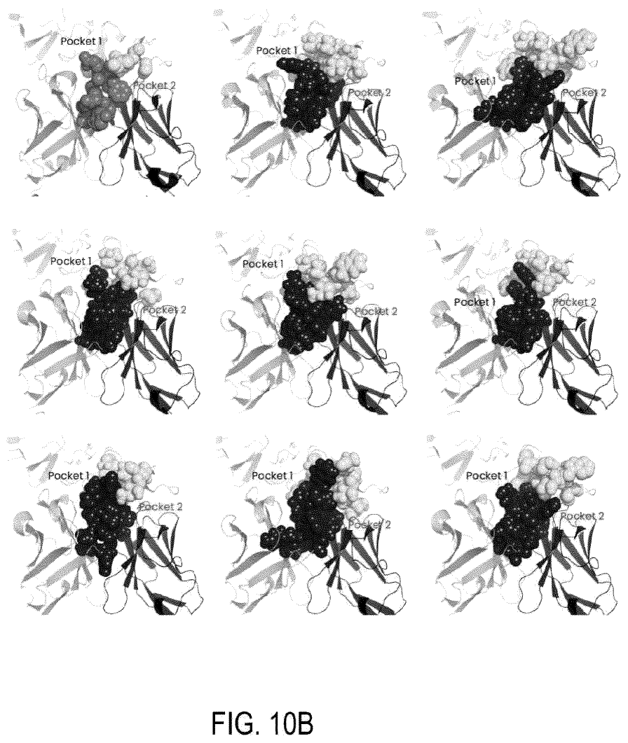

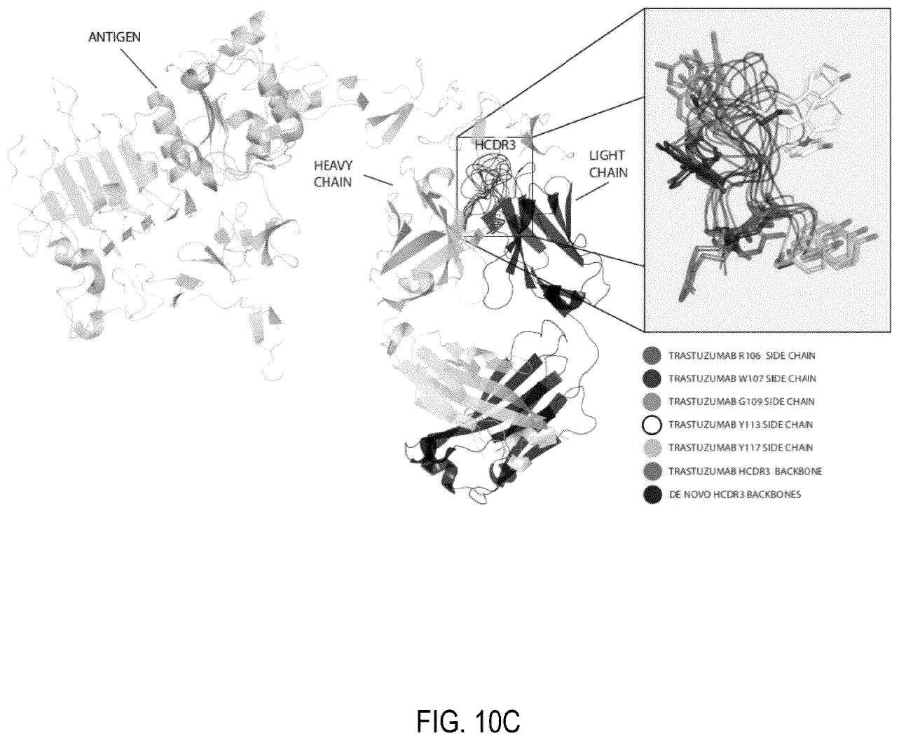

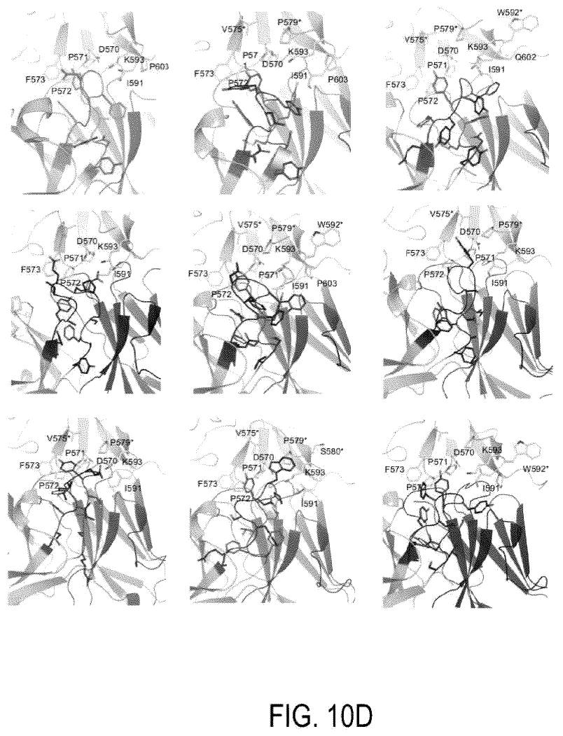

The eight de novo Fab-HER2 structural models are globally similar to trastuzumab with all-atom HCDR3 RMSDs ranging from 1.9 A-2.4 Å, despite sequence dissimilarity. Minimal structural rearrangements are observed in the unmodified regions of the heavy chain, the light chain and epitope residues of the antigen (FIG. 10A). In select cases, side chains forming contacts with the HCDR3 show slight rotameric differences to account for the presence of longer loops or steric clashes from residues with larger side chains (FIG. 10B). In terms of designed HCDR3 regions, alignment with trastuzumab Fab-HER2 complex reveals a dynamic ensemble of conformations adopted by each HCDR3 (FIG. 10C). HCDR3 loop structural differences are broad, with RMSDs ranging from 1.1-6.7 Å when aligned over all main chain and side chain atoms (Table 2). In FIG. 10C, despite the sequence and length diversity there are key residues conserved in space, corresponding to the trastuzumab residues W107, G109, and Y113 (IMGT numbering scheme).

TABLE 2
Multi-step AI designed Trastuzumab variant binders
to HER2 with all three HCDRs designed. ED indicates
edit distance from an HCDR to the Trastuzumab value.
Note the model occasionally recovers the native
Trastuzumab HCDR1 and HCDR2. We display nine variants
here and open source the entire set of 23 designs in
accompanying sequence data.

| | | | | KD |
|---|---|---|---|---|
| | HCDR1 (ED) | HCDR2 (ED) | HCDR3 (ED) | (nM) |
| Trastuzumab | GFNIKDTY (0) | IYPTNGYT (0) | SRWGGDGFYAMDY (0) | 1.94 |
| | SEQ ID NO: 54 | SEQ ID NO: 55 | SEQ ID NO: 20 | |
| AI Design 1 | GFNVSSSY (4) | IYPENGST (2) | SRYSYYGGYAFDY (6) | 224.9 |
| | SEQ ID NO: 56 | SEQ ID NO: 57 | SEQ ID NO: 58 | |
| AI Design 2 | GFNISDYY (2) | IYPSSGYT (2) | ARDGGVGSYSMDV (6) | 1323.0 |
| | SEQ ID NO: 60 | SEQ ID NO: 60 | SEQ ID NO: 61 | |
| AI Design 3 | GFNIKDYY (1) | IYPSRGYT (2) | ARHGSEGRYTMDV (7) | 192.1 |
| | SEQ ID NO: 62 | SEQ ID NO: 63 | SEQ ID NO: 64 | |
| AI Design 4 | GFNIKDTW (1) | IDPANGYT (2) | ARVGGRGYYYYDY (6) | 394.1 |
| | SEQ ID NO: 65 | SEQ ID NO: 66 | SEQ ID NO: 67 | |
| AI Design 5 | GFNFKDTY (1) | IYPANGYT (1) | ASDGGSYSYAFDY (7) | 642.2 |
| | SEQ ID NO: 68 | SEQ ID NO: 69 | SEQ ID NO: 70 | |
| AI Design 6 | GFNIKDTY (0) | IDPADGYT (3) | AREGGYGYYAMDY (4) | 160.1 |
| | SEQ ID NO: 54 | SEQ ID NO: 71 | SEQ ID NO: 72 | |
| AI Design 7 | GFNIKDTY (0) | IDPANGYT (2) | ARVGGRGYYYYDY (6) | 134.0 |
| | SEQ ID NO: 54 | SEQ ID NO: 66 | SEQ ID NO: 67 | |
| AI Design 8 | GFNIKDTY (0) | IYPSNGYT (1) | ARYGGSYAYYFYY (7) | 18.6 |
| | SEQ ID NO: 54 | SEQ ID NO: 73 | SEQ ID NO: 74 | |
| AI Design 9 | GFNIKDTY (0) | IYPTNGYT (0) | ARYYGSSSYYFAY (9) | 60.6 |
| | SEQ ID NO: 54 | SEQ ID NO: 55 | SEQ ID NO: 75 | |

Even though de novo HCDR3s adopt distinct conformations there are important positional similarities among all structures. A closer analysis of the spatial orientation of side chains conformers reveals conservation of identical side chains at five discrete spatial locations, two of these locations corresponding to IMGT residue position R103 and Y117 in trastuzumab which are highly conserved in most antibodies. However, there is physiochemical conservation in all structures corresponding to the spatial positions of IMGT residue numbers 109, 113, 117 of trastuzumab, which contribute to the paratope of the trastuzumab-Her2 complex. Although conserved spatially, these side chains originate from a diversity of residue positions which highlight the conformational flexibility observed may be required for orienting key paratope side chains towards making identical important protein-protein interaction with epitope of HER2.

Although the overall binding region is identical, each designed HCDR3 exhibits dis-tinct binding modes between HCDR3 and epitope. In most cases, novel interactions not observed in the trastuzumab-HER2 complex are formed between designed HCDR3 and domain IV HER2 (FIG. 10B, FIG. 10D). These interactions are diverse and consist of a novel hydrogen bonding interactions, nonpolar interactions, aromatic interactions, and electrostatic interactions formed with each HCDR3 and Her2 epitope. To further decipher the determinants of binding the present techniques calculated the surface area buried by each HCDR3 variant Fab when bound to HER2. The surface area buried when antibody Fab binds to Her2 is defined as the binding interface area between paratope and epitope (Table 2).

In several cases, de novo HCDR3 variants show larger binding interface area than trastuzumab which could suggest novel interactions with HER2 epitope. Interestingly, no correlation is observed between binding interface area to the affinity of binding. This finding would suggest that hydrophobic contributions and surface area burial are not key determinants of binding. Moreover, specific contacts formed between each designed HCDR3 and the epitope are critical to the binding stability of the Fab-Her2 complex. Furthermore, the present techniques calculated the grand average of hydropathy values (GRAVY) of each HCDR3 variant, which defines the collective hydrophobic properties summed over each residue, and compare to the binding affinities. The present techniques observe no correlation between affinity and hydrophobicity which further confirms the hydrophobic effect is not the major determinant of binding for de novo designed HCDR3. (Table 2). Combined, these results suggest that binding affinity of designed HCDR3 is intrinsic to the sequence design and is not driven by a common binding mechanism. The high dependence of binding on sequence attributes agree with a low probability of designing binders by chance.

### Validation on Additional Targets

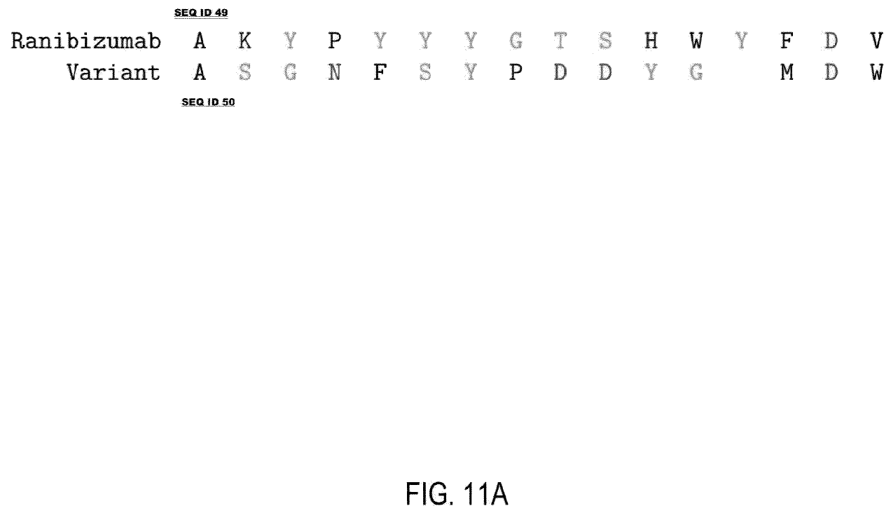

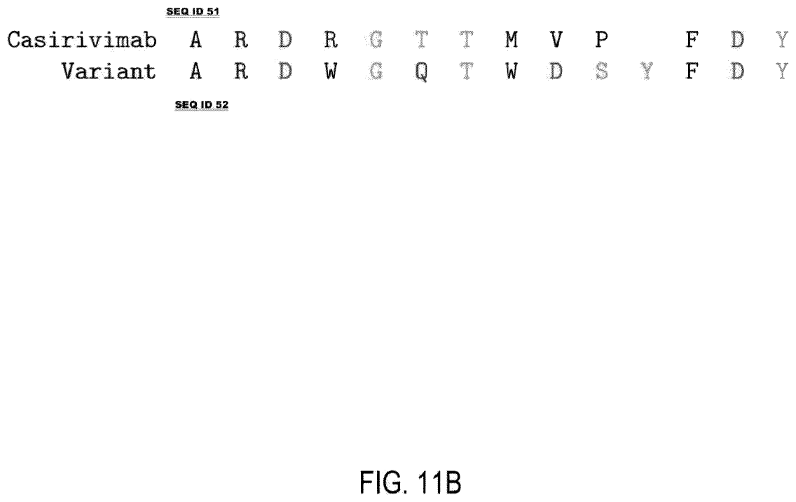

The present techniques next conduct a pilot study to demonstrate the applicability of our approach to a broader set of antigens. For these additional targets, the present techniques do not pre-screen by the ACE assay. Rather, the present techniques sample a small number of sequences and validate binding by SPR. The present techniques first to successfully design HCDR3 variants of the therapeutic ranibizumab, which binds to human vascular endothelial growth factor A (VEGF-A), as shown in FIG. 11A. The binder has an affinity of 48.2 nM, as measured by SPR, compared to sub-nanomolar binding of ranibizumab (0.37 nM). Additionally, the designed HCDR3 is highly divergent from ranibizumab, with an edit distance of 13, and novel, with a minimum of 4 mutations separating it from any HCDR3 in OAS.

The present techniques next design a set of HCDR3 variants of casirivimab, conditioned on omicron spike protein binding. Casirivimab binds to multiple COVID spike protein variants and in particular binds weakly to Omicron. The present techniques measured casirivimab affinity to Omicron via SPR at 240.0 nM (Table S1). The present techniques identify one AI designed variant that binds with similar affinity to Omicron at 179.7 nM (FIG. 11B, Table S1). Interestingly, the present techniques observe no binding to other spike protein variants for our AI-design, suggesting the potential for controllability of target specificity among homologous antigens. The designed variant has a distinct HCDR3 sequence compared to Casirivimab, with an edit distance of 6 and a minimum edit distance of 2 from any HCDR3 in OAS. Additionally, the HCDR3 has at least an edit distance of 4 from any HCDR3 in CoV-AbDab. These binding designs to two additional antigens highlight the extensibility of our zero-shot design approach and indicate the potential for selective antigen controllability with generative AI.

TABLE S1
Measured binding affinities of casirivimab and AI-designed variant
to COVID antigens. N/A indicates a lack of binding. In this setting,
the model was instructed to generate HCDR3 variants targeting Omicron
that fit within the casirivimab framework. These results indicate
that the model successfully designed an HCDR3 variant that losses
activity to COVID Spike Wildtype/Beta/Delta, yet maintains activity
to Omicron. These results are a first step toward controllability
of antibody design to specific protein variants.

| | Wildtype | Beta | Delta | Omicron |
|---|---|---|---|---|
| Casirivimab | 8.3 nM | 470.0 nM | 4.2 nM | 240.0 nM |
| AI-Designed Binder | N/A | N/A | N/A | 179.7 nM |

### Extension to Multiple CDRs

Expanding antibody design beyond HCDR3 allows for increased sequence diversity and controllability and, represents the next step toward fully de novo design. To this end we applied an alternative multi-step AI design method that is distinct from our described zero-shot approach to generate variants of all heavy chain variable regions (HCDR1, HCDR2, HCDR3) simultaneously. The present techniques report multiple binding designs to HER2 identified within a library of less than 500 multi-step designed variants (Table 2). The present techniques find that these binders again are distinct from examples in the model's training data and antibodies in the SAbDab and OAS databases (FIG. 9C, FIG. 9I).

### Exemplary Materials and Methods

### Library Designs

For the de novo design of HCDR3 trastuzumab variants, the present techniques may begin by designing two high-diversity libraries (HDLs), denoted HDL1, consisting of, for example, 223,046 and 217308 designs, respectively. HDL1 may be screened using the ACE assay and the dnACE assay described herein, and the results are used to design low-diversity libraries (LDLs), each consisting of 1,000 or fewer sequences that are screened using SPR. HDL2 is screened using ACE and the results are again used to design LDLs for SPR screening. In total, 199 binders are confirmed from HDL1 based on ACE scores reveal an estimate of an additional 3765 binders in HDL2 (Table S5). The ACE assay may not be used to screen variants of HCDR3 ranibizumab, HCDR3 casirivimab nor HCDR123 trastuzumab. Instead, small LDLs may be screened directly with SPR.

TABLE S4
Estimated number of binders according to ACE “bins”
in HDL2. In addition to the 224 confirmed binders, an additional
3,780 designs are estimated to bind from HDL2 based on the
distribution of sequences in ACE bins. Note the total number
of identified designs (41,942) is less than the total designs
(217,308). The difference corresponds to designs that did
not sort in ACE, indicating they are unlikely to bind.

| | | Number of | Number of |
|---|---|---|---|
| ACE Bin | Binding Rate in SPR | Identified Designs | Expected Binders |
| 0 | 0.045 | 35,484 | 1,597 |
| 1 | 0.194 | 4,459 | 865 |
| 2 | 0.522 | 1,359 | 709 |
| 3 | 0.932 | 368 | 343 |
| 4 | 0.914 | 200 | 183 |
| 5 | 0.942 | 72 | 68 |
| Aggregate | | 41,942 | 3,765 |

### Naturalness Score

The Naturalness score used in this study may be computed using the pre-trained antibody language model discussed herein and introduced previously. This model is based on the pseudo-perplexity across the extended CDRs (e.g., defined by a union of the IMGT and Martin definitions) of an antibody heavy chain under the language model. This metric may be predictive of desirable antibody therapeutic properties such as developability or lack of immunogenicity. The present techniques may place variant HCDR3s into the wildtype scaffold by replacing the wildtype HCDR3. In addition to computing naturalness for the present de novo binders, the present techniques may further include several controls:

1. OAS: Consists of 1,000 HCDR3s randomly sampled from OAS. Naturalness scores are computed over a grafting of the HCDR3 into the trastuzumab scaffold. We expect sequences from OAS to have high Naturalness scores, given that the Naturalness model is pretrained on OAS, so we treat this as a positive control.
2. Frequency Baseline: These are 1000 sequences generated by randomly sampling from a length-conditioned frequency distribution of OAS. The present techniques may compute PL( [symbol] ), the probability that an HCDR3 in OAS has length f, and then compute the probability of sampling a particular sequence with length f, using an independent factorization based on amino acid frequencies, defined as:

$${ℙ}_{H} ( {a}_{1} {a}_{2} … {α}_{l} \vert L = ℓ ) = ℙ ( {a}_{1} \vert L = ℓ ) ℙ ( {a}_{2} \vert L = ℓ ) … ℙ ( {a}_{l} \vert L = ℓ ) .$$

The present techniques may include sampling a number (e.g., 1000) of lengths [symbol] 1, [symbol] 2, . . . , [symbol] 1000˜PL and then sample 1000 sequences according to sᵢ˜ [symbol]
3. Phage Display Baseline: These are 1000 HCDR3s randomly sampled from a first round of phage display panning (Liu et al.). Antibody heavy chain sequences are sampled and the HCDR3s are extracted. The collection of antibodies sampled from antibodies sampled from consist of both non-binders and binders.
4. Scrambled OAS: This consists of permuted versions of the 1000 HCDR3s in the OAS control. For each such HCDR3 the present techniques may include permuting the respective sequence 5 different times, computing naturalness using the permuted HCDR3, and reporting the average across the 5 permutations. In some aspects, the motivation for this computation as a negative control is that permuting a protein sequence destroys positional information. Lower Naturalness scores of this baseline compared to the first OAS baseline implies that the Natu-ralness model is able to capture positional information, and is not just considering amino acid com-position.

The naturalness scores of the present de novo designs may be compared to these controls using two-sample t-tests (H₀:μ₁=μ₂, Hₐ:μ₁!=μ₂) and compared to wildtype using one-sample t-tests with the wildtype naturalness as the population mean (H₀:μ₁=μ, Hₐ:μ₁!H μ). A table of the mean naturalness scores is shown below, across the different groups as well as p-values for the relevant statistical comparisons to the de novo binders.

TABLE 2

| | | p-value (comparison |
|---|---|---|
| Group | Mean Naturalness | to de novo) |
| De Novo | 0.175 | N/A |
| Wildtype | 0.235 | 3.66 · 10⁻⁹⁵ |
| OAS | 0.194 | 3.63 · 10⁻¹⁶ |
| Frequency Baseline | 0.143 | 4.67 · 10⁻⁵³ |
| Phage Display Baseline | 0.136 | 1.11 · 10⁻⁸⁷ |
| Scrambled OAS | 0.101 | 8.05 · 10⁻³⁰⁰ |

Table 2 depicts mean naturalness scores across different groups, and p-values using trastuzumab scaffolding.

Further, the present techniques previously noted the diversity of designs relative to OAS, the pre-training data used for the model that was used to computed naturalness herein, in some aspects. Taking samples far away from the training set could lead to lower naturalness. Indeed, as shown in FIG. 9B, empirical evidence shows that naturalness for the designs tends to decrease as distance from OAS increases. Of particular interest is the fact that the average naturalness for the present designs contained in OAS may be higher than the average OAS control naturalness (p=9.45e-19) and higher than trastuzumab's naturalness (p=1.31e-4). In fact, the present designs that are 1 mutation away from any HCDR3 in OAS have significantly higher naturalness than the OAS control sequences (p=9.83e-5).

### Exemplary Binder Data

### Common Sequences

As discussed, the present techniques include training models using training data. For example, the present techniques may use the following binder data for training, in some aspects. Below are Tables E1-E4 including binder data for, respectively, De Novo Trastuzumab HCDR3 Variant HER2 Binders, De Novo Ranibizumab HCDR3 Variant VEGF-A Binders, De Novo Casirivimab HCDR3 Variant COVID-Omicron Binders De Novo Trastuzumab HCDR123 Variant HER2 Binders. The following common sequences apply to the binder data.

### HER2 Binders

For the HER2 binders the following information is relevant but does not change for each binder:

Parent Antibody: Trastuzumab
Antigen: HER2
Parent PDB: 1N8Z
Parent Heavy Chain Sequence (SEQ ID NO: 76):
EVOLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEP
Parent HCDR1 (SEQ ID NO: 54):
GFNIKDTY
Parent HCDR2 (SEQ ID NO: 55):
IYPTNGYT
Parent HCDR3 (SEQ ID NO: 20):
SRWGGDGFYAMDY
Parent Light Chain Sequence (SEQ ID NO: 77):
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Antigen Chain Sequence (SEQ ID NO: 78):
TQVCTGTDMKLRLPASPETHLDMLRHLYQGCQVVQGNLELTYLPTNASLS
FLQDIQEVQGYVLIAHNQVRQVPLQRLRIVRGTQLFEDNYALAVLDNGDP
LNNTTPVTGASPGGLRELQLRSLTEILKGGVLIQRNPQLCYQDTILWKDI
FHKNNQLALTLIDTNRSRACHPCSPMCKGSRCWGESSEDCQSLTRTVCAG
GCARCKGPLPTDCCHEQCAAGCTGPKHSDCLACLHFNHSGICELHCPALV
TYNTDTFESMPNPEGRYTFGASCVTACPYNYLSTDVGSCTLVCPLHNQEV
TAEDGTQRCEKCSKPCARVCYGLGMEHLREVRAVTSANIQEFAGCKKIFG
SLAFLPESFDGDPASNTAPLQPEQLQVFETLEEITGYLYISAWPDSLPDL
SVFQNLQVIRGRILHNGAYSLTLQGLGISWLGLRSLRELGSGLALIHHNT
HLCFVHTVPWDQLFRNPHQALLHTANRPEDECVGEGLACHQLCARGHCWG
PGPTQCVNCSQFLRGQECVEECRVLQGLPREYVNARHCLPCHPECQPQNG
SVTCFGPEADQCVACAHYKDPPFCVARCPSGVKPDLSYMPIWKFPDEEGA
CQPCPIN

### COVID, VEGF and HER2

Common/invariant information for the COVID, VEGF and HER2 affinity tables is as follows:

### COVID:

Parent Antibody: Casirivimab
Parent Antibody PDB: 6XDG
Antigens for which binding affinity data was
collected via SPR: SARS-COV2 Spike RBD
(Wildtype, Beta, Delta, Omicron)
Parent Heavy Chain Sequence (SEQ ID NO: 79):
QVQLVESGGGLVKPGGSLRLSCAASGFTFSDYYMSWIRQAPGKGLEWVSY
ITYSGSTIYYADSVKGRFTISRDNAKSSLYLQMNSLRAEDTAVYYCARDR
GTTMVPFDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKT
Parent HCDR3 (SEQ ID NO: 51):
ARDRGTTMVPFDY
Parent Light Chain Sequence (SEQ ID NO: 80):
DIQMTQSPSSLSASVGDRVTITCQASQDITNYLNWYQQKPGKAPKLLIYA
ASNLETGVPSRFSGSGSGTDFTFTISGLQPEDIATYYCQQYDNLPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Antigen for which model was conditioned on:
SARS-COV-2 Omicron Spike RBD PDB: 7Q09
Sequence (SEQ ID NO: 81):
MFVFLVLLPLVSSQCVNLTTRTQLPPAYTNSFTRGVYYPDKVFRSSVLHS
TQDLFLPFFSNVTWFHVISGTNGTKRFDNPVLPFNDGVYFASIEKSNIIR
GWIFGTTLDSKTQSLLIVNNATNVVIKVCEFQFCNDPFLDHKNNKSWMES
EFRVYSSANNCTFEYVSQPFLMDLEGKQGNFKNLREFVFKNIDGYFKIYS
KHTPIIVREPEDLPQGFSALEPLVDLPIGINITRFQTLLALHRSYLTPGD
SSSGWTAGAAAYYVGYLQPRTFLLKYNENGTITDAVDCALDPLSETKCTL
KSFTVEKGIYQTSNFRVQPTESIVRFPNITNLCPFDEVFNATRFASVYAW
NRKRISNCVADYSVLYNLAPFFTFKCYGVSPTKLNDLCFTNVYADSFVIR
GDEVRQIAPGQTGNIADYNYKLPDDFTGCVIAWNSNKLDSKVSGNYNYLY
RLFRKSNLKPFERDISTEIYQAGNKPCNGVAGENCYFPLRSYSFRPTYGV
GHQPYRVVVLSFELLHAPATVCGPKKSTNLVKNKCVNFNFNGLKGTGVLT
ESNKKFLPFQQFGRDIADTTDAVRDPQTLEILDITPCSFGGVSVITPGTN
TSNQVAVLYQGVNCTEVPVAIHADQLTPTWRVYSTGSNVFQTRAGCLIGA
EYVNNSYECDIPIGAGICASYQTQTKSHGSASSVASQSIIAYTMSLGAEN
SVAYSNNSIAIPTNFTISVTTEILPVSMTKTSVDCTMYICGDSTECSNLL
LQYGSFCTQLKRALTGIAVEQDKNTQEVFAQVKQIYKTPPIKYFGGFNFS
QILPDPSKPSKRSFIEDLLFNKVTLADAGFIKQYGDCLGDIAARDLICAQ
KFKGLTVLPPLLTDEMIAQYTSALLAGTITSGWTFGAGAALQIPFAMQMA
YRENGIGVTQNVLYENQKLIANQFNSAIGKIQDSLSSTASALGKLQDVVN
HNAQALNTLVKQLSSKFGAISSVLNDIFSRLDPPEAEVQIDRLITGRLQS
LQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWF
VTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQEL
GKYEQGSGYIPEAPRDGQAYVRKDGEWVLLSTFLGRSLEVLFQGPGHHHH
HHHHSAWSHPQFEKGGGSGGGGSGGSAWSHPQFEK
VEGF:
Parent antibody: Ranibizumab
Patent Antibody PDB: 1CZ8
Parent Heavy Chain Sequence (SEQ ID NO: 82):
EVQLVESGGGLVQPGGSLRLSCAASGYDFTHYGMNWVRQAPGKGLEWVGW
INTYTGEPTYAADFKRRFTFSLDTSKSTAYLQMNSLRAEDTAVYYCAKYP
YYYGTSHWYFDVWGQGTLVTVSSASTKGPSVFPLAPSGTAALGCLVKDYF
PEPVTVSWNSGALTSGVHTFPAVLQSSGLYSLSSVVTVPSSSLGTQTYIC
NVNHKPSNTKVDKKVEPK
Parent HCDR3 (SEQ ID NO: 49):
AKYPYYYGTSHWYFDV
Parent Light Chain Sequence (SEQ ID NO: 83):
DIQLTQSPSSLSASVGDRVTITCSASQDISNYLNWYQQKPGKAPKVLIYF
TSSLHSGVPSRFSGSGSGTDFTLTISSLQPEDFATYYCQQYSTVPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGE
Antigen Chain Sequence (SEQ ID NO: 84):
VVKFMDVYQRSYCHPIETLVDIFQEYPDEIEYIFKPSCVPLMRCGGCCND
EGLECVPTEESNITMQIMRIKPHQGQHIGEMSFLQHNKCECRPK
HER2: trastuzumab binding affinity and HCDR3
(SEQ ID NO: 20):
SRWGGDGFYAMDY, 1.940000e−09 (KD (M)), −8.71
(log(KD(M))), 1.94 (KD (nM)).

TABLE E1
De Novo Trastuzumab HCDR3
Variant HER2 Binders

| | | log(KD | KD | Seq ID |
|---|---|---|---|---|
| HCDR3 | KD (M) | (M) | (nM) | NO: |
| TRYFFNGWYYFDV | 9.40E−10 | −9.03 | 0.94 | 44 |
| ARYYYGFYYFDY | 1.21E−09 | −8.92 | 1.21 | 42 |
| ARWGNYYYYMDY | 1.69E−09 | −8.77 | 1.69 | 41 |
| ARWSYGYYNFDY | 1.98E−09 | −8.7 | 1.98 | 85 |
| AVYSPDFYYLGY | 2.06E−09 | −8.69 | 2.06 | 86 |
| ARWGYYYYYMDY | 2.28E−09 | −8.64 | 2.28 | 87 |
| ARWGDYYYYMDY | 2.36E−09 | −8.63 | 2.36 | 88 |
| TRWLEGLSYFMDY | 2.40E−09 | −8.62 | 2.4 | 89 |
| ARWPFYGPYNYDY | 2.64E−09 | −8.58 | 2.64 | 90 |
| TRYGYGYYYFDY | 2.73E−09 | −8.56 | 2.73 | 91 |
| VRYGYGYYYFDY | 2.77E−09 | −8.56 | 2.77 | 92 |
| TRYYYGLYYFDY | 3.02E−09 | −8.52 | 3.02 | 93 |
| TRYGSYYYYFDY | 3.18E−09 | −8.5 | 3.18 | 94 |
| ARYEAYGLYPFGY | 3.39E−09 | −8.47 | 3.39 | 95 |
| ARWLGEGYYNFDY | 3.42E−09 | −8.47 | 3.42 | 96 |
| ARWLAYWYAMDY | 3.55E−09 | −8.45 | 3.55 | 97 |
| TRYGGGYYYFDY | 3.59E−09 | −8.45 | 3.59 | 98 |
| ARWGGYYYYMDY | 3.62E−09 | −8.44 | 3.62 | 99 |
| VRYGGGYYYFDV | 3.96E−09 | −8.4 | 3.96 | 100 |
| ANDIYIQGYDLNR | 3.99E−09 | −8.4 | 3.99 | 46 |
| ARWGGNYYYMDY | 4.22E−09 | −8.37 | 4.22 | 101 |
| ARWGSGYYYMDY | 4.46E−09 | −8.35 | 4.46 | 102 |
| ATYSFGAYYYVF | 4.73E−09 | −8.33 | 4.73 | 103 |
| ARWPGYGSYYMDV | 4.83E−09 | −8.32 | 4.83 | 104 |
| VRWRSDFYYFDY | 4.95E−09 | −8.31 | 4.95 | 105 |
| TRYGYGYYYMDY | 5.25E−09 | −8.28 | 5.25 | 106 |
| ALWGFGWYYFDY | 5.31E−09 | −8.28 | 5.31 | 107 |
| VRYYGDGGYYFDY | 5.35E−09 | −8.27 | 5.35 | 108 |
| TRFRSFFYNFDY | 5.40E−09 | −8.27 | 5.4 | 109 |
| VRYGYGYYYMDY | 5.48E−09 | −8.26 | 5.48 | 110 |
| ARWGDGYYYMDY | 5.57E−09 | −8.25 | 5.57 | 111 |
| ARWYGAYYHMDY | 5.59E−09 | −8.25 | 5.59 | 112 |
| ARYRHWYYDYDY | 6.15E−09 | −8.21 | 6.15 | 113 |
| ARYGGFYYYFDY | 6.17E−09 | −8.21 | 6.17 | 114 |
| VRWGGYYYYFDY | 6.35E−09 | −8.2 | 6.35 | 115 |
| ARYGSYFYYMDY | 6.53E−09 | −8.19 | 6.53 | 116 |
| VRYGGWGFWYFDY | 6.76E−09 | −8.17 | 6.76 | 117 |
| TRYYYYGSYHFDY | 6.82E−09 | −8.17 | 6.82 | 118 |
| ARYYYYSYAMDY | 6.90E−09 | −8.16 | 6.9 | 119 |
| ARWGYSGWYVMDY | 6.93E−09 | −8.16 | 6.93 | 120 |
| ARWYYGYYYYFDV | 7.04E−09 | −8.15 | 7.04 | 121 |
| ARYFYDYYYYFDF | 7.11E−09 | −8.15 | 7.11 | 122 |
| ARYGDGYYYYFAY | 7.31E−09 | −8.14 | 7.31 | 123 |
| ARWYGYGGYYFDY | 7.43E−09 | −8.13 | 7.43 | 124 |
| ARWGFSMYSFDY | 7.50E−09 | −8.12 | 7.5 | 125 |
| TRNYYDYYYYFDY | 7.54E−09 | −8.12 | 7.54 | 126 |
| ATYGRDYYHFAS | 7.55E−09 | −8.12 | 7.55 | 127 |
| ARWAGDYYVMDY | 7.68E−09 | −8.11 | 7.68 | 128 |
| ARYYGYGGYYFDY | 8.05E−09 | −8.09 | 8.05 | 129 |
| ARWGGPGYYAFVW | 8.08E−09 | −8.09 | 8.08 | 130 |
| ARYYGYGGYWYDT | 8.19E−09 | −8.09 | 8.19 | 131 |
| ARWYYYGGYYFDY | 8.29E−09 | −8.08 | 8.29 | 132 |
| ARNYYDLYYYFDY | 8.41E−09 | −8.08 | 8.41 | 133 |
| ARYYYDYYYYFDY | 8.44E−09 | −8.07 | 8.44 | 134 |
| ARWPGGYYHMDY | 8.49E−09 | −8.07 | 8.49 | 135 |
| ARWFGGYYHMDY | 8.52E−09 | −8.07 | 8.52 | 136 |
| TSIFSGGYYGLDS | 8.57E−09 | −8.07 | 8.57 | 137 |
| ARWYGGYYHHDY | 8.57E−09 | −8.07 | 8.57 | 138 |
| ARYGYGYYVMDY | 8.65E−09 | −8.06 | 8.65 | 139 |
| TRYGSFHYYYFDV | 8.73E−09 | −8.06 | 8.73 | 140 |
| ARWGGYGYYYMDV | 8.78E−09 | −8.06 | 8.78 | 141 |
| ARYGGGYYYYDY | 9.11E−09 | −8.04 | 9.11 | 142 |
| TRYASGYFYAFDY | 9.14E−09 | −8.04 | 9.14 | 143 |
| APYGPGYWYGV | 9.15E−09 | −8.04 | 9.15 | 144 |
| ARWGEFGPYYFDY | 9.24E−09 | −8.03 | 9.24 | 145 |
| ARWTERGFYSFDI | 9.28E−09 | −8.03 | 9.28 | 146 |
| ARWGGDFYAMDY | 9.49E−09 | −8.02 | 9.49 | 147 |
| ARYDYGYYIYVS | 9.49E−09 | −8.02 | 9.49 | 48 |
| ARYGYAPGFYYMDV | 9.72E−09 | −8.01 | 9.72 | 148 |
| TRWGGYYYFDY | 9.79E−09 | −8.01 | 9.79 | 149 |
| ARYYGYGGYYMDY | 9.91E−09 | −8 | 9.91 | 150 |
| ARYGYYLYAMDY | 1.01E−08 | −7.99 | 10.12 | 151 |
| AKFAYYGPYHFDP | 1.01E−08 | −7.99 | 10.12 | 152 |
| ARYGYYSYAMDY | 1.02E−08 | −7.99 | 10.2 | 153 |
| VRYGYDYYWFDY | 1.04E−08 | −7.98 | 10.42 | 154 |
| ARYGLGYYYMDY | 1.05E−08 | −7.98 | 10.49 | 155 |
| ARFGNSGYQFDY | 1.05E−08 | −7.98 | 10.5 | 156 |
| AIYSDGAYFYGR | 1.06E−08 | −7.98 | 10.58 | 157 |
| VRYGYGYYAMDY | 1.07E−08 | −7.97 | 10.71 | 158 |
| ARWLGSGYYWFDY | 1.09E−08 | −7.96 | 10.9 | 159 |
| ARYTFGAYYYSD | 1.09E−08 | −7.96 | 10.94 | 160 |
| ARWYYYGRYYFDY | 1.10E−08 | −7.96 | 11 | 161 |
| ATWYGGNYHMYP | 1.12E−08 | −7.95 | 11.16 | 162 |
| ARYFGGSYAYVY | 1.12E−08 | −7.95 | 11.2 | 163 |
| ARWYGYYYYFDV | 1.12E−08 | −7.95 | 11.24 | 164 |
| TRFLIGSYYYDY | 1.16E−08 | −7.93 | 11.63 | 165 |
| SRYYAGGWYYFDY | 1.18E−08 | −7.93 | 11.75 | 166 |
| ARWYGGGYYYMDY | 1.18E−08 | −7.93 | 11.84 | 167 |
| ARWSSGYYYMDY | 1.20E−08 | −7.92 | 12 | 168 |
| TRYGGYGYYYMDY | 1.20E−08 | −7.92 | 12 | 169 |
| TRYRGPYYYYFDP | 1.21E−08 | −7.92 | 12.07 | 170 |
| VRWGVGAYYNFDY | 1.21E−08 | −7.92 | 12.1 | 171 |
| ARYGYGYYYMDY | 1.22E−08 | −7.91 | 12.22 | 172 |
| ARWYYYGGYYFDV | 1.25E−08 | −7.9 | 12.49 | 173 |
| STYARDYYHFGS | 1.26E−08 | −7.9 | 12.59 | 174 |
| ALYGNYSYGFAR | 1.26E−08 | −7.9 | 12.6 | 175 |
| ARWFYDYYYMDY | 1.26E−08 | −7.9 | 12.6 | 176 |
| ARWGGYYYYFDY | 1.28E−08 | −7.89 | 12.8 | 177 |
| ARFGSYYYYFDY | 1.30E−08 | −7.89 | 13 | 178 |
| ARWGGYYYYFDV | 1.30E−08 | −7.89 | 13 | 179 |
| ARWRGDGGYYFDY | 1.31E−08 | −7.88 | 13.1 | 180 |
| VRYGFDGSYYFDP | 1.32E−08 | −7.88 | 13.16 | 181 |
| ARYVGLGGYPLGY | 1.32E−08 | −7.88 | 13.21 | 182 |
| ARYGGGYYYMDY | 1.33E−08 | −7.88 | 13.3 | 183 |
| ARWGYYGNYYFDY | 1.34E−08 | −7.87 | 13.4 | 184 |
| ARYGSYNYYFGD | 1.36E−08 | −7.87 | 13.57 | 185 |
| ARWQGGYYYWDF | 1.38E−08 | −7.86 | 13.8 | 186 |
| ARWYYDYYYMDY | 1.39E−08 | −7.86 | 13.88 | 187 |
| ARYGSSYYYYFDY | 1.39E−08 | −7.86 | 13.9 | 188 |
| TRYGLTFDMDY | 1.39E−08 | −7.86 | 13.9 | 189 |
| ARHFGVASYSFDY | 1.39E−08 | −7.86 | 13.92 | 190 |
| ARYGGYGGYYFDY | 1.40E−08 | −7.85 | 14 | 191 |
| AKYGATGWYYFDY | 1.41E−08 | −7.85 | 14.12 | 192 |
| ARYGYYGPYYFDY | 1.43E−08 | −7.84 | 14.3 | 193 |
| ARYGGGYNMDY | 1.43E−08 | −7.84 | 14.32 | 194 |
| TRYGYGGPYWFDY | 1.43E−08 | −7.84 | 14.34 | 195 |
| ARYRGVWYYDFDN | 1.45E−08 | −7.84 | 14.5 | 196 |
| ARYGWGYYYFDY | 1.47E−08 | −7.83 | 14.65 | 197 |
| ARYLQLGPYYFDY | 1.47E−08 | −7.83 | 14.7 | 198 |
| AKYLGQEAYAYFS | 1.49E−08 | −7.83 | 14.9 | 199 |
| ARWWGGGAYYFDY | 1.49E−08 | −7.83 | 14.9 | 200 |
| ARYYYGYGGYYFDY | 1.50E−08 | −7.82 | 14.96 | 201 |
| ARWDSGGPYYFDS | 1.54E−08 | −7.81 | 15.4 | 202 |
| VRYGGTYYYFDY | 1.54E−08 | −7.81 | 15.4 | 203 |
| AKYGDDNYGFSH | 1.54E−08 | −7.81 | 15.43 | 204 |
| ATYGYGYYYFDY | 1.55E−08 | −7.81 | 15.49 | 205 |
| ARYGLYYYYFDY | 1.55E−08 | −7.81 | 15.51 | 206 |
| ARYGSGYYYYFDY | 1.57E−08 | −7.8 | 15.7 | 207 |
| ARYGSRYYYYFDY | 1.57E−08 | −7.8 | 15.7 | 208 |
| ARYGLGYYYFDY | 1.57E−08 | −7.8 | 15.7 | 209 |
| ARWLAGYNYYFDV | 1.57E−08 | −7.8 | 15.74 | 210 |
| ARYTYFGSFDFDY | 1.58E−08 | −7.8 | 15.76 | 211 |
| ARWLRALGYYMDY | 1.59E−08 | −7.8 | 15.85 | 212 |
| ARYGGYYYYMDY | 1.63E−08 | −7.79 | 16.3 | 213 |
| ARYWGLGGYYFDH | 1.65E−08 | −7.78 | 16.48 | 214 |
| VRYGFYGYYYFDY | 1.65E−08 | −7.78 | 16.5 | 215 |
| AKYINAYSYAFFD | 1.70E−08 | −7.77 | 16.95 | 216 |
| ATYSPGAYYYGP | 1.73E−08 | −7.76 | 17.25 | 217 |
| ARYSGYGLYPYAY | 1.74E−08 | −7.76 | 17.41 | 218 |
| AKYAYYGDFWGSV | 1.75E−08 | −7.76 | 17.47 | 219 |
| ARWYVSYDYDV | 1.76E−08 | −7.75 | 17.6 | 220 |
| ARYPGYYYYFDY | 1.79E−08 | −7.75 | 17.86 | 221 |
| TRYGAPYFYFDY | 1.80E−08 | −7.75 | 17.97 | 222 |
| TRYEYQDYYHFDY | 1.85E−08 | −7.73 | 18.46 | 223 |
| ARYGAYFYAMDA | 1.86E−08 | −7.73 | 18.61 | 224 |
| AKYGSNYYYLGS | 1.87E−08 | −7.73 | 18.74 | 225 |
| TRNSFGFYAMDY | 1.90E−08 | −7.72 | 19 | 226 |
| ARWYGGYYMYDY | 1.91E−08 | −7.72 | 19.06 | 227 |
| ARWGYGGYYYFDY | 1.91E−08 | −7.72 | 19.1 | 228 |
| AKYSFGGYYYGD | 1.91E−08 | −7.72 | 19.13 | 229 |
| ASYGLDGYYYFDV | 1.93E−08 | −7.71 | 19.29 | 230 |
| ARWGYDYYYAMDY | 1.94E−08 | −7.71 | 19.4 | 231 |
| ARYYSYGQYAFDP | 1.94E−08 | −7.71 | 19.42 | 232 |
| ARYGGGGRYYFDY | 1.96E−08 | −7.71 | 19.6 | 233 |
| ARYWYGGYYYFDY | 1.97E−08 | −7.71 | 19.66 | 234 |
| ARYAHYGPYYFDF | 1.98E−08 | −7.7 | 19.8 | 235 |
| AAYTNTFYYLVY | 1.98E−08 | −7.7 | 19.84 | 236 |
| VRYGSDVYYYFDI | 1.99E−08 | −7.7 | 19.87 | 237 |
| ARYYGYGYYYFHY | 2.02E−08 | −7.7 | 20.18 | 238 |
| ARYGVVRYDFAY | 2.05E−08 | −7.69 | 20.47 | 239 |
| ARWGSIGYYAFDH | 2.05E−08 | −7.69 | 20.5 | 240 |
| ALYNYYYYAFGF | 2.06E−08 | −7.69 | 20.56 | 241 |
| ARWGYGYSYYFDV | 2.11E−08 | −7.68 | 21.1 | 242 |
| ARYWMGVYEFDY | 2.17E−08 | −7.66 | 21.67 | 243 |
| ARWEDYYYMDY | 2.18E−08 | −7.66 | 21.82 | 244 |
| VRYGGDYYYFDY | 2.19E−08 | −7.66 | 21.87 | 245 |
| ARYGYGYFYMDY | 2.19E−08 | −7.66 | 21.9 | 246 |
| ARYGYDGYYFAY | 2.22E−08 | −7.65 | 22.2 | 247 |
| ARYPSVVYAFGI | 2.23E−08 | −7.65 | 22.27 | 248 |
| ARYGGGFYAMDY | 2.24E−08 | −7.65 | 22.36 | 249 |
| ATWHYGGYFMSG | 2.25E−08 | −7.65 | 22.45 | 250 |
| ARWGYYYYAMDY | 2.26E−08 | −7.65 | 22.58 | 251 |
| ATYSTYYNYAFGI | 2.26E−08 | −7.65 | 22.6 | 252 |
| TRYLHDYYYYFDY | 2.27E−08 | −7.64 | 22.7 | 253 |
| AVSADIMYGVSV | 2.29E−08 | −7.64 | 22.87 | 254 |
| VRYFYDGNYYFDL | 2.29E−08 | −7.64 | 22.93 | 255 |
| ARYRGYVLYDFAY | 2.31E−08 | −7.64 | 23.07 | 256 |
| ARENDGSPGSFEY | 2.31E−08 | −7.64 | 23.14 | 257 |
| ARYGYYGYYMDY | 2.33E−08 | −7.63 | 23.3 | 258 |
| AVTDLDGLHSYRY | 2.33E−08 | −7.63 | 23.3 | 259 |
| ARYGGGYWYFDY | 2.34E−08 | −7.63 | 23.4 | 260 |
| ARYPPGYYIMDF | 2.35E−08 | −7.63 | 23.53 | 261 |
| ARFGGYYYYFDY | 2.35E−08 | −7.63 | 23.54 | 262 |
| TRYYDGAGPYYMDY | 2.37E−08 | −7.63 | 23.65 | 263 |
| ARYGTIYFHFDY | 2.39E−08 | −7.62 | 23.89 | 264 |
| VTYGTTYYHFGS | 2.41E−08 | −7.62 | 24.08 | 265 |
| ARYDPYGFYHLDY | 2.42E−08 | −7.62 | 24.24 | 266 |
| VRYGYGDYYYFDY | 2.51E−08 | −7.6 | 25.08 | 267 |
| AKYGGVGYYAMDY | 2.52E−08 | −7.6 | 25.2 | 268 |
| ARWGYGYYYFDY | 2.52E−08 | −7.6 | 25.2 | 269 |
| ARYSAYGLYDFAY | 2.55E−08 | −7.59 | 25.5 | 270 |
| ARYGTGNYAMDY | 2.63E−08 | −7.58 | 26.3 | 271 |
| ARWGYDYYYFAY | 2.65E−08 | −7.58 | 26.5 | 272 |
| ARWGYDYYYYFDV | 2.67E−08 | −7.57 | 26.7 | 273 |
| ARYGRGYYYFDY | 2.68E−08 | −7.57 | 26.8 | 274 |
| VRYGNSYYYDY | 2.69E−08 | −7.57 | 26.88 | 275 |
| VRFYYDYYFDY | 2.69E−08 | −7.57 | 26.9 | 276 |
| ARYDIGYYDMDK | 2.70E−08 | −7.57 | 26.97 | 277 |
| AATYGGLGDMSSI | 2.70E−08 | −7.57 | 27 | 278 |
| ARYGVYPFYYFDY | 2.70E−08 | −7.57 | 27 | 279 |
| ARNYVYWYGMTV | 2.75E−08 | −7.56 | 27.51 | 280 |
| AKYGTSNYGFSH | 2.76E−08 | −7.56 | 27.6 | 281 |
| ARWERYGYYVLDV | 2.79E−08 | −7.55 | 27.9 | 282 |
| ARYGAYSSYYFGY | 2.80E−08 | −7.55 | 27.97 | 283 |
| ARYFGGSYAYGR | 2.80E−08 | −7.55 | 27.99 | 284 |
| ARWLYYGYYFDY | 2.80E−08 | −7.55 | 28.04 | 285 |
| ARYLSYSYAWNY | 2.89E−08 | −7.54 | 28.92 | 286 |
| TRYYGDGYYDFDY | 2.92E−08 | −7.53 | 29.24 | 287 |
| ARYFYDHYYYFDV | 2.97E−08 | −7.53 | 29.72 | 288 |
| ARYYDDTIYSFY | 2.99E−08 | −7.53 | 29.85 | 289 |
| ARYYYDYGYYFDV | 3.00E−08 | −7.52 | 30 | 290 |
| ARGYYYYFYYNY | 3.03E−08 | −7.52 | 30.31 | 291 |
| ARWGSYGYYYFDY | 3.09E−08 | −7.51 | 30.9 | 292 |
| ARHYDYGRYYMDV | 3.09E−08 | −7.51 | 30.94 | 293 |
| ARWGGFGYYYFDY | 3.10E−08 | −7.51 | 31 | 294 |
| ARYGGGYYYYFDV | 3.14E−08 | −7.5 | 31.4 | 295 |
| TRTTLGFYAMDY | 3.23E−08 | −7.49 | 32.3 | 296 |
| ARYGGAGYYLDY | 3.24E−08 | −7.49 | 32.39 | 297 |
| ARWGSEAFYWFDY | 3.28E−08 | −7.48 | 32.84 | 298 |
| APWWSGWYYFFDT | 3.30E−08 | −7.48 | 33 | 299 |
| TRYYDGYYAMDY | 3.31E−08 | −7.48 | 33.1 | 300 |
| ARYPNYGFYMFDY | 3.33E−08 | −7.48 | 33.31 | 301 |
| ARGYYYYQWYYY | 3.36E−08 | −7.47 | 33.57 | 302 |
| ARWRDYYYYFDY | 3.38E−08 | −7.47 | 33.8 | 303 |
| ARWVSGARYYMDV | 3.38E−08 | −7.47 | 33.8 | 304 |
| AAYGPQGYAFAV | 3.38E−08 | −7.47 | 33.82 | 305 |
| ARYYGGLYYFDY | 3.44E−08 | −7.46 | 34.38 | 306 |
| ARFGGAGGYYFDY | 3.50E−08 | −7.46 | 35.04 | 307 |
| AGYWYGSYSFDY | 3.54E−08 | −7.45 | 35.36 | 308 |
| TRYRGAHGYYFDY | 3.55E−08 | −7.45 | 35.47 | 309 |
| ARYGDADFYAFDP | 3.58E−08 | −7.45 | 35.82 | 310 |
| ARYIFGGPYYFDY | 3.60E−08 | −7.44 | 36.04 | 311 |
| ASYYDYYYAFGT | 3.61E−08 | −7.44 | 36.08 | 312 |
| ARFPVSSYAYGF | 3.62E−08 | −7.44 | 36.2 | 313 |
| VRYPEVYGYYFDY | 3.63E−08 | −7.44 | 36.28 | 314 |
| ANVGSSLGIAALD | 3.67E−08 | −7.44 | 36.72 | 315 |
| SYPDFGYYVYFAY | 3.68E−08 | −7.43 | 36.76 | 316 |
| AKYAFGGWSYSP | 3.77E−08 | −7.42 | 37.7 | 317 |
| ARASWYWYGGDK | 3.78E−08 | −7.42 | 37.78 | 318 |
| AKYTRGFSYGFGY | 3.78E−08 | −7.42 | 37.82 | 319 |
| ARWGYGYYYSFDY | 3.80E−08 | −7.42 | 37.95 | 320 |
| ARYGRNGSYSYDY | 3.82E−08 | −7.42 | 38.2 | 321 |
| AKFERGYYSFDFG | 3.83E−08 | −7.42 | 38.28 | 322 |
| ARYGYGYYYYFDY | 3.91E−08 | −7.41 | 39.1 | 323 |
| ARYADYQFHFDN | 3.96E−08 | −7.4 | 39.59 | 324 |
| AASADFMYGVIR | 3.96E−08 | −7.4 | 39.62 | 325 |
| ARYPGGGIYDFDP | 3.99E−08 | −7.4 | 39.91 | 326 |
| ARYGGYGYYWFDY | 4.00E−08 | −7.4 | 40 | 327 |
| ARWLRGLGYYWDY | 4.01E−08 | −7.4 | 40.11 | 328 |
| ARYPEDFYYFDV | 4.02E−08 | −7.4 | 40.24 | 329 |
| ARYGDSYYYYFDY | 4.06E−08 | −7.39 | 40.6 | 330 |
| ARYGPYYYYAMDY | 4.13E−08 | −7.38 | 41.25 | 331 |
| AINLRGYWMDN | 4.15E−08 | −7.38 | 41.5 | 332 |
| TRYYGGYGYYFDY | 4.16E−08 | −7.38 | 41.57 | 333 |
| ARYGGYGNYWFDT | 4.21E−08 | −7.38 | 42.12 | 334 |
| ARGGYGYYTWFDY | 4.21E−08 | −7.38 | 42.13 | 335 |
| ARYGTGTYYYFLH | 4.23E−08 | −7.37 | 42.25 | 336 |
| AGSSASGWYHYTT | 4.23E−08 | −7.37 | 42.25 | 337 |
| ARWEGYGSYEFAY | 4.25E−08 | −7.37 | 42.45 | 338 |
| ARYLSYSYAWDN | 4.26E−08 | −7.37 | 42.61 | 339 |
| ARYYYDFGYYFDY | 4.28E−08 | −7.37 | 42.75 | 340 |
| ARYGGLGGYTLAY | 4.29E−08 | −7.37 | 42.87 | 341 |
| AQNWDRGSYFYTY | 4.31E−08 | −7.37 | 43.05 | 342 |
| AANAAIFAAWGA | 4.31E−08 | −7.37 | 43.07 | 343 |
| TRTRSGAYFFDY | 4.34E−08 | −7.36 | 43.4 | 344 |
| ARYGARSGYAYEY | 4.51E−08 | −7.35 | 45.1 | 345 |
| ARDGHYFTYDY | 4.51E−08 | −7.35 | 45.12 | 346 |
| ARNYYYGAYYFDY | 4.55E−08 | −7.34 | 45.51 | 347 |
| ASVDLGGFYDFRY | 4.61E−08 | −7.34 | 46.1 | 348 |
| ARYYGYYYAFDY | 4.63E−08 | −7.33 | 46.3 | 349 |
| ARWQGRSGYYMDY | 4.83E−08 | −7.32 | 48.33 | 350 |
| ARMSYYFYYMDY | 4.89E−08 | −7.31 | 48.88 | 351 |
| AAWYSTYYFDY | 4.94E−08 | −7.31 | 49.4 | 352 |
| AYTGAPHGGYHYYAY | 4.94E−08 | −7.31 | 49.44 | 353 |
| ARSGGHGVNGHWRSF | 4.98E−08 | −7.3 | 49.8 | 354 |
| ARYGYDYYYYFDV | 5.00E−08 | −7.3 | 50 | 355 |
| AKYVLVGSYGV | 5.02E−08 | −7.3 | 50.15 | 356 |
| SIYGTDYYWFAD | 5.04E−08 | −7.3 | 50.39 | 357 |
| ARFGPAYFYGFYY | 5.09E−08 | −7.29 | 50.85 | 358 |
| ARYMRPAFYPFLY | 5.09E−08 | −7.29 | 50.86 | 359 |
| ARGGYYGYWYYFDY | 5.12E−08 | −7.29 | 51.23 | 360 |
| ARYGSGYYGLDY | 5.15E−08 | −7.29 | 51.48 | 361 |
| SRWYGDYFAMDY | 5.18E−08 | −7.29 | 51.78 | 362 |
| ATNAFGLWSYGF | 5.28E−08 | −7.28 | 52.8 | 363 |
| ARYGVYGYAMDY | 5.33E−08 | −7.27 | 53.32 | 364 |
| ASYGSGSYFSFPL | 5.39E−08 | −7.27 | 53.93 | 365 |
| ARYGYGAYYYFDY | 5.40E−08 | −7.27 | 54.01 | 366 |
| ARYLSYSYAWDY | 5.48E−08 | −7.26 | 54.78 | 367 |
| ARHGSYYYDFDF | 5.66E−08 | −7.25 | 56.64 | 368 |
| TRYFYDFPYMDY | 5.68E−08 | −7.25 | 56.81 | 369 |
| ARYGGLSYYSFDL | 5.81E−08 | −7.24 | 58.1 | 370 |
| AKFHWGEDWLSDN | 5.91E−08 | −7.23 | 59.1 | 371 |
| ARYGYYGYWFDY | 5.94E−08 | −7.23 | 59.39 | 372 |
| ARYLSYSFAWDY | 5.99E−08 | −7.22 | 59.91 | 373 |
| VKHGIDYYWYGD | 6.01E−08 | −7.22 | 60.14 | 374 |
| AGSVAEGWYHYRD | 6.03E−08 | −7.22 | 60.3 | 375 |
| ARSGYPGYGSFAY | 6.07E−08 | −7.22 | 60.72 | 376 |
| ARYHNTGYFWGA | 6.10E−08 | −7.22 | 60.95 | 377 |
| ARYYDYGYTEDY | 6.12E−08 | −7.21 | 61.21 | 378 |
| ASYMGIVGYYPG | 6.23E−08 | −7.21 | 62.3 | 379 |
| ARTVVGWYGRFDY | 6.36E−08 | −7.2 | 63.64 | 380 |
| ARNSGTGIWAFAY | 6.50E−08 | −7.19 | 65 | 381 |
| ARYGTYGYAMDY | 6.55E−08 | −7.18 | 65.47 | 382 |
| ARWPPYYWWAFDY | 6.56E−08 | −7.18 | 65.58 | 383 |
| ARAGGTSISLGHG | 6.70E−08 | −7.17 | 67 | 384 |
| ARYGDAFIMSFGV | 6.74E−08 | −7.17 | 67.4 | 385 |
| APWGTYRYGYFDH | 6.76E−08 | −7.17 | 67.6 | 386 |
| ARYSYGAFYYSI | 7.04E−08 | −7.15 | 70.39 | 387 |
| AVYGPYGGFSYDS | 7.07E−08 | −7.15 | 70.7 | 388 |
| ARYYVGYYAMDY | 7.16E−08 | −7.15 | 71.57 | 389 |
| AIWSMYDVGYYPF | 7.18E−08 | −7.14 | 71.77 | 390 |
| ARYGLYGSYYFRY | 7.18E−08 | −7.14 | 71.8 | 391 |
| ARYYGYYYYMDY | 7.43E−08 | −7.13 | 74.3 | 392 |
| ARYSYYRYYFE | 7.46E−08 | −7.13 | 74.61 | 393 |
| ARYLTDADFRLHY | 7.51E−08 | −7.12 | 75.07 | 394 |
| ARYPYGYYAMDY | 7.81E−08 | −7.11 | 78.13 | 395 |
| TRWRWGYGYYFDF | 7.81E−08 | −7.11 | 78.14 | 396 |
| ARYWYGSYVFDY | 7.83E−08 | −7.11 | 78.32 | 397 |
| AADGYRGTYSF | 7.92E−08 | −7.1 | 79.19 | 398 |
| SRYGRGVYYYFDY | 7.97E−08 | −7.1 | 79.66 | 399 |
| AQSGSYFYPFSY | 8.32E−08 | −7.08 | 83.2 | 400 |
| ARPSYDVGGDMLF | 8.33E−08 | −7.08 | 83.25 | 401 |
| ARYVTANTYSYDP | 8.36E−08 | −7.08 | 83.55 | 402 |
| ARYYYSGYYMSV | 8.52E−08 | −7.07 | 85.23 | 403 |
| ALYKRYDYAFGY | 8.66E−08 | −7.06 | 86.63 | 404 |
| ARWGTYTYGYFDY | 8.81E−08 | −7.05 | 88.13 | 405 |
| ARWARGAGYYWDY | 8.96E−08 | −7.05 | 89.59 | 406 |
| ARYPSGYYYYFDY | 9.01E−08 | −7.05 | 90.13 | 407 |
| ARASWNWYGGDY | 9.29E−08 | −7.03 | 92.91 | 408 |
| AFPWAGHYMER | 9.50E−08 | −7.02 | 94.98 | 409 |
| ARGYYHYYHYQY | 9.51E−08 | −7.02 | 95.07 | 410 |
| VRYYRGYYYFDY | 9.64E−08 | −7.02 | 96.44 | 411 |
| ARYGDGTYYFFAH | 9.73E−08 | −7.01 | 97.3 | 412 |
| ARGYSGDWPYETFYV | 9.74E−08 | −7.01 | 97.35 | 47 |
| AFADSGAYGIWSF | 9.84E−08 | −7.01 | 98.42 | 45 |
| ARYDYYGYYYFDY | 9.90E−08 | −7 | 98.95 | 413 |
| ARYQAYDNSYGV | 1.02E−07 | −6.99 | 101.66 | 414 |
| ARSYAYLYGSWDY | 1.02E−07 | −6.99 | 102 | 415 |
| ARYHFSGYFMDA | 1.03E−07 | −6.99 | 103.01 | 416 |
| ARYYSYGGYYFDY | 1.06E−07 | −6.98 | 105.85 | 417 |
| ARYGRDYYYFDV | 1.06E−07 | −6.97 | 105.96 | 418 |
| AKYVASGYSLA | 1.09E−07 | −6.96 | 108.95 | 419 |
| ARYYSDYSYTFSY | 1.12E−07 | −6.95 | 112.19 | 420 |
| ARYHYSGYFMDS | 1.13E−07 | −6.95 | 112.72 | 421 |
| AAYWYGVYVFRY | 1.13E−07 | −6.95 | 112.85 | 422 |
| ARSEWFWGSFAY | 1.14E−07 | −6.94 | 114 | 423 |
| ARSHYGYYDYYTDF | 1.16E−07 | −6.94 | 115.5 | 424 |
| TLNGPLYWMWTL | 1.16E−07 | −6.94 | 115.95 | 425 |
| ARWWYGGRNYFDI | 1.16E−07 | −6.94 | 116.13 | 426 |
| ARYPDYYYAMDY | 1.17E−07 | −6.93 | 117.26 | 427 |
| ATWPHINTRIYAFDP | 1.17E−07 | −6.93 | 117.3 | 428 |
| ATGPYTVYSYPY | 1.20E−07 | −6.92 | 119.72 | 429 |
| ARYRGGYGGYVFDP | 1.20E−07 | −6.92 | 119.82 | 430 |
| ARNYYGSYYFDY | 1.20E−07 | −6.92 | 119.89 | 431 |
| ARFSGYGAYDYAY | 1.25E−07 | −6.9 | 124.99 | 432 |
| ARDGYPQYGGYGY | 1.27E−07 | −6.9 | 126.77 | 433 |
| AGYEGYGLYPFAY | 1.28E−07 | −6.89 | 128.1 | 434 |
| ARGYFGGYYFAY | 1.30E−07 | −6.88 | 130.44 | 435 |
| ARWWTPANYPFLY | 1.33E−07 | −6.88 | 133 | 436 |
| AKYGRSDYGFEH | 1.33E−07 | −6.88 | 133 | 437 |
| ARFLYGSYYFDY | 1.35E−07 | −6.87 | 134.93 | 438 |
| ASYYAPGSYGFDP | 1.35E−07 | −6.87 | 135.02 | 439 |
| ARYGLNHYYVYPY | 1.35E−07 | −6.87 | 135.31 | 440 |
| ARSYFRFTGDFFY | 1.35E−07 | −6.87 | 135.37 | 441 |
| SRYYDYNYYFMDY | 1.41E−07 | −6.85 | 141.43 | 442 |
| VRGYGYNFYYHGFSY | 1.47E−07 | −6.83 | 146.5 | 443 |
| ARHHYSGYFHDV | 1.48E−07 | −6.83 | 147.86 | 444 |
| ARTTSSIPSSFDY | 1.49E−07 | −6.83 | 148.77 | 445 |
| ARFDLVGAFDY | 1.51E−07 | −6.82 | 151.44 | 446 |
| ARDGYPFYSGMDY | 1.54E−07 | −6.81 | 153.52 | 447 |
| ATSGSVDRYDYVY | 1.55E−07 | −6.81 | 154.68 | 448 |
| ARYNPFDGFYFDV | 1.56E−07 | −6.81 | 155.5 | 449 |
| ATGYSYLRFYD | 1.64E−07 | −6.79 | 163.5 | 450 |
| ARGYYQDSQSWGY | 1.66E−07 | −6.78 | 165.5 | 451 |
| ASSGRLDLYDFRL | 1.71E−07 | −6.77 | 170.52 | 452 |
| ATGYGYLLYSDHD | 1.71E−07 | −6.77 | 171.22 | 453 |
| ARGYWWLYGFAY | 1.72E−07 | −6.77 | 171.68 | 454 |
| ARWSWPGYEFEF | 1.77E−07 | −6.75 | 177 | 455 |
| ARSTDYWHASQY | 1.79E−07 | −6.75 | 178.9 | 456 |
| ATGSGGGGFSYGY | 1.82E−07 | −6.74 | 181.9 | 457 |
| ARLAFDSVSFYSDY | 1.87E−07 | −6.73 | 186.77 | 458 |
| AQYGRGGYWYFDY | 1.88E−07 | −6.72 | 188.46 | 459 |
| ARSYAFLYGRMDY | 1.89E−07 | −6.72 | 188.5 | 460 |
| ASYAGYSPFDY | 2.02E−07 | −6.7 | 201.51 | 461 |
| ARSYYGAYYFDY | 2.02E−07 | −6.7 | 201.83 | 462 |
| ARDSPYYGSLYLY | 2.05E−07 | −6.69 | 205.07 | 463 |
| AKYGISDYGFDH | 2.07E−07 | −6.68 | 207.46 | 464 |
| ARSGEGYYAFDY | 2.10E−07 | −6.68 | 210.41 | 465 |
| ARYLEYTYAWDY | 2.18E−07 | −6.66 | 218.28 | 466 |
| ASGDLTAGSSQT | 2.19E−07 | −6.66 | 219.4 | 467 |
| ARWYGLGSYGLTY | 2.23E−07 | −6.65 | 222.67 | 468 |
| ARWEQASVGSFGY | 2.26E−07 | −6.65 | 225.85 | 469 |
| ARWRYGGAYQFAL | 2.31E−07 | −6.64 | 230.51 | 470 |
| ARDGGMYRYGLYG | 2.31E−07 | −6.64 | 231.3 | 471 |
| TRNGPLYWHWDL | 2.34E−07 | −6.63 | 234.09 | 472 |
| ARTTISSSSYTG | 2.48E−07 | −6.61 | 248 | 473 |
| ARGHYSLGYSFDF | 2.56E−07 | −6.59 | 256 | 474 |
| ALLYGYYPSGVG | 2.62E−07 | −6.58 | 262.02 | 475 |
| ATGYSYLRYYD | 2.73E−07 | −6.56 | 273 | 476 |
| ARDVYVGSPVFDY | 2.78E−07 | −6.56 | 277.82 | 477 |
| ARSNNWYSYSFQF | 2.78E−07 | −6.56 | 278 | 478 |
| AYMGSHTIYSFEH | 3.01E−07 | −6.52 | 301 | 479 |
| ASDRYGSYSLVS | 3.14E−07 | −6.5 | 314.28 | 480 |
| ATSGTASWQHYGV | 3.22E−07 | −6.49 | 321.95 | 481 |
| ARYYGWYYYFDY | 3.23E−07 | −6.49 | 322.69 | 482 |
| AVEGGTRALEP | 3.26E−07 | −6.49 | 325.59 | 483 |
| ARLGASHGYLYDD | 3.28E−07 | −6.48 | 327.57 | 484 |
| ARLYYSSFNGM | 3.39E−07 | −6.47 | 339 | 485 |
| ARDYDFGLSVGAV | 3.58E−07 | −6.45 | 357.6 | 486 |
| ARYHYRTIGSFGY | 3.67E−07 | −6.44 | 367 | 487 |
| AWASGYGGYGMDY | 3.68E−07 | −6.43 | 368.48 | 488 |
| ARYHYSGFFMDA | 4.33E−07 | −6.36 | 433.14 | 489 |
| ARYLSDSYAWDY | 4.34E−07 | −6.36 | 433.96 | 490 |
| ATGLSPSGSNGPY | 4.44E−07 | −6.35 | 444.26 | 491 |
| VRSGYDYYSWFAY | 4.65E−07 | −6.33 | 465.34 | 492 |
| ARGYAYYVAGREY | 4.96E−07 | −6.3 | 496 | 493 |
| TRRDFGYYMAY | 5.47E−07 | −6.26 | 546.94 | 494 |
| ARNYYGAAYYFDY | 5.70E−07 | −6.24 | 569.89 | 495 |
| ARYAGVERPGSFAY | 5.74E−07 | −6.24 | 574.18 | 43 |
| ARGYGVGASFVY | 6.30E−07 | −6.2 | 630 | 496 |
| ARTGWGYDNISGYEY | 1.46E−06 | −5.84 | 1461.56 | 497 |

TABLE E2
De Novo Ranibizumab HCDR3 Variant
VEGF-A Binders

| | | log(KD | KD | SEQ ID |
|---|---|---|---|---|
| HCDR3 | KD (M) | (M)) | (nM) | NO: |
| AKYPYYYGTSHWYFDV | 3.70E−10 | −9.43 | 0.37 | 49 |
| ASGNFSYPDDYGMDW | 4.82E−08 | −7.32 | 48.2 | 50 |

TABLE E3
De Novo Casirivimab HCDR3 Variant
COVID-Omicron Binders

| | KD | KD | KD | KD | |
|---|---|---|---|---|---|
| | (nM) - | (nM) - | (nM) - | (nM) - | SEQ |
| | Alpha | Beta | Delta | Omicron | ID |
| HCDR3 | COVID | COVID | COVID | COVID | NO: |
| ARDRGTTMVPFDY | 8.3 | 470 | 4.2 | 240 | 51 |
| ARDWGQTWDSYFD | | | | 179.7 | 52 |
| Y | | | | | |

TABLE E4
De Novo Trastuzumab HCDR123 Variant HER2 Binders

| | | | | | log(KD | KD |
|---|---|---|---|---|---|---|
| Category | HCDR1 | HCDR2 | HCDR3 | KD (M) | (M)) | (nM) |
| HCDR1\|2\|3 | GFNVSSSY (SEQ | IYPENG | SRYSYYGGYAFDY | 2.25E−07 | −6.65 | 224.9 |
| Variant | ID NO: 56) | ST (SEQ | (SEQ ID NO: 58) | | | |
| | | ID NO: | | | | |
| | | 57) | | | | |
| HCDR1\|2\|3 | GFNISDYY (SEQ | IYPSSG | ARDGGVGSYSMDV | 1.32E−06 | −5.88 | 1322.98 |
| Variant | ID NO: 60) | YT (SEQ | (SEQ ID NO: 61) | | | |
| | | ID NO: | | | | |
| | | 60) | | | | |
| HCDR1\|2\|3 | GFNIKDTW (SEQ | IDPANG | ARVGGRGYYYYDY | 3.94E−07 | −6.4 | 394.14 |
| Variant | ID NO: 65) | YT (SEQ | (SEQ ID NO: 67) | | | |
| | | ID NO: | | | | |
| | | 66) | | | | |
| HCDR1\|2\|3 | GFNIKDYY (SEQ | IYPSRG | ARHGSEGRYTMDV | 1.92E−07 | −6.72 | 192.08 |
| Variant | ID NO: 62) | YT (SEQ | (SEQ ID NO: 64) | | | |
| | | ID NO: | | | | |
| | | 63) | | | | |
| HCDR1\|2\|3 | GFNFKDTY (SEQ | IYPANG | ASDGGSYSYAFDY | 6.42E−07 | −6.19 | 642.24 |
| Variant | ID NO: 68) | YT (SEQ | (SEQ ID NO: 70) | | | |
| | | ID NO: | | | | |
| | | 69) | | | | |
| HCDR2\|3 | GFNIKDTY (SEQ | IYPSNG | ARSGYYYSYSFDY | 3.48E−07 | −6.46 | 347.56 |
| Variant | ID NO: 54) | YT (SEQ | (SEQ ID NO: 498) | | | |
| | | ID NO: | | | | |
| | | 73) | | | | |
| HCDR2\|3 | GFNIKDTY (SEQ | IYPSNG | ARYRGSSSYYFDY | 9.07E−07 | −6.04 | 906.53 |
| Variant | ID NO: 54) | YT (SEQ | (SEQ ID NO: 499) | | | |
| | | ID NO: | | | | |
| | | 73) | | | | |
| HCDR2\|3 | GFNIKDTY (SEQ | IYPSNG | ARYYGGSMYYYGY | 1.09E−07 | −6.96 | 109.22 |
| Variant | ID NO: 54) | YT (SEQ | (SEQ ID NO: 500) | | | |
| | | ID NO: | | | | |
| | | 73) | | | | |
| HCDR2\|3 | GFNIKDTY (SEQ | IYPSNG | ARSSGYYSYSYDY | 2.30E−07 | −6.64 | 230.09 |
| Variant | ID NO: 54) | YT (SEQ | (SEQ ID NO: 501) | | | |
| | | ID NO: | | | | |
| | | 73) | | | | |
| HCDR2\|3 | GFNIKDTY (SEQ | IYPANG | AREGGVGYYSFAY | 2.13E−07 | −6.67 | 213.14 |
| Variant | ID NO: 54) | YT (SEQ | (SEQ ID NO: 502) | | | |
| | | ID NO: | | | | |
| | | 69) | | | | |
| HCDR2\|3 | GFNIKDTY (SEQ | IYPANG | ASSGGGYSYYYTY | 4.76E−07 | −6.32 | 475.78 |
| Variant | ID NO: 54) | YT (SEQ | (SEQ ID NO: 503) | | | |
| | | ID NO: | | | | |
| | | 69) | | | | |
| HCDR2\|3 | GFNIKDTY (SEQ | IDPENG | ARVGGYGYYYYDY | 2.77E−07 | −6.56 | 277.34 |
| Variant | ID NO: 54) | YT (SEQ | (SEQ ID NO: 505) | | | |
| | | ID NO: | | | | |
| | | 504) | | | | |
| HCDR2\|3 | GFNIKDTY (SEQ | IYPANG | ASSGGSSSYYYNY | 5.54E−07 | −6.26 | 553.82 |
| Variant | ID NO: 54) | YT (SEQ | (SEQ ID NO: 506) | | | |
| | | ID NO: | | | | |
| | | 69) | | | | |
| HCDR2\|3 | GFNIKDTY (SEQ | IDPANG | ARVGGRGYYYYDY | 1.34E−07 | −6.87 | 133.97 |
| Variant | ID NO: 54) | YT (SEQ | (SEQ ID NO: 67) | | | |
| | | ID NO: | | | | |
| | | 66) | | | | |
| HCDR2\|3 | GFNIKDTY (SEQ | IYSSDG | ARSGGSSYYYYDY | 8.10E−07 | −6.09 | 809.63 |
| Variant | ID NO: 54) | YT (SEQ | (SEQ ID NO: 508) | | | |
| | | ID NO: | | | | |
| | | 507) | | | | |
| HCDR2\|3 | GFNIKDTY (SEQ | IDPADG | AREGGYGYYAMDY | 1.60E−07 | −6.8 | 160.15 |
| Variant | ID NO: 54) | YT (SEQ | (SEQ ID NO: 72) | | | |
| | | ID NO: | | | | |
| | | 71) | | | | |
| HCDR2\|3 | GFNIKDTY (SEQ | IYPEDG | ARSGGSSTYAYDY | 6.69E−07 | −6.17 | 669.18 |
| Variant | ID NO: 54) | ST (SEQ | (SEQ ID NO: 510) | | | |
| | | ID NO: | | | | |
| | | 509) | | | | |
| HCDR2\|3 | GFNIKDTY (SEQ | IYPSSG | ASSGGRYYYYYDY | 5.91E−07 | −6.23 | 590.53 |
| Variant | ID NO: 54) | YT (SEQ | (SEQ ID NO: 511) | | | |
| | | ID NO: | | | | |
| | | 60) | | | | |
| HCDR2\|3 | GFNIKDTY (SEQ | IYPSNG | ARYGGSYAYYFYY | 1.86E−08 | −7.73 | 18.6 |
| Variant | ID NO: 54) | YT (SEQ | (SEQ ID NO: 74) | | | |
| | | ID NO: | | | | |
| | | 73) | | | | |
| HCDR3 | GFNIKDTY (SEQ | IYPTNG | ARSRGSSSYMMDY | 5.66E−07 | −6.25 | 566.15 |
| Variant | ID NO: 54) | YT (SEQ | (SEQ ID NO: 512) | | | |
| | | ID NO: | | | | |
| | | 55) | | | | |
| HCDR3 | GFNIKDTY (SEQ | IYPTNG | ASSGGSSSYSYYY | 1.72E−06 | −5.76 | 1724.82 |
| Variant | ID NO: 54) | YT (SEQ | (SEQ ID NO: 513) | | | |
| | | ID NO: | | | | |
| | | 55) | | | | |
| HCDR3 | GFNIKDTY (SEQ | IYPTNG | ARSYGSGSYSFDY | 4.95E−07 | −6.31 | 494.72 |
| Variant | ID NO: 54) | YT (SEQ | (SEQ ID NO: 514) | | | |
| | | ID NO: | | | | |
| | | 55) | | | | |
| HCDR3 | GFNIKDTY (SEQ | IYPTNG | ARYSYYGGGYFDY | 4.78E−07 | −6.32 | 477.55 |
| Variant | ID NO: 54) | YT (SEQ | (SEQ ID NO: 515) | | | |
| | | ID NO: | | | | |
| | | 55) | | | | |
| HCDR3 | GFNIKDTY (SEQ | IYPTNG | ARDRGVSGYYLYY | 5.67E−07 | −6.25 | 566.77 |
| Variant | ID NO: 54) | YT (SEQ | (SEQ ID NO: 516) | | | |
| | | ID NO: | | | | |
| | | 55) | | | | |
| HCDR3 | GFNIKDTY (SEQ | IYPTNG | ARYGGSYFYYYSY | 7.31E−09 | −8.14 | 7.31 |
| Variant | ID NO: 54) | YT (SEQ | (SEQ ID NO: 517) | | | |
| | | ID NO: | | | | |
| | | 55) | | | | |
| HCDR3 | GFNIKDTY (SEQ | IYPTNG | ARYGGSSNYYYDY | 4.41E−07 | −6.36 | 440.64 |
| Variant | ID NO: 54) | YT (SEQ | (SEQ ID NO: 518) | | | |
| | | ID NO: | | | | |
| | | 55) | | | | |
| HCDR3 | GFNIKDTY (SEQ | IYPTNG | ARSGGSSFYGMDY | 6.80E−07 | −6.17 | 680.03 |
| Variant | ID NO: 54) | YT (SEQ | (SEQ ID NO: 519) | | | |
| | | ID NO: | | | | |
| | | 55) | | | | |
| HCDR3 | GFNIKDTY (SEQ | IYPTNG | ARTGGSSYYAYDY | 9.01E−07 | −6.05 | 901.24 |
| Variant | ID NO: 54) | YT (SEQ | (SEQ ID NO: 520) | | | |
| | | ID NO: | | | | |
| | | 55) | | | | |
| HCDR3 | GFNIKDTY (SEQ | IYPTNG | SRYGYSGYGAFDY | 1.05E−06 | −5.98 | 1052.08 |
| Variant | ID NO: 54) | YT (SEQ | (SEQ ID NO: 521) | | | |
| | | ID NO: | | | | |
| | | 55) | | | | |
| HCDR3 | GFNIKDTY (SEQ | IYPTNG | ARSGGSYSYSFDV | 1.07E−06 | −5.97 | 1065.31 |
| Variant | ID NO: 54) | YT (SEQ | (SEQ ID NO: 522) | | | |
| | | ID NO: | | | | |
| | | 55) | | | | |
| HCDR3 | GFNIKDTY (SEQ | IYPTNG | ARSGGSSYYYYDY | 1.07E−06 | −5.97 | 1067.07 |
| Variant | ID NO: 54) | YT (SEQ | (SEQ ID NO: 508) | | | |
| | | ID NO: | | | | |
| | | 55) | | | | |
| HCDR3 | GFNIKDTY (SEQ | IYPTNG | ARSGGSGYYSYNY | 6.03E−07 | −6.22 | 603.04 |
| Variant | ID NO: 54) | YT (SEQ | (SEQ ID NO: 523) | | | |
| | | ID NO: | | | | |
| | | 55) | | | | |
| HCDR3 | GFNIKDTY (SEQ | IYPTNG | ARSGGSGYYYYDY | 1.83E−06 | −5.74 | 1830.41 |
| Variant | ID NO: 54) | YT (SEQ | (SEQ ID NO: 524) | | | |
| | | ID NO: | | | | |
| | | 55) | | | | |
| HCDR3 | GFNIKDTY (SEQ | IYPTNG | ARDGGGGVYAFTY | 3.64E−07 | −6.44 | 363.6 |
| Variant | ID NO: 54) | YT (SEQ | (SEQ ID NO: 525) | | | |
| | | ID NO: | | | | |
| | | 55) | | | | |
| HCDR3 | GFNIKDTY (SEQ | IYPTNG | ARSSGSSYYGFDY | 6.68E−07 | −6.18 | 667.96 |
| Variant | ID NO: 54) | YT (SEQ | (SEQ ID NO: 526) | | | |
| | | ID NO: | | | | |
| | | 55) | | | | |
| HCDR3 | GFNIKDTY (SEQ | IYPTNG | ARYGGDYYYYYDY | 5.70E−08 | −7.24 | 56.99 |
| Variant | ID NO: 54) | YT (SEQ | (SEQ ID NO: 527) | | | |
| | | ID NO: | | | | |
| | | 55) | | | | |
| HCDR3 | GFNIKDTY (SEQ | IYPTNG | ARYGGSSYYYFDY | 9.72E−08 | −7.01 | 97.17 |
| Variant | ID NO: 54) | YT (SEQ | (SEQ ID NO: 528) | | | |
| | | ID NO: | | | | |
| | | 55) | | | | |
| HCDR3 | GFNIKDTY (SEQ | IYPTNG | ARYGGSSYYYYDY | 7.23E−08 | −7.14 | 72.34 |
| Variant | ID NO: 54) | YT (SEQ | (SEQ ID NO: 529) | | | |
| | | ID NO: | | | | |
| | | 55) | | | | |
| HCDR3 | GFNIKDTY (SEQ | IYPTNG | ARYYGSSSYYFAY | 6.06E−08 | −7.22 | 60.62 |
| Variant | ID NO: 54) | YT (SEQ | (SEQ ID NO: 75) | | | |
| | | ID NO: | | | | |
| | | 55) | | | | |
| HCDR3 | GFNIKDTY (SEQ | IYPTNG | TRYGSFGYYALDS | 5.76E−08 | −7.24 | 57.63 |
| Variant | ID NO: 54) | YT (SEQ | (SEQ ID NO: 530) | | | |
| | | ID NO: | | | | |
| | | 55) | | | | |
| HCDR3 | GFNIKDTY (SEQ | IYPTNG | ARYGGGYYYYYDY | 5.76E−08 | −7.24 | 57.56 |
| Variant | ID NO: 54) | YT (SEQ | (SEQ ID NO: 531) | | | |
| | | ID NO: | | | | |
| | | 55) | | | | |
| HCDR3 | GFNIKDTY (SEQ | IYPTNG | ARYRGRGFYSFDY | 2.20E−08 | −7.66 | 21.96 |
| Variant | ID NO: 54) | YT (SEQ | (SEQ ID NO: 532) | | | |
| | | ID NO: | | | | |
| | | 55) | | | | |
| HCDR3 | GFNIKDTY (SEQ | IYPTNG | ARYGGRGYYSFDY | 2.62E−08 | −7.58 | 26.24 |
| Variant | ID NO: 54) | YT (SEQ | (SEQ ID NO: 533) | | | |
| | | ID NO: | | | | |
| | | 55) | | | | |
| HCDR3 | GFNIKDTY (SEQ | IYPTNG | ARDGGGGFYAYDY | 1.24E−07 | −6.91 | 124.16 |
| Variant | ID NO: 54) | YT (SEQ | (SEQ ID NO: 534) | | | |
| | | ID NO: | | | | |
| | | 55) | | | | |
| HCDR3 | GFNIKDTY (SEQ | IYPTNG | ARDGGSSSYSYSY | 1.46E−07 | −6.83 | 146.32 |
| Variant | ID NO: 54) | YT (SEQ | (SEQ ID NO: 535) | | | |
| | | ID NO: | | | | |
| | | 55) | | | | |
| HCDR3 | GFNIKDTY (SEQ | IYPTNG | ARYSGSSYYYYDY | 4.23E−07 | −6.37 | 422.65 |
| Variant | ID NO: 54) | YT (SEQ | (SEQ ID NO: 536) | | | |
| | | ID NO: | | | | |
| | | 55) | | | | |
| HCDR3 | GFNIKDTY (SEQ | IYPTNG | SRYSYSGGGSFDY | 3.62E−07 | −6.44 | 361.85 |
| Variant | ID NO: 54) | YT (SEQ | (SEQ ID NO: 537) | | | |
| | | ID NO: | | | | |
| | | 55) | | | | |
| HCDR3 | GFNIKDTY (SEQ | IYPTNG | ARDGGRSYYSFYY | 1.04E−07 | −6.98 | 103.61 |
| Variant | ID NO: 54) | YT (SEQ | (SEQ ID NO: 538) | | | |
| | | ID NO: | | | | |
| | | 55) | | | | |
| HCDR3 | GFNIKDTY (SEQ | IYPTNG | ARVGGYGYYSYDY | 1.69E−07 | −6.77 | 168.68 |
| Variant | ID NO: 54) | YT (SEQ | (SEQ ID NO: 539) | | | |
| | | ID NO: | | | | |
| | | 55) | | | | |
| HCDR3 | GFNIKDTY (SEQ | IYPTNG | ARSSGYYYYGFVY | 1.57E−07 | −6.8 | 157.39 |
| Variant | ID NO: 54) | YT (SEQ | (SEQ ID NO: 540) | | | |
| | | ID NO: | | | | |
| | | 55) | | | | |
| HCDR3 | GFNIKDTY (SEQ | IYPTNG | ARDGGRGRYAMDY | 4.02E−07 | −6.4 | 402.38 |
| Variant | ID NO: 54) | YT (SEQ | (SEQ ID NO: 541) | | | |
| | | ID NO: | | | | |
| | | 55) | | | | |
| HCDR3 | GFNIKDTY (SEQ | IYPTNG | ARYSSSGGSAFDY | 1.99E−06 | −5.7 | 1986.37 |
| Variant | ID NO: 54) | YT (SEQ | (SEQ ID NO: 542) | | | |
| | | ID NO: | | | | |
| | | 55) | | | | |
| HCDR3 | GFNIKDTY (SEQ | IYPTNG | ARYSYYGGGAFDY | 1.91E−07 | −6.72 | 190.9 |
| Variant | ID NO: 54) | YT (SEQ | (SEQ ID NO: 543) | | | |
| | | ID NO: | | | | |
| | | 55) | | | | |
| HCDR3 | GFNIKDTY (SEQ | IYPTNG | ARSGGYYHYGFVY | 2.28E−07 | −6.64 | 227.95 |
| Variant | ID NO: 54) | YT (SEQ | (SEQ ID NO: 544) | | | |
| | | ID NO: | | | | |
| | | 55) | | | | |
| HCDR3 | GFNIKDTY (SEQ | IYPTNG | ARYGGRSSYYFDY | 2.57E−07 | −6.59 | 257.12 |
| Variant | ID NO: 54) | YT (SEQ | (SEQ ID NO: 545) | | | |
| | | ID NO: | | | | |
| | | 55) | | | | |
| HCDR3 | GFNIKDTY (SEQ | IYPTNG | ARSGGGSYYYYDY | 3.08E−07 | −6.51 | 307.53 |
| Variant | ID NO: 54) | YT (SEQ | (SEQ ID NO: 546) | | | |
| | | ID NO: | | | | |
| | | 55) | | | | |
| HCDR3 | GFNIKDTY (SEQ | IYPTNG | ARSTGSGFYSYDY | 3.30E−07 | −6.48 | 329.97 |
| Variant | ID NO: 54) | YT (SEQ | (SEQ ID NO: 547) | | | |
| | | ID NO: | | | | |
| | | 55) | | | | |
| HCDR3 | GFNIKDTY (SEQ | IYPTNG | ARSGGYYTYGFDY | 3.38E−07 | −6.47 | 337.65 |
| Variant | ID NO: 54) | YT (SEQ | (SEQ ID NO: 548) | | | |
| | | ID NO: | | | | |
| | | 55) | | | | |
| HCDR3 | GFNIKDTY (SEQ | IYPTNG | TRYSYYGYHAMDY | 3.54E−07 | −6.45 | 353.55 |
| Variant | ID NO: 54) | YT (SEQ | (SEQ ID NO: 549) | | | |
| | | ID NO: | | | | |
| | | 55) | | | | |
| HCDR3 | GFNIKDTY (SEQ | IYPTNG | ARYSGDSYYYYDY | 3.66E−07 | −6.44 | 366.22 |
| Variant | ID NO: 54) | YT (SEQ | (SEQ ID NO: 550) | | | |
| | | ID NO: | | | | |
| | | 55) | | | | |
| HCDR3 | GFNIKDTY (SEQ | IYPTNG | ARSGGRYTYGFDY | 1.41E−06 | −5.85 | 1407.65 |
| Variant | ID NO: 54) | YT (SEQ | (SEQ ID NO: 551) | | | |
| | | ID NO: | | | | |
| | | 55) | | | | |
| HCDR3 | GFNIKDTY (SEQ | IYPTNG | ARVGGRGYYAYDY | 2.29E−06 | −5.64 | 2286.4 |
| Variant | ID NO: 54) | YT (SEQ | (SEQ ID NO: 552) | | | |
| | | ID NO: | | | | |
| | | 55) | | | | |

### Cloning

In some aspects, antibody variants may be cloned and expressed in Fab format. For example, to produce ACE assay and SPR datasets, the present techniques may include synthesizing DNA variants spanning HCDR3, HCDR1 to HCDR3 across different libraries in an oligonucleotide format using ssDNA oligo pools (Twist Bioscience) as well as a double stranded DNA format using eBlocks (IDT). Codons may be randomly selected from the two most common in E. coli B strain for each variant. Amplification of the ssDNA oligo pools may be carried out by PCR according to Twist Bioscience's recommendations, with the exception that Q5 high fidelity DNA polymerase (NEB) may be used in place of KAPA polymerase. Briefly, 25 μL reactions may consist of 1×Q5 Mastermix, 0.3 M each of forward and reverse primers, and 10 ng oligo pool. Reactions may be initially denatured for 3 min at 95° C., followed by 13 cycles of: 95° C. for 20 s; 66° C. for 20 s; 72° C. for 15 s; and a final extension of 72° C. for 1 min. DNA amplification may be confirmed by agarose gel electrophoresis, and amplified DNA was subsequently purified (DNA Clean and Concentrate Kit, Zymo Research).

To build libraries meant for SPR validation of model designs in independent experiments, oligonucleotides spanning appropriate CDR(s) and the immediate upstream/downstream flanking nucleotides were synthesized by Integrated DNA Technologies (IDT).

To generate linearized vectors, the present techniques may include performing a two-step PCR to split the present proprietor's plasmid vector carrying Fab format trastuzumab into two fragments in a manner that provides cloning overlaps of approximately 25 nucleotides (nt) on the 5′ and 3′ ends of the amplified ssDNA oligo pool libraries, or 40 nt on the 5′ and 3′ ends of IDT eBlocks. Vector linearization reactions may be digested with DpnI (New England Bioloabs) and purified from a 0.8% agarose gel (Gel DNA Recovery Kit, Zymo Research) to eliminate parental vector carry through.

Cloning reactions may consist of 50 fmol of each purified vector fragment, either 100 fmol purified library (Twist Bioscience) or 10 pmol gBlock insert (IDT), and 1× final concentration NEBuilder HiFi DNA Assembly (New England Biolabs). Reactions may be incubated at 50° C. for either two hours (Twist Bioscience libraries) or 25 min (IDT library), and subsequently purified (DNA Clean and Concentrate Kit, Zymo Research).

For HDLs, Transformax EPI300 (Lucigen) E. coli may be transformed by electroporation (BioRad MicroPulser) with the purified assembly reactions and grown overnight at 30° C. in 20 mL of Teknova LB Broth with 50 μg/mL Kanamycin at 30° C. and 80% humidity with 270 rpm shaking for 18 h. Plasmids may be extracted (Plasmid Midi Kit, Zymo Research) and submitted for QC sequencing. Absci's SoluPro™ host strain may be transformed with 1 ng QC plasmid and grown at 30° C. in 20 mL of Teknova LB Broth with 50 μg/mL Kanamycin at 30° C. and 80% humidity with 270 rpm shaking for 18 hours.

For LDLs, Absci's SoluPro™ host strain may be transformed with the purified assembly reactions and grown overnight at 30° C. on agar plates containing 50 g/ml kanamycin and 1% glucose. Colonies may be picked for QC analysis prior to cultivation for induction. The foregoing experimental parameters and process flow may be modified, in some aspects.

### Exemplary OC Analysis

In the present techniques, quality of high diversity variant libraries may be assessed by deep sequencing. Briefly, library plasmid pools may be amplified by PCR across the HCDR3 region and sequenced with 2×150 nt reads using the Illumina MiSeq platform with 20% PhiX, for example. The PCR reaction may be 10 nM primer concentration, Q5 2× master mix (NEB) and 1 ng of input DNA diluted in H2O. Reactions may be initially denatured at 98° C. for 3 min; followed by 30 cycles of 98° C. for 10 s, 59° C. for 30 s, 72° C. for 15 s; with a final extension of 72° C. for 2 min. Sequencing results may be analyzed for distribution of mutations, variant representation, library complexity and recovery of expected sequences. Metrics may include coefficient of variation of sequence representation, read share of top 1% most prevalent sequences and percentage of designed library sequences observed within the library. Quality of low diversity variant libraries may be assessed by performing rolling circle amplification (Equiphi29, Thermo Fisher Scientific) on 24 colonies and sequencing using the Illumina DNA Prep, Tagmentation Kit (Illumina Inc.). Each colony may be analyzed for single nucleotide polymorphisms (SNPs), presence of multiple variants, misassembly, and/or matching to a library sequence (Geneious Prime).

Exemplary Antibody Expression in SoluPro™ E. coli B Strain

After transformation and 8 hour recovery, HDLs may be grown in 50 mL of Teknova LB Broth with 50 μg/mL Kanamycin at 30° C. and 80% humidity with 270 rpm shaking for 24 hours. At the end of the 24 hours, the preculture may be OD600 normalized to 0.1 in induction media (IBM) (4.5 g/L Potassium Phosphate monobasic, 13.8 g/L Ammonium Sulfate, 20.5 g/L yeast extract, 20.5 g/L glycerol, 1.95 g/L Citric Acid) containing inducers and supplements (250 μM Arabinose, 50 μg/mL Kanamycin, 8 mM Magnesium Sulfate, 1 mM Propionate, 1× Korz trace metals) and grown for 16 hours in a 500 mL baffled flask at 26° C. and 80% humidity with 270 rpm shaking. At the end of the 16 hours, 500 mL aliquots may be adjusted to 20% v/v glycerol and stored at −80° C.

After transformation and QC of LDLs, individual colonies may be picked into deep well plates containing 400 μL of Teknova LB Broth 50 μg/mL Kanamycin and incubated at 30° C. and 80% humidity with 1000 rpm shaking for 24 hours. At the end of the 24 hours, 150 μL samples may be centrifuged (3300 g, 7 min) and supernatant decanted from the preculture plate for sequence analysis. 80 μL of the preculture was transferred to 400 μL of IBM containing inducers and supplements as described above. The culture may be grown for 16 hours at 26° C. and 80% humidity with 270 rpm shaking. At the end of the 16 hours, 150 μL samples may be taken and centrifuged (3300 g, 7 min) into pellets with supernatant decanting prior to being stored at −80° C.

### Activity-Specific Cell-Enrichment (ACE) Assay

### Cell Preparation

High-throughput quantitative selection of antigen-specific Fab-expressing cells may be adapted from conventional approaches, in some aspects. For staining, an OD600=2 of thawed glycerol stocks from induced cultures may be transferred to 0.7 ml matrix tubes, centrifuged (4000 g, 5 min) and resulting pelleted cells washed three times with PBS (pH 7.4, 1 mM EDTA). Washed cells may be thoroughly resuspended in 250 μL of phosphate buffer (32 mM, pH 7.4) by pipetting prior to fixation by the addition of 250 μL of 0.6% paraformaldehyde and 0.04% glutaraldehyde in phosphate buffer (32 mM, pH 7.4). After 40 min incubation on ice, samples may be centrifuged (4000 g, 5 min) and pellets washed three times with PBS (pH 7.4, 1 mM EDTA), resuspended in permeabilization buffer (20 mM Tris, 50 mM glucose, 10 mM EDTA, 5 μg/mL rLysozyme) and incubated for 8 min on ice. Fixed and permeabilized cells may then centrifuged (4000 g, 5 min) and washed a number (e.g., three) of times with staining buffer (Perkin Elmer AlphaLISA immunoassay buffer, 25 mM HEPES, 0.1% casein, 1 mg/mL dextran-500, 0.5% Triton X-100, 0.05% Kathon).

### Staining

Prior to library staining, the HER2 probe may be titrated against the reference strain to determine the 75% effective concentration (EC75). Following cell preparation, the library may be resuspended in 500 μL staining buffer containing 100 nM either His/Avi tagged human HER2 (Acro Biosystems) conjugated to 50 nM streptavidin-AF647 (Invitrogen) or tag-free human Her2 (Acro Biosystems) directly conjugated to AF647 via free amines. Libraries may8 be incubated with the probe overnight (16 h) with end-to-end rotation at 4° C., centrifuged (4000 g, 5 min), and pellets washed three times with PBS. Pellets may be resuspended in 500 μL of staining buffer containing 26.5 nM anti-kappa light chain:BV421 (BioLegend) and incubated for 2 hours with end-to-end rotation at 4° C. prior to centrifugation (4000 g, 5 min), three washes with PBS and resuspension in 200 μL of PBS for sorting.

### Sorting

Libraries may be sorted by one of two methods based on binding in aquantitative ACE assay or a binary version of the ACE Assay as described herein. For either method, libraries may be sorted on FACSymphony S6 (BD Biosciences) instruments. Immediately prior to sorting, 50 μL of stained sample was transferred to a flow tube containing 1 mL PBS+3 μL propidium iodide.

Aggregates, debris, and impermeable cells may be removed with singlets, size, and PI+ parent gating, respectively. Cells may then be gated to include only those with kappa light chain expression (BV421). For the quantitative ACE assay, collection gates may be drawn to sample across the log range of binding signal. The far right may be set to collect the brightest 0.1% of the library and the far left gate may be set to collect at the low end of the positive binding signal based on stained control strains. Four additional gates of the same width may be distributed in between, with each set to be approximately half the gMFI of the gate to the right.

For the binary version of the ACE assay, a total of three collection gates may be set to sample at the high end of the binding range (top 0.1%), the remaining positive binding signal events, and a negative gate containing the events with no binding signal. Libraries may be sorted simultaneously on two instruments with photomultipliers adjusted to normalize fluorescence intensity, and the collected events were processed independently as technical replicates.

### Next-Generation Sequencing

### Sorted Material Sample Preparation

Sample preparation for sequencing may follow the same protocol for both the previously described quantitative ACE assay and the binary version of the ACE assay. Specifically, cell material from sorted gates may be collected in a diluted PBS mixture (VWR), in 1.5 mL tubes (Eppendorf). A sample of the unsorted library material may be processed for QC and ACE metric calculations. Post-sort samples may be centrifuged (3,800×g) and tube volume normalized to 20 μl. Amplicons encompassing the HCDR3 region may be generated by PCR. The reaction may use 10 nM primer concentration, Q5 2× master mix (NEB) and 20 μl of sorted cell material input suspended in diluted PBS (VWR). Reactions may be initially denatured at 98° C. for 3 min, followed by 30 cycles of 98° C. for 10 s; 59° C. for 30 s; 72° C. for 15 s; with a final extension of 72° C. for 2 min. After amplification, samples may be cleaned enzymatically using ExoSAP-IT (Applied Biosystems). Resulting DNA samples may be quantified by Qubit luorometer (Invitrogen), prepped for sequencing with the ThruPLEX DNA-Seq Kit (Takara Bio), normalized and pooled. Pool size may be verified via Tapestation 1000 HS and may be sequenced on an Illumina NextSeq 1000 P2 (2×150 nt) with 20% PhiX.

### In Silico Structural Modeling

Rosetta's FastRelax application is run with flexible backbone and side-chain degrees of freedom. Prior to the relax procedure, we first idealize all candidate structures using Rosetta's Idealize protocol to avoid steric clashes and improper geometry. We relax using the maximum number of rotamers by passing -EX1, -EX2, -EX3 and -EX4 flags at initialization. We also include flags -packing:repack_only to disable design as well as flags -no_his_his_pairE and -multi_cool_annealer 10 to set the number of annealing iterations. For ranking of conformations in FastRelax, we use Rosetta's ref2015 energy function. It is well known that running relax on a structure will often move the backbone a few Angstroms1, so we include an additional term containing harmonic distance constraints for all pairs of C/3 atoms that are either not part of a CDR loop or not within distance 10 to any atom in a CDR loop, based on the conformation of the initial structure. These constraints are given weight 10⁻⁴. The protocol is run ten times for each target, and the decoy with the lowest energy in the HCDR3 loop region is eventually selected.

### Antibodies

As described herein, the present techniques may include a computer system, computer-implemented method and/or computer-readable medium/media that predicts or generates structural information of a biomolecule such as an antibody. The term “antibody” as used herein refers to whole antibodies that interact with (e.g., by binding, steric hindrance, stabilizing/destabilizing, spatial distribution) an epitope on a target antigen. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes for example, monoclonal antibodies, human antibodies, humanized antibodies, camelised antibodies, chimeric antibodies, single-chain Fvs (scFv), disulfide-linked Fvs (sdFv), Fab fragments, F (ab′) fragments, and anti-idiotypic (anti-Id) antibodies (including, e.g., anti-Id antibodies to antibodies of the invention), and epitope-binding fragments of any of the above. The antibodies can be of any isotype (e.g., IgG, IgE, IgM, IgD, IgA and IgY), class (e.g., IgG1, IgG2, IgG3, IgG4, IgA1 and IgA2) or subclass. The antibody or epitope-binding fragments may be, or be a component of, a multi-specific molecule.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively.

The phrase “antibody fragment”, as used herein, refers to one or more portions of an antibody that retain the ability to specifically interact with (e.g., by binding, steric hindrance, stabilizing/destabilizing, spatial distribution) a target epitope. Examples of binding fragments include, but are not limited to, a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies.

As described herein, antibodies may include biologically active derivatives or variants or fragments. As used herein “biologically active derivative” or “biologically active variant” includes any derivative or variant of an antibody having substantially the same functional and/or biological properties of said antibody (e.g., a WT antibody), such as binding properties, and/or the same structural basis, such as a peptidic backbone or a basic polymeric unit, including framework regions.

An “analog,” such as a “variant” or a “derivative,” is an antibody substantially similar in structure and having the same biological activity, albeit in certain instances to a differing degree, to a naturally-occurring antibody or a WT antibody or another reference antibody as will be understood by those of skill in the art. For example, an antibody variant refers to an antibody sharing substantially similar structure and having the same biological activity as a reference antibody. Variants or analogs differ in the composition of their amino acid sequences compared to the reference antibody from which the analog is derived, based on one or more mutations involving (i) deletion of one or more amino acid residues at one or more termini of the antibody and/or one or more internal regions of the antibody sequence (e.g., fragments), (ii) insertion or addition of one or more amino acids at one or more termini (typically an “addition” or “fusion”) of the antibody and/or one or more internal regions (typically an “insertion”) of the antibody sequence or (iii) substitution of one or more amino acids for other amino acids in the antibody sequence. By way of example, a “derivative” is a type of analog and refers to an antibody sharing the same or substantially similar structure as a reference antibody that has been modified, e.g., chemically.

In some aspects, the variants or sequence variants are mutants wherein 1, 2, 3, 4, 5, 6 or more amino acids within one or more CDR are mutated relative to a reference antibody. In some aspects, CDRs on the light chain, heavy chain, or both heavy and light chain, are mutated. In some aspects, one or more framework amino acid residues are mutated relative to a reference antibody.

In substitution variants, one or more amino acid residues, e.g., in a CDR region, of an antibody are removed and replaced with alternative residues. In one aspect, the substitutions are conservative in nature and conservative substitutions of this type are well known in the art. Alternatively, the disclosure embraces substitutions that are also non-conservative. Exemplary conservative substitutions are described in Lehninger, [Biochemistry, 2nd Edition; Worth Publishers, Inc., New York (1975), pp.71-77].

Antibodies contemplated herein include full-length antibodies, biologically active subunits or fragments of full length antibodies, as well as biologically active derivatives and variants of any of these forms of therapeutic proteins. Thus, antibodies include those that (1) have an amino acid sequence that has greater than about 60%, about 65%, about 70%, about 75%, about 80%, about 85%, about 90%, about 91%, about 92%, about 93%, about 94%, about 95%, about 96%, about 97%, about 98% or about 99% or greater amino acid sequence identity, over a region of at least about 25, about 50, about 100, about 200, about 300, about 400, or more amino acids, to a reference antibody (e.g., encoded by a referenced nucleic acid or an amino acid sequence described herein). According to the present disclosure, the term “recombinant protein” or “recombinant antibody” includes any protein obtained via recombinant DNA technology. In certain aspects, the term encompasses antibodies as described herein.

In some embodiments, the antibodies or antibody variants described herein are expressed from one or more expression construct and/or in a cell or strains as described herein.

Exemplary wild-type or reference antibodies include commercially available or other known antibodies, including therapeutic monoclonal antibodies. Reference antibodies according to the present disclosure may include any antibodies now known or later developed, including those that are not clinically and/or commercially available.

### Cells and Expression Constructs

### Cells

Antibodies of the present disclosure, including wild-type (WT) antibodies and variant antibodies, are produced in some embodiments in cells. Cells comprising one or more of the expression constructs described herein are contemplated in various embodiments of the present disclosure.

Prokaryotic host cells. In some embodiments of the disclosure, expression constructs designed for expression of gene products, including fusion proteins as described herein, are provided in host cells, such as prokaryotic host cells. Prokaryotic host cells can include archaea (such as Haloferax volcanii, Sulfolobus solfataricus), Gram-positive bacteria (such as Bacillus subtilis, Bacillus licheniformis, Brevibacillus choshinensis, Lactobacillus brevis, Lactobacillus buchneri, Lactococcus lactis, and Streptomyces lividans), or Gram-negative bacteria, including Alphaproteobacteria (Agrobacterium tumefaciens, Caulobacter crescentus, Rhodobacter sphaeroides, and Sinorhizobium meliloti), Betaproteobacteria (Alcaligenes eutrophus), and Gammaproteobacteria (Acinetobacter calcoaceticus, Azotobacter vinelandii, Escherichia coli, Pseudomonas aeruginosa, and Pseudomonas putida). Preferred host cells include Gammaproteobacteria of the family Enterobacteriaceae, such as Enterobacter, Erwinia, Escherichia (including E. coli), Klebsiella, Proteus, Salmonella (including Salmonella typhimurium), Serratia (including Serratia marcescans), and Shigella.

Eukaryotic host cells. Many additional types of host cells can be used for the expression systems of the present disclosure, including eukaryotic cells such as yeast (Candida shehatae, Kluyveromyces lactis, Kluyveromyces fragilis, other Kluyveromyces species, Pichia pastoris, Saccharomyces cerevisiae, Saccharomyces pastorianus also known as Saccharomyces carlsbergensis, Schizosaccharomyces pombe, Dekkera/Brettanomyces species, and Yarrowia lipolyticd); other fungi (Aspergillus nidulans, Aspergillus niger, Neurospora crassa, Penicillium, Tolypocladium, Trichoderma reesia); insect cell lines (Drosophila melanogaster Schneider 2 cells and Spodoptera frugiperda Sf9 cells); and mammalian cell lines including immortalized cell lines (Chinese hamster ovary (CHO) cells, HeLa cells, baby hamster kidney (BHK) cells, monkey kidney cells (COS), human embryonic kidney (HEK, 293, or HEK-293) cells, and human hepatocellular carcinoma cells (Hep G2)). The above host cells are available from the American Type Culture Collection.

As described in WO/2017/106583, incorporated by reference in its entirety herein, producing gene products such as therapeutic proteins at commercial scale and in soluble form is addressed by providing suitable host cells capable of growth at high cell density in fermentation culture, and which can produce soluble gene products in the oxidizing host cell cytoplasm through highly controlled inducible gene expression. Host cells of the present disclosure with these qualities are produced by combining some or all of the following characteristics. (1) The host cells are genetically modified to have an oxidizing cytoplasm, through increasing the expression or function of oxidizing polypeptides in the cytoplasm, and/or by decreasing the expression or function of reducing polypeptides in the cytoplasm. Specific examples of such genetic alterations are provided herein. Optionally, host cells can also be genetically modified to express chaperones and/or cofactors that assist in the production of the desired gene product(s), and/or to glycosylate polypeptide gene products. (2) The host cells comprise one or more expression constructs designed for the expression of one or more gene products of interest; in certain embodiments, at least one expression construct comprises an inducible promoter and a polynucleotide encoding a gene product to be expressed from the inducible promoter. (3) The host cells contain additional genetic modifications designed to improve certain aspects of gene product expression from the expression construct(s). In particular embodiments, the host cells (A) have an alteration of gene function of at least one gene encoding a transporter protein for an inducer of at least one inducible promoter, and as another example, wherein the gene encoding the transporter protein is selected from the group consisting of araE, araE, araG, araH, rhaT, xylF, xylG, and xylH, or particularly is araE, or wherein the alteration of gene function more particularly is expression of araE from a constitutive promoter; and/or (B) have a reduced level of gene function of at least one gene encoding a protein that metabolizes an inducer of at least one inducible promoter, and as further examples, wherein the gene encoding a protein that metabolizes an inducer of at least one said inducible promoter is selected from the group consisting of araA, araB, araD, prpB, prpD, rhaA, rhaB, rhaD, xylA, and xylB; and/or (C) have a reduced level of gene function of at least one gene encoding a protein involved in biosynthesis of an inducer of at least one inducible promoter, which gene in further embodiments is selected from the group consisting of scpA/sbm, argK/ygfD, scpB/ygfG, scpC/ygfH, rmlA, rmlB, rmlC, and rmlD.

Host Cells with Oxidizing Cytoplasm. The expression systems of the present disclosure are designed to express gene products; in certain embodiments of the disclosure, the gene products are expressed in a host cell. Examples of host cells are provided that allow for the efficient and cost-effective expression of gene products, including components of multimeric products. Host cells can include, in addition to isolated cells in culture, cells that are part of a multicellular organism, or cells grown within a different organism or system of organisms. In certain embodiments of the disclosure, the host cells are microbial cells such as yeasts (Saccharomyces, Schizosaccharomyces, etc.) or bacterial cells, or are gram-positive bacteria or gram-negative bacteria, or are E. coli, or are an E. coli B strain, or are E. coli (B strain) EB0001 cells (also called E. coli ASE(DGH) cells), or are E. coli (B strain) EB0002 cells. In growth experiments with E. coli host cells having oxidizing cytoplasm, specifically the E. coli B strains SHuffle® Express (NEB Catalog No. C3028H) and SHuffle® T7 Express (NEB Catalog No. C3029H) and the E. coli K strain SHuffle® T7 (NEB Catalog No. C3026H), these E. coli B strains with oxidizing cytoplasm are able to grow to much higher cell densities than the most closely corresponding E. coli K strain (WO/2017/106583).

Alterations to host cell gene functions. Certain alterations can be made to the gene functions of host cells comprising inducible expression constructs, to promote efficient and homogeneous induction of the host cell population by an inducer. Preferably, the combination of expression constructs, host cell genotype, and induction conditions results in at least 75% (more preferably at least 85%, and most preferably, at least 95%) of the cells in the culture expressing gene product from each induced promoter, as measured by the method of Khlebnikov et al. described in Example 9 of WO/2017/106583. For host cells other than E. coli, these alterations can involve the function of genes that are structurally similar to an E. coli gene, or genes that carry out a function within the host cell similar to that of the E. coli gene. Alterations to host cell gene functions include eliminating or reducing gene function by deleting the gene protein-coding sequence in its entirety, or deleting a large enough portion of the gene, inserting sequence into the gene, or otherwise altering the gene sequence so that a reduced level of functional gene product is made from that gene. Alterations to host cell gene functions also include increasing gene function by, for example, altering the native promoter to create a stronger promoter that directs a higher level of transcription of the gene, or introducing a missense mutation into the protein-coding sequence that results in a more highly active gene product. Alterations to host cell gene functions include altering gene function in any way, including for example, altering a native inducible promoter to create a promoter that is constitutively activated. In addition to alterations in gene functions for the transport and metabolism of inducers, as described herein with relation to inducible promoters, and/or an altered expression of chaperone proteins, it is also possible to alter the reduction-oxidation environment of the host cell.

Host cell reduction-oxidation environment. In bacterial cells such as E. coli, proteins that need disulfide bonds are typically exported into the periplasm where disulfide bond formation and isomerization is catalyzed by the Dsb system, comprising DsbABCD and DsbG. Increased expression of the cysteine oxidase DsbA, the disulfide isomerase DsbC, or combinations of the Dsb proteins, which are all normally transported into the periplasm, has been utilized in the expression of heterologous proteins that require disulfide bonds (Makino et al., Microb Cell Fact 2011 May 14; 10: 32). It is also possible to express cytoplasmic forms of these Dsb proteins, such as a cytoplasmic version of DsbA and/or of DsbC (‘cDsbA or ‘cDsbC’), that lacks a signal peptide and therefore is not transported into the periplasm. Cytoplasmic Dsb proteins such as cDsbA and/or cDsbC are useful for making the cytoplasm of the host cell more oxidizing and thus more conducive to the formation of disulfide bonds in heterologous proteins produced in the cytoplasm. The host cell cytoplasm can also be made less reducing and thus more oxidizing by altering the thioredoxin and the glutaredoxin/glutathione enzyme systems directly: mutant strains defective in glutathione reductase (gor) or glutathione synthetase (gshB), together with thioredoxin reductase (trxB), render the cytoplasm oxidizing. These strains are unable to reduce ribonucleotides and therefore cannot grow in the absence of exogenous reductant, such as dithiothreitol (DTT). Suppressor mutations (such as ahpC* and ahpCA, Lobstein et al., Microb Cell Fact 2012 May 8; 11: 56; doi: 10.1186/1475-2859-11-56) in the gene ahpC, which encodes the peroxiredoxin AhpC, convert it to a disulfide reductase that generates reduced glutathione, allowing the channeling of electrons onto the enzyme ribonucleotide reductase and enabling the cells defective in gor and trxB, or defective in gshB and trxB, to grow in the absence of DTT. A different class of mutated forms of AhpC can allow strains, defective in the activity of gamma-glutamylcysteine synthetase (gshA) and defective in trxB, to grow in the absence of DTT; these include AhpC V164G, AhpC S71F, AhpC E173/S71F, AhpC E171Ter, and AhpC dupl62-169 (Faulkner et al., Proc Natl Acad Sci USA 2008 May 6; 105(18): 6735-6740, Epub 2008 May 2). In such strains with oxidizing cytoplasm, exposed protein cysteines become readily oxidized in a process that is catalyzed by thioredoxins, in a reversal of their physiological function, resulting in the formation of disulfide bonds. Other proteins that may be helpful to reduce the oxidative stress effects in host cells of an oxidizing cytoplasm are HPI (hydroperoxidase I) catalase-peroxidase encoded by E. coli katG and HPII (hydroperoxidase II) catalase-peroxidase encoded by E. coli katE, which disproportionate peroxide into water and 02 (Farr and Kogoma, Microbiol Rev. 1991 December; 55(4): 561-585; Review). Increasing levels of KatG and/or KatE protein in host cells through induced coexpression or through elevated levels of constitutive expression is an aspect of some embodiments of the disclosure.

Another alteration that can be made to host cells is to express the sulfhydryl oxidase Ervlp from the inner membrane space of yeast mitochondria in the host cell cytoplasm, which has been shown to increase the production of a variety of complex, disulfide-bonded proteins of eukaryotic origin in the cytoplasm of E. coli, even in the absence of mutations in gor or trxB (Nguyen et al, Microb Cell Fact 2011 Jan. 7; 10: 1).

Host cells comprising expression constructs preferably also express cDsbA and/or cDsbC and/or Ervlp; are deficient in trxB gene function; are also deficient in the gene function of either gor, gshB, or gshA; optionally have increased levels of katG and/or katE gene function; and express an appropriate mutant form of AhpC so that the host cells can be grown in the absence of DTT.

Chaperones. In some embodiments, desired gene products are coexpressed with other gene products, such as chaperones, that are beneficial to the production of the desired gene product. Chaperones are proteins that assist the non-covalent folding or unfolding, and/or the assembly or disassembly, of other gene products, but do not occur in the resulting monomeric or multimeric gene product structures when the structures are performing their normal biological functions (having completed the processes of folding and/or assembly). Chaperones can be expressed from an inducible promoter or a constitutive promoter within an expression construct, or can be expressed from the host cell chromosome; preferably, expression of chaperone protein(s) in the host cell is at a sufficiently high level to produce coexpressed gene products that are properly folded and/or assembled into the desired product. Examples of chaperones present in E. coli host cells are the folding factors DnaK/DnaJ/GrpE, DsbC/DsbG, GroEL/GroES, IbpA/IbpB, Skp, Tig (trigger factor), and FkpA, which have been used to prevent protein aggregation of cytoplasmic or periplasmic proteins. DnaK/DnaJ/GrpE, GroEL/GroES, and ClpB can function synergistically in assisting protein folding and therefore expression of these chaperones in combinations has been shown to be beneficial for protein expression (Makino et al., Microb Cell Fact 2011 May 14; 10: 32). When expressing eukaryotic proteins in prokaryotic host cells, a eukaryotic chaperone protein, such as protein disulfide isomerase (PDI) from the same or a related eukaryotic species, is in certain embodiments of the disclosure coexpressed or inducibly coexpressed with the desired gene product.

One chaperone that can be expressed in host cells is a protein disulfide isomerase from Humicola insolens, a soil hyphomycete (soft-rot fungus). An amino acid sequence of Humicola insolens PDI is shown as SEQ ID NO: 1 of WO/2017/106583; it lacks the signal peptide of the native protein so that it remains in the host cell cytoplasm. The nucleotide sequence encoding PDI was optimized for expression in E. coli; the expression construct for PDI is shown as SEQ ID NO: 2 of WO/2017/106583. SEQ ID NO: 2 contains a GCTAGC Nhel restriction site at its 5′ end, an AGGAGG ribosome binding site at nucleotides 7 through 12, the PDI coding sequence at nucleotides 21 through 1478, and a GTCGAC Sail restriction site at its 3′ end. The nucleotide sequence of SEQ ID NO: 2 was designed to be inserted immediately downstream of a promoter, such as an inducible promoter. The Nhel and Sail restriction sites in SEQ ID NO: 2 can be used to insert it into a vector multiple cloning site, such as that of the pSOL expression vector (SEQ ID NO: 3 of WO/2017/106583), described in published US patent application US2015353940A1, which is incorporated by reference in its entirety herein. Other PDI polypeptides can also be expressed in host cells, including PDI polypeptides from a variety of species (Saccharomyces cerevisiae (UniProtKB PI 7967), Homo sapiens (UniProtKB P07237), Mus musculus (UniProtKB P09103), Caenorhabditis elegans (UniProtKB Q 17770 and Q 17967), Arabdopsis thaliana (UniProtKB 048773, Q9XI01, Q9S G3, Q9LJU2, Q9MAU6, Q94F09, and Q9T042), Aspergillus niger (UniProtKB Q12730) and also modified forms of such PDI polypeptides. In certain embodiments of the disclosure, a PDI polypeptide expressed in host cells of the disclosure shares at least 70%, or 80%, or 90%, or 95% amino acid sequence identity across at least 50% (or at least 60%, or at least 70%, or at least 80%, or at least 90%) of the length of SEQ ID NO: 1 of WO/2017/106583, where amino acid sequence identity is determined according to Example 10 of WO/2017/106583.

Cellular transport of cofactors. When using the expression systems of the disclosure to produce enzymes that require cofactors for function, it is helpful to use a host cell capable of synthesizing the cofactor from available precursors, or taking it up from the environment. Common cofactors include ATP, coenzyme A, flavin adenine dinucleotide (FAD), NAD+/NADH, and heme. Polynucleotides encoding cofactor transport polypeptides and/or cofactor synthesizing polypeptides can be introduced into host cells, and such polypeptides can be constitutively expressed, or inducibly coexpressed with the gene products to be produced by methods of the disclosure.

Glycosylation of polypeptide gene products. Host cells can have alterations in their ability to glycosylate polypeptides. For example, eukaryotic host cells can have eliminated or reduced gene function in glycosyltransf erase and/or oligo-saccharyltransferase genes, impairing the normal eukaryotic glycosylation of polypeptides to form glycoproteins. Prokaryotic host cells such as E. coli, which do not normally glycosylate polypeptides, can be altered to express a set of eukaryotic and prokaryotic genes that provide a glycosylation function (DeLisa et al., WO2009089154A2, 2009 Jul. 16).

Available host cell strains with altered gene functions. To create preferred strains of host cells to be used in the expression systems and methods of the disclosure, it is useful to start with a strain that already comprises desired genetic alterations (Table A; WO/2017/106583).

TABLE A
Exemplary host cell strains

| Strain | Genotype | Source |
|---|---|---|
| E, coli | F- mcrA Δ(mrr-hsdRMS-mcrBC) | Invitrogen Life |
| TOP10 | Φ80lacZ ΔM15 Δlacx74 recA1 araD139 | Technologies |
| | Δ(ara-leu)7697 galU galK rpsL (Str^{R}) | Catalog Nos. |
| | endA1 nupG λ | C404-10, |
| | | C4040-03, |
| | | C4040-06, |
| | | C4040-50, and |
| | | C4040-52 |
| E, coli | Δ(ara-leu)7697 Δlacx74 ΔphoA PvuII | Merck (EMD |
| Origami ™ | phoR araD139 ahpC gale galK rpsL | Millipore |
| 2 | F′[lac⁺ lacI^{q} pro] gor522::Tn10 trxB | Chemicals) |
| | (Str^{R}, Tet^{R}) | Catalog No. |
| | | 71344 |
| E, coli | fhuA2 [lon] ompT ahpC gal λatt::pNEB3- | New England |
| SHuffle ® | r1-cDsbc (Spec, lacI) ΔtrxB sulA11 | Biolabs |
| Express | R(mcr-73::miniTn10-Tet^{S})2 [dcm] | Catalog |
| | R(zgb-210::Tn10-Tet^{S}) endA1 Δgor | No. C3028H |
| | Δ(mcrC-mrr)114::IS10 | |

### Expression Constructs

In some embodiments of the present disclosure, inducible promoters are contemplated for use with the expression constructs. Exemplary promoters are described herein and are also described in WO/2016/205570, incorporated by reference in its entirety herein. As described herein, the cells comprising one or more expression constructs may optionally include one or more inducible promoters to express antibodies of the present disclosure, including wild-type antibodies and variant antibodies.

Expression Constructs. Expression constructs are polynucleotides designed for the expression of one or more antibodies, and thus are not naturally occurring molecules. Expression constructs can be integrated into a host cell chromosome, or maintained within the host cell as polynucleotide molecules replicating independently of the host cell chromosome, such as plasmids or artificial chromosomes. An example of an expression construct is a polynucleotide resulting from the insertion of one or more polynucleotide sequences into a host cell chromosome, where the inserted polynucleotide sequences alter the expression of chromosomal coding sequences. An expression vector is a plasmid expression construct specifically used for the expression of one or more gene products, such as one or more antibodies. One or more expression constructs can be integrated into a host cell chromosome or be maintained on an extrachromosomal polynucleotide such as a plasmid or artificial chromosome. The following are descriptions of particular types of polynucleotide sequences that can be used in expression constructs for the expression or coexpression of antibodies.

Origins of replication. Expression constructs must comprise an origin of replication, also called a replicon, in order to be maintained within the host cell as independently replicating polynucleotides. Different replicons that use the same mechanism for replication cannot be maintained together in a single host cell through repeated cell divisions. As a result, plasmids can be categorized into incompatibility groups depending on the origin of replication that they contain, as shown in Table 2 of WO/2016/205570. Origins of replication can be selected for use in expression constructs on the basis of incompatibility group, copy number, and/or host range, among other criteria. As described above, if two or more different expression constructs are to be used in the same host cell for the coexpression of multiple antibodies or components of antibodies (e.g., heavy and light chains, including fragments, as described herein), in one embodiment the different expression constructs contain origins of replication from different incompatibility groups: a pMBl replicon in one expression construct and a p15A replicon in another, for example. The average number of copies of an expression construct in the cell, relative to the number of host chromosome molecules, is determined by the origin of replication contained in that expression construct. Copy number can range from a few copies per cell to several hundred (Table 2 of WO/2016/205570). In one embodiment of the disclosure, different expression constructs are used which comprise inducible promoters that are activated by the same inducer, but which have different origins of replication. By selecting origins of replication that maintain each different expression construct at a certain approximate copy number in the cell, it is possible to adjust the levels of overall production of an antibody component or fragment (e.g., a heavy or light chain) expressed from one expression construct, relative to another antibody component or fragment (e.g., a heavy or light chain) expressed from a different expression construct. As an example, to coexpress subunits A and B of a multimeric protein (including, for example, a heavy chain and a light chain), an expression construct is created which comprises the colEl replicon, the am promoter, and a coding sequence for subunit A expressed from the am promoter: ‘colEl-Para-A.

Another expression construct is created comprising the pl 5A replicon, the am promoter, and a coding sequence for subunit B: ‘pl5A-Para-B’. These two expression constructs can be maintained together in the same host cells, and expression of both subunits A and B is induced by the addition of one inducer, arabinose, to the growth medium. If the expression level of subunit A needed to be significantly increased relative to the expression level of subunit B, in order to bring the stoichiometric ratio of the expressed amounts of the two subunits closer to a desired ratio, for example, a new expression construct for subunit A could be created, having a modified μMB 1 replicon as is found in the origin of replication of the pUC9 plasmid (‘pUC9ori’): pUC9ori-Para-A. Expressing subunit A from a high-copy-number expression construct such as pUC9ori-Para-A should increase the amount of subunit A produced relative to expression of subunit B from pl5A-Para-B. In a similar fashion, use of an origin of replication that maintains expression constructs at a lower copy number, such as pSOOl (WO/2016/205570), could reduce the overall level of a gene product expressed from that construct. Selection of an origin of replication can also determine which host cells can maintain an expression construct comprising that replicon. For example, expression constructs comprising the colEl origin of replication have a relatively narrow range of available hosts, species within the Enterobacteriaceae family, while expression constructs comprising the RK2 replicon can be maintained in E. coli, Pseudomonas aeruginosa, Pseudomonas putida, Azotobacter vinelandii, and Alcaligenes eutrophus, and if an expression construct comprises the RK2 replicon and some regulator genes from the RK2 plasmid, it can be maintained in host cells as diverse as Sinorhizobium meliloti, Agrobacterium tumefaciens, Caulobacter crescentus, Acinetobacter calcoaceticus, and Rhodobacter sphaeroides (Kiies and Stahl, Microbiol Rev 1989 December; 53(4): 491-516).

Similar considerations can be employed to create expression constructs for inducible expression or coexpression in eukaryotic cells. For example, the 2-micron circle plasmid of Saccharomyces cerevisiae is compatible with plasmids from other yeast strains, such as pSRl (ATCC Deposit Nos. 48233 and 66069; Araki et al., J Mol Biol 1985 Mar. 20; 182(2): 191-203) and pKDl (ATCC Deposit No. 37519; Chen et al, Nucleic Acids Res 1986 Jun. 11; 14(11): 4471-4481).

Selectable markers. Expression constructs usually comprise a selection gene, also termed a selectable marker, which encodes a protein necessary for the survival or growth of host cells in a selective culture medium. Host cells not containing the expression construct comprising the selection gene will not survive in the culture medium. Typical selection genes encode proteins that confer resistance to antibiotics or other toxins, or that complement auxotrophic deficiencies of the host cell. One example of a selection scheme utilizes a drug such as an antibiotic to arrest growth of a host cell. Those cells that contain an expression construct comprising the selectable marker produce a protein conferring drug resistance and survive the selection regimen. Some examples of antibiotics that are commonly used for the selection of selectable markers (and abbreviations indicating genes that provide antibiotic resistance phenotypes) are: ampicillin (AmpR), chloramphenicol (CmlR or CmR), kanamycin (KanR), spectinomycin (SpcR), streptomycin (StrR), and tetracycline (TetR). Many of the plasmids in Table 2 of WO/2016/205570 comprise selectable markers, such as pBR322 (AmpR, TetR); pMOB45 (CmR, TetR); pACYClW (AmpR, KanR); and pGBMl (SpcR, StrR). The native promoter region for a selection gene is usually included, along with the coding sequence for its gene product, as part of a selectable marker portion of an expression construct. Alternatively, the coding sequence for the selection gene can be expressed from a constitutive promoter.

In various aspects, suitable selectable markers include, but are not limited to, neomycin phosphotransferase (npt II), hygromycin phosphotransferase (hpt), dihydrofolate reductase (dhfr), zeocin, phleomycin, bleomycin resistance gene (ble), gentamycin acetyltransferase, streptomycin phosphotransferase, mutant form of acetolactate synthase (als), bromoxynil nitrilase, phosphinothricin acetyl transferase (bar), enolpyruvylshikimate-3-phosphate (EPSP) synthase (aro A), muscle specific tyrosine kinase receptor molecule (MuSK-R), copper-zinc superoxide dismutase (sodl), metallothioneins (cup1, MT1), beta-lactamase (BLA), puromycin N-acetyl-transferase (pac), blasticidin acetyl transferase (bls), blasticidin deaminase (bsr), histidinol dehydrogenase (HDH), N-succinyl-5-aminoimidazole-4-carboxamide ribotide (SAICAR) synthetase (adel), argininosuccinate lyase (arg4), beta-isopropylmalate dehydrogenase (leu2), invertase (suc2), orotidine-5′-phosphate (OMP) decarboxylase (ura3), and orthologs of any of the foregoing.

Inducible promoter. As described herein, there are several different inducible promoters that can be included in expression constructs as part of the inducible coexpression systems of the disclosure. Preferred inducible promoters share at least 80% polynucleotide sequence identity (more preferably, at least 90% identity, and most preferably, at least 95% identity) to at least 30 (more preferably, at least 40, and most preferably, at least 50) contiguous bases of a promoter polynucleotide sequence as defined in Table 1 of WO/2016/205570 by reference to the E. coli K-12 substrain MG1655 genomic sequence, where percent polynucleotide sequence identity is determined using the methods of Example 11 of WO/2016/205570. Under ‘standard’ inducing conditions (see Example 5 of WO/2016/205570), preferred inducible promoters have at least 75% (more preferably, at least 100%, and most preferably, at least 110%) of the strength of the corresponding ‘wild-type’ inducible promoter of E. coli K-12 substrain MG1655, as determined using the quantitative PCR method of De Mey et al. (Example 6 of WO/2016/205570). Within the expression construct, an inducible promoter is placed 5′ to (or ‘upstream of) the coding sequence for the gene product (e.g., antibody or antibody fragment) that is to be inducibly expressed, so that the presence of the inducible promoter will direct transcription of the gene product coding sequence in a 5′ to 3′ direction relative to the coding strand of the polynucleotide encoding the gene product.

Ribosome binding site. For some antibodies or antibody fragments, the nucleotide sequence of the region between the transcription initiation site and the initiation codon of the coding sequence of the gene product that is to be inducibly expressed corresponds to the 5′ untranslated region (‘UTR’) of the mRNA for the polypeptide gene product. Preferably, the region of the expression construct that corresponds to the 5′ UT comprises a polynucleotide sequence similar to the consensus ribosome binding site (RBS, also called the Shine-Dalgarno sequence) that is found in the species of the host cell. In prokaryotes (archaea and bacteria), the RBS consensus sequence is GGAGG or GGAGGU, and in bacteria such as E. coli, the RBS consensus sequence is AGGAGG or AGGAGGU. The RBS is typically separated from the initiation codon by 5 to 10 intervening nucleotides. In expression constructs, the RBS sequence is preferably at least 55% identical to the AGGAGGU consensus sequence, more preferably at least 70% identical, and most preferably at least 85% identical, and is separated from the initiation codon by 5 to 10 intervening nucleotides, more preferably by 6 to 9 intervening nucleotides, and most preferably by 6 or 7 intervening nucleotides. The ability of a given RBS to produce a desirable translation initiation rate can be calculated at the website salis.psu.edu/software/RBSLibraryCalculatorSearchMode, using the RBS Calculator; the same tool can be used to optimize a synthetic RBS for a translation rate across a 100,000+ fold range (Salis, Methods Enzymol 2011; 498: 19-42).

Multiple cloning site. A multiple cloning site (MCS), also called a polylinker, is a polynucleotide that contains multiple restriction sites in close proximity to or overlapping each other. The restriction sites in the MCS typically occur once within the MCS sequence, and preferably do not occur within the rest of the plasmid or other polynucleotide construct, allowing restriction enzymes to cut the plasmid or other polynucleotide construct only within the MCS. Examples of MCS sequences are those in the pBAD series of expression vectors, including pBAD18, pBAD18-Cm, pBAD18-Kan, pBAD24, pBAD28, pBAD30, and pBAD33 (Guzman et al., J Bacteriol 1995 July; 177(14): 4121-4130); or those in the pPRO series of expression vectors derived from the pBAD vectors, such as pPR018, pPR018-Cm, pPR018-Kan, pPR024, pPRO30, and pPR033 (U.S. Pat. No. 8,178,338 B2; May 15, 2012; Keasling, Jay). A multiple cloning site can be used in the creation of an expression construct: by placing a multiple cloning site 3′ to (or downstream of) a promoter sequence, the MCS can be used to insert the coding sequence for a gene product to be expressed or coexpressed into the construct, in the proper location relative to the promoter so that transcription of the coding sequence will occur. Depending on which restriction enzymes are used to cut within the MCS, there may be some part of the MCS sequence remaining within the expression construct after the coding sequence or other polynucleotide sequence is inserted into the expression construct. Any remaining MCS sequence can be upstream or, or downstream of, or on both sides of the inserted sequence. A ribosome binding site can be placed upstream of the MCS, preferably immediately adjacent to or separated from the MCS by only a few nucleotides, in which case the RBS would be upstream of any coding sequence inserted into the MCS. Another alternative is to include a ribosome binding site within the MCS, in which case the choice of restriction enzymes used to cut within the MCS will determine whether the RBS is retained, and in what relation to, the inserted sequences. A further alternative is to include a RBS within the polynucleotide sequence that is to be inserted into the expression construct at the MCS, preferably in the proper relation to any coding sequences to stimulate initiation of translation from the transcribed messenger RNA.

Expression from constitutive promoters. Expression constructs of the disclosure can also comprise coding sequences that are expressed from constitutive promoters. Unlike inducible promoters, constitutive promoters initiate continual gene product production under most growth conditions. One example of a constitutive promoter is that of the Tn3 bla gene, which encodes beta-lactamase and is responsible for the ampicillin-resistance (AmpR) phenotype conferred on the host cell by many plasmids, including pBR322 (ATCC 31344), pACYClW (ATCC 37031), and pBAD24 (ATCC 87399). Another constitutive promoter that can be used in expression constructs is the promoter for the E. coli lipoprotein gene, Ipp, which is located at positions 1755731-1755406 (plus strand) in E. coli K-12 substrain MG1655 (Inouye and Inouye, Nucleic Acids Res 1985 May 10; 13(9): 3101-3110). A further example of a constitutive promoter that has been used for heterologous gene expression in E. coli is the trμLEDCBA promoter, located at positions 1321169-1321133 (minus strand) in E. coli K-12 substrain MG1655 (Windass et al., Nucleic Acids Res 1982 Nov. 11; 10(21): 6639-6657). Constitutive promoters can be used in expression constructs for the expression of selectable markers, as described herein, and also for the constitutive expression of other gene products useful for the coexpression of the desired product. For example, transcriptional regulators of the inducible promoters, such as AraC, PrpR, RhaR, and XylR, if not expressed from a bidirectional inducible promoter, can alternatively be expressed from a constitutive promoter, on either the same expression construct as the inducible promoter they regulate, or a different expression construct. Similarly, gene products useful for the production or transport of the inducer, such as PrpEC, AraE, or Rha, or proteins that modify the reduction-oxidation environment of the cell, as a few examples, can be expressed from a constitutive promoter within an expression construct. Gene products useful for the production of coexpressed gene products, and the resulting desired product, also include chaperone proteins, cofactor transporters, etc.

Signal Peptides. Antibodies or antibody fragments expressed or coexpressed by the methods of the disclosure can contain signal peptides or lack them, depending on whether it is desirable for such gene products to be exported from the host cell cytoplasm into the periplasm, or to be retained in the cytoplasm, respectively. Signal peptides (also termed signal sequences, leader sequences, or leader peptides) are characterized structurally by a stretch of hydrophobic amino acids, approximately five to twenty amino acids long and often around ten to fifteen amino acids in length, that has a tendency to form a single alpha-helix. This hydrophobic stretch is often immediately preceded by a shorter stretch enriched in positively charged amino acids (particularly lysine). Signal peptides that are to be cleaved from the mature polypeptide typically end in a stretch of amino acids that is recognized and cleaved by signal peptidase. Signal peptides can be characterized functionally by the ability to direct transport of a polypeptide, either co-translationally or post-translationally, through the plasma membrane of prokaryotes (or the inner membrane of gram negative bacteria like E. coli), or into the endoplasmic reticulum of eukaryotic cells. The degree to which a signal peptide enables a polypeptide to be transported into the periplasmic space of a host cell like E. coli, for example, can be determined by separating periplasmic proteins from proteins retained in the cytoplasm, using a method such as described in Example 12 of WO/2016/205570.

The following is a description of inducible promoters that can be used in expression constructs for expression or coexpression of gene products, along with some of the genetic modifications that can be made to host cells that contain such expression constructs. Examples of these inducible promoters and related genes are, unless otherwise specified, from Escherichia coli (E. coli) strain MG1655 (American Type Culture Collection deposit ATCC 700926), which is a substrain of E. coli K-12 (American Type Culture Collection deposit ATCC 10798). Table 1 of WO/2016/205570 lists the genomic locations, in E. coli MG1655, of the nucleotide sequences for these examples of inducible promoters and related genes. Nucleotide and other genetic sequences, referenced by genomic location as in Table 1 of WO/2016/205570, are expressly incorporated by reference herein. Additional information about E. coli promoters, genes, and strains described herein can be found in many public sources, including the online EcoliWiki resource, located at ecoliwiki.net.

Arabinose promoter. (As used herein, ‘arabinose’ means L-arabinose.) Several E. coli operons involved in arabinose utilization are inducible by arabinose-araBAD, araC, arciE, and araFGH but the terms ‘arabinose promoter’ and ‘ara promoter’ are typically used to designate the araBAD promoter. Several additional terms have been used to indicate the E. coli araBAD promoter, such as Para, ParaB, ParaBAD, and PBAD- The use herein of ‘ara promoter’ or any of the alternative terms given above, means the E. coli araBAD promoter. As can be seen from the use of another term, ‘araC-araBAD promoter’, the araBAD promoter is considered to be part of a bidirectional promoter, with the araBAD promoter controlling expression of the araBAD operon in one direction, and the araC promoter, in close proximity to and on the opposite strand from the araBAD promoter, controlling expression of the araC coding sequence in the other direction. The AraC protein is both a positive and a negative transcriptional regulator of the araBAD promoter. In the absence of arabinose, the AraC protein represses transcription from PBAD, but in the presence of arabinose, the AraC protein, which alters its conformation upon binding arabinose, becomes a positive regulatory element that allows transcription from PBAD- The araBAD operon encodes proteins that metabolize L-arabinose by converting it, through the intermediates L-ribulose and L-ribulose-phosphate, to D-xylulose-5-phosphate. For the purpose of maximizing induction of expression from an arabinose-inducible promoter, it is useful to eliminate or reduce the function of AraA, which catalyzes the conversion of L-arabinose to L-ribulose, and optionally to eliminate or reduce the function of at least one of AraB and AraD, as well. Eliminating or reducing the ability of host cells to decrease the effective concentration of arabinose in the cell, by eliminating or reducing the cell's ability to convert arabinose to other sugars, allows more arabinose to be available for induction of the arabinose-inducible promoter. The genes encoding the transporters which move arabinose into the host cell are araE, which encodes the low-affinity L-arabinose proton symporter, and the araFGH operon, which encodes the subunits of an ABC superfamily high-affinity L-arabinose transporter. Other proteins which can transport L-arabinose into the cell are certain mutants of the LacY lactose permease: the LacY(AlWC) and the LacY(AlWV) proteins, having a cysteine or a valine amino acid instead of alanine at position 177, respectively (Morgan-Kiss et al., Proc Natl Acad Sci USA 2002 May 28; 99(11): 7373-7377). In order to achieve homogenous induction of an arabinose-inducible promoter, it is useful to make transport of arabinose into the cell independent of regulation by arabinose. This can be accomplished by eliminating or reducing the activity of the AraFGH transporter proteins and altering the expression of araE so that it is only transcribed from a constitutive promoter. Constitutive expression of araE can be accomplished by eliminating or reducing the function of the native araE gene, and introducing into the cell an expression construct which includes a coding sequence for the AraE protein expressed from a constitutive promoter. Alternatively, in a cell lacking AraFGH function, the promoter controlling expression of the host cell's chromosomal araE gene can be changed from an arabinose-inducible promoter to a constitutive promoter. In similar manner, as additional alternatives for homogenous induction of an arabinose-inducible promoter, a host cell that lacks AraE function can have any functional AraFGH coding sequence present in the cell expressed from a constitutive promoter. As another alternative, it is possible to express both the araE gene and the araFGH operon from constitutive promoters, by replacing the native araE and araFGH promoters with constitutive promoters in the host chromosome. It is also possible to eliminate or reduce the activity of both the AraE and the AraFGH arabinose transporters, and in that situation to use a mutation in the LacY lactose permease that allows this protein to transport arabinose. Since expression of the lacY gene is not normally regulated by arabinose, use of a LacY mutant such as LacY(A177C) or LacY(A177V), will not lead to the ‘all or none’ induction phenomenon when the arabinose-inducible promoter is induced by the presence of arabinose. Because the LacY(A177C) protein appears to be more effective in transporting arabinose into the cell, use of polynucleotides encoding the LacY(A177C) protein is preferred to the use of polynucleotides encoding the LacY(A177V) protein.

Propionate promoter. The ‘propionate promoter’ or ‘prp promoter’ is the promoter for the E. coli prpBCDE operon, and is also called PPΦB· Like the ara promoter, the prp promoter is part of a bidirectional promoter, controlling expression of the prpBCDE operon in one direction, and with the prpR promoter controlling expression of the prpR coding sequence in the other direction. The PrpR protein is the transcriptional regulator of the prp promoter, and activates transcription from the prp promoter when the PrpR protein binds 2-methylcitrate (‘2-MC’). Propionate (also called propanoate) is the ion, CH3CH2COO—, of propionic acid (or ‘propanoic acid’), and is the smallest of the ‘fatty’ acids having the general formula H(CH2), COOH that shares certain properties of this class of molecules: producing an oily layer when salted out of water and having a soapy potassium salt. Commercially available propionate is generally sold as a monovalent cation salt of propionic acid, such as sodium propionate (CH3CH2COONa), or as a divalent cation salt, such as calcium propionate (Ca(CH3CH2COO)2). Propionate is membrane-permeable and is metabolized to 2-MC by conversion of propionate to propionyl-CoA by PrpE (propionyl-CoA synthetase), and then conversion of propionyl-CoA to 2-MC by PrpC (2-methylcitrate synthase). The other proteins encoded by the prpBCDE operon, PrpD (2-methylcitrate dehydratase) and PrpB (2-methylisocitrate lyase), are involved in further catabolism of 2-MC into smaller products such as pyruvate and succinate. In order to maximize induction of a propionate-inducible promoter by propionate added to the cell growth medium, it is therefore desirable to have a host cell with PrpC and PrpE activity, to convert propionate into 2-MC, but also having eliminated or reduced PrpD activity, and optionally eliminated or reduced PrpB activity as well, to prevent 2-MC from being metabolized. Another operon encoding proteins involved in 2-MC biosynthesis is the scpA-argK-scpBC operon, also called the sbm-yg/DGH operon. These genes encode proteins required for the conversion of succinate to propionyl-CoA, which can then be converted to 2-MC by PrpC. Elimination or reduction of the function of these proteins would remove a parallel pathway for the production of the 2-MC inducer, and thus might reduce background levels of expression of a propionate-inducible promoter, and increase sensitivity of the propionate-inducible promoter to exogenously supplied propionate. It has been found that a deletion of sbm-ygfD-ygfG-ygfH-ygfl, introduced into E. coli BL21(DE3) to create strain JSB (Lee and Keasling, Appl Environ Microbiol 2005 November; 71(11): 6856-6862), was helpful in reducing background expression in the absence of exogenously supplied inducer, but this deletion also reduced overall expression from the prp promoter in strain JSB. It should be noted, however, that the deletion sbm-ygfD-ygfG-ygfH-ygfl also apparently affects ygfl, which encodes a putative LysR-family transcriptional regulator of unknown function. The genes sbm-yg/DGH are transcribed as one operon, and ygfl is transcribed from the opposite strand. The 3′ ends of the ygfti and ygfl coding sequences overlap by a few base pairs, so a deletion that takes out all of the sbm- yg/DGH operon apparently takes out ygfl coding function as well. Eliminating or reducing the function of a subset of the sbm-ygfDGH gene products, such as YgfG (also called ScpB, methylmalonyl-CoA decarboxylase), or deleting the majority of the sbm-yg/DGH (or scpA-argK-scpBC) operon while leaving enough of the 3′ end of the ygfli (or scpC) gene so that the expression of ygfl is not affected, could be sufficient to reduce background expression from a propionate-inducible promoter without reducing the maximal level of induced expression.

Rhamnose promoter. (As used herein, ‘rhamnose’ means L-rhamnose.) The ‘rhamnose promoter‘ or’rha promoter’, or PrhaSR, is the promoter for the E. coli rhaSR operon. Like the ara and prp promoters, the rha promoter is part of a bidirectional promoter, controlling expression of the rhaSR operon in one direction, and with the rhaBAD promoter controlling expression of the rhaBAD operon in the other direction. The rha promoter, however, has two transcriptional regulators involved in modulating expression: RhaR and RhaS. The RhaR protein activates expression of the rhaSR operon in the presence of rhamnose, while RhaS protein activates expression of the L-rhamnose catabolic and transport operons, rhaBAD and rhaT, respectively (Wickstrum et al, J Bacteriol 2010 January; 192(1): 225-232). Although the RhaS protein can also activate expression of the rhaSR operon, in effect RhaS negatively autoregulates this expression by interfering with the ability of the cyclic AMP receptor protein (CRP) to coactivate expression with RhaR to a much greater level. The rhaBAD operon encodes the rhamnose catabolic proteins RhaA (L-rhamnose isomerase), which converts L-rhamnose to L-rhamnulose; RhaB (rhamnulokinase), which phosphorylates L-rhamnulose to form L-rhamnulose-1-P; and RhaD (rhamnulose-1-phosphate aldolase), which converts L-rhamnulose-1-P to L-lactaldehyde and DHAP (dihydroxy acetone phosphate). To maximize the amount of rhamnose in the cell available for induction of expression from a rhamnose-inducible promoter, it is desirable to reduce the amount of rhamnose that is broken down by catalysis, by eliminating or reducing the function of RhaA, or optionally of RhaA and at least one of RhaB and RhaD. E. coli cells can also synthesize L-rhamnose from alpha-D-glucose-1-P through the activities of the proteins RmlA, Rm1B, RmlC, and RmlD (also called RfbA, RfbB, RfbC, and RfbD, respectively) encoded by the rmlBDACX (or rfbBDACX) operon. To reduce background expression from a rhamnose-inducible promoter, and to enhance the sensitivity of induction of the rhamnose-inducible promoter by exogenously supplied rhamnose, it could be useful to eliminate or reduce the function of one or more of the Rm1A, RmlB, RmlC, and

RmlD proteins. L-rhamnose is transported into the cell by RhaT, the rhamnose permease or L-rhamnose:proton symporter. As noted above, the expression of RhaT is activated by the transcriptional regulator RhaS. To make expression of RhaT independent of induction by rhamnose (which induces expression of RhaS), the host cell can be altered so that all functional RhaT coding sequences in the cell are expressed from constitutive promoters. Additionally, the coding sequences for RhaS can be deleted or inactivated, so that no functional RhaS is produced. By eliminating or reducing the function of RhaS in the cell, the level of expression from the rhaSR promoter is increased due to the absence of negative autoregulation by RhaS, and the level of expression of the rhamnose catalytic operon rhaBAD is decreased, further increasing the ability of rhamnose to induce expression from the rha promoter.

Xylose promoter. (As used herein, ‘xylose’ means D-xylose.) The xylose promoter, or ‘xyl promoter’, or PxyiA, means the promoter for the E. coli xylAB operon. The xylose promoter region is similar in organization to other inducible promoters in that the xylAB operon and the xylFGHR operon are both expressed from adjacent xylose-inducible promoters in opposite directions on the E. coli chromosome (Song and Park, J Bacteriol. 1997 November; 179(22): 7025-7032). The transcriptional regulator of both the PxyiA and PxyiF promoters is XylR, which activates expression of these promoters in the presence of xylose. The xylR gene is expressed either as part of the xylFGHR operon or from its own weak promoter, which is not inducible by xylose, located between the xylH and xylR protein-coding sequences. D-xylose is catabolized by XylA (D-xylose isomerase), which converts D-xylose to D-xylulose, which is then phosphorylated by XylB (xylulokinase) to form D-xylulose-5-P. To maximize the amount of xylose in the cell available for induction of expression from a xylose-inducible promoter, it is desirable to reduce the amount of xylose that is broken down by catalysis, by eliminating or reducing the function of at least XylA, or optionally of both XylA and XylB. The xylFGHR operon encodes XylF, XylG, and XylH, the subunits of an ABC super-family high-affinity D-xylose transporter. The xylE gene, which encodes the E. coli low-affinity xylose-proton symporter, represents a separate operon, the expression of which is also inducible by xylose. To make expression of a xylose transporter independent of induction by xylose, the host cell can be altered so that all functional xylose transporters are expressed from constitutive promoters. For example, the xylFGHR operon could be altered so that the xylFGH coding sequences are deleted, leaving XylR as the only active protein expressed from the xylose-inducible PxyiF promoter, and with the xylE coding sequence expressed from a constitutive promoter rather than its native promoter. As another example, the xylR coding sequence is expressed from the PxyiA or the promoter in an expression construct, while either the xylFGHR operon is deleted and xylE is constitutively expressed, or alternatively an xylFGH operon (lacking the xylR coding sequence since that is present in an expression construct) is expressed from a constitutive promoter and the xylE coding sequence is deleted or altered so that it does not produce an active protein.

Lactose promoter. The term ‘lactose promoter’ refers to the lactose-inducible promoter for the lacZYA operon, a promoter which is also called lacZpl; this lactose promoter is located at ca. 365603-365568 (minus strand, with the NA polymerase binding (‘−35’) site at ca. 365603-365598, the Pribnow box (‘-10’) at 365579-365573, and a transcription initiation site at 365567) in the genomic sequence of the E. coli K-12 substrain MG1655 (NCBI Reference Sequence NC 000913.2, 11 Jan. 2012). In some embodiments, inducible coexpression systems of the disclosure can comprise a lactose-inducible promoter such as the lacZYA promoter. In other embodiments, the inducible coexpression systems of the disclosure comprise one or more inducible promoters that are not lactose-inducible promoters.

Alkaline phosphatase promoter. The terms ‘alkaline phosphatase promoter’ and ‘phoA promoter’ refer to the promoter for the phoApsiF operon, a promoter which is induced under conditions of phosphate starvation. The phoA promoter region is located at ca. 401647-401746 (plus strand, with the Pribnow box (‘-10’) at 401695-401701 (Kikuchi et al., Nucleic Acids Res 1981 Nov. 11; 9(21): 5671-5678)) in the genomic sequence of the E. coli K-12 substrain MG1655 (NCBI Reference Sequence NC 000913.3, 16 Dec. 2014). The transcriptional activator for the phoA promoter is PhoB, a transcriptional regulator that, along with the sensor protein PhoR, forms a two-component signal transduction system in E. coli. PhoB and PhoR are transcribed from the phoBR operon, located at ca. 417050-419300 (plus strand, with the PhoB coding sequence at 417,142-417,831 and the PhoR coding sequence at 417,889-419,184) in the genomic sequence of the E. coli K-12 substrain MG1655 (NCBI Reference Sequence NC 000913.3, 16 Dec. 2014). The phoA promoter differs from the inducible promoters described above in that it is induced by the lack of a substance—intracellular phosphate—rather than by the addition of an inducer. For this reason the phoA promoter is generally used to direct transcription of gene products that are to be produced at a stage when the host cells are depleted for phosphate, such as the later stages of fermentation. In some embodiments, inducible coexpression systems of the disclosure can comprise a phoA promoter. In other embodiments, the inducible coexpression systems of the disclosure comprise one or more inducible promoters that are not phoA promoters.

### Affinity Assays

As described herein, the present techniques may include a computer system, computer-implemented method and/or computer-readable medium/media that predicts or generates structural information of a biomolecule such as an antibody. In some aspects, the computer system predicts binding affinity between, for example, an antibody and an antigen. Nevertheless, wet lab techniques are contemplated to (i) confirm a predicted affinity or (ii) generate data to train an affinity model as described herein. Antibody binding and antibody affinity determination assays are well known in the art.

In one embodiment, an activity-specific cell-enrichment method (ACE) can be used to identify host cells that express “active” antibodies rather than “inactive material.” Active antibodies can be distinguished from inactive antibodies by the ability of active antibodies to specifically bind a binding partner molecule (e.g., an antigen or epitope). The ACE assay protocol is described in WO/2021/146626, incorporated by reference herein. It will be appreciated by those of ordinary skill in the art that ACE can not only discriminate between active/inactive in a binary fashion, but can also compute a score that is proportional to affinity. Thus, ACE provides quantitative assay information, not merely binary/Boolean information, which enables the modeling techniques herein to perform regression techniques. This richer modeling represents an advantageous improvement over the limited binary classification of conventional techniques.

In another embodiment, the HiPR Bind assay described in WO/2021/163349 and incorporated by reference herein is used in conjunction with the methods provided herein.

Binding assays, for example assays that measure protein-protein interactions, including antibody-antigen interactions and including measuring binding affinity, are well known in the art. By way of example, Surface plasmon resonance (SPR), Dual polarisation interferometry (DPI), Static light scattering (SLS), Dynamic light scattering (DLS), Flow-induced dispersion analysis (FIDA), Fluorescence polarization/anisotropy, Fluorescence resonance energy transfer (FRET), Bio-layer interferometry (BLI), Isothermal titration calorimetry (ITC), Microscale thermophoresis (MST), Single colour reflectometry (SCORE) are contemplated. Additionally, Bimolecular fluorescence complementation (BiFC), affinity electrophoresis, label transfer, phage display, Tandem affinity purification (TAP), cross-linking, Quantitative immunoprecipitation combined with knock-down (QUICK) and Proximity ligation assay (PLA) are other well-known assays that provide protein-protein interaction information.

In some embodiments, the binding affinities of the antibodies described herein are measured by array surface plasmon resonance (SPR), according to standard techniques (Abdiche, et al. (2016) MAbs 8:264-277). Briefly, antibodies were immobilized on a HC 30M chip at four different densities/antibody concentrations. Varying concentrations (0-500 nM) of antibody target are then bound to the captured antibodies. Kinetic analysis is performed using Carterra software to extract association and dissociation rate constants (kₐ and k_{d}, respectively) for each antibody. Apparent affinity constants (K_{D}) are calculated from the ratio of k_{d}/kₐ. In some embodiments, the Carterra LSA Platform is used to determine kinetics and affinity. In other embodiments, binding affinity can be measured, e.g., by surface plasmon resonance (e.g., BIAcore™) using, for example, the IBIS MX96 SPR system from IBIS Technologies or the Carterra LSA SPR platform, or by Bio-Layer Interferometry, for example using the Octet™ system from ForteBio. In some embodiments, a biosensor instrument such as Octet RED384, ProteOn XPR36, IBIS MX96 and Biacore T100 is used (Yang, D., et al., J. Vis. Exp., 2017, 122:55659).

K_{D} is the equilibrium dissociation constant, a ratio of k_{off}/kₒₙ, between the antibody and its antigen. K_{D} and affinity are inversely related. The K_{D} value relates to the concentration of antibody and so the lower the K_{D} value (lower concentration) and thus the higher the affinity of the antibody. Antibody, including reference antibody and variant antibody, K_{D} according to various embodiments of the present disclosure can be, for example, in the micromolar range (10⁻⁴ to 10⁻⁶), the nanomolar range (10⁻⁷ to 10⁻⁹), the picomolar range (10⁻¹ to 10⁻²) or the femtomolar range (10⁻¹³ to 10⁻¹⁵). In some embodiments, antibody affinity of a variant antibody is improved, relative to a reference antibody, by approximately 5, 10, 15, 20, 25, 30, 35, 40, 45, or 50% or more. The improvement may also be expressed relative to a fold change (e.g., 2×, 4×, 6×, or 2-, 3-, 4-, 5-, 6-, 7-, 8-, 9-, 10-fold or more improvement in binding activity, etc.) and/or an order of magnitude (e.g., 10⁷, 10⁸, 10⁹, etc.).

The data generated from the antibodies and assays described herein is, in some embodiments, used to train one or more models, as will be described next.

### Additional Considerations

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible. This is intended to provide support for all such combinations.

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed:

1. A computing system for training a machine learning model to generate structural information of a target biomolecule, comprising:
one or more processors; and
one or more non-transitory computer-readable media having stored thereon instructions that, when executed by the one or more processors, cause the computing system to:
receive one or more training inputs, including one or more of (i) input biomolecule structural information, (ii) input biomolecule binding partner structural information or (iii) input biomolecule-input binding partner binding complex structural information;
process the one or more training inputs with a machine-learned biomolecule prediction model to generate predicted biomolecule structural information;
evaluate a loss function that compares the predicted biomolecule structural information to a ground truth value; and
modify one or more values of one or more parameters of the machine-learned model based at least in part on the loss function.

2. The computing system of claim 1, wherein the target biomolecule is an antibody or protein.

3. The computing system of claim 1, wherein the input biomolecule structural information comprises three-dimensional coordinates of a primary sequence.

4. The computing system of claim 1, wherein the input biomolecule binding partner is an antigen, receptor, ligand or a cell membrane.

5. The computing system of claim 1, wherein the training inputs are represented, respectively, in the non-transitory computer-readable media as at least one of:
(i) a protein data bank (PDB) data format, (ii) a JSON data format or (iii) an XML data format.

5a. The computing system of claim 1, wherein the training inputs include at least one, at least five, at least 10 or at least 100 of the binders in Table E1, Table E2, Table E3 and/or Table E4, above.

6. The computing system of claim 1, wherein the input biomolecule binding partner is a protein, and the input biomolecule binding partner structural information comprises three-dimensional structure.

7. The computing system of claim 1, the one or more non-transitory computer-readable media having stored thereon further instructions that, when executed by the one or more processors, cause the computing system to:
receive the training inputs from a database comprising antibody structures and/or antigen structures.

8. The computing system of claim 1, the one or more non-transitory computer-readable media having stored thereon further instructions that, when executed by the one or more processors, cause the computing system to:
receive the training inputs from a structural antibody database (SAbDab).

9. A computing system for generating structural information of a target biomolecule, comprising:
one or more processors; and
one or more non-transitory computer-readable media having stored thereon:
a machine-learned biomolecule prediction model trained to predict structural information of biomolecules based on an input; and
instructions that, when executed by the one or more processors, cause the computing system to:
(1) receive a target input including one or more of a target binding partner primary sequence, three-dimensional coordinates of a target binding partner, a target binding partner epitope primary sequence, or three-dimensional coordinates of a target binding partner epitope primary sequence, or a fragment or portion of any of the foregoing; and
(2) predict the structural information of the target biomolecule by processing the target input with the machine-learned biomolecule prediction model.

9a. The computing system of claim 9,
wherein the target input includes CDRH1 and CDRH2 of trastuzumab-Bh1, and the structure of VEGF.

9b. The computing system of claim 9,
wherein the target input includes CDRH1 and CDRH2 of trastuzumab, and the structure of HER2.

10. The computing system of claim 9, wherein the structural information is represented in the computer-readable media as at least one of:
(i) a protein data bank (PDB) data format, (ii) a JSON data format or (iii) an XML data format.

11. The computing system of claim 1 or claim 9, wherein the machine-learned biomolecule prediction model is an artificial neural network.

12. The computing system of claim 11, wherein the artificial neural network includes at least one of a geometric neural network, a transformer network or a geometric vector perceptron network.

13. The computing system of claim 9, wherein the structural information of the target biomolecule includes data representing one or more of:
(i) an amino acid of the target biomolecule;
(ii) a peptide sequence of the target biomolecule;
(iii) a polypeptide sequence of the target biomolecule;
(iv) a primary sequence of the target biomolecule;
(v) one or more secondary structures of the target biomolecule;
(vi) one or more tertiary structures of the target biomolecule;
(vii) one or more quaternary structures of the target biomolecule; or
(viii) three-dimensional coordinates of a primary sequence of the target biomolecule.

14. The computing system of claim 13, the one or more non-transitory computer-readable media having stored thereon further instructions that, when executed by the one or more processors, cause the computing system to:
output the data representing the one or more of:
(i) the amino acid of the target biomolecule;
(ii) the peptide sequence of the target biomolecule;
(iii) the polypeptide sequence of the target biomolecule;
(iv) the primary sequence of the target biomolecule;
(v) the one or more secondary structures of the target biomolecule;
(vi) the one or more tertiary structures of the target biomolecule;
(vii) the one or more quaternary structures of the target biomolecule; or
(viii) the three-dimensional coordinates of a primary sequence of the target biomolecule.

15. The computing system of claim 14, wherein the data is encoded in (i) a protein data bank (PDB) data format, (ii) a JSON data format, (iii) an XML data format, or (iv) another suitable data format.

16. The computing system of claim 14, wherein the polypeptide sequence of the target biomolecule corresponds to a complementarity determining region of an antibody.

17. The computing system of claim 14, wherein the structural information of the target biomolecule is selected from the group consisting of: an alpha helix, a beta pleated sheet, and a coil.

18. The computing system of claim 9, wherein the target biomolecule is an antibody and the target binding partner is an antigen.

19. The computing system of claim 9, the one or more non-transitory computer-readable media having stored thereon further instructions that, when executed by the one or more processors, cause the computing system to:
repeatedly perform step 2 until the structural information of the target biomolecule is complete.

20. The computing system of claim 19, wherein the repetition is performed iteratively.

21. The computing system of claim 9, the one or more non-transitory computer-readable media having stored thereon further instructions that, when executed by the one or more processors, cause the computing system to:
receive an input parameter specifying a desired length of amino acids of the target biomolecule; and
generate a target biomolecule having the desired length.

22. The computing system of claim 9, the one or more non-transitory computer-readable media having stored thereon further instructions that, when executed by the one or more processors, cause the computing system to:
update the structural information of the target biomolecule by folding one or more proteins.

23. The computing system of claim 9, wherein the target binding partner is at least one of (i) a primary amino acid sequence, (ii) an antigen epitope and/or (iii) a known three-dimensional structure of an antigen.

24. The computing system of claim 9, wherein the structural information of the target biomolecule is predicted in at least one of (i)N-terminus to C-terminus order, (ii) C-terminus to N-terminus order, (iii) via random sampling; or (iv) via non-random sampling.

25. The computing system of claim 9,
the one or more non-transitory computer-readable media having further stored thereon:
a machine-learned docking model trained to generate docked complexes from two or more input three-dimensional biomolecules; and
the one or more non-transitory computer-readable media having stored thereon further instructions that, when executed by the one or more processors, cause the computing system to:
process the target input and the predicted structural information of the target biomolecule using the docking model to generate a docked complex comprising the target input and the target biomolecule.

26. The computing system of claim 25, the one or more non-transitory computer-readable media having stored thereon further instructions that, when executed by the one or more processors, cause the computing system to:
provide the docked complex as a docked complex output.

27. The computing system of claim 25,
the one or more non-transitory computer-readable media having further stored thereon:
a machine-learned binding affinity prediction model trained to predict binding affinity from input docked complexes; and
the one or more non-transitory computer-readable media having stored thereon further instructions that, when executed by the one or more processors, cause the computing system to:
process the docked complex using the trained affinity prediction model to generate a binding affinity value.

28. The computing system of claim 27, wherein the machine-learned model is an artificial neural network.

29. The computing system of claim 28, wherein the artificial neural network is at least one of a graph convolutional neural network, a message passing neural network, or a geometric vector perceptron network.

30. The computing system of claim 28, the one or more non-transitory computer-readable media having stored thereon further instructions that, when executed by the one or more processors, cause the computing system to:
summarize node information contained within the artificial neural network using a pooling operation to generate the binding affinity value.

31. The computing system of claim 28, the one or more non-transitory computer-readable media having stored thereon further instructions that, when executed by the one or more processors, cause the computing system to:
provide the docked complex as a docked complex output.

32. The computing system of claim 9, the one or more non-transitory computer-readable media having stored thereon further instructions that, when executed by the one or more processors, cause the computing system to:
train the machine-learned biomolecule prediction model by providing sequence data as input to teacher force the biomolecule prediction model.

33. The computing system of claim 25, the one or more non-transitory computer-readable media having stored thereon further instructions that, when executed by the one or more processors, cause the computing system to:
train the docking model by providing antibody structure data as input to teacher force the docking model.

34. The computing system of claim 27, the one or more non-transitory computer-readable media having stored thereon further instructions that, when executed by the one or more processors, cause the computing system to:
train the affinity prediction model by providing sequence data as input to teacher force the affinity prediction model.

34a. The computing system of claim 27, the one or more non-transitory computer-readable media having stored thereon further instructions that, when executed by the one or more processors, cause the computing system to:
train the machine-learned biomolecule prediction model using at least one, at least five, at least 10 or at least 100 of the binders in Table E1, Table E2, Table E3 and/or Table E4, above.

35. A computing system for predicting an affinity of a target biomolecule, comprising:
one or more processors; and
one or more non-transitory computer-readable media having stored thereon:
a machine-learned affinity prediction artificial neural network, including:
(i) one or more biomolecule prediction layers trained to predict biomolecule structural information from target inputs;
(ii) one or more docking layers trained to generate docked complexes from two or more input three-dimensional biomolecules; and
(iii) one or more affinity prediction layers trained to predict affinity from input docked complexes;
wherein the one or more biomolecule prediction layers, the one or more docking layers, and the one or more affinity prediction layers are connected; and
instructions that, when executed by the one or more processors, cause the computing system to:
receive a target input comprising one or more of a target binding partner sequence, a target binding partner, or a target epitope; and
process the target input using the affinity prediction artificial neural network to generate a docked complex corresponding to the target input and a corresponding structural affinity value.

36. The computing system of claim 35, the one or more non-transitory computer-readable media having stored thereon further instructions that, when executed by the one or more processors, cause the computing system to:
receive a sequence affinity prediction value from an affinity sequencing prediction model; and
average the structural affinity value with the sequence affinity prediction value to generate an ensemble affinity prediction value.

37. The computing system of claim 35, the one or more non-transitory computer-readable media having stored thereon further instructions that, when executed by the one or more processors, cause the computing system to:
pre-train the one or more biomolecule prediction layers using bound or unbound structures.

38. The computing system of claim 35, the one or more non-transitory computer-readable media having stored thereon further instructions that, when executed by the one or more processors, cause the computing system to:
pre-train the one or more biomolecule prediction layers and the one or more docking layers using bound structures.

39. The computing system of claim 35, the one or more non-transitory computer-readable media having stored thereon further instructions that, when executed by the one or more processors, cause the computing system to:
pre-train the machine-learned affinity prediction artificial neural network using affinity training data.

40. The computing system of claim 35, wherein the affinity training data includes an affinity score proportional to activity.

41. The computing system of claim 35, the one or more non-transitory computer-readable media having stored thereon further instructions that, when executed by the one or more processors, cause the computing system to:
receive an external computationally docked complex corresponding to the target input; and
compare the external computationally docked complex to the generated docked complex.

42. The computing system of claim 35, the one or more non-transitory computer-readable media having stored thereon further instructions that, when executed by the one or more processors, cause the computing system to:
control a gradient flow of the machine-learned affinity prediction artificial neural network by applying a stop gradient function in at least one of the one or more biomolecule prediction layers, the one or more docking layers, or the one or more affinity prediction layers.

43. The computing system of claim 35, the one or more non-transitory computer-readable media having stored thereon further instructions that, when executed by the one or more processors, cause the computing system to:
train the neural network using at least one, at least five, at least 10 or at least 100 of the binders in Table E1, Table E2, Table E3 and/or Table E4, above.
